(19) United States
(12) Patent Application Publication
(10) Pub. No.: US 2023/0090778 A1
(43) Pub. Date: Mar. 23, 2023

(54) LARGE GENE VECTORS AND DELIVERY AND USES THEREOF

(71) Applicant: The Children's Medical Center Corporation, Boston, MA (US)

(72) Inventors: Jeffrey R. HOLT, Boston, MA (US); Olga SHUBINA-OLEINIK, Boston, MA (US)

(73) Assignee: The Children's Medical Center Corporation, Boston, MA (US)

(21) Appl. No.: 17/798,009

(22) PCT Filed: Feb. 5, 2021
(86) PCT No.: PCT/US2021/016720

§ 371 Date: Aug. 5, 2022

Related U.S. Application Data
(60) Provisional application No. 62/971,555, filed on Feb. 7, 2020.

(51) Int. Cl. C12N 15/86; C07K 14/47; C12N 15/62; A61P 27/00

(52) U.S. Cl. CPC C12N 15/86; C07K 14/47; C12N 15/625; A61P 27/00; C12N 2750/14143; C12N 2800/40

(57) ABSTRACT

The disclosure provides a dual-vector intein-mediated protein trans-splicing system, cells, compositions, and methods of using the same for gene therapy. In some embodiments, the disclosure provides methods and compositions for treating an autosomal recessive type of non-syndromic deafness, DFNB16, by delivering a STRC gene, encoding a STRC protein, using the dual-vector system described herein.

### CROSS-REFERENCE TO RELATED APPLICATIONS

This PCT International application claims the benefit of and priority to U.S. Provisional Application No. 62/971,555, filed Feb. 7, 2020, which is hereby incorporated by reference in its entirety for all purposes.

### BACKGROUND

Hearing loss is one of the most common neurological disorders in developed or industrialized countries and the most prevalent sensorineural disorder accounting for over 466 million cases worldwide. Non-syndromic deafness or non-syndromic genetic deafness is hearing loss that is not associated with any other signs or symptoms. There are four types of non-syndromic deafness: DFNA (autosomal dominant), DFNB (autosomal recessive), DFNX (X-linked), and mitochondrial non-syndromic deafness. The sixteenth described autosomal recessive type of non-syndromic deafness, DFNB16, is a monogenic, non-syndromic, recessive hearing loss caused by mutations in the STRC gene, which encodes an extracellular structural protein known as stereocilin. Normal expression of STRC in the inner ear is essential for auditory function. Stereocilin, which is found at the top of modified microvilli at the apex of sensory hair cells in the inner ear, is associated with hair-like structures known as stereocilia, which project from specialized cells in the inner ear. Mutations in STRC cause moderate to severe hearing loss and affect an estimated ˜50,000 patients in the U.S. and is thus an attractive candidate for gene therapy. Stereocilin functions to maintain a cohesive bundle of microvilli and to couple the bundle to the overlying tectorial membrane, which is in the cochlea of the inner ear. Worldwide statistics suggest that DFNB16 constitutes a significant proportion of genetic deafness, especially in those with moderate hearing impairment. Based on data from the Partners Laboratory of Molecular Medicine, 19% of genetic hearing loss patients tested in Boston have mutations in STRC, as such, it is the second most common form of genetic hearing loss and the most common form that affects sensory hair cells of the inner ear. About forty different mutations (primarily recessive) have been identified in the STRC gene, the majority lead to synthesis of defective stereocilin or completely prevent its synthesis. Lack of normal STRC protein decouples sensory hair bundles from the overlying tectorial membrane which is required for proper sound evoked stimulation. DFNB16 patients have moderate to severe hearing loss and are typically treated with hearing aids or cochlear implants. However, there are currently no biological treatments for DFNB16 hearing loss.

AAV provides an attractive vector system for gene therapy treatments of inherited disorders. These and gene delivery in view of its safety. Recombinant AAV (rAAV) is derived from non-pathogenic and replication-defective viruses, it is non-cytotoxic to its host cells. Moreover, rAAVs lack all viral DNA sequences except the inverted terminal repeats (ITRs), presenting another safety feature. The ITRs are necessary for AAV DNA replication, packaging, chromosomal integration, and pro-virus rescue. AAV vectors have also been demonstrated to be powerful tools for effective transgene delivery and durable expression in, for example, inner ear cells. However, many proteins critical for inner ear function have coding sequences that exceed the cargo capacity of AAV vectors (˜4.5 kB), including that of STRC (˜5.8 kB). Accordingly, there is a need for methods of delivery and expression of proteins encoded by large genes (e.g., larger than 4 kB) as an effective form of gene therapy, constructs, and vectors of any of the aforementioned.

### SUMMARY

It is therefore an object of this disclosure to provide gene therapy for large genes (e.g., larger than 4 kB) for treating subjects suffering from a genetic mutation. The gene therapy may allow for the prevention and/or restoration of hearing in children and adults having, for example, DFNB16 hearing loss.

It is another object to provide a method for delivering a large gene sequence (e.g., larger than 4 kB; STRC), and vectors and constructs for delivering the large gene sequences, where the method overcomes vector size limitations.

One aspect provides a vector system (e.g., dual-vector system) for expressing a protein of interest in a cell, the dual-vector system comprising:
a) a first vector comprising a first nucleotide sequence (e.g., SEQ ID NO:5; SEQ ID NO:7) comprising, in a 5′ to 3′ direction:
   a signal sequence (e.g., SEQ ID NO:9; SEQ ID NO:11) at the 5′-end of a partial coding sequence encoding an amino terminal (N-terminal) portion of the protein of interest;
   the partial coding sequence encoding the N-terminal portion of the protein of interest (e.g., N-STRC; SEQ ID NO:15; SEQ ID NO:16);
   a sequence encoding a splice donor sequence (e.g., an N-terminal fragment of intein (N-intein), also known as a split intein-N); SEQ ID NO:13 encoding SEQ ID NO:14) adjacent to and downstream of the partial coding sequence; and
b) a second vector comprising a second nucleotide sequence (e.g., SEQ ID NO:17; SEQ ID NO:19) comprising, in a 5′ to 3′ direction:
   a signal sequence (e.g., SEQ ID NO: 9; SEQ ID NO:11) at the 5′-end of a partial coding sequence encoding a carboxy terminal (C-terminal) portion of the protein of interest;
   a sequence encoding splice acceptor sequence (e.g., a C-terminal fragment of intein (C-intein), also known as a split intein-C); SEQ ID NO:21 encoding SEQ ID NO:22), wherein the splice acceptor sequence is flanked by the signal sequence and the partial coding sequence encoding the C-terminal portion of the protein of interest;
   the partial coding sequence encoding the C-terminal portion of the protein of interest (e.g., C-STRC; SEQ ID NO:23; SEQ ID NO:24).

Another aspect provides a dual-vector system for expressing a protein of interest in a cell, the dual-vector system comprising:
a) a first vector comprising a first nucleotide sequence comprising, in a 5′ to 3′ direction:
   a 5′-inverted terminal repeat (5′-ITR) sequence;
   a promoter sequence;
   a signal sequence, wherein the signal sequence is operably linked to and under control of the promoter;
   a partial coding sequence encoding an amino terminal (N-terminal) portion of the protein of interest (e.g., N-STRC), wherein the partial coding sequence is operably linked to and under control of the promoter;
   a sequence encoding an amino terminal fragment of intein (N-intein), wherein the sequence encoding N-intein is operably linked to and under control of the promoter;
   a poly-adenylation (polyA) signal sequence;
   a 3′-inverted terminal repeat (3′-ITR) sequence; and
b) a second vector comprising a second nucleotide sequence comprising, in a 5′ to 3′ direction:
   a 5′-inverted terminal repeat (5′-ITR) sequence;
   a promoter sequence;
   a signal sequence, wherein the signal sequence is operably linked to and under control of the promoter;
   a sequence encoding a carboxy terminal fragment of intein (C-intein), wherein the sequence encoding C-intein is operably linked to and under control of the promoter;
   a partial coding sequence encoding a carboxy terminal (C-terminal) portion of the protein of interest (e.g., C-STRC), wherein the partial coding sequence is operably linked to and under control of the promoter;
   a poly-adenylation (polyA) signal sequence;
   a 3′-inverted terminal repeat (3′-ITR) sequence.

Another aspect of the dual-vector system provides the first vector and the second vector in the cell, express respectively:
a) a first protein sequence comprising in an N-terminal to C-terminal direction:
   a signal peptide sequence linked to an N-terminal portion of the protein of interest (e.g., STRC) sequence fused at its C-terminal end to an N-intein protein sequence; and
b) a second protein sequence comprising in an N-terminal to C-terminal direction:
   a signal peptide sequence linked to a C-intein protein sequence fused to the N-terminal end of a C-terminal portion of the protein of interest (e.g., STRC) sequence.

Yet another aspect provides the N-terminal portion of the protein of interest (e.g., N-STRC) and the C-terminal portion of the protein of interest (e.g., C-STRC) form a full-length protein of interest (e.g., STRC). In some aspects, the signal peptide sequence of the first protein sequence and the signal peptide sequence of the second protein sequence are the same or different or the signal peptide sequence of the first protein sequence and the signal peptide sequence of the second protein sequence are configured to transport the first protein sequence and the second protein sequence to the same cellular compartment. A further aspect may be directed to a signal sequence comprising a nucleic acid sequence at least 80% identity to SEQ ID NO:9 or SEQ ID NO:11, and encoding a signal peptide sequence having an amino acid sequence of at least 80% identity to SEQ ID NO:10 or SEQ ID NO:12. Other aspects provide a vector (e.g., a first vector and a second vector) that may be a viral vector, where the viral vector may be an adeno-associated virus (AAV) vector or a lentivirus. One aspect may be directed to viral vectors having the same or different serotypes. Another aspect of the dual-vector system provides intein-mediated trans-splicing of the protein of interest, where an N-terminal portion of the protein of interest (e.g., N-STRC) and a C-terminal portion of the protein of interest (e.g., C-STRC) may form the full-length protein of interest (e.g., STRC) through a peptide bond, where the protein of interest may be the STRC protein, which is encoded by the STRC gene. A nucleotide sequence encoding an N-terminal portion of the protein of interest (e.g., N-STRC; human SEQ ID NO:15 or murine SEQ ID NO:16) comprises a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the nucleotide sequence of interest (e.g., STRC; SEQ ID NO:5 or SEQ ID NO:7), which encodes an amino acid sequence of interest (e.g., STRC; SEQ ID NO:6; SEQ ID NO:8; SEQ ID NO:15 or SEQ ID NO:16 of at least 5% (e.g., 6%, 7%, 8%, 9%, 10%, 20%, 30%, 40%, 50%, 60%, 70%, 80%, 85%, 90%, 95%, 97%, 99%, 100%) or less than 54% of (e.g., 53%, 52%, 51%, 50%, 45%, 43%, 41%) and/or less than 54% identity to and/or less than 54% in length of the N-terminal portion of a full-length protein of interest (e.g., STRC; SEQ ID NO:25 or SEQ ID NO:26). Another aspect provides for an N-terminal portion of the protein of interest (e.g., STRC) comprising an amino acid sequence of 41% or greater (e.g., 42%, 43%, 44%, 45%, 50%, 51%, 52%, 53%) of and/or 41% or greater identity to and/or 41% or greater in length of the N-terminal portion of a full-length protein of interest (e.g., SEQ ID NO:25 or SEQ ID NO:26).

A further aspect provides a nucleotide sequence comprising a signal sequence having a nucleic acid sequence of at least 80% (e.g., 85%, 90%, 95%, 97%, 99%, 100%) identity to a desired signal sequence (e.g., SEQ ID NO:9; SEQ ID NO:11), which encodes a signal peptide sequence having an amino acid sequence of at least 80% (e.g., 85%, 90%, 95%, 97%, 99%, 100%) identity to the desired signal peptide sequence (e.g., SEQ ID NO:10; SEQ ID NO:12).

Yet another aspect may provide a desired N-intein sequence comprising a nucleic acid sequence of at least 80% (e.g., 85%, 90%, 95%, 97%, 99%, 100%) identity to the desired N-intein nucleotide sequence (e.g., SEQ ID NO:13), which encodes a desired N-intein amino acid sequence of at least 80% (e.g., 85%, 90%, 95%, 97%, 99%, 100%) identity to the desired N-intein amino acid sequence (e.g., SEQ ID NO:14). A further aspect may be directed to a desired C-intein sequence comprises a nucleic acid sequence of at least 80% (e.g., 85%, 90%, 95%, 97%, 99%, 100%) identity to the desired C-intein nucleotide sequence (e.g., SEQ ID NO:21), which encodes a desired C-intein amino acid sequence of at least 80% (e.g., 85%, 90%, 95%, 97%, 99%, 100%) identity to the desired C-intein amino acid sequence (e.g., SEQ ID NO:22).

In yet another aspect of the dual-vector system of the disclosure, the C-terminal portion of the protein of interest (e.g., STRC) may comprise a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the nucleotide sequence (e.g., STRC; SEQ ID NO: 17; SEQ ID NO:19), which encodes an amino acid sequence of interest (e.g., STRC; SEQ ID NO:18; SEQ ID NO:20; SEQ ID NO:23; SEQ ID NO:24) of at least 5% (e.g., 6%, 7%, 8%, 9%, 10%, 20%, 30%, 40%, 50%, 60%, 70%, 80%, 85%, 90%, 95%, 97%, 99%, 100%) or 46% or greater of and/or 46% or greater identity to and/or 46% or greater in length of the C-terminal portion of a full-length protein of interest (e.g., STRC; SEQ ID NO:25; SEQ ID NO:26). Another aspect may provide for a C-terminal portion of the protein of interest (e.g., STRC) comprising an amino acid sequence of 60% or less identity to and/or 60% or less in length of the C-terminal portion of a full-length protein of interest (e.g., STRC; SEQ ID NO:25; SEQ ID NO:26).

A further aspect may provide a vector system for expressing a coding sequence of a STRC gene in a host cell, wherein the coding sequence comprises at least one vector comprising the STRC nucleotide coding sequence of, for example, human STRC: SEQ ID NO:1 or SEQ ID NO:30 or murine STRC: SEQ ID NO:3 or SEQ ID NO:32, wherein the STRC nucleotide coding sequence encodes the STRC protein of, for example, SEQ ID NO:2 or SEQ ID NO:25 or SEQ ID NO:4 or SEQ ID NO:26. Another aspect may be directed to a nucleotide sequence encoding a desired full-length protein, where the nucleotide sequence comprises, e.g., human STRC: SEQ ID NO:1 or SEQ ID NO:33, or murine STRC: SEQ ID NO:3 or SEQ ID NO:39, which encodes a desired protein, e.g., human STRC: SEQ ID NO:2 or SEQ ID NO:25 or murine STRC: SEQ ID NO:4 or SEQ ID NO: 26.

One aspect of the vector system comprising a dual-vector system for expressing a coding sequence of the STRC gene in a host cell as described herein, where the dual-vector system provides for a first vector comprising a first nucleotide sequence comprising the desired nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the desired nucleic acid sequence of interest (e.g., SEQ ID NO:5; SEQ ID NO:7). In one aspect, the first vector (e.g., plasmid, transplicing plasmid, viral vector, Adenovirus, AAV, AAV genome) comprises a first nucleotide sequence (e.g., SEQ ID NO:5 encoding SEQ ID NO:6; SEQ ID NO:7 encoding SEQ ID NO:8) comprising, in a 5′ to 3′ direction: a signal sequence (e.g., SEQ ID NO:9 encoding SEQ ID NO: 10; or SEQ ID NO:11 encoding SEQ ID NO: 12) at the 5′-end of the partial coding sequence, where the partial coding sequence may be flanked by or adjacent to a downstream sequence encoding a splice donor sequence (e.g., an N-terminal intein (N-intein, also known as a split intein-N); SEQ ID NO:13 encoding SEQ ID NO: 14); a partial coding sequence encoding an amino terminal (N-terminal) portion of the protein of interest (e.g., STRC; SEQ ID NO:15; SEQ ID NO:16). Another aspect comprises a first nucleotide sequence comprising an N-terminal portion of a protein of interest and also contains a signal sequence and a sequence encoding the desired N-intein protein, as well as inverted terminal repeat (ITR), promoter, and poly-adenylation (polyA) sequences. The first nucleotide sequence may encode an amino acid sequence of interest of at least 5% (e.g., 6%, 7%, 8%, 9%, 10%, 20%, 30%, 40%, 50%, 60%, 70%, 80%, 85%, 90%, 95%, 97%, 99%, 100%) identity to the desired amino acid sequence (e.g., SEQ ID NO:5; SEQ ID NO:16) or to the full-length amino acid sequence of interest (e.g., SEQ ID NO: 25; SEQ ID NO:26).

Another aspect of the dual-vector system of the disclosure also provides a second nucleotide sequence comprises the remaining portion of the desired nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the desired nucleic acid sequence of interest (e.g., SEQ ID NO:17; SEQ ID NO:19). In one aspect, the second vector e.g., plasmid, transplicing plasmid, viral vector, Adenovirus, AAV, AAV genome) comprises a second nucleotide sequence (e.g., SEQ ID NO:17 encoding SEQ ID NO:18; SEQ ID NO:19 encoding SEQ ID NO:20) comprising, in a 5′ to 3′ direction: a signal sequence (e.g., SEQ ID NO:9 encoding SEQ ID NO:10; or SEQ ID NO:11 encoding SEQ ID NO:12) that may be upstream of a splice acceptor sequence (e.g., a C-terminal intein (C-intein); SEQ ID NO:21 encoding SEQ ID NO:22) positioned immediately adjacent to or flanking a downstream partial coding sequence encoding the remaining portion of the full-length coding sequence of the protein of interest, i.e., the C-terminal portion of the protein of interest (e.g., STRC; SEQ ID NO:23; SEQ ID NO:24). Another aspect comprises a second nucleotide sequence comprising a C-terminal portion of a protein of interest, a sequence encoding a signal sequence, and a sequence encoding the desired C-intein protein, as well as inverted terminal repeat (ITR), promoter, and poly-adenylation (polyA) sequences. In some aspects, the second nucleotide sequence may also contain a linker sequence and myc tag sequence. The second nucleotide sequence may encode an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the desired amino acid sequence (e.g., SEQ ID NO:18; SEQ ID NO:20; SEQ ID NO:23; SEQ ID NO:24) or to the full-length amino acid sequence of interest (e.g., SEQ ID NO: 25; SEQ ID NO:26).

Other aspects may provide a cell(s) or a host cell(s) containing the vector system (e.g., dual-vector system) described herein for delivering the desired gene or its desired protein (e.g., STRC protein).

A further aspect may be directed to a pharmaceutical composition comprising the vector system (e.g., dual-vector system) of the disclosure for delivering the desired gene or its desired protein (e.g., STRC protein), and a pharmaceutically acceptable vehicle (e.g., diluent, excipient).

In another aspect, a method for treating a disease or condition in a subject suffering from a genetic mutation of the disease or condition, comprising administering to the subject in need thereof, an effective amount of the vector system (e.g., dual-vector system) of the disclosure, where the method delivers a desired wild-type or corrected gene or a desired wild-type or corrected protein (e.g., STRC) to the subject suffering from the disease or condition caused by a genetic mutation in the same gene, thereby treating the disease or condition in the subject. Yet another aspect provides a method for treating a disease or condition in a subject suffering from an autosomal recessive hearing loss, comprising administering to the subject in need thereof, an effective amount of the dual-vector system described herein that delivers a desired wild-type or corrected gene or a desired wild-type or corrected protein (e.g., STRC). The method of treating an autosomal recessive hearing loss in a subject, comprising administering to the subject in need thereof, a cell(s) or a host cell(s) or a pharmaceutical composition (with a pharmaceutically acceptable vehicle (e.g., diluent, excipient)) containing the dual-vector system described herein for delivering the desired gene or its desired protein (e.g., STRC protein). Another aspect may provide for an autosomal recessive hearing loss, DFNB16.

In one aspect, a method of the disclosure may comprise: contacting a cell of a subject with the composition comprising the vector system (e.g., dual-vector system) of the disclosure for delivering the desired gene or its desired protein (e.g., STRC protein), and a pharmaceutically acceptable vehicle (e.g., diluent, excipient), where the contacting results in the delivery of the first nucleotide sequence and the second nucleotide sequence into the cell, where the cell may express an N-terminal portion of the desired protein and a C-terminal portion of the desired protein joined by a peptide bond to form the full-length desired protein. Another aspect provides for a method for treating and/or preventing a pathology or disease characterized by a hearing loss comprising administering to a subject in need thereof an effective amount of the dual-vector system described herein, or the cell or the pharmaceutical composition (with a pharmaceutically acceptable vehicle (e.g., diluent, excipient)) containing the dual-vector system described herein for delivering the desired gene or its desired protein (e.g., STRC protein). In some aspects, the cell may be an inner ear cell, an inner hair cell or an outer hair cell of the ear, where the cell or method of administering to the cell may occur in vivo, ex vivo, and/or in vitro. A further aspect of the disclosure where any of the methods described herein, results in improvement or restoration of auditory function in the subject.

Other features and advantages of the disclosure will be apparent from the detailed description and from the claims.

### BRIEF DESCRIPTION OF FIGURES

FIG. 1 shows a schematic representation of a construct for a single vector system for expression of a desired full-length protein (e.g., STRC), where the construct has AAV2 Inverted Terminal Repeats (ITRs), promoters, and poly-adenylation (polyA) sequences.

FIGS. 2A-2C show the nucleotide sequence (SEQ ID NO:33) encoding the human STRC protein, the signal peptide sequence, linker sequence, sequence encoding a Myc tag, and Start and Stop codons.

FIG. 3 shows the amino acid sequence (SEQ ID NO:36) containing the signal peptide sequence, human STRC protein sequence, linker sequence, and myc tag encoded by the nucleotide sequence presented in FIGS. 2A-2C.

FIGS. 4A-4D show the nucleotide sequence (SEQ ID NO:38) encoding the murine STRC protein, the signal peptide sequence, linker sequence, sequence encoding a Myc tag, and Start and Stop codons.

FIG. 5 shows the amino acid sequence (SEQ ID NO:39) containing the signal peptide sequence, murine STRC protein sequence, linker sequence, and Myc tag encoded by the nucleotide sequence presented in FIGS. 4A and 4D.

FIG. 6 shows a schematic representation of dual AAV intein-mediated stereocilin protein trans-splicing using AAV2 Inverted Terminal Repeats, promoters, and poly-adenylation sequences. The intein fragments mediate protein recombination, excising themselves, and joining the remaining STRC fragments (exteins) with a peptide bond.

FIGS. 7A and 7B show the nucleotide sequence (SEQ ID NO:5) encoding the N-terminal portion of a human STRC protein, the signal peptide sequence, and splice donor sequence (e.g., N-intein; CFS-N-Strc-N-Int, Construct 2, N-portion). The nucleotide sequence contains a signal sequence, a 5′ Strc (5′ fragment of the wild-type Strc coding sequence), and an N-intein sequence (encoding N-terminal fragment of the intein protein).

FIGS. 8A and 8B show the nucleotide sequence (SEQ ID NO:7) encoding the N-terminal portion of a murine STRC protein, the signal peptide sequence, and N-intein (e.g., CFS-N-Strc-N-Int, Construct 2, N-portion). The nucleotide sequence contains a signal sequence, a 5′ Strc (5′ fragment of the wild-type Strc coding sequence), and an N-intein sequence (encoding N-terminal fragment of the intein protein).

FIG. 9 shows a schematic representation of the construct containing a nucleotide sequence of FIGS. 8A and 8B encoding the signal peptide sequence, N-terminal portion of STRC protein, and N-intein used in dual-AAV intein-mediated protein trans-splicing described herein.

FIG. 10 shows the amino acid sequence (SEQ ID NO: 6) containing the N-terminal portion of the human STRC protein, signal peptide sequence, and N-intein encoded by the nucleotide sequence presented in FIGS. 7A and 7B.

FIG. 11 shows the amino acid sequence (SEQ ID NO:8) containing the N-terminal portion of the murine STRC protein, signal peptide sequence, and N-intein encoded by the nucleotide sequence presented in FIGS. 8A and 8B.

FIGS. 12A and 12B show the nucleotide sequence (SEQ ID NO:17) encoding the C-terminal portion of a human STRC protein, the signal peptide sequence, and C-intein (e.g., CFS-C-Strc-C-Int, Construct 2, C-portion). The nucleotide sequence contains a signal sequence, a C-intein sequence (encoding C-terminal fragment of the intein protein), a 3′ Strc (3′ fragment of the wild-type Strc coding sequence), a linker sequence, and a myc tag sequence.

FIGS. 13A and 13B show the nucleotide sequence (SEQ ID NO:19) encoding the C-terminal portion of a murine STRC protein, the signal peptide sequence, and C-intein (e.g., CFS-C-Strc-C-Int, Construct 2, C-portion). The nucleotide sequence contains a signal sequence, a C-intein sequence (encoding C-terminal fragment of the intein protein), a 3′ Strc (3′ fragment of the wild-type Strc coding sequence), a linker sequence, and a myc tag sequence.

FIG. 14 shows a schematic representation of the construct containing a nucleotide sequence of FIGS. 13A and 13B encoding the signal peptide sequence, C-intein, and C-terminal portion of STRC protein used in dual-AAV intein-mediated protein trans-splicing described herein.

FIG. 15 shows the amino acid sequence (SEQ ID NO:18) containing the signal peptide sequence, C-intein, C-terminal portion of the human STRC protein, linker sequence, and myc tag encoded by the nucleotide sequence presented in FIGS. 12A and 12B.

FIG. 16 shows the amino acid sequence (SEQ ID NO:20) containing the signal peptide sequence, C-intein, C-terminal portion of the murine STRC protein, linker sequence, and myc tag encoded by the nucleotide sequence presented in FIGS. 13A and 13B.

FIG. 17 shows a predicted structure of stereocilin (STRC) protein with the specified CFS split site containing cysteine (C; Cys), phenylalanine (F, Phe), and serine (S; Ser) of FIGS. 11 and 16 produced by the sequences of FIGS. 8A, 8B, 13A, and 13B and as depicted by constructs of FIGS. 9 and 14.

FIGS. 18A and 18B show the nucleotide sequence (SEQ ID NO:51) encoding the N-terminal portion of STRC protein, the signal peptide sequence, and N-intein (e.g., CFS-N-Strc-N-Int, Construct 1, N-portion). The nucleotide sequence contains a signal sequence, a 5′ Strc (5′ fragment of the wild-type Strc coding sequence), and an N-intein sequence (encoding N-terminal fragment of the intein protein).

FIG. 19 shows a schematic representation of the construct containing a nucleotide sequence of FIGS. 18A and 18B encoding the signal peptide sequence, N-terminal portion of STRC protein, and N-intein used in dual-AAV intein-mediated protein trans-splicing described herein.

FIG. 20 shows the amino acid sequence (SEQ ID NO:52) containing the N-terminal portion of the STRC protein, signal peptide sequence, and N-intein encoded by the nucleotide sequence presented in FIGS. 18A and 18B.

FIGS. 21A and 21B show the nucleotide sequence (SEQ ID NO:53) encoding the C-terminal portion of STRC protein, the signal peptide sequence, and C-intein (e.g., CFS-C-Strc-C-Int, Construct 1, C-portion). The nucleotide sequence contains a signal sequence, a C-intein sequence (encoding C-terminal fragment of the intein protein), a 3′ Strc (3′ fragment of the wild-type Strc coding sequence), a linker sequence, and a myc tag sequence.

FIG. 22 shows a schematic representation of the construct containing a nucleotide sequence FIGS. 21A and 21B encoding the signal peptide sequence, C-intein, and C-terminal portion of STRC protein used in dual-AAV intein-mediated protein trans-splicing described herein.

FIG. 23 shows the amino acid sequence (SEQ ID NO:54) containing the signal peptide sequence, C-intein, C-terminal portion of the STRC protein, linker sequence, and myc tag encoded by the nucleotide sequence presented in FIGS. 21A and 21B.

FIG. 24 confirms dual-AAV intein-mediated protein trans-splicing and processing described herein as demonstrated by a Western blot of the isolated STRC protein in Lane 4 using the sequences of FIGS. 8A, 8B, 13A, and 13B and as depicted by constructs of FIGS. 9 and 14 (AAV2/AAV9-Php.B-STRC-Construct 2).

FIG. 25 confirms the usefulness of signal sequences in the dual-AAV intein-mediated protein trans-splicing and processing described herein as demonstrated by a Western blot of the isolated STRC protein in Lane 6 using the sequences of FIGS. 8A, 8B, 13A, and 13B and as depicted by constructs of FIGS. 9 and 14 (AAV2/AAV9-Php.B-STRC-Construct 2) with a signal sequence as opposed to Lane 4 which lacked signal sequences.

FIG. 26 shows recovery of hearing loss using the dual-AAV intein-mediated protein trans-splicing and processing described herein as demonstrated by the recovery of sound pressure levels (decibels, dB) in STRC knockout mice (Strc^{-/-}) infected with the constructs of FIGS. 9 and 14 compared to wild-type (WT) mice (Strc^{WT/WT}) and STRC knockout mice (Strc^{-/-}).

FIG. 27 shows ABR and DPOAE results demonstrating recovery of auditory function with treatment with the dual-AAV intein-mediated protein trans-splicing system described herein (Construct 2: AAV2/AAV9-PHP.B-CMV-Strc-N; AAV2/AAV9-PHP.B-CMV-Strc-C) in Strc knockout mice.

FIG. 28 shows ABR and DPOAE results demonstrating a lack of auditory function recovery in Strc knockout mice using only the construct encoding the N-terminal portion of STRC protein depicted in FIG. 9.

FIG. 29 shows ABR and DPOAE results demonstrating a lack of auditory function recovery in Strc knockout mice using only the construct encoding the C-terminal portion of STRC protein depicted in FIG. 14.

FIG. 30 shows ABR and DPOAE results over time in vivo after treatment of Strc knockout mice with the dual-AAV intein-mediated protein trans-splicing system described herein (Construct 2: AAV2/AAV9-PHP.B-CMV-Strc-N; AAV2/AAV9-PHP.B-CMV-Strc-C).

FIGS. 31A-31C provide the dual vector strategy using intein-mediated protein recombination. FIG. 31A provides eight AAV2 plasmids that were generated and included four different dual vector variants. N-terminal and C-terminal inteins were fused in-frame at the indicated sites for each of the four variants. Variants 1 and 2 differ in their split sites, where native cysteines are located at position 747 (variant 1) and position 970 (variant 2). Variants 3 and 4 had identical split sites 1 and 2, respectively. In addition, variants 3 and 4 had the signal sequence found at the N-terminus of STRC fused to the N-terminus of the C-terminal fragments, upstream of the C-intein sequence. Intein-mediated protein recombination was predicted to yield the full length STRC and an excised intein fragment. A Myc tag (not shown) was fused to the C-terminus of all C-terminal fragments. FIG. 31B shows the split sites and surrounding amino acid sequences for the four variants. FIG. 31C provides a representative Western Blot analysis of lysates from human embryonic kidney (HEK) 293 cells transfected with: a plasmid encoding full-length STRC (Lane 1), non-transfected control (Lane 2), plasmids encoding the C-terminal fragments of variant 1 (Lane 3) and variant 3 (Lane 5) and co-transfection of both N- and C-fragments for variant 1 (Lane 4) and variant 3 (Lane 6). An anti-Myc antibody was used to identify C-terminal fragments (120 kD) and full-length STRC (220 kD).

FIGS. 32A-32G show the generation and characterization of Strc^{Δ/Δ} mice. FIG. 32A illustrates the CRISPR/Cas9 strategy for disruption of WT Strc. Three guide RNAs (sgRNA) were designed to target exon 4. The gene disruption strategy yielded a deletion of 249 nucleotides and two transpositions and inversions (947-1139—purple and 1758-1835—yellow), which introduced a premature stop codon to in the mutant Strc allele. FIG. 32B provides the results of PCR used to amplify genomic DNA, which when run on a gel yielded clear bands for WT (1 kB) and mutant Strc (751 bp) alleles. FIGS. 32C-32D illustrate confocal images of WT and Strc^{Δ/Δ} cochleas taken from tissue stained with an anti-STRC antibody, an Alexa488 secondary and phalloidin-Alexa555 to illuminate hair bundles. Inner hair cells (IHCs) and three rows of outer hair cells (OHCs) are visible. Green STRC stain presents on the OHCs of the WT, while red Actin stain presents on the IHCs of both WT and Strc^{Δ/Δ}. Scale bar=10 mm. FIG. 32E shows mean±S.D. sensory transduction current amplitudes measured from IHCs and OHCs of Strc^{Δ/+} (Het-black circles) and Strc^{Δ/Δ} (Homo-red diamonds) mice. FIG. 32F shows mean±S.D. DPOAE thresholds obtained from Strc^{D/D} mice (n=6; red) and WT mice (n=5; black). FIG. 32G illustrates mean±S.D. ABR thresholds obtained from Strc^{Δ/Δ} mice (n=6; red) and WT mice (n=5; black).

FIGS. 33A-B demonstrates that dual AAV delivery restores STRC expression and hair bundle morphology. FIG. 33A provides confocal images of WT (left), Strc^{Δ/Δ} middle), and dual vector injected Strc^{Δ/Δ} (right) cochleas stained with an anti-STRC antibody with Alexa488 conjugated secondary (green) and Alexa546-phalloidin (red). Scale bar=10 mm. The upper row of images shows the two channels merged. The lower row shows STRC localization alone. FIG. 33B shows scanning electron microscopy images of WT (top), Strc^{Δ/Δ} (middle), and dual vector injected Strc^{Δ/Δ} (bottom) outer hair cell bundles. Tissues were harvested, fixed, and imaged at 4 or 12 weeks as indicated above. Scale bar=5 mm (left & middle) or 2 mm (right).

FIGS. 34A-34D shows that dual AAV delivery restores DPOAE and ABR thresholds. FIG. 34A provides a Fourier analysis of DPOAE waveforms revealed two frequency components at the stimulus frequencies f1 (13.3 kHz) and f2 (16 kHz) and a distortion product at the predicted frequency 2f1-f2 (10.6 kHz) in a WT mouse cochlea (upper trace). Traces below show the distortion product for sound pressure levels from 10 to 50 dB on an expanded frequency and amplitude scale for WT (left), Strc^{Δ/Δ} (middle), and dual vector injected Strc^{Δ/Δ} (right) cochleas. The bold traces indicate the DPOAE threshold. FIG. 34B provides DPOAE thresholds as function of (f2) stimulus frequency for WT (black) and dual vector injected Strc^{Δ/Δ} mice with (purple) and without recovery (Strc^{Δ/Δ}; red; n=20; top horizontal line). Red and purple lines represent data from individuals. Mean±S.E. are shown for WT (black; n=5; lowest line), 20 dual vector injected Strc^{Δ/Δ} mice with recovery (purple; n=20; 2^{nd} line from top), and the five best recoveries (green; n=5; 3^{rd} line from top). FIG. 34C illustrates families of ABR traces recorded from WT (left), Strc^{Δ/Δ} (middle), and dual vector injected Strc^{Δ/Δ} (right) cochleas, evoked by sound pressure levels between 25 and 110 dB. Bold traces indicate ABR thresholds. FIG. 34D provides ABR thresholds plotted as a function of stimulus frequency for WT (black; n=5; lowest line) and dual vector injected Strc^{Δ/Δ} mice with (purple; n=20; 2^{nd} from top), and without recovery (Strc^{Δ/Δ}; red; n=20; top line). Purple lines represent data from individuals. Mean±S.E. are shown for WT (black; n=5; lowest line), dual vector injected Strc^{Δ/Δ} mice with (purple; n=20; 2^{nd} line from top) and without (Strc^{Δ/Δ}; red; n=20; top line) recovery and the mice with the best recoveries (green; n=5; 3^{rd} line from top).

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

Compositions and methods of restoring hearing through expression of Stereocilin (STRC) are provided here. Treatment with the gene of interest using two separate AAV particles or vectors, where one comprises a signal sequence, a 5′ end fragment of the gene coding sequence, and a sequence encoding an amino terminal fragment of intein (N-intein, also known as a split intein-N) and one comprises a signal sequence, a sequence encoding a carboxy terminal fragment of intein (C-intein, also known as a split intein-C), and a 3′ end fragment of the gene coding sequence.

Unless defined otherwise, all technical and scientific terms used here have the meaning commonly understood by a person skilled in the art to which this invention belongs. The following references provide one of ordinary skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

### Definitions

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

“Adeno-associated virus” (AAV) is a small virus that may infect humans and some other primate species. Vectors using AAV may infect dividing and quiescent cells without integrating into the genome of the host cell. These features make AAV an attractive candidate for gene therapy viral vectors.

By “AAV9-php.b vector” is meant a viral vector, an adeno-associated virus serotype 9, comprising an AAV9-php.b polynucleotide or fragment thereof that may transfect a cell, for example, a cell of the inner ear. In one embodiment, the AAV9-php.b vector transfects at least 70% or greater (e.g., 75%, 80%, 85%, 90%, 95%, 97%, 99%, 100%) of cells. Another embodiment may be directed to an AAV9-php.b vector comprising an AAV9-php.b polynucleotide or fragment thereof that may transfect at least 70% or greater (e.g., 75%, 80%, 85%, 90%, 95%, 97%, 99%, 100%) of inner hair cells and/or outer hair cells following administration of the AAV9-php.b vector to the inner ear of a subject or contact of the AAV9-php.b vector with a cell derived from an inner ear in vitro. In other embodiments, at least 85% (e.g., 90%, 95%, 100%) of inner hair cells and/or at least 85% (e.g., 90%, 95%, 100%) of outer hair cells are transfected with the AAV9-php.b vector. The transfection efficiency may be assessed using a label or tag (e.g., a gene encoding green fluorescent protein (GFP)) in a mouse model.

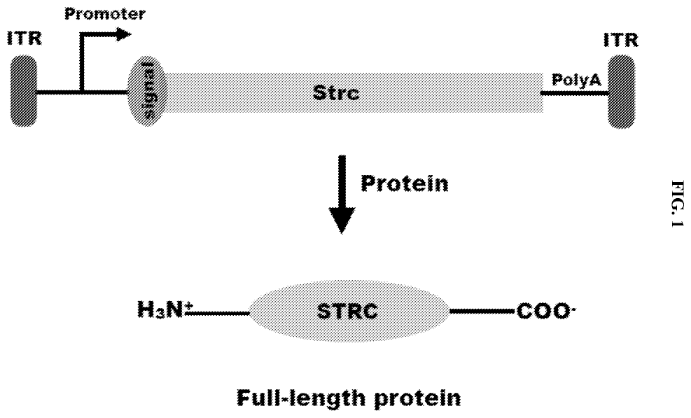

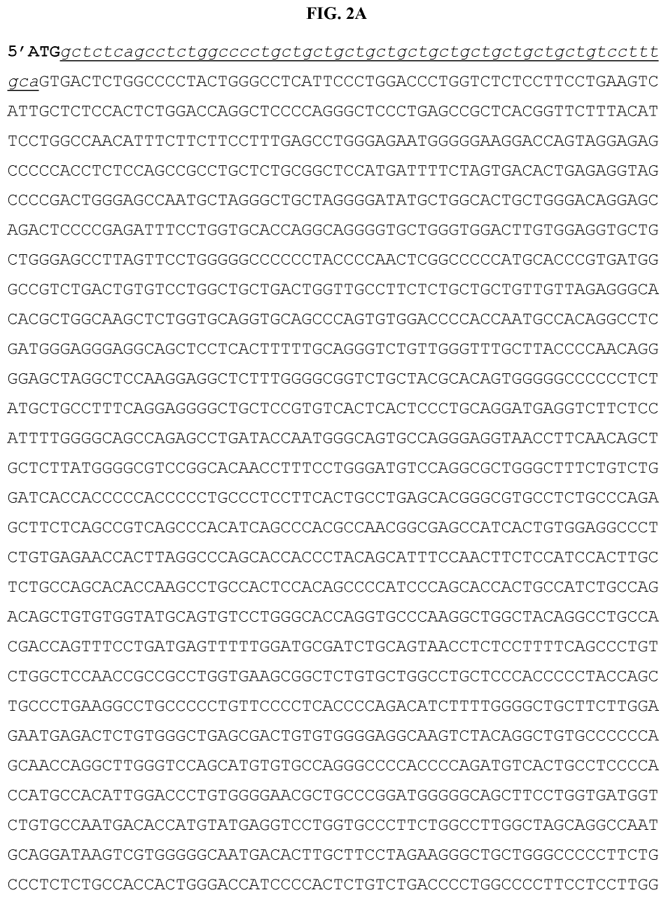

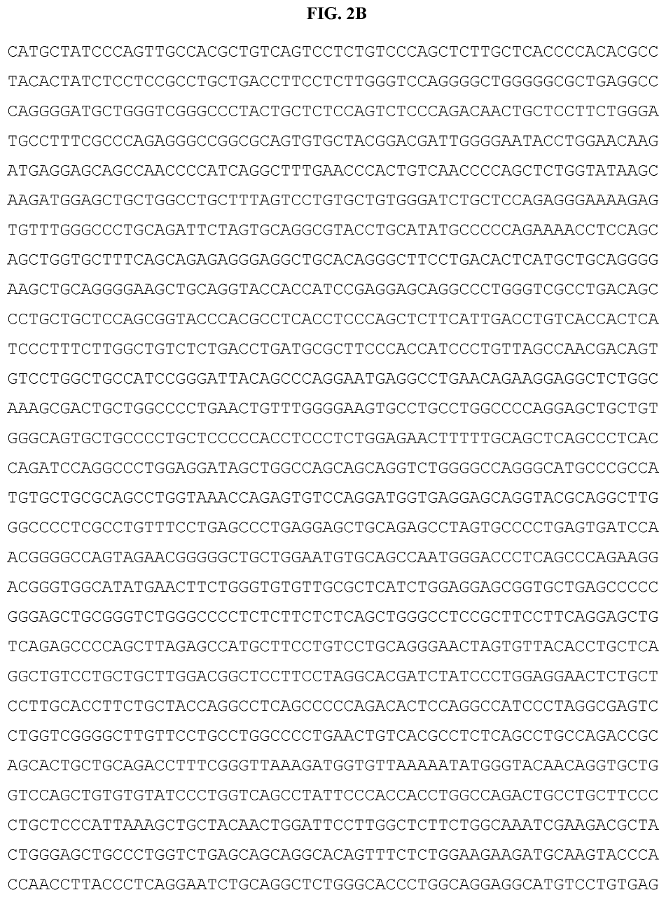

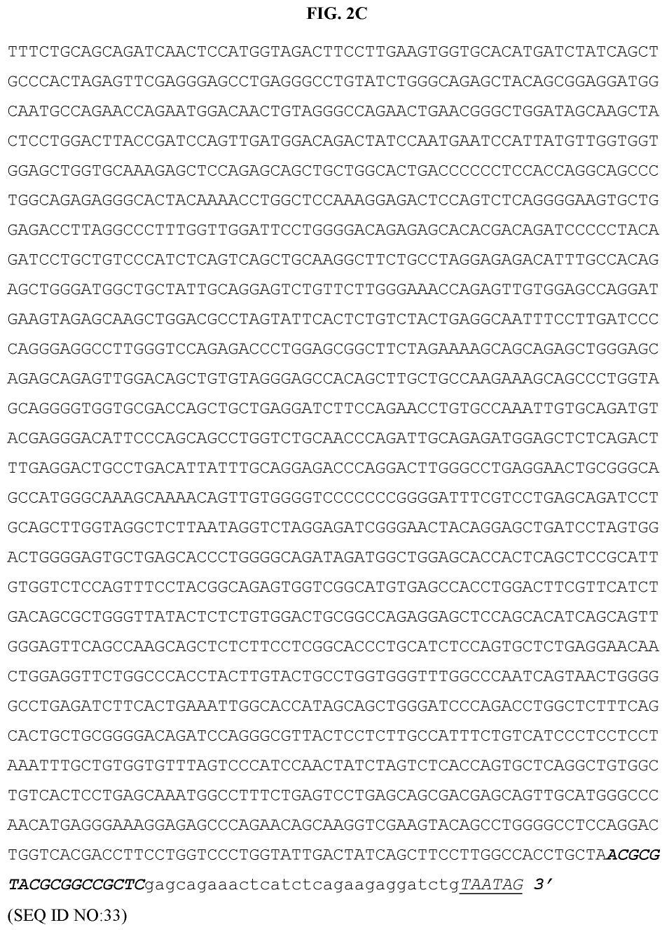

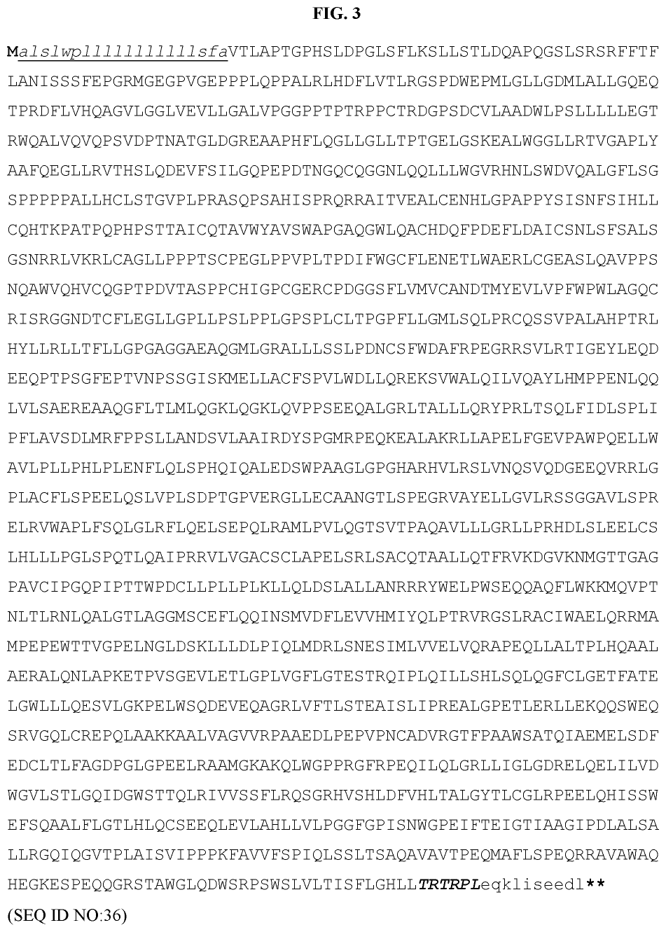

One embodiment of the disclosure may be directed to at least one vector (e.g., plasmid, transplicing plasmid, viral vector (e.g., lentivirus), Adenovirus, AAV, AAV genome) comprising a nucleotide sequence encoding a desired protein (FIG. 1). FIGS. 2A-2C show a nucleotide sequence (SEQ ID NO:33) containing the human STRC gene coding sequence (SEQ ID NO:1) in a 5′ to 3′ direction (encoding the human STRC protein sequence (upper case), SEQ ID NO:2 in FIG. 3) is as follows:

(SEQ ID NO: 1)
5′-GTGACTCTGGCCCCTACTGGGCCTCATTCCCTGGACCCTGGTCTCTCCTTCCTGAAGTC
ATTGCTCTCCACTCTGGACCAGGCTCCCCAGGGCTCCCTGAGCCGCTCACGGTTCTTTACAT
TCCTGGCCAACATTTCTTCTTCCTTTGAGCCTGGGAGAATGGGGGAAGGACCAGTAGGAGAG
CCCCCACCTCTCCAGCCGCCTGCTCTGCGGCTCCATGATTTTCTAGTGACACTGAGAGGTAG
CCCCGACTGGGAGCCAATGCTAGGGCTGCTAGGGGATATGCTGGCACTGCTGGGACAGGAGC
AGACTCCCCGAGATTTCCTGGTGCACCAGGCAGGGGTGCTGGGTGGACTTGTGGAGGTGCTG
CTGGGAGCCTTAGTTCCTGGGGGCCCCCCTACCCCAACTCGGCCCCCATGCACCCGTGATGG
GCCGTCTGACTGTGTCCTGGCTGCTGACTGGTTGCCTTCTCTGCTGCTGTTGTTAGAGGGCA
CACGCTGGCAAGCTCTGGTGCAGGTGCAGCCCAGTGTGGACCCCACCAATGCCACAGGCCTC
GATGGGAGGGAGGCAGCTCCTCACTTTTTGCAGGGTCTGTTGGGTTTGCTTACCCCAACAGG
GGAGCTAGGCTCCAAGGAGGCTCTTTGGGGCGGTCTGCTACGCACAGTGGGGGCCCCCCTCT
ATGCTGCCTTTCAGGAGGGGCTGCTCCGTGTCACTCACTCCCTGCAGGATGAGGTCTTCTCC
ATTTTGGGGCAGCCAGAGCCTGATACCAATGGGCAGTGCCAGGGAGGTAACCTTCAACAGCT
GCTCTTATGGGGCGTCCGGCACAACCTTTCCTGGGATGTCCAGGCGCTGGGCTTTCTGTCTG
GATCACCACCCCCACCCCCTGCCCTCCTTCACTGCCTGAGCACGGGCGTGCCTCTGCCCAGA
GCTTCTCAGCCGTCAGCCCACATCAGCCCACGCCAACGGCGAGCCATCACTGTGGAGGCCCT
CTGTGAGAACCACTTAGGCCCAGCACCACCCTACAGCATTTCCAACTTCTCCATCCACTTGC
TCTGCCAGCACACCAAGCCTGCCACTCCACAGCCCCATCCCAGCACCACTGCCATCTGCCAG
ACAGCTGTGTGGTATGCAGTGTCCTGGGCACCAGGTGCCCAAGGCTGGCTACAGGCCTGCCA
CGACCAGTTTCCTGATGAGTTTTTGGATGCGATCTGCAGTAACCTCTCCTTTTCAGCCCTGT
CTGGCTCCAACCGCCGCCTGGTGAAGCGGCTCTGTGCTGGCCTGCTCCCACCCCCTACCAGC
TGCCCTGAAGGCCTGCCCCCTGTTCCCCTCACCCCAGACATCTTTTGGGGCTGCTTCTTGGA
GAATGAGACTCTGTGGGCTGAGCGACTGTGTGGGGAGGCAAGTCTACAGGCTGTGCCCCCCA
GCAACCAGGCTTGGGTCCAGCATGTGTGCCAGGGCCCCACCCCAGATGTCACTGCCTCCCCA
CCATGCCACATTGGACCCTGTGGGGAACGCTGCCCGGATGGGGGCAGCTTCCTGGTGATGGT
CTGTGCCAATGACACCATGTATGAGGTCCTGGTGCCCTTCTGGCCTTGGCTAGCAGGCCAAT
GCAGGATAAGTCGTGGGGGCAATGACACTTGCTTCCTAGAAGGGCTGCTGGGCCCCCTTCTG
CCCTCTCTGCCACCACTGGGACCATCCCCACTCTGTCTGACCCCTGGCCCCTTCCTCCTTGG
CATGCTATCCCAGTTGCCACGCTGTCAGTCCTCTGTCCCAGCTCTTGCTCACCCCACACGCC
TACACTATCTCCTCCGCCTGCTGACCTTCCTCTTGGGTCCAGGGGCTGGGGGCGCTGAGGCC
CAGGGGATGCTGGGTCGGGCCCTACTGCTCTCCAGTCTCCCAGACAACTGCTCCTTCTGGGA
TGCCTTTCGCCCAGAGGGCCGGCGCAGTGTGCTACGGACGATTGGGGAATACCTGGAACAAG
ATGAGGAGCAGCCAACCCCATCAGGCTTTGAACCCACTGTCAACCCCAGCTCTGGTATAAGC
AAGATGGAGCTGCTGGCCTGCTTTAGTCCTGTGCTGTGGGATCTGCTCCAGAGGGAAAAGAG
TGTTTGGGCCCTGCAGATTCTAGTGCAGGCGTACCTGCATATGCCCCCAGAAAACCTCCAGC
AGCTGGTGCTTTCAGCAGAGAGGGAGGCTGCACAGGGCTTCCTGACACTCATGCTGCAGGGG
AAGCTGCAGGGGAAGCTGCAGGTACCACCATCCGAGGAGCAGGCCCTGGGTCGCCTGACAGC
CCTGCTGCTCCAGCGGTACCCACGCCTCACCTCCCAGCTCTTCATTGACCTGTCACCACTCA
TCCCTTTCTTGGCTGTCTCTGACCTGATGCGCTTCCCACCATCCCTGTTAGCCAACGACAGT
GTCCTGGCTGCCATCCGGGATTACAGCCCAGGAATGAGGCCTGAACAGAAGGAGGCTCTGGC
AAAGCGACTGCTGGCCCCTGAACTGTTTGGGGAAGTGCCTGCCTGGCCCCAGGAGCTGCTGT
GGGCAGTGCTGCCCCTGCTCCCCCACCTCCCTCTGGAGAACTTTTTGCAGCTCAGCCCTCAC
CAGATCCAGGCCCTGGAGGATAGCTGGCCAGCAGCAGGTCTGGGGCCAGGGCATGCCCGCCA
TGTGCTGCGCAGCCTGGTAAACCAGAGTGTCCAGGATGGTGAGGAGCAGGTACGCAGGCTTG
GGCCCCTCGCCTGTTTCCTGAGCCCTGAGGAGCTGCAGAGCCTAGTGCCCCTGAGTGATCCA
ACGGGGCCAGTAGAACGGGGGCTGCTGGAATGTGCAGCCAATGGGACCCTCAGCCCAGAAGG
ACGGGTGGCATATGAACTTCTGGGTGTGTTGCGCTCATCTGGAGGAGCGGTGCTGAGCCCCC
GGGAGCTGCGGGTCTGGGCCCCTCTCTTCTCTCAGCTGGGCCTCCGCTTCCTTCAGGAGCTG
TCAGAGCCCCAGCTTAGAGCCATGCTTCCTGTCCTGCAGGGAACTAGTGTTACACCTGCTCA
GGCTGTCCTGCTGCTTGGACGGCTCCTTCCTAGGCACGATCTATCCCTGGAGGAACTCTGCT
CCTTGCACCTTCTGCTACCAGGCCTCAGCCCCCAGACACTCCAGGCCATCCCTAGGCGAGTC
CTGGTCGGGGCTTGTTCCTGCCTGGCCCCTGAACTGTCACGCCTCTCAGCCTGCCAGACCGC
AGCACTGCTGCAGACCTTTCGGGTTAAAGATGGTGTTAAAAATATGGGTACAACAGGTGCTG
GTCCAGCTGTGTGTATCCCTGGTCAGCCTATTCCCACCACCTGGCCAGACTGCCTGCTTCCC
CTGCTCCCATTAAAGCTGCTACAACTGGATTCCTTGGCTCTTCTGGCAAATCGAAGACGCTA
CTGGGAGCTGCCCTGGTCTGAGCAGCAGGCACAGTTTCTCTGGAAGAAGATGCAAGTACCCA
CCAACCTTACCCTCAGGAATCTGCAGGCTCTGGGCACCCTGGCAGGAGGCATGTCCTGTGAG
TTTCTGCAGCAGATCAACTCCATGGTAGACTTCCTTGAAGTGGTGCACATGATCTATCAGCT
GCCCACTAGAGTTCGAGGGAGCCTGAGGGCCTGTATCTGGGCAGAGCTACAGCGGAGGATGG
CAATGCCAGAACCAGAATGGACAACTGTAGGGCCAGAACTGAACGGGCTGGATAGCAAGCTA
CTCCTGGACTTACCGATCCAGTTGATGGACAGACTATCCAATGAATCCATTATGTTGGTGGT
GGAGCTGGTGCAAAGAGCTCCAGAGCAGCTGCTGGCACTGACCCCCCTCCACCAGGCAGCCC
TGGCAGAGAGGGCACTACAAAACCTGGCTCCAAAGGAGACTCCAGTCTCAGGGGAAGTGCTG
GAGACCTTAGGCCCTTTGGTTGGATTCCTGGGGACAGAGAGCACACGACAGATCCCCCTACA
GATCCTGCTGTCCCATCTCAGTCAGCTGCAAGGCTTCTGCCTAGGAGAGACATTTGCCACAG
AGCTGGGATGGCTGCTATTGCAGGAGTCTGTTCTTGGGAAACCAGAGTTGTGGAGCCAGGAT
GAAGTAGAGCAAGCTGGACGCCTAGTATTCACTCTGTCTACTGAGGCAATTTCCTTGATCCC
CAGGGAGGCCTTGGGTCCAGAGACCCTGGAGCGGCTTCTAGAAAAGCAGCAGAGCTGGGAGC
AGAGCAGAGTTGGACAGCTGTGTAGGGAGCCACAGCTTGCTGCCAAGAAAGCAGCCCTGGTA
GCAGGGGTGGTGCGACCAGCTGCTGAGGATCTTCCAGAACCTGTGCCAAATTGTGCAGATGT
ACGAGGGACATTCCCAGCAGCCTGGTCTGCAACCCAGATTGCAGAGATGGAGCTCTCAGACT
TTGAGGACTGCCTGACATTATTTGCAGGAGACCCAGGACTTGGGCCTGAGGAACTGCGGGCA
GCCATGGGCAAAGCAAAACAGTTGTGGGGTCCCCCCCGGGGATTTCGTCCTGAGCAGATCCT
GCAGCTTGGTAGGCTCTTAATAGGTCTAGGAGATCGGGAACTACAGGAGCTGATCCTAGTGG
ACTGGGGAGTGCTGAGCACCCTGGGGCAGATAGATGGCTGGAGCACCACTCAGCTCCGCATT
GTGGTCTCCAGTTTCCTACGGCAGAGTGGTCGGCATGTGAGCCACCTGGACTTCGTTCATCT
GACAGCGCTGGGTTATACTCTCTGTGGACTGCGGCCAGAGGAGCTCCAGCACATCAGCAGTT
GGGAGTTCAGCCAAGCAGCTCTCTTCCTCGGCACCCTGCATCTCCAGTGCTCTGAGGAACAA
CTGGAGGTTCTGGCCCACCTACTTGTACTGCCTGGTGGGTTTGGCCCAATCAGTAACTGGGG
GCCTGAGATCTTCACTGAAATTGGCACCATAGCAGCTGGGATCCCAGACCTGGCTCTTTCAG
CACTGCTGCGGGGACAGATCCAGGGCGTTACTCCTCTTGCCATTTCTGTCATCCCTCCTCCT
AAATTTGCTGTGGTGTTTAGTCCCATCCAACTATCTAGTCTCACCAGTGCTCAGGCTGTGGC
TGTCACTCCTGAGCAAATGGCCTTTCTGAGTCCTGAGCAGCGACGAGCAGTTGCATGGGCCC
AACATGAGGGAAAGGAGAGCCCAGAACAGCAAGGTCGAAGTACAGCCTGGGGCCTCCAGGAC
TGGTCACGACCTTCCTGGTCCCTGGTATTGACTATCAGCTTCCTTGGCCACCTGCTA-3′.

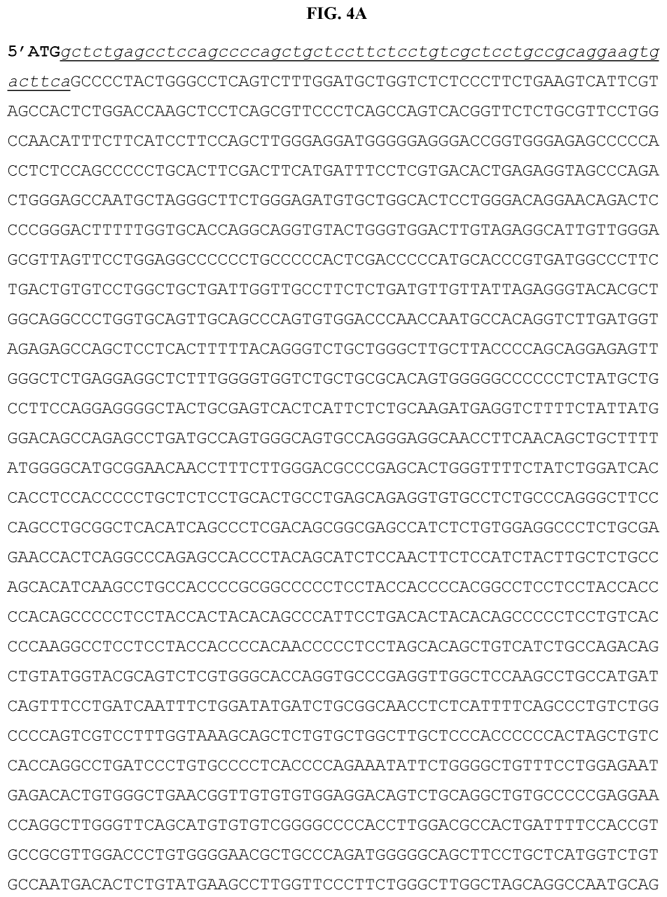

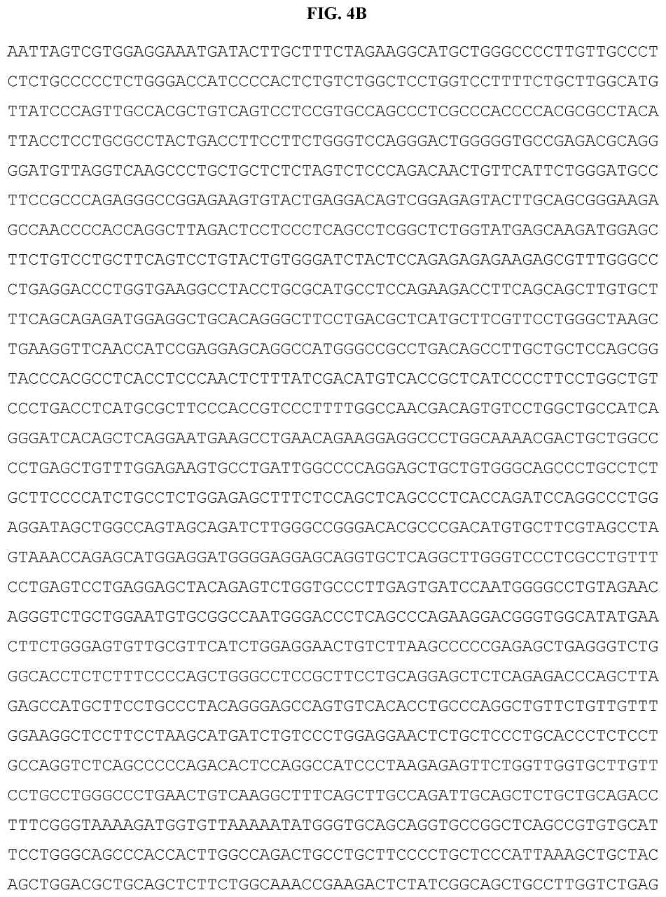

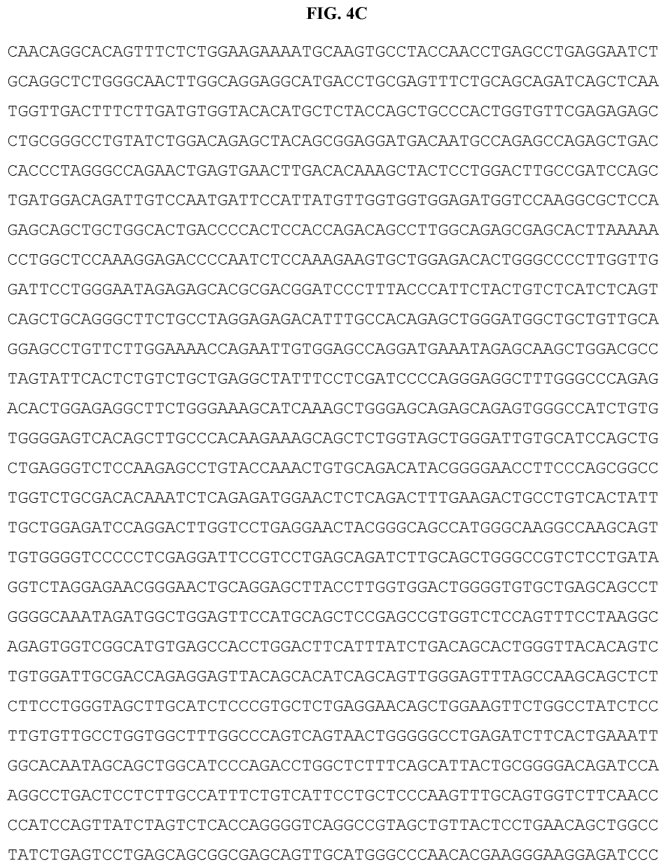

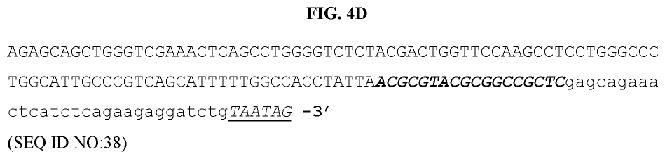

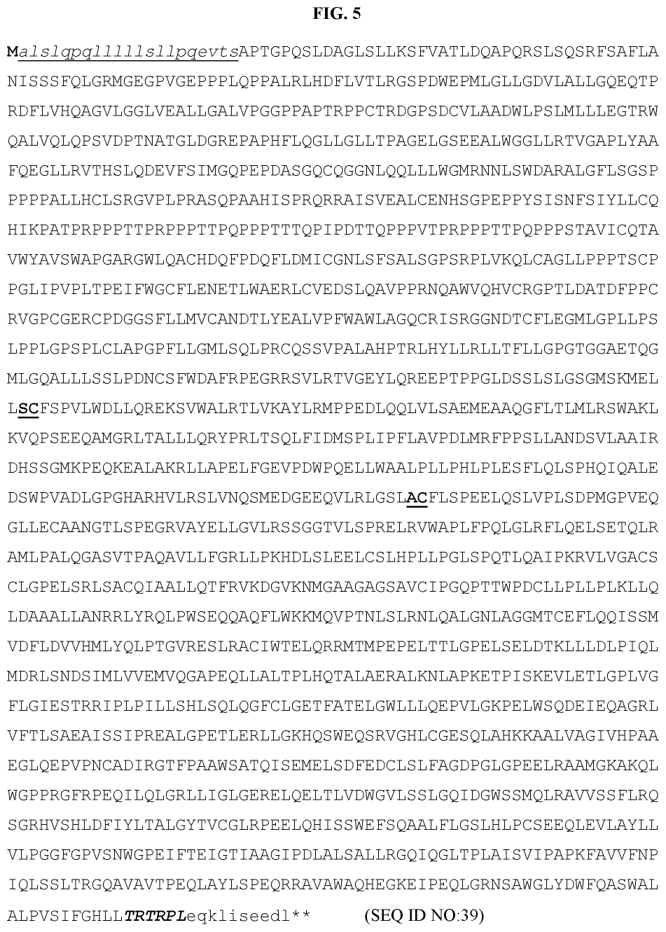

A further embodiment may be directed to at least one vector (e.g., plasmid, transplicing plasmid, viral vector, Adenovirus, AAV, AAV genome) (see, e.g., FIGS. 4A-4D; SEQ ID NO:38) comprising a murine STRC gene coding sequence (SEQ ID NO:3) in a 5′ to 3′ direction (encoding the murine STRC protein sequence, SEQ ID NO:4 in FIG. 5) that is as follows:

(SEQ ID NO: 3)
5′-GCCCCTACTGGGCCTCAGTCTTTGGATGCTGGTCTCTCCCTTCTGAA
GTCATTCGTAGCCACTCTGGACCAAGCTCCTCAGCGTTCCCTCAGCCAGT
CACGGTTCTCTGCGTTCCTGGCCAACATTTCTTCATCCTTCCAGCTTGGG
AGGATGGGGGAGGGACCGGTGGGAGAGCCCCCACCTCTCCAGCCCCCTGC
ACTTCGACTTCATGATTTCCTCGTGACACTGAGAGGTAGCCCAGACTGGG
AGCCAATGCTAGGGCTTCTGGGAGATGTGCTGGCACTCCTGGGACAGGAA
CAGACTCCCCGGGACTTTTTGGTGCACCAGGCAGGTGTACTGGGTGGACT
TGTAGAGGCATTGTTGGGAGCGTTAGTTCCTGGAGGCCCCCCTGCCCCCA
CTCGACCCCCATGCACCCGTGATGGCCCTTCTGACTGTGTCCTGGCTGCT
GATTGGTTGCCTTCTCTGATGTTGTTATTAGAGGGTACACGCTGGCAGGC
CCTGGTGCAGTTGCAGCCCAGTGTGGACCCAACCAATGCCACAGGTCTTG
ATGGTAGAGAGCCAGCTCCTCACTTTTTACAGGGTCTGCTGGGCTTGCTT
ACCCCAGCAGGAGAGTTGGGCTCTGAGGAGGCTCTTTGGGGTGGTCTGCT
GCGCACAGTGGGGGCCCCCCTCTATGCTGCCTTCCAGGAGGGGCTACTGC
GAGTCACTCATTCTCTGCAAGATGAGGTCTTTTCTATTATGGGACAGCCA
GAGCCTGATGCCAGTGGGCAGTGCCAGGGAGGCAACCTTCAACAGCTGCT
TTTATGGGGCATGCGGAACAACCTTTCTTGGGACGCCCGAGCACTGGGTT
TTCTATCTGGATCACCACCTCCACCCCCTGCTCTCCTGCACTGCCTGAGC
AGAGGTGTGCCTCTGCCCAGGGCTTCCCAGCCTGCGGCTCACATCAGCCC
TCGACAGCGGCGAGCCATCTCTGTGGAGGCCCTCTGCGAGAACCACTCAG
GCCCAGAGCCACCCTACAGCATCTCCAACTTCTCCATCTACTTGCTCTGC
CAGCACATCAAGCCTGCCACCCCGCGGCCCCCTCCTACCACCCCACGGCC
TCCTCCTACCACCCCACAGCCCCCTCCTACCACTACACAGCCCATTCCTG
ACACTACACAGCCCCCTCCTGTCACCCCAAGGCCTCCTCCTACCACCCCA
CAACCCCCTCCTAGCACAGCTGTCATCTGCCAGACAGCTGTATGGTACGC
AGTCTCGTGGGCACCAGGTGCCCGAGGTTGGCTCCAAGCCTGCCATGATC
AGTTTCCTGATCAATTTCTGGATATGATCTGCGGCAACCTCTCATTTTCA
GCCCTGTCTGGCCCCAGTCGTCCTTTGGTAAAGCAGCTCTGTGCTGGCTT
GCTCCCACCCCCCACTAGCTGTCCACCAGGCCTGATCCCTGTGCCCCTCA
CCCCAGAAATATTCTGGGGCTGTTTCCTGGAGAATGAGACACTGTGGGCT
GAACGGTTGTGTGTGGAGGACAGTCTGCAGGCTGTGCCCCCGAGGAACCA
GGCTTGGGTTCAGCATGTGTGTCGGGGCCCCACCTTGGACGCCACTGATT
TTCCACCGTGCCGCGTTGGACCCTGTGGGGAACGCTGCCCAGATGGGGGC
AGCTTCCTGCTCATGGTCTGTGCCAATGACACTCTGTATGAAGCCTTGGT
TCCCTTCTGGGCTTGGCTAGCAGGCCAATGCAGAATTAGTCGTGGAGGAA
ATGATACTTGCTTTCTAGAAGGCATGCTGGGCCCCTTGTTGCCCTCTCTG
CCCCCTCTGGGACCATCCCCACTCTGTCTGGCTCCTGGTCCTTTTCTGCT
TGGCATGTTATCCCAGTTGCCACGCTGTCAGTCCTCCGTGCCAGCCCTCG
CCCACCCCACGCGCCTACATTACCTCCTGCGCCTACTGACCTTCCTTCTG
GGTCCAGGGACTGGGGGTGCCGAGACGCAGGGGATGTTAGGTCAAGCCCT
GCTGCTCTCTAGTCTCCCAGACAACTGTTCATTCTGGGATGCCTTCCGCC
CAGAGGGCCGGAGAAGTGTACTGAGGACAGTCGGAGAGTACTTGCAGCGG
GAAGAGCCAACCCCACCAGGCTTAGACTCCTCCCTCAGCCTCGGCTCTGG
TATGAGCAAGATGGAGCTTCTGTCCTGCTTCAGTCCTGTACTGTGGGATC
TACTCCAGAGAGAGAAGAGCGTTTGGGCCCTGAGGACCCTGGTGAAGGCC
TACCTGCGCATGCCTCCAGAAGACCTTCAGCAGCTTGTGCTTTCAGCAGA
GATGGAGGCTGCACAGGGCTTCCTGACGCTCATGCTTCGTTCCTGGGCTA
AGCTGAAGGTTCAACCATCCGAGGAGCAGGCCATGGGCCGCCTGACAGCC
TTGCTGCTCCAGCGGTACCCACGCCTCACCTCCCAACTCTTTATCGACAT
GTCACCGCTCATCCCCTTCCTGGCTGTCCCTGACCTCATGCGCTTCCCAC
CGTCCCTTTTGGCCAACGACAGTGTCCTGGCTGCCATCAGGGATCACAGC
TCAGGAATGAAGCCTGAACAGAAGGAGGCCCTGGCAAAACGACTGCTGGC
CCCTGAGCTGTTTGGAGAAGTGCCTGATTGGCCCCAGGAGCTGCTGTGGG
CAGCCCTGCCTCTGCTTCCCCATCTGCCTCTGGAGAGCTTTCTCCAGCTC
AGCCCTCACCAGATCCAGGCCCTGGAGGATAGCTGGCCAGTAGCAGATCT
TGGGCCGGGACACGCCCGACATGTGCTTCGTAGCCTAGTAAACCAGAGCA
TGGAGGATGGGGAGGAGCAGGTGCTCAGGCTTGGGTCCCTCGCCTGTTTC
CTGAGTCCTGAGGAGCTACAGAGTCTGGTGCCCTTGAGTGATCCAATGGG
GCCTGTAGAACAGGGTCTGCTGGAATGTGCGGCCAATGGGACCCTCAGCC
CAGAAGGACGGGTGGCATATGAACTTCTGGGAGTGTTGCGTTCATCTGGA
GGAACTGTCTTAAGCCCCCGAGAGCTGAGGGTCTGGGCACCTCTCTTTCC
CCAGCTGGGCCTCCGCTTCCTGCAGGAGCTCTCAGAGACCCAGCTTAGAG
CCATGCTTCCTGCCCTACAGGGAGCCAGTGTCACACCTGCCCAGGCTGTT
CTGTTGTTTGGAAGGCTCCTTCCTAAGCATGATCTGTCCCTGGAGGAACT
CTGCTCCCTGCACCCTCTCCTGCCAGGTCTCAGCCCCCAGACACTCCAGG
CCATCCCTAAGAGAGTTCTGGTTGGTGCTTGTTCCTGCCTGGGCCCTGAA
CTGTCAAGGCTTTCAGCTTGCCAGATTGCAGCTCTGCTGCAGACCTTTCG
GGTAAAAGATGGTGTTAAAAATATGGGTGCAGCAGGTGCCGGCTCAGCCG
TGTGCATTCCTGGGCAGCCCACCACTTGGCCAGACTGCCTGCTTCCCCTG
CTCCCATTAAAGCTGCTACAGCTGGACGCTGCAGCTCTTCTGGCAAACCG
AAGACTCTATCGGCAGCTGCCTTGGTCTGAGCAACAGGCACAGTTTCTCT
GGAAGAAAATGCAAGTGCCTACCAACCTGAGCCTGAGGAATCTGCAGGCT
CTGGGCAACTTGGCAGGAGGCATGACCTGCGAGTTTCTGCAGCAGATCAG
CTCAATGGTTGACTTTCTTGATGTGGTACACATGCTCTACCAGCTGCCCA
CTGGTGTTCGAGAGAGCCTGCGGGCCTGTATCTGGACAGAGCTACAGCGG
AGGATGACAATGCCAGAGCCAGAGCTGACCACCCTAGGGCCAGAACTGAG
TGAACTTGACACAAAGCTACTCCTGGACTTGCCGATCCAGCTGATGGACA
GATTGTCCAATGATTCCATTATGTTGGTGGTGGAGATGGTCCAAGGCGCT
CCAGAGCAGCTGCTGGCACTGACCCCACTCCACCAGACAGCCTTGGCAGA
GCGAGCACTTAAAAACCTGGCTCCAAAGGAGACCCCAATCTCCAAAGAAG
TGCTGGAGACACTGGGCCCCTTGGTTGGATTCCTGGGAATAGAGAGCACG
CGACGGATCCCTTTACCCATTCTACTGTCTCATCTCAGTCAGCTGCAGGG
CTTCTGCCTAGGAGAGACATTTGCCACAGAGCTGGGATGGCTGCTGTTGC
AGGAGCCTGTTCTTGGAAAACCAGAATTGTGGAGCCAGGATGAAATAGAG
CAAGCTGGACGCCTAGTATTCACTCTGTCTGCTGAGGCTATTTCCTCGAT
CCCCAGGGAGGCTTTGGGCCCAGAGACACTGGAGAGGCTTCTGGGAAAGC
ATCAAAGCTGGGAGCAGAGCAGAGTGGGCCATCTGTGTGGGGAGTCACAG
CTTGCCCACAAGAAAGCAGCTCTGGTAGCTGGGATTGTGCATCCAGCTGC
TGAGGGTCTCCAAGAGCCTGTACCAAACTGTGCAGACATACGGGGAACCT
TCCCAGCGGCCTGGTCTGCGACACAAATCTCAGAGATGGAACTCTCAGAC
TTTGAAGACTGCCTGTCACTATTTGCTGGAGATCCAGGACTTGGTCCTGA
GGAACTACGGGCAGCCATGGGCAAGGCCAAGCAGTTGTGGGGTCCCCCTC
GAGGATTCCGTCCTGAGCAGATCTTGCAGCTGGGCCGTCTCCTGATAGGT
CTAGGAGAACGGGAACTGCAGGAGCTTACCTTGGTGGACTGGGGTGTGCT
GAGCAGCCTGGGGCAAATAGATGGCTGGAGTTCCATGCAGCTCCGAGCCG
TGGTCTCCAGTTTCCTAAGGCAGAGTGGTCGGCATGTGAGCCACCTGGAC
TTCATTTATCTGACAGCACTGGGTTACACAGTCTGTGGATTGCGACCAGA
GGAGTTACAGCACATCAGCAGTTGGGAGTTTAGCCAAGCAGCTCTCTTCC
TGGGTAGCTTGCATCTCCCGTGCTCTGAGGAACAGCTGGAAGTTCTGGCC
TATCTCCTTGTGTTGCCTGGTGGCTTTGGCCCAGTCAGTAACTGGGGGCC
TGAGATCTTCACTGAAATTGGCACAATAGCAGCTGGCATCCCAGACCTGG
CTCTTTCAGCATTACTGCGGGGACAGATCCAAGGCCTGACTCCTCTTGCC
ATTTCTGTCATTCCTGCTCCCAAGTTTGCAGTGGTCTTCAACCCCATCCA
GTTATCTAGTCTCACCAGGGGTCAGGCCGTAGCTGTTACTCCTGAACAGC
TGGCCTATCTGAGTCCTGAGCAGCGGCGAGCAGTTGCATGGGCCCAACAC
GAAGGGAAGGAGATCCCAGAGCAGCTGGGTCGAAACTCAGCCTGGGGTCT
CTACGACTGGTTCCAAGCCTCCTGGGCCCTGGCATTGCCCGTCAGCATTT
TTGGCCACCTATTA-3′.

Another embodiment of the disclosure may provide a first vector (e.g., plasmid, transplicing plasmid, viral vector, Adenovirus, AAV, AAV genome) comprising a first nucleotide sequence (e.g., SEQ ID NO:5 encoding SEQ ID NO:6; SEQ ID NO:7 encoding SEQ ID NO:8) comprising, in a 5′ to 3′ direction: a signal sequence (e.g., SEQ ID NO:9 encoding SEQ ID NO: 10; or SEQ ID NO:11 encoding SEQ ID NO: 12) at the 5′-end of the partial coding sequence, where the partial coding sequence may be flanked by or adjacent to a downstream sequence encoding a splice donor sequence (e.g., an N-terminal intein (N-intein); SEQ ID NO:13 encoding SEQ ID NO:14); a partial coding sequence encoding an amino terminal (N-terminal) portion of the protein of interest (e.g., STRC; SEQ ID NO:15; SEQ ID NO:16); and a second vector (e.g., plasmid, transplicing plasmid, viral vector, Adenovirus, AAV, AAV genome) comprising a second nucleotide sequence (e.g., SEQ ID NO:17 encoding SEQ ID NO:18; SEQ ID NO:19 encoding SEQ ID NO:20) comprising, in a 5′ to 3′ direction: a signal sequence (e.g., human SEQ ID NO:9 encoding SEQ ID NO:10; or murine SEQ ID NO:11 encoding SEQ ID NO: 12) that may be upstream of a splice acceptor sequence (e.g., a C-terminal intein (C-intein); SEQ ID NO:21 encoding SEQ ID NO:22) positioned immediately adjacent to or flanking a downstream partial coding sequence encoding the remaining portion of the full-length coding sequence of the protein of interest, i.e., the C-terminal portion of the protein of interest (e.g., STRC; human SEQ ID NO:23; SEQ ID NO:24). When expressed in a cell (e.g., host cell, mammalian (e.g., human, canine, feline, equine, murine)), the first vector and the second vector may each express their respective portions of proteins of interest (e.g., N-STRC, C-STRC), which form a full-length protein of interest (e.g., STRC; SEQ ID NO:25; SEQ ID NO:26).

Another embodiment may provide a first vector (e.g., plasmid, transplicing plasmid, viral vector, Adenovirus, AAV, AAV genome) comprising a first nucleotide sequence containing: a partial coding sequence encoding an amino terminal (N-terminal) portion of a protein of interest (e.g., STRC), including a signal sequence at the 5′-end of the partial coding sequence, where the partial coding sequence may be flanked by or adjacent to a downstream sequence encoding a splice donor sequence (e.g., an N-terminal intein (N-intein)), where the splice donor sequence is flanked by or adjacent to a downstream 3′ITR sequence (e.g., AAV9-php.B-Prot-trans/donor). A second vector (e.g., plasmid, transplicing plasmid, viral vector, Adenovirus, AAV, AAV genome) comprising a second nucleotide sequence containing: a 5′ITR sequence upstream of a signal sequence that may be upstream of a splice acceptor sequence (e.g., a C-terminal intein (C-intein)) positioned immediately adjacent to or flanking a downstream partial coding sequence encoding the remaining C-terminal portion of the protein of interest (e.g., STRC), where the second nucleotide sequence may further contain a C-terminal myc tag downstream of the partial coding sequence encoding the C-terminal portion of the protein of interest (e.g., AAV9-phpB-Prot-trans/acceptor). A full-length mRNA of interest (e.g., STRC mRNA) may form by a head-to-tail recombination between the two transplicing plasmids (5′ to 3′ end to 5′ to 3′ end), transcription, and subsequent splicing across the inverted terminal repeat (ITR) junctions in cells co-infected with the two vectors (e.g., plasmid, transplicing plasmid, viral vector, Adenovirus, AAV, AAV genome).

By “sequence encoding an N-terminal portion or fragment of a protein of interest,” where the protein of interest is, for example, a Stereocilin (STRC) protein, is meant a partial coding sequence of an N-terminal portion or fragment of STRC, where in some instances, the “sequence encoding an N-terminal portion or fragment of STRC” may include a sequence encoding a signal peptide coding sequence (lower case, italicized, and underlined) upstream of the partial coding sequence of an N-terminal portion or fragment of STRC (upper case). In some embodiments, the “sequence encoding an N-terminal fragment of Stereocilin (STRC)” may not include a nucleotide sequence encoding a signal peptide coding sequence. Another embodiment may provide a nucleotide sequence comprising a “sequence encoding an N-terminal fragment or portion of STRC” fused at its C-terminal end to an N-terminal portion or fragment of intein (N-intein) (bold and underlined), where the nucleotide sequence at the 5′ end begins with an “ATG” start codon (bold) and ends with a stop codon (upper case, italicized, and underlined).

An exemplary human nucleotide sequence comprising a “sequence encoding an N-terminal portion or fragment of STRC” may be as follows:

(SEQ ID NO: 5)
5′ATGgctctcagcctctggcccctgctgctgctgctgctgctgctgctg
ctgctgtcctttgcaGTGACTCTGGCCCCTACTGGGCCTCATTCCCTGGA
CCCTGGTCTCTCCTTCCTGAAGTCATTGCTCTCCACTCTGGACCAGGCTC
CCCAGGGCTCCCTGAGCCGCTCACGGTTCTTTACATTCCTGGCCAACATT
TCTTCTTCCTTTGAGCCTGGGAGAATGGGGGAAGGACCAGTAGGAGAGCC
CCCACCTCTCCAGCCGCCTGCTCTGCGGCTCCATGATTTTCTAGTGACAC
TGAGAGGTAGCCCCGACTGGGAGCCAATGCTAGGGCTGCTAGGGGATATG
CTGGCACTGCTGGGACAGGAGCAGACTCCCCGAGATTTCCTGGTGCACCA
GGCAGGGGTGCTGGGTGGACTTGTGGAGGTGCTGCTGGGAGCCTTAGTTC
CTGGGGGCCCCCCTACCCCAACTCGGCCCCCATGCACCCGTGATGGGCCG
TCTGACTGTGTCCTGGCTGCTGACTGGTTGCCTTCTCTGCTGCTGTTGTT
AGAGGGCACACGCTGGCAAGCTCTGGTGCAGGTGCAGCCCAGTGTGGACC
CCACCAATGCCACAGGCCTCGATGGGAGGGAGGCAGCTCCTCACTTTTTG
CAGGGTCTGTTGGGTTTGCTTACCCCAACAGGGGAGCTAGGCTCCAAGGA
GGCTCTTTGGGGCGGTCTGCTACGCACAGTGGGGGCCCCCCTCTATGCTG
CCTTTCAGGAGGGGCTGCTCCGTGTCACTCACTCCCTGCAGGATGAGGTC
TTCTCCATTTTGGGGCAGCCAGAGCCTGATACCAATGGGCAGTGCCAGGG
AGGTAACCTTCAACAGCTGCTCTTATGGGGCGTCCGGCACAACCTTTCCT
GGGATGTCCAGGCGCTGGGCTTTCTGTCTGGATCACCACCCCCACCCCCT
GCCCTCCTTCACTGCCTGAGCACGGGCGTGCCTCTGCCCAGAGCTTCTCA
GCCGTCAGCCCACATCAGCCCACGCCAACGGCGAGCCATCACTGTGGAGG
CCCTCTGTGAGAACCACTTAGGCCCAGCACCACCCTACAGCATTTCCAAC
TTCTCCATCCACTTGCTCTGCCAGCACACCAAGCCTGCCACTCCACAGCC
CCATCCCAGCACCACTGCCATCTGCCAGACAGCTGTGTGGTATGCAGTGT
CCTGGGCACCAGGTGCCCAAGGCTGGCTACAGGCCTGCCACGACCAGTTT
CCTGATGAGTTTTTGGATGCGATCTGCAGTAACCTCTCCTTTTCAGCCCT
GTCTGGCTCCAACCGCCGCCTGGTGAAGCGGCTCTGTGCTGGCCTGCTCC
CACCCCCTACCAGCTGCCCTGAAGGCCTGCCCCCTGTTCCCCTCACCCCA
GACATCTTTTGGGGCTGCTTCTTGGAGAATGAGACTCTGTGGGCTGAGCG
ACTGTGTGGGGAGGCAAGTCTACAGGCTGTGCCCCCCAGCAACCAGGCTT
GGGTCCAGCATGTGTGCCAGGGCCCCACCCCAGATGTCACTGCCTCCCCA
CCATGCCACATTGGACCCTGTGGGGAACGCTGCCCGGATGGGGGCAGCTT
CCTGGTGATGGTCTGTGCCAATGACACCATGTATGAGGTCCTGGTGCCCT
TCTGGCCTTGGCTAGCAGGCCAATGCAGGATAAGTCGTGGGGGCAATGAC
ACTTGCTTCCTAGAAGGGCTGCTGGGCCCCCTTCTGCCCTCTCTGCCACC
ACTGGGACCATCCCCACTCTGTCTGACCCCTGGCCCCTTCCTCCTTGGCA
TGCTATCCCAGTTGCCACGCTGTCAGTCCTCTGTCCCAGCTCTTGCTCAC
CCCACACGCCTACACTATCTCCTCCGCCTGCTGACCTTCCTCTTGGGTCC
AGGGGCTGGGGGCGCTGAGGCCCAGGGGATGCTGGGTCGGGCCCTACTGC
TCTCCAGTCTCCCAGACAACTGCTCCTTCTGGGATGCCTTTCGCCCAGAG
GGCCGGCGCAGTGTGCTACGGACGATTGGGGAATACCTGGAACAAGATGA
GGAGCAGCCAACCCCATCAGGCTTTGAACCCACTGTCAACCCCAGCTCTG
GTATAAGCAAGATGGAGCTGCTGGCC
TGCCTGTCATACGAAACCGAGATAC
TGACAGTAGAATATGGCCTTCTGCC
AATCGGGAAGATTGTGGAGAAACGG
ATAGAATGCACAGTTTACTCTGTCG
ATAACAATGGTAACATTTATACTCA
GCCAGTTGCCCAGTGGCACGACCGG
GGAGAGCAGGAAGTATTCGAATACT
GTCTGGAGGATGGAAGTCTCATTAG
GGCCACTAAGGACCACAAATTTATG
ACAGTCGATGGCCAGATGCTGCCTA
TAGACGAAATCTTTGAGCGAGAGTT
GGACCTCATGCGAGTTGACAACCTT
CCTAATTAATAG 3′.

Another exemplary murine nucleotide sequence comprising a “sequence encoding an N-terminal portion or fragment of STRC” may be as follows:

(SEQ ID NO: 7)

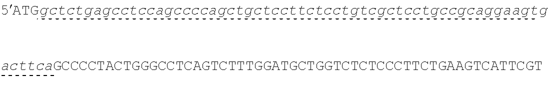

AGCCACTCTGGACCAAGCTCCTCAGCGTTCCCTCAGCCAGTCACGGTTCTCTGCGTTCCTGG
CCAACATTTCTTCATCCTTCCAGCTTGGGAGGATGGGGGAGGGACCGGTGGGAGAGCCCCCA
CCTCTCCAGCCCCCTGCACTTCGACTTCATGATTTCCTCGTGACACTGAGAGGTAGCCCAGA
CTGGGAGCCAATGCTAGGGCTTCTGGGAGATGTGCTGGCACTCCTGGGACAGGAACAGACTC
CCCGGGACTTTTTGGTGCACCAGGCAGGTGTACTGGGTGGACTTGTAGAGGCATTGTTGGGA
GCGTTAGTTCCTGGAGGCCCCCCTGCCCCCACTCGACCCCCATGCACCCGTGATGGCCCTTC
TGACTGTGTCCTGGCTGCTGATTGGTTGCCTTCTCTGATGTTGTTATTAGAGGGTACACGCT
GGCAGGCCCTGGTGCAGTTGCAGCCCAGTGTGGACCCAACCAATGCCACAGGTCTTGATGGT
AGAGAGCCAGCTCCTCACTTTTTACAGGGTCTGCTGGGCTTGCTTACCCCAGCAGGAGAGTT
GGGCTCTGAGGAGGCTCTTTGGGGTGGTCTGCTGCGCACAGTGGGGGCCCCCCTCTATGCTG
CCTTCCAGGAGGGGCTACTGCGAGTCACTCATTCTCTGCAAGATGAGGTCTTTTCTATTATG
GGACAGCCAGAGCCTGATGCCAGTGGGCAGTGCCAGGGAGGCAACCTTCAACAGCTGCTTTT
ATGGGGCATGCGGAACAACCTTTCTTGGGACGCCCGAGCACTGGGTTTTCTATCTGGATCAC
CACCTCCACCCCCTGCTCTCCTGCACTGCCTGAGCAGAGGTGTGCCTCTGCCCAGGGCTTCC
CAGCCTGCGGCTCACATCAGCCCTCGACAGCGGCGAGCCATCTCTGTGGAGGCCCTCTGCGA
GAACCACTCAGGCCCAGAGCCACCCTACAGCATCTCCAACTTCTCCATCTACTTGCTCTGCC
AGCACATCAAGCCTGCCACCCCGCGGCCCCCTCCTACCACCCCACGGCCTCCTCCTACCACC
CCACAGCCCCCTCCTACCACTACACAGCCCATTCCTGACACTACACAGCCCCCTCCTGTCAC
CCCAAGGCCTCCTCCTACCACCCCACAACCCCCTCCTAGCACAGCTGTCATCTGCCAGACAG
CTGTATGGTACGCAGTCTCGTGGGCACCAGGTGCCCGAGGTTGGCTCCAAGCCTGCCATGAT
CAGTTTCCTGATCAATTTCTGGATATGATCTGCGGCAACCTCTCATTTTCAGCCCTGTCTGG
CCCCAGTCGTCCTTTGGTAAAGCAGCTCTGTGCTGGCTTGCTCCCACCCCCCACTAGCTGTC
CACCAGGCCTGATCCCTGTGCCCCTCACCCCAGAAATATTCTGGGGCTGTTTCCTGGAGAAT
GAGACACTGTGGGCTGAACGGTTGTGTGTGGAGGACAGTCTGCAGGCTGTGCCCCCGAGGAA
CCAGGCTTGGGTTCAGCATGTGTGTCGGGGCCCCACCTTGGACGCCACTGATTTTCCACCGT
GCCGCGTTGGACCCTGTGGGGAACGCTGCCCAGATGGGGGCAGCTTCCTGCTCATGGTCTGT
GCCAATGACACTCTGTATGAAGCCTTGGTTCCCTTCTGGGCTTGGCTAGCAGGCCAATGCAG
AATTAGTCGTGGAGGAAATGATACTTGCTTTCTAGAAGGCATGCTGGGCCCCTTGTTGCCCT
CTCTGCCCCCTCTGGGACCATCCCCACTCTGTCTGGCTCCTGGTCCTTTTCTGCTTGGCATG
TTATCCCAGTTGCCACGCTGTCAGTCCTCCGTGCCAGCCCTCGCCCACCCCACGCGCCTACA
TTACCTCCTGCGCCTACTGACCTTCCTTCTGGGTCCAGGGACTGGGGGTGCCGAGACGCAGG
GGATGTTAGGTCAAGCCCTGCTGCTCTCTAGTCTCCCAGACAACTGTTCATTCTGGGATGCC
TTCCGCCCAGAGGGCCGGAGAAGTGTACTGAGGACAGTCGGAGAGTACTTGCAGCGGGAAGA
GCCAACCCCACCAGGCTTAGACTCCTCCCTCAGCCTCGGCTCTGGTATGAGCAAGATGGAGC
TTCTGTCCTGCCTGTCATACGAAACCGAGATACTGACAGTAGAATATGGCCTTCTGCCAATC
GGGAAGATTGTGGAGAAACGGATAGAATGCACAGTTTACTCTGTCGATAACAATGGTAACAT
TTATACTCAGCCAGTTGCCCAGTGGCACGACCGGGGAGAGCAGGAAGTATTCGAATACTGTC
TGGAGGATGGAAGTCTCATTAGGGCCACTAAGGACCACAAATTTATGACAGTCGATGGCCAG
ATGCTGCCTATAGACGAAATCTTTGAGCGAGAGTTGGACCTCATGCGAGTTGACAACCTTCC
TAATTAATAG 3′.

By “N-terminal portion or fragment of a protein of interest,” where the protein of interest is, for example, a STRC protein, is meant an amino acid sequence of an N-terminal portion or fragment of the Stereocilin (STRC) polypeptide. For example, an amino acid sequence of an N-terminal fragment of STRC may comprise a signal peptide sequence (e.g., of 22 amino acids) (lower case, italicized, and underlined) at the N-terminal end beginning with a methionine (M) (bold at N-terminal end) encoded by the ATG start codon. The amino acid sequence comprising the “N-terminal portion or fragment of a STRC protein” may further comprise downstream and/or adjacent thereto, an N-terminal fragment of intein (N-intein) (bold and underlined). In yet another embodiment, by “N-terminal portion or fragment of STRC protein” is meant an amino acid sequence of an N-terminal fragment of STRC without the signal peptide sequence.

An exemplary amino acid sequence comprising a human “N-terminal portion or fragment of STRC protein” may be as follows:

(SEQ ID NO: 6)
MALSLWPLLLLLLLLLLLSFAVTLAPTGPHSLDPGLSFLKSLLSTLDQAP
QGSLSRSRFFTFLANISSSFEPGRMGEGPVGEPPPLQPPALRLHDFLVTL
RGSPDWEPMLGLLGDMLALLGQEQTPRDFLVHQAGVLGGLVEVLLGALVP
GGPPTPTRPPCTRDGPSDCVLAADWLPSLLLLLEGTRWQALVQVQPSVDP
TNATGLDGREAAPHFLQGLLGLLTPTGELGSKEALWGGLLRTVGAPLYAA
FQEGLLRVTHSLQDEVFSILGQPEPDTNGQCQGGNLQQLLLWGVRHNLSW
DVQALGFLSGSPPPPPALLHCLSTGVPLPRASQPSAHISPRQRRAITVEA
LCENHLGPAPPYSISNFSIHLLCQHTKPATPQPHPSTTAICQTAVWYAVS
WAPGAQGWLQACHDQFPDEFLDAICSNLSFSALSGSNRRLVKRLCAGLLP
PPTSCPEGLPPVPLTPDIFWGCFLENETLWAERLCGEASLQAVPPSNQAW
VQHVCQGPTPDVTASPPCHIGPCGERCPDGGSFLVMVCANDTMYEVLVPF
WPWLAGQCRISRGGNDTCFLEGLLGPLLPSLPPLGPSPLCLTPGPFLLGM
LSQLPRCQSSVPALAHPTRLHYLLRLLTFLLGPGAGGAEAQGMLGRALLL
SSLPDNCSFWDAFRPEGRRSVLRTIGEYLEQDEEQPTPSGFEPTVNPSSG
ISKMELLACLSYETEILTVEYGLLPIGKIVEKRIECT
VYSVDNNGNIYTQPVAQWHDRGEQEVFEYCLEDGSLI
RATKDHKFMTVDGQMLPIDEIFERELDLMRVDNLPN**.

Another exemplary amino acid sequence comprising a murine “N-terminal portion or fragment of STRC protein” may be as follows:

(SEQ ID NO: 8)
MALSLQPQLLLLLSLLPQEVTSAPTGPQSLDAGLSLLKSFVATLDQAPQR
SLSQSRFSAFLANISSSFQLGRMGEGPVGEPPPLQPPALRLHDFLVTLRG
SPDWEPMLGLLGDVLALLGQEQTPRDFLVHQAGVLGGLVEALLGALVPGG
PPAPTRPPCTRDGPSDCVLAADWLPSLMLLLEGTRWQALVQLQPSVDPTN
ATGLDGREPAPHFLQGLLGLLTPAGELGSEEALWGGLLRTVGAPLYAAFQ
EGLLRVTHSLQDEVFSIMGQPEPDASGQCQGGNLQQLLLWGMRNNLSWDA
RALGFLSGSPPPPPALLHCLSRGVPLPRASQPAAHISPRQRRAISVEALC
ENHSGPEPPYSISNFSIYLLCQHIKPATPRPPPTTPRPPPTTPQPPPTTT
QPIPDTTQPPPVTPRPPPTTPQPPPSTAVICQTAVWYAVSWAPGARGWLQ
ACHDQFPDQFLDMICGNLSFSALSGPSRPLVKQLCAGLLPPPTSCPPGLI
PVPLTPEIFWGCFLENETLWAERLCVEDSLQAVPPRNQAWVQHVCRGPTL
DATDFPPCRVGPCGERCPDGGSFLLMVCANDTLYEALVPFWAWLAGQCRI
SRGGNDTCFLEGMLGPLLPSLPPLGPSPLCLAPGPFLLGMLSQLPRCQSS
VPALAHPTRLHYLLRLLTFLLGPGTGGAETQGMLGQALLLSSLPDNCSFW
DAFRPEGRRSVLRTVGEYLQREEPTPPGLDSSLSLGSGMSKMELLS
CLSYETEILTVEYGLLPIGKIVEKRIECTVYSVDNNG
NIYTQPVAQWHDRGEQEVFEYCLEDGSLIRATKDHKF
MTVDGQMLPIDEIFERELDLMRVDNLPN**.

Exemplary “N-terminal STRC polypeptide” sequences are provided below (N-terminal to C-terminal direction) for human and murine, respectively.

An exemplary human N-terminal STRC polypeptide sequence (N-terminal to C-terminal direction), which does not include the Methionine encoded by the ATG start codon or signal peptide sequence is provided below (N-terminal to C-terminal direction):

(SEQ ID NO: 15)
VTLAPTGPHSLDPGLSFLKSLLSTLDQAPQGSLSRSRFFTFLANISSSFE
PGRMGEGPVGEPPPLQPPALRLHDFLVTLRGSPDWEPMLGLLGDMLALLG
QEQTPRDFLVHQAGVLGGLVEVLLGALVPGGPPTPTRPPCTRDGPSDCVL
AADWLPSLLLLLEGTRWQALVQVQPSVDPTNATGLDGREAAPHFLQGLLG
LLTPTGELGSKEALWGGLLRTVGAPLYAAFQEGLLRVTHSLQDEVFSILG
QPEPDTNGQCQGGNLQQLLLWGVRHNLSWDVQALGFLSGSPPPPPALLHC
LSTGVPLPRASQPSAHISPRQRRAITVEALCENHLGPAPPYSISNFSIHL
LCQHTKPATPQPHPSTTAICQTAVWYAVSWAPGAQGWLQACHDQFPDEFL
DAICSNLSFSALSGSNRRLVKRLCAGLLPPPTSCPEGLPPVPLTPDIFWG
CFLENETLWAERLCGEASLQAVPPSNQAWVQHVCQGPTPDVTASPPCHIG
PCGERCPDGGSFLVMVCANDTMYEVLVPFWPWLAGQCRISRGGNDTCFLE
GLLGPLLPSLPPLGPSPLCLTPGPFLLGMLSQLPRCQSSVPALAHPTRLH
YLLRLLTFLLGPGAGGAEAQGMLGRALLLSSLPDNCSFWDAFRPEGRRSV
LRTIGEYLEQDEEQPTPSGFEPTVNPSSGISKMELLA.

Another exemplary murine N-terminal STRC polypeptide sequence (N-terminal to C-terminal direction), which does not include the Methionine encoded by the ATG start codon or signal peptide sequence, and the 17-residue hydrophobic regions are underlined (N-terminal to C-terminal direction):

(SEQ ID NO: 16)
APTGPQSLDAGLSLLKSEVATLDQAPQRSLSQSRFSAFLANISSSFQLGR
MGEGPVGEPPPLQPPALRLHDFLVTLRGSPDWEPMLGLLGDVLALLGQEQ
TPRDFLVHQAGVLGGLVEALLGALVPGGPPAPTRPPCTRDGPSDCVLAAD
WLPSLMLLLEGTRWQALVQLQPSVDPTNATGLDGREPAPHFLQGLLGLLT
PAGELGSEEALWGGLLRTVGAPLYAAFQEGLLRVTHSLQDEVFSIMGQPE
PDASGQCQGGNLQQLLLWGMRNNLSWDARALGFLSGSPPPPPALLHCLSR
GVPLPRASQPAAHISPRQRRAISVEALCENHSGPEPPYSISNFSIYLLCQ
HIKPATPRPPPTTPRPPPTTPQPPPTTTQPIPDTTQPPPVTPRPPPTTPQ
PPPSTAVICQTAVWYAVSWAPGARGWLQACHDQFPDQFLDMICGNLSFSA
LSGPSRPLVKQLCAGLLPPPTSCPPGLIPVPLTPEIFWGCFLENETLWAE
RLCVEDSLQAVPPRNQAWVQHVCRGPTLDATDFPPCRVGPCGERCPDGGS
FLLMVCANDTLYEALVPFWAWLAGQCRISRGGNDTCFLEGMLGPLLPSLP
PLGPSPLCLAPGPFLLGMLSQLPRCQSSVPALAHPTRLHYLLRLLTFLLG
PGTGGAETQGMLGQALLLSSLPDNCSFWDAFRPEGRRSVLRTVGEYLQRE
EPTPPGLDSSLSLGSGMSKMELLS.

By “sequence encoding a C-terminal portion or fragment of Stereocilin (STRC)” is meant a partial coding sequence of a C-terminal portion or fragment of STRC. An embodiment may provide a nucleotide sequence comprising at the 5′ end an “ATG” start codon (bold at 5′ end), a sequence encoding a signal peptide coding sequence (lower case, italicized, and underlined) upstream and flanking a sequence encoding a C-terminal fragment of intein (C-intein) (bold and underlined), which is upstream and flanking a “nucleotide sequence encoding a C-terminal portion or fragment of a STRC protein.” Another embodiment may further provide the nucleotide sequence encoding a C-terminal portion or fragment of STRC with a downstream linker sequence (bold and italicized), a Myc tag (lower case), and stop codon (upper case, italicized, and underlined).

An exemplary human nucleotide sequence comprising a “sequence encoding a C-terminal portion or fragment of STRC” may be as follows:

(SEQ ID NO: 17)
5′ATGgctctcagcctctggcccctgctgctgctgctgctgctgctg
ctgctgctgtcctttgca
ATCAAGATAGCTACAAGGAAGTATCTTGGCAAACAA
AACGTTTATGATATTGGAGTCGAAAGAGATCACAAC
TTTGCTCTGAAGAACGGATTCATAGCTTCTAAT
TGCTTTAGTCCTGTGC
TGTGGGATCTGCTCCAGAGGGAAAAGAGTGTTTGGGCCCTGCAGATT
CTAGTGCAGGCGTACCTGCATATGCCCCCAGAAAACCTCCAGCAGCT
GGTGCTTTCAGCAGAGAGGGAGGCTGCACAGGGCTTCCTGACACTCA
TGCTGCAGGGGAAGCTGCAGGGGAAGCTGCAGGTACCACCATCCGAG
GAGCAGGCCCTGGGTCGCCTGACAGCCCTGCTGCTCCAGCGGTACCC
ACGCCTCACCTCCCAGCTCTTCATTGACCTGTCACCACTCATCCCTT
TCTTGGCTGTCTCTGACCTGATGCGCTTCCCACCATCCCTGTTAGCC
AACGACAGTGTCCTGGCTGCCATCCGGGATTACAGCCCAGGAATGAG
GCCTGAACAGAAGGAGGCTCTGGCAAAGCGACTGCTGGCCCCTGAAC
TGTTTGGGGAAGTGCCTGCCTGGCCCCAGGAGCTGCTGTGGGCAGTG
CTGCCCCTGCTCCCCCACCTCCCTCTGGAGAACTTTTTGCAGCTCAG
CCCTCACCAGATCCAGGCCCTGGAGGATAGCTGGCCAGCAGCAGGTC
TGGGGCCAGGGCATGCCCGCCATGTGCTGCGCAGCCTGGTAAACCAG
AGTGTCCAGGATGGTGAGGAGCAGGTACGCAGGCTTGGGCCCCTCGC
CTGTTTCCTGAGCCCTGAGGAGCTGCAGAGCCTAGTGCCCCTGAGTG
ATCCAACGGGGCCAGTAGAACGGGGGCTGCTGGAATGTGCAGCCAAT
GGGACCCTCAGCCCAGAAGGACGGGTGGCATATGAACTTCTGGGTGT
GTTGCGCTCATCTGGAGGAGCGGTGCTGAGCCCCCGGGAGCTGCGGG
TCTGGGCCCCTCTCTTCTCTCAGCTGGGCCTCCGCTTCCTTCAGGAG
CTGTCAGAGCCCCAGCTTAGAGCCATGCTTCCTGTCCTGCAGGGAAC
TAGTGTTACACCTGCTCAGGCTGTCCTGCTGCTTGGACGGCTCCTTC
CTAGGCACGATCTATCCCTGGAGGAACTCTGCTCCTTGCACCTTCTG
CTACCAGGCCTCAGCCCCCAGACACTCCAGGCCATCCCTAGGCGAGT
CCTGGTCGGGGCTTGTTCCTGCCTGGCCCCTGAACTGTCACGCCTCT
CAGCCTGCCAGACCGCAGCACTGCTGCAGACCTTTCGGGTTAAAGAT
GGTGTTAAAAATATGGGTACAACAGGTGCTGGTCCAGCTGTGTGTAT
CCCTGGTCAGCCTATTCCCACCACCTGGCCAGACTGCCTGCTTCCCC
TGCTCCCATTAAAGCTGCTACAACTGGATTCCTTGGCTCTTCTGGCA
AATCGAAGACGCTACTGGGAGCTGCCCTGGTCTGAGCAGCAGGCACA
GTTTCTCTGGAAGAAGATGCAAGTACCCACCAACCTTACCCTCAGGA
ATCTGCAGGCTCTGGGCACCCTGGCAGGAGGCATGTCCTGTGAGTTT
CTGCAGCAGATCAACTCCATGGTAGACTTCCTTGAAGTGGTGCACAT
GATCTATCAGCTGCCCACTAGAGTTCGAGGGAGCCTGAGGGCCTGTA
TCTGGGCAGAGCTACAGCGGAGGATGGCAATGCCAGAACCAGAATGG
ACAACTGTAGGGCCAGAACTGAACGGGCTGGATAGCAAGCTACTCCT
GGACTTACCGATCCAGTTGATGGACAGACTATCCAATGAATCCATTA
TGTTGGTGGTGGAGCTGGTGCAAAGAGCTCCAGAGCAGCTGCTGGCA
CTGACCCCCCTCCACCAGGCAGCCCTGGCAGAGAGGGCACTACAAAA
CCTGGCTCCAAAGGAGACTCCAGTCTCAGGGGAAGTGCTGGAGACCT
TAGGCCCTTTGGTTGGATTCCTGGGGACAGAGAGCACACGACAGATC
CCCCTACAGATCCTGCTGTCCCATCTCAGTCAGCTGCAAGGCTTCTG
CCTAGGAGAGACATTTGCCACAGAGCTGGGATGGCTGCTATTGCAGG
AGTCTGTTCTTGGGAAACCAGAGTTGTGGAGCCAGGATGAAGTAGAG
CAAGCTGGACGCCTAGTATTCACTCTGTCTACTGAGGCAATTTCCTT
GATCCCCAGGGAGGCCTTGGGTCCAGAGACCCTGGAGCGGCTTCTAG
AAAAGCAGCAGAGCTGGGAGCAGAGCAGAGTTGGACAGCTGTGTAGG
GAGCCACAGCTTGCTGCCAAGAAAGCAGCCCTGGTAGCAGGGGTGGT
GCGACCAGCTGCTGAGGATCTTCCAGAACCTGTGCCAAATTGTGCAG
ATGTACGAGGGACATTCCCAGCAGCCTGGTCTGCAACCCAGATTGCA
GAGATGGAGCTCTCAGACTTTGAGGACTGCCTGACATTATTTGCAGG
AGACCCAGGACTTGGGCCTGAGGAACTGCGGGCAGCCATGGGCAAAG
CAAAACAGTTGTGGGGTCCCCCCCGGGGATTTCGTCCTGAGCAGATC
CTGCAGCTTGGTAGGCTCTTAATAGGTCTAGGAGATCGGGAACTACA
GGAGCTGATCCTAGTGGACTGGGGAGTGCTGAGCACCCTGGGGCAGA
TAGATGGCTGGAGCACCACTCAGCTCCGCATTGTGGTCTCCAGTTTC
CTACGGCAGAGTGGTCGGCATGTGAGCCACCTGGACTTCGTTCATCT
GACAGCGCTGGGTTATACTCTCTGTGGACTGCGGCCAGAGGAGCTCC
AGCACATCAGCAGTTGGGAGTTCAGCCAAGCAGCTCTCTTCCTCGGC
ACCCTGCATCTCCAGTGCTCTGAGGAACAACTGGAGGTTCTGGCCCA
CCTACTTGTACTGCCTGGTGGGTTTGGCCCAATCAGTAACTGGGGGC
CTGAGATCTTCACTGAAATTGGCACCATAGCAGCTGGGATCCCAGAC
CTGGCTCTTTCAGCACTGCTGCGGGGACAGATCCAGGGCGTTACTCC
TCTTGCCATTTCTGTCATCCCTCCTCCTAAATTTGCTGTGGTGTTTA
GTCCCATCCAACTATCTAGTCTCACCAGTGCTCAGGCTGTGGCTGTC
ACTCCTGAGCAAATGGCCTTTCTGAGTCCTGAGCAGCGACGAGCAGT
TGCATGGGCCCAACATGAGGGAAAGGAGAGCCCAGAACAGCAAGGTC
GAAGTACAGCCTGGGGCCTCCAGGACTGGTCACGACCTTCCTGGTCC
CTGGTATTGACTATCAGCTTCCTTGGCCACCTGCTA [symbol]
gagcagaaactcatctca
ggaaaggatctgTAATAG.

An exemplary murine nucleotide sequence comprising a “sequence encoding a C-terminal portion or fragment of STRC” may be as follows:

(SEQ ID NO: 19)
5′ATGgctctgagcctccagccccagctgctccttctcctgtcgctc
ctgccgcaggaagtgacttca
ATCAAGATAGCTACAAGGAAGTATCTTGGCAAACAA
AACGTTTATGATATTGGAGTCGAAAGAGATCACAA
CTTTGCTCTGAAGAACGGATTCATAGCTTCTAAT
TGCTTGAGTCCTGTACTGTGGGATCTACTCCAGAGAGAGAAGAGCG
TTTGGGCCCTGAGGACCCTGGTGAAGGCCTACCTGCGCATGCCTCC
AGAAGACCTTCAGCAGCTTGTGCTTTCAGCAGAGATGGAGGCTGCA
CAGGGCTTCCTGACGCTCATGCTTCGTTCCTGGGCTAAGCTGAAGG
TTCAACCATCCGAGGAGCAGGCCATGGGCCGCCTGACAGCCTTGCT
GCTCCAGCGGTACCCACGCCTCACCTCCCAACTCTTTATCGACATG
TCACCGCTCATCCCCTTCCTGGCTGTCCCTGACCTCATGCGCTTCC
CACCGTCCCTTTTGGCCAACGACAGTGTCCTGGCTGCCATCAGGGA
TCACAGCTCAGGAATGAAGCCTGAACAGAAGGAGGCCCTGGCAAAA
CGACTGCTGGCCCCTGAGCTGTTTGGAGAAGTGCCTGATTGGCCCC
AGGAGCTGCTGTGGGCAGCCCTGCCTCTGCTTCCCCATCTGCCTCT
GGAGAGCTTTCTCCAGCTCAGCCCTCACCAGATCCAGGCCCTGGAG
GATAGCTGGCCAGTAGCAGATCTTGGGCCGGGACACGCCCGACATG
TGCTTCGTAGCCTAGTAAACCAGAGCATGGAGGATGGGGAGGAGCA
GGTGCTCAGGCTTGGGTCCCTCGCCTGTTTCCTGAGTCCTGAGGAG
CTACAGAGTCTGGTGCCCTTGAGTGATCCAATGGGGCCTGTAGAAC
AGGGTCTGCTGGAATGTGCGGCCAATGGGACCCTCAGCCCAGAAGG
ACGGGTGGCATATGAACTTCTGGGAGTGTTGCGTTCATCTGGAGGA
ACTGTCTTAAGCCCCCGAGAGCTGAGGGTCTGGGCACCTCTCTTTC
CCCAGCTGGGCCTCCGCTTCCTGCAGGAGCTCTCAGAGACCCAGCT
TAGAGCCATGCTTCCTGCCCTACAGGGAGCCAGTGTCACACCTGCC
CAGGCTGTTCTGTTGTTTGGAAGGCTCCTTCCTAAGCATGATCTGT
CCCTGGAGGAACTCTGCTCCCTGCACCCTCTCCTGCCAGGTCTCAG
CCCCCAGACACTCCAGGCCATCCCTAAGAGAGTTCTGGTTGGTGCT
TGTTCCTGCCTGGGCCCTGAACTGTCAAGGCTTTCAGCTTGCCAGA
TTGCAGCTCTGCTGCAGACCTTTCGGGTAAAAGATGGTGTTAAAAA
TATGGGTGCAGCAGGTGCCGGCTCAGCCGTGTGCATTCCTGGGCAG
CCCACCACTTGGCCAGACTGCCTGCTTCCCCTGCTCCCATTAAAGC
TGCTACAGCTGGACGCTGCAGCTCTTCTGGCAAACCGAAGACTCTA
TCGGCAGCTGCCTTGGTCTGAGCAACAGGCACAGTTTCTCTGGAAG
AAAATGCAAGTGCCTACCAACCTGAGCCTGAGGAATCTGCAGGCTC
TGGGCAACTTGGCAGGAGGCATGACCTGCGAGTTTCTGCAGCAGAT
CAGCTCAATGGTTGACTTTCTTGATGTGGTACACATGCTCTACCAG
CTGCCCACTGGTGTTCGAGAGAGCCTGCGGGCCTGTATCTGGACAG
AGCTACAGCGGAGGATGACAATGCCAGAGCCAGAGCTGACCACCCT
AGGGCCAGAACTGAGTGAACTTGACACAAAGCTACTCCTGGACTTG
CCGATCCAGCTGATGGACAGATTGTCCAATGATTCCATTATGTTGG
TGGTGGAGATGGTCCAAGGCGCTCCAGAGCAGCTGCTGGCACTGAC
CCCACTCCACCAGACAGCCTTGGCAGAGCGAGCACTTAAAAACCTG
GCTCCAAAGGAGACCCCAATCTCCAAAGAAGTGCTGGAGACACTGG
GCCCCTTGGTTGGATTCCTGGGAATAGAGAGCACGCGACGGATCCC
TTTACCCATTCTACTGTCTCATCTCAGTCAGCTGCAGGGCTTCTGC
CTAGGAGAGACATTTGCCACAGAGCTGGGATGGCTGCTGTTGCAGG
AGCCTGTTCTTGGAAAACCAGAATTGTGGAGCCAGGATGAAATAGA
GCAAGCTGGACGCCTAGTATTCACTCTGTCTGCTGAGGCTATTTCC
TCGATCCCCAGGGAGGCTTTGGGCCCAGAGACACTGGAGAGGCTTC
TGGGAAAGCATCAAAGCTGGGAGCAGAGCAGAGTGGGCCATCTGTG
TGGGGAGTCACAGCTTGCCCACAAGAAAGCAGCTCTGGTAGCTGGG
ATTGTGCATCCAGCTGCTGAGGGTCTCCAAGAGCCTGTACCAAACT
GTGCAGACATACGGGGAACCTTCCCAGCGGCCTGGTCTGCGACACA
AATCTCAGAGATGGAACTCTCAGACTTTGAAGACTGCCTGTCACTA
TTTGCTGGAGATCCAGGACTTGGTCCTGAGGAACTACGGGCAGCCA
TGGGCAAGGCCAAGCAGTTGTGGGGTCCCCCTCGAGGATTCCGTCC
GTAGCAGATCTTGCAGCTGGGCCGTCTCCTGATAGGTCTAGGAGAA
CGGGAACTGCAGGAGCTTACCTTGGTGGACTGGGGTGTGCTGAGCA
GCCTGGGGCAAATAGATGGCTGGAGTTCCATGCAGCTCCGAGCCGT
GGTCTCCAGTTTCCTAAGGCAGAGTGGTCGGCATGTGAGCCACCTG
GACTTCATTTATCTGACAGCACTGGGTTACACAGTCTGTGGATTGC
GACCAGAGGAGTTACAGCACATCAGCAGTTGGGAGTTTAGCCAAGC
AGCTCTCTTCCTGGGTAGCTTGCATCTCCCGTGCTCTGAGGAACAG
CTGGAAGTTCTGGCCTATCTCCTTGTGTTGCCTGGTGGCTTTGGCC
CAGTCAGTAACTGGGGGCCTGAGATCTTCACTGAAATTGGCACAAT
AGCAGCTGGCATCCCAGACCTGGCTCTTTCAGCATTACTGCGGGGA
CAGATCCAAGGCCTGACTCCTCTTGCCATTTCTGTCATTCCTGCTC
CCAAGTTTGCAGTGGTCTTCAACCCCATCCAGTTATCTAGTCTCAC
CAGGGGTCAGGCCGTAGCTGTTACTCCTGAACAGCTGGCCTATCTG
AGTCCTGAGCAGCGGCGAGCAGTTGCATGGGCCCAACACGAAGGGA
AGGAGATCCCAGAGCAGCTGGGTCGAAACTCAGCCTGGGGTCTCTA
CGACTGGTTCCAAGCCTCCTGGGCCCTGGCATTGCCCGTCAGCAT
TTTTGGCCACCTATTA [symbol]
gagcagaaactcatctc
agaagaggatctgTAATAG-3′.

By “C-terminal portion or fragment of STRC protein” is meant an amino acid sequence of a C-terminal portion or fragment of the Stereocilin (STRC) polypeptide. An amino acid sequence comprising a “C-terminal portion or fragment of STRC protein” may be preceded with, in a direction from the N-terminal to C-terminal, a methionine (M) (bold at N-terminal end) encoded by the ATG start codon, a signal peptide sequence (lower case, italicized, and underlined), and a C-terminal fragment of intein (C-intein) (bold and underlined). The amino acid sequence may further comprise a linker sequence (bold and italicized) and a Myc tag (lower case) downstream of the “C-terminal portion or fragment of STRC protein.”

An exemplary amino acid sequence comprising a human “C-terminal portion or fragment of STRC protein” may be as follows:

(SEQ ID NO: 18)
Malslwplllllllllllsfa
IKIATRKYLGKQNVYDIGVERDHNFALKNGFIASN
CFSPVLWDLLQREKSVWALQILVQAYLHMPPENLQQLVLSAEREAA
QGFLTLMLQGKLQGKLQVPPSEEQALGRLTALLLQRYPRLTSQLFI
DLSPLIPFLAVSDLMRFPPSLLANDSVLAAIRDYSPGMRPEQKEAL
AKRLLAPELFGEVPAWPQELLWAVLPLLPHLPLENFLQLSPHQIQA
LEDSWPAAGLGPGHARHVLRSLVNQSVQDGEEQVRRLGPLACFLSP
EELQSLVPLSDPTGPVERGLLECAANGTLSPEGRVAYELLGVLRSS
GGAVLSPRELRVWAPLFSQLGLRFLQELSEPQLRAMLPVLQGTSVT
PAQAVLLLGRLLPRHDLSLEELCSLHLLLPGLSPQTLQAIPRRVLV
GACSCLAPELSRLSACQTAALLQTFRVKDGVKNMGTTGAGPAVCIP
GQPIPTTWPDCLLPLLPLKLLQLDSLALLANRRRYWELPWSEQQAQ
FLWKKMQVPTNLTLRNLQALGTLAGGMSCEFLQQINSMVDFLEVVH
MIYQLPTRVRGSLRACIWAELQRRMAMPEPEWTTVGPELNGLDSKL
LLDLPIQLMDRLSNESIMLVVELVQRAPEQLLALTPLHQAALAERA
LQNLAPKETPVSGEVLETLGPLVGFLGTESTRQIPLQILLSHLSQL
QGFCLGETFATELGWLLLQESVLGKPELWSQDEVEQAGRLVFTLST
EAISLIPREALGPETLERLLEKQQSWEQSRVGQLCREPQLAAKKAA
LVAGVVRPAAEDLPEPVPNCADVRGTFPAAWSATQIAEMELSDFED
CLTLFAGDPGLGPEELRAAMGKAKQLWGPPRGFRPEQILQLGRLLI
GLGDRELQELILVDWGVLSTLGQIDGWSTTQLRIVVSSFLRQSGRH
VSHLDFVHLTALGYTLCGLRPEELQHISSWEFSQAALFLGTLHLQC
SEEQLEVLAHLLVLPGGFGPISNWGPEIFTEIGTIAAGIPDLALSA
LLRGQIQGVTPLAISVIPPPKFAVVFSPIQLSSLTSAQAVAVTPEQ
MAFLSPEQRRAVAWAQHEGKESPEQQGRSTAWGLQDWSRPSWSLVL
TISFLGHLL [symbol] eqkliseedl**.

Another exemplary amino acid sequence comprising a murine “C-terminal portion or fragment of STRC protein” may be as follows:

(SEQ ID NO: 20)
Malslqpqlllllsllpqevts
IKIATRKYLGKQNVYDIGVERDHNFALKNGFIASN
CFSPVLWDLLQREKSVWALRTLVKAYLRMPPEDLQQLVLSAEMEAA
QGFLTLMLRSWAKLKVQPSEEQAMGRLTALLLQRYPRLTSQLFIDM
SPLIPFLAVPDLMRFPPSLLANDSVLAAIRDHSSGMKPEQKEALAK
RLLAPELFGEVPDWPQELLWAALPLLPHLPLESFLQLSPHQIQALE
DSWPVADLGPGHARHVLRSLVNQSMEDGEEQVLRLGSLACFLSPEE
LQSLVPLSDPMGPVEQGLLECAANGTLSPEGRVAYELLGVLRSSGG
TVLSPRELRVWAPLFPQLGLRFLQELSETQLRAMLPALQGASVTPA
QAVLLFGRLLPKHDLSLEELCSLHPLLPGLSPQTLQAIPKRVLVGA
CSCLGPELSRLSACQIAALLQTFRVKDGVKNMGAAGAGSAVCIPGQ
PTTWPDCLLPLLPLKLLQLDAAALLANRRLYRQLPWSEQQAQFLWK
KMQVPTNLSLRNLQALGNLAGGMTCEFLQQISSMVDFLDVVHMLYQ
LPTGVRESLRACIWTELQRRMTMPEPELTTLGPELSELDTKLLLDL
PIQLMDRLSNDSIMLVVEMVQGAPEQLLALTPLHQTALAERALKNL
APKETPISKEVLETLGPLVGFLGIESTRRIPLPILLSHLSQLQGFC
LGETFATELGWLLLQEPVLGKPELWSQDEIEQAGRLVFTLSAEAIS
SIPREALGPETLERLLGKHQSWEQSRVGHLCGESQLAHKKAALVAG
IVHPAAEGLQEPVPNCADIRGTFPAAWSATQISEMELSDFEDCLSL
FAGDPGLGPEELRAAMGKAKQLWGPPRGFRPEQILQLGRLLIGLGE
RELQELTLVDWGVLSSLGQIDGWSSMQLRAVVSSFLRQSGRHVSHL
DFIYLTALGYTVCGLRPEELQHISSWEFSQAALFLGSLHLPCSEEQ
LEVLAYLLVLPGGFGPVSNWGPEIFTEIGTIAAGIPDLALSALLRG
QIQGLTPLAISVIPAPKFAVVFNPIQLSSLTRGQAVAVTPEQLAYL
SPEQRRAVAWAQHEGKEIPEQLGRNSAWGLYDWFQASWALALPVSI
FGHLL [symbol] eqkliseedl*.

Exemplary “C-terminal STRC polypeptide” sequences are provided below (in the N-terminal to C-terminal direction) for human and murine, respectively.

An exemplary human C-terminal portion of the STRC protein, which does not include the signal peptide sequence, may be as follows:

(SEQ ID NO: 23)
CFSPVLWDLLQREKSVWALQILVQAYLHMPPENLQQLVLSAEREAAQGFL
TLMLQGKLQGKLQVPPSEEQALGRLTALLLQRYPRLTSQLFIDLSPLIPF
LAVSDLMRFPPSLLANDSVLAAIRDYSPGMRPEQKEALAKRLLAPELFGE
VPAWPQELLWAVLPLLPHLPLENFLQLSPHQIQALEDSWPAAGLGPGHAR
HVLRSLVNQSVQDGEEQVRRLGPLACFLSPEELQSLVPLSDPTGPVERGL
LECAANGTLSPEGRVAYELLGVLRSSGGAVLSPRELRVWAPLFSQLGLRF
LQELSEPQLRAMLPVLQGTSVTPAQAVLLLGRLLPRHDLSLEELCSLHLL
LPGLSPQTLQAIPRRVLVGACSCLAPELSRLSACQTAALLQTFRVKDGVK
NMGTTGAGPAVCIPGQPIPTTWPDCLLPLLPLKLLQLDSLALLANRRRYW
ELPWSEQQAQFLWKKMQVPTNLTLRNLQALGTLAGGMSCEFLQQINSMVD
FLEVVHMIYQLPTRVRGSLRACIWAELQRRMAMPEPEWTTVGPELNGLDS
KLLLDLPIQLMDRLSNESIMLVVELVQRAPEQLLALTPLHQAALAERALQ
NLAPKETPVSGEVLETLGPLVGFLGTESTRQIPLQILLSHLSQLQGFCLG
ETFATELGWLLLQESVLGKPELWSQDEVEQAGRLVFTLSTEAISLIPREA
LGPETLERLLEKQQSWEQSRVGQLCREPQLAAKKAALVAGVVRPAAEDLP
EPVPNCADVRGTFPAAWSATQIAEMELSDFEDCLTLFAGDPGLGPEELRA
AMGKAKQLWGPPRGFRPEQILQLGRLLIGLGDRELQELILVDWGVLSTLG
QIDGWSTTQLRIVVSSFLRQSGRHVSHLDFVHLTALGYTLCGLRPEELQH
ISSWEFSQAALFLGTLHLQCSEEQLEVLAHLLVLPGGFGPISNWGPEIFT
EIGTIAAGIPDLALSALLRGQIQGVTPLAISVIPPPKFAVVFSPIQLSSL
TSAQAVAVTPEQMAFLSPEQRRAVAWAQHEGKESPEQQGRSTAWGLQDWS
RPSWSLVLTISFLGHLL.

An exemplary murine C-terminal portion of the STRC protein, which does not include the signal peptide sequence but does include hydrophobic regions of at least 16 residues as underlined, may be as follows:

(SEQ ID NO: 24)
CFSPVLWDLLQREKSVWALRTLVKAYLRMPPEDLQQLVLSAEMEAAQGFL
TLMLRSWAKLKVQPSEEQAMGRLTALLLQRYPRLTSQLFIDMSPLIPFLA
VPDLMRFPPSLLANDSVLAAIRDHSSGMKPEQKEALAKRLLAPELFGEVP
DWPQELLWAALPLLPHLPLESFLQLSPHQIQALEDSWPVADLGPGHARHV
LRSLVNQSMEDGEEQVLRLGSLACFLSPEELQSLVPLSDPMGPVEQGLLE
CAANGTLSPEGRVAYELLGVLRSSGGTVLSPRELRVWAPLFPQLGLRFLQ
ELSETQLRAMLPALQGASVTPAQAVLLFGRLLPKHDLSLEELCSLHPLLP
GLSPQTLQAIPKRVLVGACSCLGPELSRLSACQIAALLQTFRVKDGVKNM
GAAGAGSAVCIPGQPTTWPDCLLPLLPLKLLQLDAAALLANRRLYRQLPW
SEQQAQFLWKKMQVPTNLSLRNLQALGNLAGGMTCEFLQQISSMVDFLDV
VHMLYQLPTGVRESLRACIWTELQRRMTMPEPELTTLGPELSELDTKLLL
DLPIQLMDRLSNDSIMLVVEMVQGAPEQLLALTPLHQTALAERALKNLAP
KETPISKEVLETLGPLVGFLGIESTRRIPLPILLSHLSQLQGFCLGETFA
TELGWLLLQEPVLGKPELWSQDEIEQAGRLVFTLSAEAISSIPREALGPE
TLERLLGKHQSWEQSRVGHLCGESQLAHKKAALVAGIVHPAAEGLQEPVP
NCADIRGTFPAAWSATQISEMELSDFEDCLSLFAGDPGLGPEELRAAMGK
AKQLWGPPRGFRPEQILQLGRLLIGLGERELQELTLVDWGVLSSLGQIDG
WSSMQLRAVVSSFLRQSGRHVSHLDFIYLTALGYTVCGLRPEELQHISSW
EFSQAALFLGSLHLPCSEEQLEVLAYLLVLPGGFGPVSNWGPEIFTEIGT
IAAGIPDLALSALLRGQIQGLTPLAISVIPAPKFAVVFNPIQLSSLTRGQ
AVAVTPEQLAYLSPEQRRAVAWAQHEGKEIPEQLGRNSAWGLYDWFQASW
ALALPVSIFGHLL.

Ligation of the N-terminal portion of STRC protein and the C-terminal portion of STRC protein may occur, such as through a peptide bond, thereby resulting in a full-length STRC protein.

An “intein” is a fragment of a protein that is able to excise itself and join the remaining fragments (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Other intein systems may also be used. For example, a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

By “myc tag” is meant a polypeptide protein derived from the c-myc gene, where the synthetic peptide sequence (i.e., EQKLISEEDL (SEQ ID NO:27)) corresponds to the C-terminal amino acids (410-419) of human c-myc protein. This tag allows for further studies such as but not limited to protein isolation (e.g., Western blotting, immunofluorescence, immunoprecipitation).

The sequence of an exemplary AAV9-php.b vector is provided below. An AAV9-php.b vector (5′ to 3′) may provide in some embodiments a nucleotide sequence of at least 70% or greater (e.g., 75%, 80%, 85%, 90%, 95%, 97%, 100%) identity to:

(SEQ ID NO: 28)
5′-CCAATGATACGCGTCGGTGCGGGCCTCTTCGCTATTACGCCAGCTGGCGAAAGGGGGAT
GTGCTGCAAGGCGATTAAGTTGGGTAACGCCAGGGTTTTCCCAGTCACGACGTTGTAAAACG
ACGGCCAGTGAGCGCGCGTAATACGACTCACTATAGGGCGAATTGGGTACATCGACGGTATC
GGGGGAGCTCGCAGGGTCTCCATTTTGAAGCGGGAGGTTTGAACGCGCAGCCGCCATGCCGG
GGTTTTACGAGATTGTGATTAAGGTCCCCAGCGACCTTGACGAGCATCTGCCCGGCATTTCT
GACAGCTTTGTGAACTGGGTGGCCGAGAAGGAATGGGAGTTGCCGCCAGATTCTGACATGGA
TCTGAATCTGATTGAGCAGGCACCCCTGACCGTGGCCGAGAAGCTGCAGCGCGACTTTCTGA
CGGAATGGCGCCGTGTGAGTAAGGCCCCGGAGGCTCTTTTCTTTGTGCAATTTGAGAAGGGA
GAGAGCTACTTCCACATGCACGTGCTCGTGGAAACCACCGGGGTGAAATCCATGGTTTTGGG
ACGTTTCCTGAGTCAGATTCGCGAAAAACTGATTCAGAGAATTTACCGCGGGATCGAGCCGA
CTTTGCCAAACTGGTTCGCGGTCACAAAGACCAGAAATGGCGCCGGAGGCGGGAACAAGGTG
GTGGATGAGTGCTACATCCCCAATTACTTGCTCCCCAAAACCCAGCCTGAGCTCCAGTGGGC
GTGGACTAATATGGAACAGTATTTAAGCGCCTGTTTGAATCTCACGGAGCGTAAACGGTTGG
TGGCGCAGCATCTGACGCACGTGTCGCAGACGCAGGAGCAGAACAAAGAGAATCAGAATCCC
AATTCTGATGCGCCGGTGATCAGATCAAAAACTTCAGCCAGGTACATGGAGCTGGTCGGGTG
GCTCGTGGACAAGGGGATTACCTCGGAGAAGCAGTGGATCCAGGAGGACCAGGCCTCATACA
TCTCCTTCAATGCGGCCTCCAACTCGCGGTCCCAAATCAAGGCTGCCTTGGACAATGCGGGA
AAGATTATGAGCCTGACTAAAACCGCCCCCGACTACCTGGTGGGCCAGCAGCCCGTGGAGGA
CATTTCCAGCAATCGGATTTATAAAATTTTGGAACTAAACGGGTACGATCCCCAATATGCGG
CTTCCGTCTTTCTGGGATGGGCCACGAAAAAGTTCGGCAAGAGGAACACCATCTGGCTGTTT
GGGCCTGCAACTACCGGGAAGACCAACATCGCGGAGGCCATAGCCCACACTGTGCCCTTCTA
CGGGTGCGTAAACTGGACCAATGAGAACTTTCCCTTCAACGACTGTGTGGACAAGATGGTGA
TCTGGTGGGAGGAGGGGAAGATGACCGCCAAGGTCGTGGAGTCGGCCAAAGCCATTCTCGGA
GGAAGCAAGGTGCGCGTGGACCAGAAATGCAAGTCCTCGGCCCAGATAGACCCGACTCCCGT
GATCGTCACCTCCAACACCAATATGTGCGCCGTGATTGACGGGAACTCAACGACCTTCGAAC
ACCAGCAGCCGTTGCAAGACCGGATGTTCAAATTTGAACTCACCCGCCGTCTGGATCATGAC
TTTGGGAAGGTCACCAAGCAGGAAGTCAAAGACTTTTTCCGGTGGGCAAAGGATCACGTGGT
TGAGGTGGAGCATGAATTCTACGTCAAAAAGGGTGGAGCCAAGAAAAGACCCGCCCCCAGTG
ACGCAGATATAAGTGAGCCCAAACGGGTGCGCGAGTCAGTTGCGCAGCCATCGACGTCAGAC
GCGGAAGCTTCGATCAACTACGCGGACAGGTACCAAAACAAATGTTCTCGTCACGTGGGCAT
GAATCTGATGCTGTTTCCCTGCAGACAATGCGAGAGACTGAATCAGAATTCAAATATCTGCT
TCACTCACGGTGTCAAAGACTGTTTAGAGTGCTTTCCCGTGTCAGAATCTCAACCCGTTTCT
GTCGTCAAAAAGGCGTATCAGAAACTGTGCTACATTCATCACATCATGGGAAAGGTGCCAGA
CGCTTGCACTGCTTGCGACCTGGTCAATGTGGACTTGGATGACTGTGTTTCTGAACAATAAA
TGACTTAAACCAGGTATGAGTCGGCTGGATAAATCTAAAGTCATAAACGGCGCTCTGGAATT
ACTCAATGAAGTCGGTATCGAAGGCCTGACGACAAGGAAACTCGCTCAAAAGCTGGGAGTTG
AGCAGCCTACCCTGTACTGGCACGTGAAGAACAAGCGGGCCCTGCTCGATGCCCTGGCCATC
GAGATGCTGGACAGGCATCATACCCACTTCTGCCCCCTGGAAGGCGAGTCATGGCAAGACTT
TCTGCGGAACAACGCCAAGTCATTCCGCTGTGCTCTCCTCTCACATCGCGACGGGGCTAAAG
TGCATCTCGGCACCCGCCCAACAGAGAAACAGTACGAAACCCTGGAAAATCAGCTCGCGTTC
CTGTGTCAGCAAGGCTTCTCCCTGGAGAACGCACTGTACGCTCTGTCCGCCGTGGGCCACTT
TACACTGGGCTGCGTATTGGAGGAACAGGAGCATCAAGTAGCAAAAGAGGAAAGAGAGACAC
CTACCACCGATTCTATGCCCCCACTTCTGAGACAAGCAATTGAGCTGTTCGACCGGCAGGGA
GCCGAACCTGCCTTCCTTTTCGGCCTGGAACTAATCATATGTGGCCTGGAGAAACAGCTAAA
GTGCGAAAGCGGCGGGCCGGCCGACGCCCTTGACGATTTTGACTTAGACATGCTCCCAGCCG
ATGCCCTTGACGACTTTGACCTTGATATGCTGCCTGCTGACGCTCTTGACGATTTTGACCTT
GACATGCTCCCCGGGTAAATGCATGAATTCGATCTAGAGGGCCCTATTCTATAGTGTCACCT
AAATGCTAGAGCTCGCTGATCAGCCTCGACTGTGCCTTCTAGTTGCCAGCCATCTGTTGTTT
GCCCCTCCCCCGTGCCTTCCTTGACCCTGGAAGGTGCCACTCCCACTGTCCTTTCCTAATAA
AATGAGGAAATTGCATCGCATTGTCTGAGTAGGTGTCATTCTATTCTGGGGGGTGGGGTGGG
GCAGGACAGCAAGGGGGAGGATTGGGAAGACAATAGCAGGCATGCTGGGGATGCGGTGGGCT
CTATGGCTTCTGAGGCGGAAAGAACCAGCTGGGGCTCGAATCAAGCTATCAAGTGCCACCTG
ACGTCTCCCTATCAGTGATAGAGAAGTCGACACGTCTCGAGCTCCCTATCAGTGATAGAGAA
GGTACGTCTAGAACGTCTCCCTATCAGTGATAGAGAAGTCGACACGTCTCGAGCTCCCTATC
AGTGATAGAGAAGGTACGTCTAGAACGTCTCCCTATCAGTGATAGAGAAGTCGACACGTCTC
GAGCTCCCTATCAGTGATAGAGAAGGTACGTCTAGAACGTCTCCCTATCAGTGATAGAGAAG
TCGACACGTCTCGAGCTCCCTATCAGTGATAGAGAAGGTACCCCCTATATAAGCAGAGAGAT
CTGTTCAAATTTGAACTGACTAAGCGGCTCCCGCCAGATTTTGGCAAGATTACTAAGCAGGA
AGTCAAGGACTTTTTTGCTTGGGCAAAGGTCAATCAGGTGCCGGTGACTCACGAGTTTAAAG
TTCCCAGGGAATTGGCGGGAACTAAAGGGGCGGAGAAATCTCTAAAACGCCCACTGGGTGAC
GTCACCAATACTAGCTATAAAAGTCTGGAGAAGCGGGCCAGGCTCTCATTTGTTCCCGAGAC
GCCTCGCAGTTCAGACGTGACTGTTGATCCCGCTCCTCTGCGACCGCTAGCTTCGATCAACT
ACGCAGACAGGTACCAAAACAAGTGTTCTCGTCACGTGGGCATTAATCTGATTCTGTTTCCC
TGCAGACAATGCGAGAGAATGAATCAGAACTCAAATATCTGCTTCACTCACGGACAGAAAGA
CTGTTTAGAGTGCTTTCCCGTGTCAGAATCTCAACCCGTTTCTGTCGTCAAAAAGGCGTATC
AGAAACTGTGCTACATTCATCATATCATGGGAAAGGTGCCAGACGCTTGCACTGCCTGCGAT
CTGGTCAATGTGGATTTGGATGACTGCATCTTTGAACAATAAATGACTTAAGCCAGGTATGG
CTGCCGATGGTTATCTTCCAGATTGGCTCGAGGACAACCTTAGTGAAGGAATTCGCGAGTGG
TGGGCTTTGAAACCTGGAGCCCCTCAACCCAAGGCAAATCAACAACATCAAGACAACGCTAG
AGGTCTTGTGCTTCCGGGTTACAAATACCTTGGACCCGGCAACGGACTCGACAAGGGGGAGC
CGGTCAACGCAGCAGACGCGGCGGCCCTCGAGCACGACAAAGCCTACGACCAGCAGCTCAAG
GCCGGAGACAACCCGTACCTCAAGTACAACCACGCCGACGCCGAGTTCCAGGAGCGGCTCAA
AGAAGATACGTCTTTTGGGGGCAACCTCGGGCGAGCAGTCTTCCAGGCCAAAAAGAGGCTTC
TTGAACCTCTTGGTCTGGTTGAGGAAGCGGCTAAGACGGCTCCTGGAAAGAAGAGGCCTGTA
GAGCAGTCTCCTCAGGAACCGGACTCCTCCGCGGGTATTGGCAAATCGGGTGCACAGCCCGC
TAAAAAGAGACTCAATTTCGGTCAGACTGGCGACACAGAGTGAGTCCCAGACCCTCAACCAA
TCGGAGAACCTCCCGCAGCCCCCTCAGGTGTGGGATCTCTTACAATGGCTTCAGGTGGTGGC
GCACCAGTGGCAGACAATAACGAAGGTGCCGATGGAGTGGGTAGTTCCTCGGGAAATTGGCA
TTGCGATTCCCAATGGCTGGGGGACAGAGTCATCACCACCAGCACCCGAACCTGGGCCCTGC
CCACCTACAACAATCACCTCTACAAGCAAATCTCCAACAGCACATCTGGAGGATCTTCAAAT
GACAACGCCTACTTCGGCTACAGCACCCCCTGGGGGTATTTTGACTTCAACAGATTCCACTG
CCACTTCTCACCACGTGACTGGCAGCGACTCATCAACAACAACTGGGGATTCCGGCCTAAGC
GACTCAACTTCAAGCTCTTTAACATTCAGGTCAAAGAGGTTACGGACAACAATGGAGTCAAG
ACCATCGCCAATAACCTTACCAGCACGGTCCAGGTCTTCACGGACTCAGACTATCAGCTCCC
GTACGTGCTCGGGTCGGCTCACGAGGGCTGCCTCCCGCCGTTCCCAGCGGACGTTTTCATGA
TTCCTCAGTACGGGTATCTGACGCTTAATGATGGAAGCCAGGCCGTGGGTCGTTCGTCCTTT
TACTGCCTGGAATATTTCCCGTCGCAAATGCTAAGAACGGGTAACAACTTCCAGTTCAGCTA
CGAGTTTGAGAACGTACCTTTCCATAGCAGCTACGCTCACAGCCAAAGCCTGGACCGACTAA
TGAATCCACTCATCGACCAATACTTGTACTATCTCTCTAGAACTATTAACGGTTCTGGACAG
AATCAACAAACGCTAAAATTCAGTGTGGCCGGACCCAGCAACATGGCTGTCCAGGGAAGAAA
CTAGATACCTGGACCCAGCTACCGACAACAACGTGTCTCAACCACTGTGACTCAAAACAACA
ACAGCGAATTTGCTTGGCCTGGAGCTTCTTCTTGGGCTCTCAATGGACGTAATAGCTTGATG
AATCCTGGACCTGCTATGGCCTCTCACAAAGAAGGAGAGGACCGTTTCTTTCCTTTGTCTGG
ATCTTTAATTTTTGGCAAACAAGGTACTGGCAGAGACAACGTGGATGCGGACAAAGTCATGA
TAACCAACGAAGAAGAAATTAAAACTAGTAACCCGGTAGCAACGGAGTCCTATGGACAAGTG
GCCACAAACCACCAGAGTGCCCAAACTTTGGCGGTGCCTTTTAAGGCACAGGCGCAGACCGG
TTGGGTTCAAAACCAAGGAATACTTCCGGGTATGGTTTGGCAGGACAGAGATGTGTACCTGC
AAGGACCCATTTGGGCCAAAATTCCTCACACGGACGGCAACTTTCACCCTTCTCCGCTGATG
GGAGGGTTTGGAATGAAGCACCCGCCTCCTCAGATCCTCATCAAAAACACACCTGTACCTGC
GGATCCTCCAACGGCCTTCAACAAGGACAAGCTGAACTCTTTCATCACCCAGTATTCTACTG
GTCAAGTCAGCGTGGAGATCGAGTGGGAGCTGCAGAAGGAAAACAGCAAGCGCTGGAACCCG
GAGATCCAGTAGACTTCCAACTATTAGAAGTCTAATAATGTTGAATTTGCTGTTAATACTGA
AGGTGTATATAGTGAACCCCGCCCCATTGGCACCAGATACCTGACTCGTAATCTGTAAGTCG
ACTTGCTTGTTAATCAATAAACCGTTTAATTCGTTTCAGTTGAACTTTGGTCTCTGCGAAGG
GCAATTCGTTTAAACCTGCAGGACTAGAGGTCCTGTATTAGAGGTCACGTGAGTGTTTTGCG
ACATTTTGCGACACCATGTGGTCACGCTGGGTATTTAAGCCCGAGTGAGCACGCAGGGTCTC
CATTTTGAAGCGGGAGGTTTGAACGCGCAGCCGCCAAGCCGAATTCTGCAGATATCACATGT
CCTAGGAACTATCGATCCATCACACTGGCGGCCGCTCGACTAGAGCGGCCGCCACCGCGGTG
GAGCTCCAGCTTTTGCGGACCGAATCGGAAAGAACATGTGAGCAAAAGGCCAGCAAAAGGCC
AGGAACCGTAAAAAGGCCGCGTTGCTGGCGTTTTTCCATAGGCTCCGCCCCCCTGACGAGCA
TCACAAAAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGAACAGTATTTGGTATCTGCGCTC
TGCTGAAGCCAGTTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAATGA
AGTTTTAAATCAATCTAAAGTATATATGAGTAAACTTGGTCTGAGAGTTACCAATGCTTAAT
CAGTGAGGCACCTATCTCAGCGATCTGTCTATTTCGTTCATCCATAGTTGCCTGACTCCCCG
TCGTGTAGATAACTACGATACGGGAGGGCTTACCATCTGGCCCCAGTGCTGCAATGATACCG
CGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAG
CTAGAGTAAGTAGTTCGCCAGTTAATAGTTTGCGCAACGTTGTTGCCATTGCTACAGGCATC
GTGGTGTCACGCTCGTCGTTTGGTATGGCTTCATTCAGCTCCGGTTCCCAACGATCAAGGCG
AGTTACATGATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATACCGCGC
CACATAGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGATCTTC
AGCATCTTTTACTTTCACCAGCGTTTCTGGGTGAGCAAAAACAGGAAGGCAAAATGCCGCAA
AAAAGGGAATAAGGGCGACACGGAAATGTTGAATACTCATACTCTTCCTTTTTCAATATTAT
TGAAGCATTTATCAGGGTTATTGTCTCATGAGCGGATACATATTTGAATGTATTTAGAAAAA
TAAACAAATAGGGGTTCCGCGCACATTTCCCCGAAAAGTGCCACCTGACGTC-3′.

By “administer” is meant providing one or more compositions, constructs, or viral vectors described herein to a subject. By way of example and without limitation, administration can be performed by injection, for example, into the cochlea. Other routes that deliver the composition to cells affected by a mutation can be employed (e.g., intravenous, direct injection, subcutaneous, vascular and/or non-vascular intravenous). Administration can be, for example, by bolus injection or by gradual perfusion over time.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard known methods such as those described herein. As used herein, an alteration may include a change in expression levels of 10% or greater (e.g., 20%, 25%, 30%, 40%, 50%).

By “ameliorate” is meant reduce, decrease, suppress, attenuate, diminish, arrest, or stabilize the development or progression of a disease or condition. Exemplary diseases or conditions may include any disease, such as including those associated with a genetic mutation. In one embodiment, the disease may be associated with a dominant mutation or a recessive mutation, for example, but not limited to, Deafness, Autosomal Recessive 1A (DFNB1A); Deafness, Autosomal Recessive 1B (DFNB1B); Deafness, Autosomal Recessive 2 (DFNB2); Deafness, Autosomal Recessive 4 (DFNB4); Deafness, Autosomal Recessive 6 (DFNB6); Deafness, Autosomal Recessive 7 (DFNB7); Deafness, Autosomal Recessive 8 (DFNB8); Deafness, Autosomal Recessive 9 (DFNB9); Deafness, Autosomal Recessive 10 (DFNB10); Deafness, Autosomal Recessive 16 (DFNB16); Deafness, Autosomal Recessive 24 (DFNB24); Deafness, Autosomal Recessive 25 (DFNB25); Deafness, Autosomal Recessive 59 (DFNB59); Deafness, Autosomal Recessive 98 (DFNB98); Deafness, Autosomal Recessive 110 (DFNB110); Dyskeratosis Congenita, Autosomal Recessive 5 (DKCB5); Deafness, Autosomal Dominant 2B (DFNA2B); Deafness, Autosomal Dominant 3A (DFNA3A); Deafness, Autosomal Dominant 9 (DFNA9); Deafness, Autosomal Dominant 30 (DFNA30); Deafness, Autosomal Dominant 36 (DFNA36); autosomal dominant isolated neurosensory deafness type (DFNA); Dyskeratosis Congenita, Autosomal Dominant 4; Deafness, Autosomal Dominant Nonsyndromic Sensorineural (NSSHL); Wolfram-Like Syndrome, Autosomal Dominant (WFSL).

Additional diseases or conditions that the dual-vector system described herein may treat may include but are not limited to, Dentinogenesis Imperfecta (DGI) 1; Deafness, Autosomal Recessive 16, Deafness-infertility syndrome (DIS), CATSPER-related male infertility; spermatogenic failure 7 (SPGF7); Usher Syndrome, Type I (USH1); Bloom Syndrome (BLM); Cloacal Exstrophy; Pendred Syndrome (PDS); Gyrate Atrophy of Choroid and Retina (GACR); Cataract 41 (CTRCT41); prostate cancer; and breast cancer.

The disclosure may provide cells (e.g., host cells) that are any cell that carries or is capable of carrying a substance of interest. Often a host cell is a mammalian cell (e.g., human, canine, feline, equine, murine). A host cell may receive, for example, an AAV construct, an AAV plasmid, a helper construct, an accessory function vector, or the like. Host cells as may be used herein include progeny of the original cell which has been transfected. A “host cell” of the disclosure may also refer to a cell that has been transfected with an exogenous DNA sequence. Progeny of a single parental cell may not be completely identical in morphology or in genomic or total DNA complement as the original parent, in view of natural, unintentional, or deliberate mutations. The term “transfection” as used here may refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

By “nucleic acid,” “nucleotide sequence,” and “polynucleotide sequence” is meant a deoxyribonucleotide or ribonucleotide polymer in either single- or double-stranded form, and unless otherwise limited, encompass known analogs of natural nucleotides that may function in a similar manner as the naturally occurring nucleotides.

The terms “substantially homologous,” “substantially identical,” or “substantially corresponds to” refers to a characteristic of a nucleic acid or an amino acid sequence, where a selected nucleic acid or amino acid sequence has at least 70% sequence identity as compared to a selected reference nucleic acid or amino acid sequence. The selected sequence and the reference sequence may have at least 75% or greater (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity.

Sequence identity or homology may be determined over the entire length of the sequences that are compared or may be determined by fragments of sequences which may total 25% or less (e.g., 20%, 15%, 10%, 5%) than that of the selected reference sequence. Reference sequences may be a portion of a larger sequence, for example, a portion of a gene or flanking sequence, or a repetitive portion of a chromosome. Two or more polynucleotide sequences may be compared to a reference sequence that typically has at least 18-25 nucleotides, at least 26-35 nucleotides, or at least 40 (e.g., 50, 60, 70, 80, 90, 100, 500, 1000, 1500, 2000) nucleotides. The sequence identity or homology may be determined using well-known sequence comparison algorithms, such as, the FASTA biological sequence alignment/comparison software program (see, e.g., Pearson and Lipman, 1985, 1988).

Polynucleotides, including vectors, plasmids, and the like, are provided here for delivering portions of gene coding sequences of interest, for example, a STRC gene that encodes the stereocilin protein, to a cell. Some embodiments may provide the coding sequences derived from a human STRC gene (see, e.g., NCBI Gene ID: 161497; AC016135.3; NG_011636.1; NCBI Nucleotide ID: NM_153700.2; AF375594) containing 29 exons of 19 kb. Other embodiments may provide a murine (Mus musculus) STRC gene (see, e.g., NCBI Gene ID: 140476; AL845466; AF375593; AK144985; NM_080459). The Stereocilin (STRC) protein or STRC precursor (see, e.g., NCBI Protein ID: NP_714544; AAL35321; BAE26168; NP_536707; UniProtKB/Swiss Prot: Q7RTU9) having 1,809 amino acids, is a large extracellular structural protein found in the stereocilia of outer hair cells in the inner ear.

The STRC gene (e.g., NCBI Accession No. NG_011636; OMIM #603720; MIM 606440) encodes the protein stereocilin (STRC), a large extracellular structural protein found in the stereocilia of outer hair cells of the inner ear, which is associated with horizontal top connectors and tectorial membrane attachment crowns critical for appropriate cohesion and positioning of the stereociliary tips. The STRC gene is located on chromosome 15q15, defines the autosomal recessive DFNB16 deafness locus, and contains 29 exons, encompassing 19 kb. By “STRC polynucleotide” is meant a nucleic acid molecule encoding a STRC polypeptide or fragment thereof.

An underlying cause of autosomal recessive non-syndromic deafness, DFNB16 hearing loss, has been associated with a mutated STRC gene. The human STRC gene sequence (Gene ID: 161497) containing a human nucleotide STRC coding sequence encoding a human stereocilin (STRC) protein (NCBI RefSeq: NP_714544), where a human STRC coding sequence without signal sequence (e.g., SEQ ID NO:29) is as follows:

5′-GTGACTCTGGCCCCTACTGGGCCTCATTCCCTGGACCCTGGTCTCTCCTTCCTGAAGTC
ATTGCTCTCCACTCTGGACCAGGCTCCCCAGGGCTCCCTGAGCCGCTCACGGTTCTTTACAT
TCCTGGCCAACATTTCTTCTTCCTTTGAGCCTGGGAGAATGGGGGAAGGACCAGTAGGAGAG
CCCCCACCTCTCCAGCCGCCTGCTCTGCGGCTCCATGATTTTCTAGTGACACTGAGAGGTAG
CCCCGACTGGGAGCCAATGCTAGGGCTGCTAGGGGATATGCTGGCACTGCTGGGACAGGAGC
AGACTCCCCGAGATTTCCTGGTGCACCAGGCAGGGGTGCTGGGTGGACTTGTGGAGGTGCTG
CTGGGAGCCTTAGTTCCTGGGGGCCCCCCTACCCCAACTCGGCCCCCATGCACCCGTGATGG
GCCGTCTGACTGTGTCCTGGCTGCTGACTGGTTGCCTTCTCTGCTGCTGTTGTTAGAGGGCA
CACGCTGGCAAGCTCTGGTGCAGGTGCAGCCCAGTGTGGACCCCACCAATGCCACAGGCCTC
GATGGGAGGGAGGCAGCTCCTCACTTTTTGCAGGGTCTGTTGGGTTTGCTTACCCCAACAGG
GGAGCTAGGCTCCAAGGAGGCTCTTTGGGGCGGTCTGCTACGCACAGTGGGGGCCCCCCTCT
ATGCTGCCTTTCAGGAGGGGCTGCTCCGTGTCACTCACTCCCTGCAGGATGAGGTCTTCTCC
ATTTTGGGGCAGCCAGAGCCTGATACCAATGGGCAGTGCCAGGGAGGTAACCTTCAACAGCT
GCTCTTATGGGGCGTCCGGCACAACCTTTCCTGGGATGTCCAGGCGCTGGGCTTTCTGTCTG
GATCACCACCCCCACCCCCTGCCCTCCTTCACTGCCTGAGCACGGGCGTGCCTCTGCCCAGA
GCTTCTCAGCCGTCAGCCCACATCAGCCCACGCCAACGGCGAGCCATCACTGTGGAGGCCCT
CTGTGAGAACCACTTAGGCCCAGCACCACCCTACAGCATTTCCAACTTCTCCATCCACTTGC
TCTGCCAGCACACCAAGCCTGCCACTCCACAGCCCCATCCCAGCACCACTGCCATCTGCCAG
ACAGCTGTGTGGTATGCAGTGTCCTGGGCACCAGGTGCCCAAGGCTGGCTACAGGCCTGCCA
CGACCAGTTTCCTGATGAGTTTTTGGATGCGATCTGCAGTAACCTCTCCTTTTCAGCCCTGT
CTGGCTCCAACCGCCGCCTGGTGAAGCGGCTCTGTGCTGGCCTGCTCCCACCCCCTACCAGC
TGCCCTGAAGGCCTGCCCCCTGTTCCCCTCACCCCAGACATCTTTTGGGGCTGCTTCTTGGA
GAATGAGACTCTGTGGGCTGAGCGACTGTGTGGGGAGGCAAGTCTACAGGCTGTGCCCCCCA
GCAACCAGGCTTGGGTCCAGCATGTGTGCCAGGGCCCCACCCCAGATGTCACTGCCTCCCCA
CCATGCCACATTGGACCCTGTGGGGAACGCTGCCCGGATGGGGGCAGCTTCCTGGTGATGGT
CTGTGCCAATGACACCATGTATGAGGTCCTGGTGCCCTTCTGGCCTTGGCTAGCAGGCCAAT
GCAGGATAAGTCGTGGGGGCAATGACACTTGCTTCCTAGAAGGGCTGCTGGGCCCCCTTCTG
CCCTCTCTGCCACCACTGGGACCATCCCCACTCTGTCTGACCCCTGGCCCCTTCCTCCTTGG
CATGCTATCCCAGTTGCCACGCTGTCAGTCCTCTGTCCCAGCTCTTGCTCACCCCACACGCC
TACACTATCTCCTCCGCCTGCTGACCTTCCTCTTGGGTCCAGGGGCTGGGGGCGCTGAGGCC
CAGGGGATGCTGGGTCGGGCCCTACTGCTCTCCAGTCTCCCAGACAACTGCTCCTTCTGGGA
TGCCTTTCGCCCAGAGGGCCGGCGCAGTGTGCTACGGACGATTGGGGAATACCTGGAACAAG
ATGAGGAGCAGCCAACCCCATCAGGCTTTGAACCCACTGTCAACCCCAGCTCTGGTATAAGC
AAGATGGAGCTGCTGGCCTGCTTTAGTCCTGTGCTGTGGGATCTGCTCCAGAGGGAAAAGAG
TGTTTGGGCCCTGCAGATTCTAGTGCAGGCGTACCTGCATATGCCCCCAGAAAACCTCCAGC
AGCTGGTGCTTTCAGCAGAGAGGGAGGCTGCACAGGGCTTCCTGACACTCATGCTGCAGGGG
AAGCTGCAGGGGAAGCTGCAGGTACCACCATCCGAGGAGCAGGCCCTGGGTCGCCTGACAGC
CCTGCTGCTCCAGCGGTACCCACGCCTCACCTCCCAGCTCTTCATTGACCTGTCACCACTCA
TCCCTTTCTTGGCTGTCTCTGACCTGATGCGCTTCCCACCATCCCTGTTAGCCAACGACAGT
GTCCTGGCTGCCATCCGGGATTACAGCCCAGGAATGAGGCCTGAACAGAAGGAGGCTCTGGC
AAAGCGACTGCTGGCCCCTGAACTGTTTGGGGAAGTGCCTGCCTGGCCCCAGGAGCTGCTGT
GGGCAGTGCTGCCCCTGCTCCCCCACCTCCCTCTGGAGAACTTTTTGCAGCTCAGCCCTCAC
CAGATCCAGGCCCTGGAGGATAGCTGGCCAGCAGCAGGTCTGGGGCCAGGGCATGCCCGCCA
TGTGCTGCGCAGCCTGGTAAACCAGAGTGTCCAGGATGGTGAGGAGCAGGTACGCAGGCTTG
GGCCCCTCGCCTGTTTCCTGAGCCCTGAGGAGCTGCAGAGCCTAGTGCCCCTGAGTGATCCA
ACGGGGCCAGTAGAACGGGGGCTGCTGGAATGTGCAGCCAATGGGACCCTCAGCCCAGAAGG
ACGGGTGGCATATGAACTTCTGGGTGTGTTGCGCTCATCTGGAGGAGCGGTGCTGAGCCCCC
GGGAGCTGCGGGTCTGGGCCCCTCTCTTCTCTCAGCTGGGCCTCCGCTTCCTTCAGGAGCTG
TCAGAGCCCCAGCTTAGAGCCATGCTTCCTGTCCTGCAGGGAACTAGTGTTACACCTGCTCA
GGCTGTCCTGCTGCTTGGACGGCTCCTTCCTAGGCACGATCTATCCCTGGAGGAACTCTGCT
CCTTGCACCTTCTGCTACCAGGCCTCAGCCCCCAGACACTCCAGGCCATCCCTAGGCGAGTC
CTGGTCGGGGCTTGTTCCTGCCTGGCCCCTGAACTGTCACGCCTCTCAGCCTGCCAGACCGC
AGCACTGCTGCAGACCTTTCGGGTTAAAGATGGTGTTAAAAATATGGGTACAACAGGTGCTG
GTCCAGCTGTGTGTATCCCTGGTCAGCCTATTCCCACCACCTGGCCAGACTGCCTGCTTCCC
CTGCTCCCATTAAAGCTGCTACAACTGGATTCCTTGGCTCTTCTGGCAAATCGAAGACGCTA
CTGGGAGCTGCCCTGGTCTGAGCAGCAGGCACAGTTTCTCTGGAAGAAGATGCAAGTACCCA
CCAACCTTACCCTCAGGAATCTGCAGGCTCTGGGCACCCTGGCAGGAGGCATGTCCTGTGAG
TTTCTGCAGCAGATCAACTCCATGGTAGACTTCCTTGAAGTGGTGCACATGATCTATCAGCT
GCCCACTAGAGTTCGAGGGAGCCTGAGGGCCTGTATCTGGGCAGAGCTACAGCGGAGGATGG
CAATGCCAGAACCAGAATGGACAACTGTAGGGCCAGAACTGAACGGGCTGGATAGCAAGCTA
CTCCTGGACTTACCGATCCAGTTGATGGACAGACTATCCAATGAATCCATTATGTTGGTGGT
GGAGCTGGTGCAAAGAGCTCCAGAGCAGCTGCTGGCACTGACCCCCCTCCACCAGGCAGCCC
TGGCAGAGAGGGCACTACAAAACCTGGCTCCAAAGGAGACTCCAGTCTCAGGGGAAGTGCTG
GAGACCTTAGGCCCTTTGGTTGGATTCCTGGGGACAGAGAGCACACGACAGATCCCCCTACA
GATCCTGCTGTCCCATCTCAGTCAGCTGCAAGGCTTCTGCCTAGGAGAGACATTTGCCACAG
AGCTGGGATGGCTGCTATTGCAGGAGTCTGTTCTTGGGAAACCAGAGTTGTGGAGCCAGGAT
GAAGTAGAGCAAGCTGGACGCCTAGTATTCACTCTGTCTACTGAGGCAATTTCCTTGATCCC
CAGGGAGGCCTTGGGTCCAGAGACCCTGGAGCGGCTTCTAGAAAAGCAGCAGAGCTGGGAGC
AGAGCAGAGTTGGACAGCTGTGTAGGGAGCCACAGCTTGCTGCCAAGAAAGCAGCCCTGGTA
GCAGGGGTGGTGCGACCAGCTGCTGAGGATCTTCCAGAACCTGTGCCAAATTGTGCAGATGT
ACGAGGGACATTCCCAGCAGCCTGGTCTGCAACCCAGATTGCAGAGATGGAGCTCTCAGACT
TTGAGGACTGCCTGACATTATTTGCAGGAGACCCAGGACTTGGGCCTGAGGAACTGCGGGCA
GCCATGGGCAAAGCAAAACAGTTGTGGGGTCCCCCCCGGGGATTTCGTCCTGAGCAGATCCT
GCAGCTTGGTAGGCTCTTAATAGGTCTAGGAGATCGGGAACTACAGGAGCTGATCCTAGTGG
ACTGGGGAGTGCTGAGCACCCTGGGGCAGATAGATGGCTGGAGCACCACTCAGCTCCGCATT
GTGGTCTCCAGTTTCCTACGGCAGAGTGGTCGGCATGTGAGCCACCTGGACTTCGTTCATCT
GACAGCGCTGGGTTATACTCTCTGTGGACTGCGGCCAGAGGAGCTCCAGCACATCAGCAGTT
GGGAGTTCAGCCAAGCAGCTCTCTTCCTCGGCACCCTGCATCTCCAGTGCTCTGAGGAACAA
CTGGAGGTTCTGGCCCACCTACTTGTACTGCCTGGTGGGTTTGGCCCAATCAGTAACTGGGG
GCCTGAGATCTTCACTGAAATTGGCACCATAGCAGCTGGGATCCCAGACCTGGCTCTTTCAG
CACTGCTGCGGGGACAGATCCAGGGCGTTACTCCTCTTGCCATTTCTGTCATCCCTCCTCCT
AAATTTGCTGTGGTGTTTAGTCCCATCCAACTATCTAGTCTCACCAGTGCTCAGGCTGTGGC
TGTCACTCCTGAGCAAATGGCCTTTCTGAGTCCTGAGCAGCGACGAGCAGTTGCATGGGCCC
AACATGAGGGAAAGGAGAGCCCAGAACAGCAAGGTCGAAGTACAGCCTGGGGCCTCCAGGAC
TGGTCACGACCTTCCTGGTCCCTGGTATTGACTATCAGCTTCCTTGGCCACCTGCTA-3′.

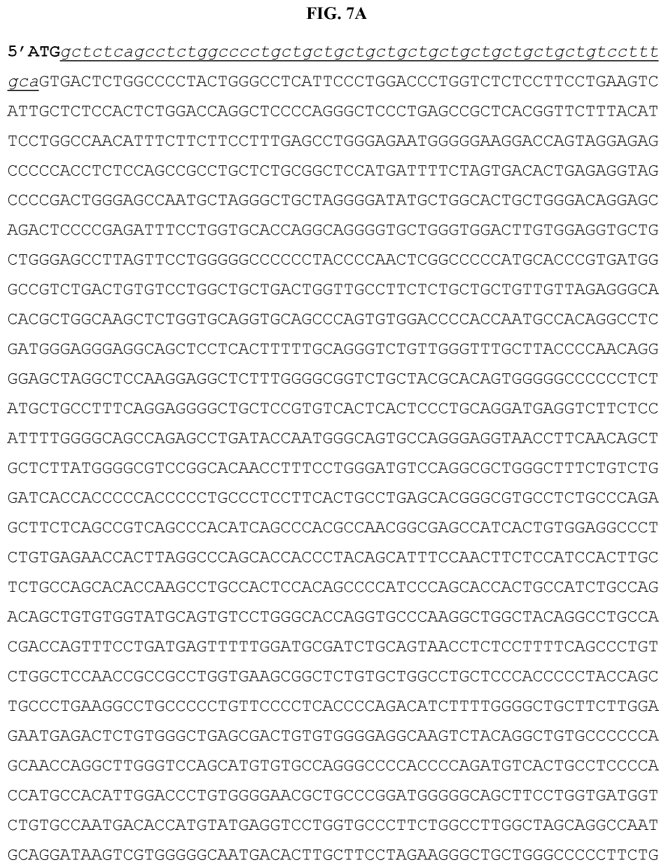

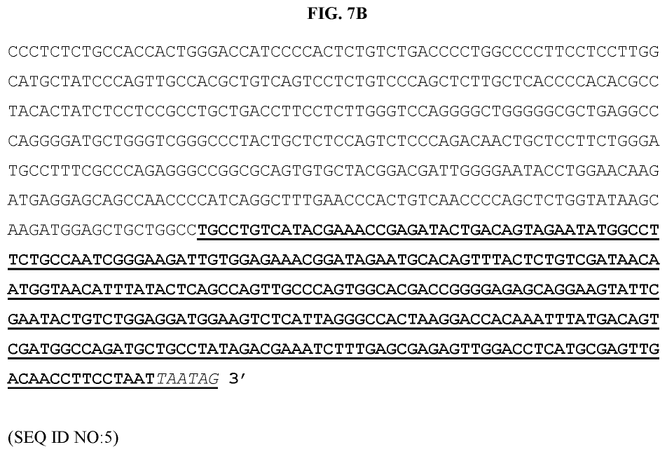

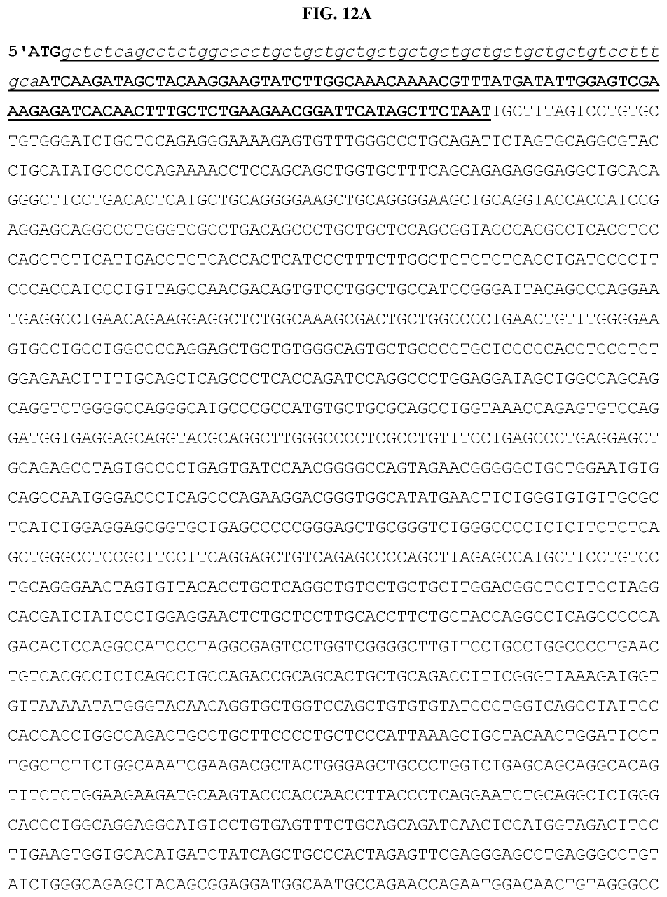

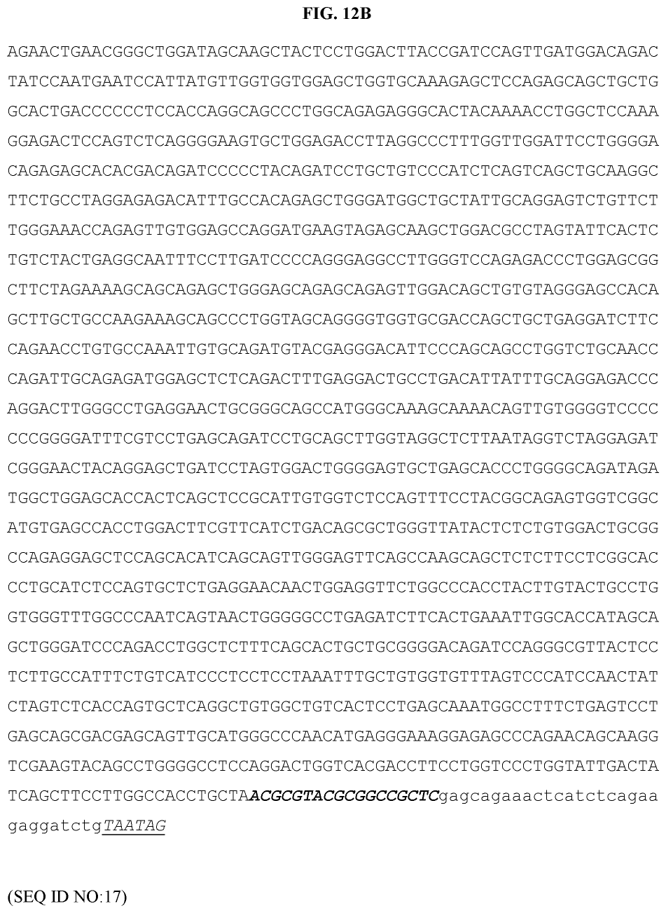

The human STRC coding sequence may be found in the construct of FIGS. 2A-2C and portions of the human STRC coding sequence may be found in the constructs of FIGS. 7A-7B and FIGS. 12A-12B.

The human mRNA sequence (NCBI RefSeq: NM153700) (SEQ ID NO:30) encoding a human STRC protein is as follows:

5′-GCCCTGCCCTCACCTGGCTATCCCACACAGGTGAGAATAACCAGAACTCACCTCCGGTA
CCAGTGTTCACTTGGAAACATGGCTCTCAGCCTCTGGCCCCTGCTGCTGCTGCTGCTGCTGC
TGCTGCTGCTGTCCTTTGCAGTGACTCTGGCCCCTACTGGGCCTCATTCCCTGGACCCTGGT
CTCTCCTTCCTGAAGTCATTGCTCTCCACTCTGGACCAGGCTCCCCAGGGCTCCCTGAGCCG
CTCACGGTTCTTTACATTCCTGGCCAACATTTCTTCTTCCTTTGAGCCTGGGAGAATGGGGG
AAGGACCAGTAGGAGAGCCCCCACCTCTCCAGCCGCCTGCTCTGCGGCTCCATGATTTTCTA
GTGACACTGAGAGGTAGCCCCGACTGGGAGCCAATGCTAGGGCTGCTAGGGGATATGCTGGC
ACTGCTGGGACAGGAGCAGACTCCCCGAGATTTCCTGGTGCACCAGGCAGGGGTGCTGGGTG
GACTTGTGGAGGTGCTGCTGGGAGCCTTAGTTCCTGGGGGCCCCCCTACCCCAACTCGGCCC
CCATGCACCCGTGATGGGCCGTCTGACTGTGTCCTGGCTGCTGACTGGTTGCCTTCTCTGCT
GCTGTTGTTAGAGGGCACACGCTGGCAAGCTCTGGTGCAGGTGCAGCCCAGTGTGGACCCCA
CCAATGCCACAGGCCTCGATGGGAGGGAGGCAGCTCCTCACTTTTTGCAGGGTCTGTTGGGT
TTGCTTACCCCAACAGGGGAGCTAGGCTCCAAGGAGGCTCTTTGGGGCGGTCTGCTACGCAC
AGTGGGGGCCCCCCTCTATGCTGCCTTTCAGGAGGGGCTGCTCCGTGTCACTCACTCCCTGC
AGGATGAGGTCTTCTCCATTTTGGGGCAGCCAGAGCCTGATACCAATGGGCAGTGCCAGGGA
GGTAACCTTCAACAGCTGCTCTTATGGGGCGTCCGGCACAACCTTTCCTGGGATGTCCAGGC
GCTGGGCTTTCTGTCTGGATCACCACCCCCACCCCCTGCCCTCCTTCACTGCCTGAGCACGG
GCGTGCCTCTGCCCAGAGCTTCTCAGCCGTCAGCCCACATCAGCCCACGCCAACGGCGAGCC
ATCACTGTGGAGGCCCTCTGTGAGAACCACTTAGGCCCAGCACCACCCTACAGCATTTCCAA
CTTCTCCATCCACTTGCTCTGCCAGCACACCAAGCCTGCCACTCCACAGCCCCATCCCAGCA
CCACTGCCATCTGCCAGACAGCTGTGTGGTATGCAGTGTCCTGGGCACCAGGTGCCCAAGGC
TGGCTACAGGCCTGCCACGACCAGTTTCCTGATGAGTTTTTGGATGCGATCTGCAGTAACCT
CTCCTTTTCAGCCCTGTCTGGCTCCAACCGCCGCCTGGTGAAGCGGCTCTGTGCTGGCCTGC
TCCCACCCCCTACCAGCTGCCCTGAAGGCCTGCCCCCTGTTCCCCTCACCCCAGACATCTTT
TGGGGCTGCTTCTTGGAGAATGAGACTCTGTGGGCTGAGCGACTGTGTGGGGAGGCAAGTCT
ACAGGCTGTGCCCCCCAGCAACCAGGCTTGGGTCCAGCATGTGTGCCAGGGCCCCACCCCAG
ATGTCACTGCCTCCCCACCATGCCACATTGGACCCTGTGGGGAACGCTGCCCGGATGGGGGC
AGCTTCCTGGTGATGGTCTGTGCCAATGACACCATGTATGAGGTCCTGGTGCCCTTCTGGCC
TTGGCTAGCAGGCCAATGCAGGATAAGTCGTGGGGGCAATGACACTTGCTTCCTAGAAGGGC
TGCTGGGCCCCCTTCTGCCCTCTCTGCCACCACTGGGACCATCCCCACTCTGTCTGACCCCT
GGCCCCTTCCTCCTTGGCATGCTATCCCAGTTGCCACGCTGTCAGTCCTCTGTCCCAGCTCT
TGCTCACCCCACACGCCTACACTATCTCCTCCGCCTGCTGACCTTCCTCTTGGGTCCAGGGG
CTGGGGGCGCTGAGGCCCAGGGGATGCTGGGTCGGGCCCTACTGCTCTCCAGTCTCCCAGAC
AACTGCTCCTTCTGGGATGCCTTTCGCCCAGAGGGCCGGCGCAGTGTGCTACGGACGATTGG
GGAATACCTGGAACAAGATGAGGAGCAGCCAACCCCATCAGGCTTTGAACCCACTGTCAACC
CCAGCTCTGGTATAAGCAAGATGGAGCTGCTGGCCTGCTTTAGTCCTGTGCTGTGGGATCTG
CTCCAGAGGGAAAAGAGTGTTTGGGCCCTGCAGATTCTAGTGCAGGCGTACCTGCATATGCC
CCCAGAAAACCTCCAGCAGCTGGTGCTTTCAGCAGAGAGGGAGGCTGCACAGGGCTTCCTGA
CACTCATGCTGCAGGGGAAGCTGCAGGGGAAGCTGCAGGTACCACCATCCGAGGAGCAGGCC
CTGGGTCGCCTGACAGCCCTGCTGCTCCAGCGGTACCCACGCCTCACCTCCCAGCTCTTCAT
TGACCTGTCACCACTCATCCCTTTCTTGGCTGTCTCTGACCTGATGCGCTTCCCACCATCCC
TGTTAGCCAACGACAGTGTCCTGGCTGCCATCCGGGATTACAGCCCAGGAATGAGGCCTGAA
CAGAAGGAGGCTCTGGCAAAGCGACTGCTGGCCCCTGAACTGTTTGGGGAAGTGCCTGCCTG
GCCCCAGGAGCTGCTGTGGGCAGTGCTGCCCCTGCTCCCCCACCTCCCTCTGGAGAACTTTT
TGCAGCTCAGCCCTCACCAGATCCAGGCCCTGGAGGATAGCTGGCCAGCAGCAGGTCTGGGG
CCAGGGCATGCCCGCCATGTGCTGCGCAGCCTGGTAAACCAGAGTGTCCAGGATGGTGAGGA
GCAGGTACGCAGGCTTGGGCCCCTCGCCTGTTTCCTGAGCCCTGAGGAGCTGCAGAGCCTAG
TGCCCCTGAGTGATCCAACGGGGCCAGTAGAACGGGGGCTGCTGGAATGTGCAGCCAATGGG
ACCCTCAGCCCAGAAGGACGGGTGGCATATGAACTTCTGGGTGTGTTGCGCTCATCTGGAGG
AGCGGTGCTGAGCCCCCGGGAGCTGCGGGTCTGGGCCCCTCTCTTCTCTCAGCTGGGCCTCC
GCTTCCTTCAGGAGCTGTCAGAGCCCCAGCTTAGAGCCATGCTTCCTGTCCTGCAGGGAACT
AGTGTTACACCTGCTCAGGCTGTCCTGCTGCTTGGACGGCTCCTTCCTAGGCACGATCTATC
CCTGGAGGAACTCTGCTCCTTGCACCTTCTGCTACCAGGCCTCAGCCCCCAGACACTCCAGG
CCATCCCTAGGCGAGTCCTGGTCGGGGCTTGTTCCTGCCTGGCCCCTGAACTGTCACGCCTC
TCAGCCTGCCAGACCGCAGCACTGCTGCAGACCTTTCGGGTTAAAGATGGTGTTAAAAATAT
GGGTACAACAGGTGCTGGTCCAGCTGTGTGTATCCCTGGTCAGCCTATTCCCACCACCTGGC
CAGACTGCCTGCTTCCCCTGCTCCCATTAAAGCTGCTACAACTGGATTCCTTGGCTCTTCTG
GCAAATCGAAGACGCTACTGGGAGCTGCCCTGGTCTGAGCAGCAGGCACAGTTTCTCTGGAA
GAAGATGCAAGTACCCACCAACCTTACCCTCAGGAATCTGCAGGCTCTGGGCACCCTGGCAG
GAGGCATGTCCTGTGAGTTTCTGCAGCAGATCAACTCCATGGTAGACTTCCTTGAAGTGGTG
CACATGATCTATCAGCTGCCCACTAGAGTTCGAGGGAGCCTGAGGGCCTGTATCTGGGCAGA
GCTACAGCGGAGGATGGCAATGCCAGAACCAGAATGGACAACTGTAGGGCCAGAACTGAACG
GGCTGGATAGCAAGCTACTCCTGGACTTACCGATCCAGTTGATGGACAGACTATCCAATGAA
TCCATTATGTTGGTGGTGGAGCTGGTGCAAAGAGCTCCAGAGCAGCTGCTGGCACTGACCCC
CCTCCACCAGGCAGCCCTGGCAGAGAGGGCACTACAAAACCTGGCTCCAAAGGAGACTCCAG
TCTCAGGGGAAGTGCTGGAGACCTTAGGCCCTTTGGTTGGATTCCTGGGGACAGAGAGCACA
CGACAGATCCCCCTACAGATCCTGCTGTCCCATCTCAGTCAGCTGCAAGGCTTCTGCCTAGG
AGAGACATTTGCCACAGAGCTGGGATGGCTGCTATTGCAGGAGTCTGTTCTTGGGAAACCAG
AGTTGTGGAGCCAGGATGAAGTAGAGCAAGCTGGACGCCTAGTATTCACTCTGTCTACTGAG
GCAATTTCCTTGATCCCCAGGGAGGCCTTGGGTCCAGAGACCCTGGAGCGGCTTCTAGAAAA
GCAGCAGAGCTGGGAGCAGAGCAGAGTTGGACAGCTGTGTAGGGAGCCACAGCTTGCTGCCA
AGAAAGCAGCCCTGGTAGCAGGGGTGGTGCGACCAGCTGCTGAGGATCTTCCAGAACCTGTG
CCAAATTGTGCAGATGTACGAGGGACATTCCCAGCAGCCTGGTCTGCAACCCAGATTGCAGA
GATGGAGCTCTCAGACTTTGAGGACTGCCTGACATTATTTGCAGGAGACCCAGGACTTGGGC
CTGAGGAACTGCGGGCAGCCATGGGCAAAGCAAAACAGTTGTGGGGTCCCCCCCGGGGATTT
CGTCCTGAGCAGATCCTGCAGCTTGGTAGGCTCTTAATAGGTCTAGGAGATCGGGAACTACA
GGAGCTGATCCTAGTGGACTGGGGAGTGCTGAGCACCCTGGGGCAGATAGATGGCTGGAGCA
CCACTCAGCTCCGCATTGTGGTCTCCAGTTTCCTACGGCAGAGTGGTCGGCATGTGAGCCAC
CTGGACTTCGTTCATCTGACAGCGCTGGGTTATACTCTCTGTGGACTGCGGCCAGAGGAGCT
CCAGCACATCAGCAGTTGGGAGTTCAGCCAAGCAGCTCTCTTCCTCGGCACCCTGCATCTCC
AGTGCTCTGAGGAACAACTGGAGGTTCTGGCCCACCTACTTGTACTGCCTGGTGGGTTTGGC
CCAATCAGTAACTGGGGGCCTGAGATCTTCACTGAAATTGGCACCATAGCAGCTGGGATCCC
AGACCTGGCTCTTTCAGCACTGCTGCGGGGACAGATCCAGGGCGTTACTCCTCTTGCCATTT
CTGTCATCCCTCCTCCTAAATTTGCTGTGGTGTTTAGTCCCATCCAACTATCTAGTCTCACC
AGTGCTCAGGCTGTGGCTGTCACTCCTGAGCAAATGGCCTTTCTGAGTCCTGAGCAGCGACG
AGCAGTTGCATGGGCCCAACATGAGGGAAAGGAGAGCCCAGAACAGCAAGGTCGAAGTACAG
CCTGGGGCCTCCAGGACTGGTCACGACCTTCCTGGTCCCTGGTATTGACTATCAGCTTCCTT
GGCCACCTGCTATGAGCCTGTCTCTACAGTAGAAGGAGATTGTGGGGAGAGAAATCTTAAGT
CATAATGAATAAAGTGCAAACAGAAGTGCATCCTGATTATTTTCAGAAGCTGATGAGGAATA
-3′.

Stereocilin expression has been found only in the sensory hair cells of the inner ear and is associated with the stereocilia, i.e., the stiff microvilli forming the structure for mechanoreception of sound stimulation. The human STRC protein (1,775 amino acids; SEQ ID NO:25), comprising a signal peptide sequence (at amino acids 1-21; underlined) and no linker sequence or Myc tag sequence, and where splice sites may be between Ala708 and Cys709 or Ala933 and Cys934 (bold, underlined), is as follows:

MALSLWPLLLLLLLLLLLSFAVTLAPTGPHSLDPGLSFLKSLLSTLDQAPQGSLSRSRFFTF
LANISSSFEPGRMGEGPVGEPPPLQPPALRLHDFLVTLRGSPDWEPMLGLLGDMLALLGQEQ
TPRDFLVHQAGVLGGLVEVLLGALVPGGPPTPTRPPCTRDGPSDCVLAADWLPSLLLLLEGT
RWQALVQVQPSVDPTNATGLDGREAAPHFLQGLLGLLTPTGELGSKEALWGGLLRTVGAPLY
AAFQEGLLRVTHSLQDEVFSILGQPEPDTNGQCQGGNLQQLLLWGVRHNLSWDVQALGFLSG
SPPPPPALLHCLSTGVPLPRASQPSAHISPRQRRAITVEALCENHLGPAPPYSISNFSIHLL
CQHTKPATPQPHPSTTAICQTAVWYAVSWAPGAQGWLQACHDQFPDEFLDAICSNLSFSALS
GSNRRLVKRLCAGLLPPPTSCPEGLPPVPLTPDIFWGCFLENETLWAERLCGEASLQAVPPS
NQAWVQHVCQGPTPDVTASPPCHIGPCGERCPDGGSFLVMVCANDTMYEVLVPFWPWLAGQC
RISRGGNDTCFLEGLLGPLLPSLPPLGPSPLCLTPGPFLLGMLSQLPRCQSSVPALAHPTRL
HYLLRLLTFLLGPGAGGAEAQGMLGRALLLSSLPDNCSFWDAFRPEGRRSVLRTIGEYLEQD

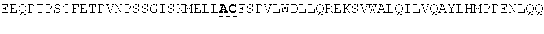

LVLSAEREAAQGFLTLMLQGKLQGKLQVPPSEEQALGRLTALLLQRYPRLTSQLFIDLSPLI
PFLAVSDLMRFPPSLLANDSVLAAIRDYSPGMRPEQKEALAKRLLAPELFGEVPAWPQELLW
AVLPLLPHLPLENFLQLSPHQIQALEDSWPAAGLGPGHARHVLRSLVNQSVQDGEEQVRRLG

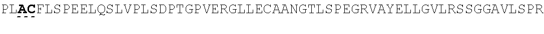

ELRVWAPLFSQLGLRFLQELSEPQLRAMLPVLQGTSVTPAQAVLLLGRLLPRHDLSLEELCS
LHLLLPGLSPQTLQAIPRRVLVGACSCLAPELSRLSACQTAALLQTFRVKDGVKNMGTTGAG
PAVCIPGQPIPTTWPDCLLPLLPLKLLQLDSLALLANRRRYWELPWSEQQAQFLWKKMQVPT
NLTLRNLQALGTLAGGMSCEFLQQINSMVDFLEVVHMIYQLPTRVRGSLRACIWAELQRRMA
MPEPEWTTVGPELNGLDSKLLLDLPIQLMDRLSNESIMLVVELVQRAPEQLLALTPLHQAAL
AERALQNLAPKETPVSGEVLETLGPLVGFLGTESTRQIPLQILLSHLSQLQGFCLGETFATE
LGWLLLQESVLGKPELWSQDEVEQAGRLVFTLSTEAISLIPREALGPETLERLLEKQQSWEQ
SRVGQLCREPQLAAKKAALVAGVVRPAAEDLPEPVPNCADVRGTFPAAWSATQIAEMELSDF
EDCLTLFAGDPGLGPEELRAAMGKAKQLWGPPRGFRPEQILQLGRLLIGLGDRELQELILVD
WGVLSTLGQIDGWSTTQLRIVVSSFLRQSGRHVSHLDFVHLTALGYTLCGLRPEELQHISSW
EFSQAALFLGTLHLQCSEEQLEVLAHLLVLPGGFGPISNWGPEIFTEIGTIAAGIPDLALSA
LLRGQIQGVTPLAISVIPPPKFAVVFSPIQLSSLTSAQAVAVTPEQMAFLSPEQRRAVAWAQ
HEGKESPEQQGRSTAWGLQDWSRPSWSLVLTISFLGHLL**.

The murine (Mus musculus) STRC gene (Gene ID: 140476; CDS at base pairs 79-5508) encoding a murine STRC protein comprising 1,809 amino acids including a putative signal peptide and several hydrophobic portions (NCBI RefSeq: NP_536707), where a murine STRC coding sequence without signal sequence (e.g., SEQ ID NO:31) is as follows:

5′-GCCCCTACTGGGCCTCAGTCTTTGGATGCTGGTCTCTCCCTTCTGAAGTCATTCGTAGC
CACTCTGGACCAAGCTCCTCAGCGTTCCCTCAGCCAGTCACGGTTCTCTGCGTTCCTGGCCA
ACATTTCTTCATCCTTCCAGCTTGGGAGGATGGGGGAGGGACCGGTGGGAGAGCCCCCACCT
CTCCAGCCCCCTGCACTTCGACTTCATGATTTCCTCGTGACACTGAGAGGTAGCCCAGACTG
GGAGCCAATGCTAGGGCTTCTGGGAGATGTGCTGGCACTCCTGGGACAGGAACAGACTCCCC
GGGACTTTTTGGTGCACCAGGCAGGTGTACTGGGTGGACTTGTAGAGGCATTGTTGGGAGCG
TTAGTTCCTGGAGGCCCCCCTGCCCCCACTCGACCCCCATGCACCCGTGATGGCCCTTCTGA
CTGTGTCCTGGCTGCTGATTGGTTGCCTTCTCTGATGTTGTTATTAGAGGGTACACGCTGGC
AGGCCCTGGTGCAGTTGCAGCCCAGTGTGGACCCAACCAATGCCACAGGTCTTGATGGTAGA
GAGCCAGCTCCTCACTTTTTACAGGGTCTGCTGGGCTTGCTTACCCCAGCAGGAGAGTTGGG
CTCTGAGGAGGCTCTTTGGGGTGGTCTGCTGCGCACAGTGGGGGCCCCCCTCTATGCTGCCT
TCCAGGAGGGGCTACTGCGAGTCACTCATTCTCTGCAAGATGAGGTCTTTTCTATTATGGGA
CAGCCAGAGCCTGATGCCAGTGGGCAGTGCCAGGGAGGCAACCTTCAACAGCTGCTTTTATG
GGGCATGCGGAACAACCTTTCTTGGGACGCCCGAGCACTGGGTTTTCTATCTGGATCACCAC
CTCCACCCCCTGCTCTCCTGCACTGCCTGAGCAGAGGTGTGCCTCTGCCCAGGGCTTCCCAG
CCTGCGGCTCACATCAGCCCTCGACAGCGGCGAGCCATCTCTGTGGAGGCCCTCTGCGAGAA
CCACTCAGGCCCAGAGCCACCCTACAGCATCTCCAACTTCTCCATCTACTTGCTCTGCCAGC
ACATCAAGCCTGCCACCCCGCGGCCCCCTCCTACCACCCCACGGCCTCCTCCTACCACCCCA
CAGCCCCCTCCTACCACTACACAGCCCATTCCTGACACTACACAGCCCCCTCCTGTCACCCC
AAGGCCTCCTCCTACCACCCCACAACCCCCTCCTAGCACAGCTGTCATCTGCCAGACAGCTG
TATGGTACGCAGTCTCGTGGGCACCAGGTGCCCGAGGTTGGCTCCAAGCCTGCCATGATCAG
TTTCCTGATCAATTTCTGGATATGATCTGCGGCAACCTCTCATTTTCAGCCCTGTCTGGCCC
CAGTCGTCCTTTGGTAAAGCAGCTCTGTGCTGGCTTGCTCCCACCCCCCACTAGCTGTCCAC
CAGGCCTGATCCCTGTGCCCCTCACCCCAGAAATATTCTGGGGCTGTTTCCTGGAGAATGAG
ACACTGTGGGCTGAACGGTTGTGTGTGGAGGACAGTCTGCAGGCTGTGCCCCCGAGGAACCA
GGCTTGGGTTCAGCATGTGTGTCGGGGCCCCACCTTGGACGCCACTGATTTTCCACCGTGCC
GCGTTGGACCCTGTGGGGAACGCTGCCCAGATGGGGGCAGCTTCCTGCTCATGGTCTGTGCC
AATGACACTCTGTATGAAGCCTTGGTTCCCTTCTGGGCTTGGCTAGCAGGCCAATGCAGAAT
TAGTCGTGGAGGAAATGATACTTGCTTTCTAGAAGGCATGCTGGGCCCCTTGTTGCCCTCTC
TGCCCCCTCTGGGACCATCCCCACTCTGTCTGGCTCCTGGTCCTTTTCTGCTTGGCATGTTA
TCCCAGTTGCCACGCTGTCAGTCCTCCGTGCCAGCCCTCGCCCACCCCACGCGCCTACATTA
CCTCCTGCGCCTACTGACCTTCCTTCTGGGTCCAGGGACTGGGGGTGCCGAGACGCAGGGGA
TGTTAGGTCAAGCCCTGCTGCTCTCTAGTCTCCCAGACAACTGTTCATTCTGGGATGCCTTC
CGCCCAGAGGGCCGGAGAAGTGTACTGAGGACAGTCGGAGAGTACTTGCAGCGGGAAGAGCC
AACCCCACCAGGCTTAGACTCCTCCCTCAGCCTCGGCTCTGGTATGAGCAAGATGGAGCTTC
TGTCCTGCTTCAGTCCTGTACTGTGGGATCTACTCCAGAGAGAGAAGAGCGTTTGGGCCCTG
AGGACCCTGGTGAAGGCCTACCTGCGCATGCCTCCAGAAGACCTTCAGCAGCTTGTGCTTTC
AGCAGAGATGGAGGCTGCACAGGGCTTCCTGACGCTCATGCTTCGTTCCTGGGCTAAGCTGA
AGGTTCAACCATCCGAGGAGCAGGCCATGGGCCGCCTGACAGCCTTGCTGCTCCAGCGGTAC
CCACGCCTCACCTCCCAACTCTTTATCGACATGTCACCGCTCATCCCCTTCCTGGCTGTCCC
TGACCTCATGCGCTTCCCACCGTCCCTTTTGGCCAACGACAGTGTCCTGGCTGCCATCAGGG
ATCACAGCTCAGGAATGAAGCCTGAACAGAAGGAGGCCCTGGCAAAACGACTGCTGGCCCCT
GAGCTGTTTGGAGAAGTGCCTGATTGGCCCCAGGAGCTGCTGTGGGCAGCCCTGCCTCTGCT
TCCCCATCTGCCTCTGGAGAGCTTTCTCCAGCTCAGCCCTCACCAGATCCAGGCCCTGGAGG
ATAGCTGGCCAGTAGCAGATCTTGGGCCGGGACACGCCCGACATGTGCTTCGTAGCCTAGTA
AACCAGAGCATGGAGGATGGGGAGGAGCAGGTGCTCAGGCTTGGGTCCCTCGCCTGTTTCCT
GAGTCCTGAGGAGCTACAGAGTCTGGTGCCCTTGAGTGATCCAATGGGGCCTGTAGAACAGG
GTCTGCTGGAATGTGCGGCCAATGGGACCCTCAGCCCAGAAGGACGGGTGGCATATGAACTT
CTGGGAGTGTTGCGTTCATCTGGAGGAACTGTCTTAAGCCCCCGAGAGCTGAGGGTCTGGGC
ACCTCTCTTTCCCCAGCTGGGCCTCCGCTTCCTGCAGGAGCTCTCAGAGACCCAGCTTAGAG
CCATGCTTCCTGCCCTACAGGGAGCCAGTGTCACACCTGCCCAGGCTGTTCTGTTGTTTGGA
AGGCTCCTTCCTAAGCATGATCTGTCCCTGGAGGAACTCTGCTCCCTGCACCCTCTCCTGCC
AGGTCTCAGCCCCCAGACACTCCAGGCCATCCCTAAGAGAGTTCTGGTTGGTGCTTGTTCCT
GCCTGGGCCCTGAACTGTCAAGGCTTTCAGCTTGCCAGATTGCAGCTCTGCTGCAGACCTTT
CGGGTAAAAGATGGTGTTAAAAATATGGGTGCAGCAGGTGCCGGCTCAGCCGTGTGCATTCC
TGGGCAGCCCACCACTTGGCCAGACTGCCTGCTTCCCCTGCTCCCATTAAAGCTGCTACAGC
TGGACGCTGCAGCTCTTCTGGCAAACCGAAGACTCTATCGGCAGCTGCCTTGGTCTGAGCAA
CAGGCACAGTTTCTCTGGAAGAAAATGCAAGTGCCTACCAACCTGAGCCTGAGGAATCTGCA
GGCTCTGGGCAACTTGGCAGGAGGCATGACCTGCGAGTTTCTGCAGCAGATCAGCTCAATGG
TTGACTTTCTTGATGTGGTACACATGCTCTACCAGCTGCCCACTGGTGTTCGAGAGAGCCTG
CGGGCCTGTATCTGGACAGAGCTACAGCGGAGGATGACAATGCCAGAGCCAGAGCTGACCAC
CCTAGGGCCAGAACTGAGTGAACTTGACACAAAGCTACTCCTGGACTTGCCGATCCAGCTGA
TGGACAGATTGTCCAATGATTCCATTATGTTGGTGGTGGAGATGGTCCAAGGCGCTCCAGAG
CAGCTGCTGGCACTGACCCCACTCCACCAGACAGCCTTGGCAGAGCGAGCACTTAAAAACCT
GGCTCCAAAGGAGACCCCAATCTCCAAAGAAGTGCTGGAGACACTGGGCCCCTTGGTTGGAT
TCCTGGGAATAGAGAGCACGCGACGGATCCCTTTACCCATTCTACTGTCTCATCTCAGTCAG
CTGCAGGGCTTCTGCCTAGGAGAGACATTTGCCACAGAGCTGGGATGGCTGCTGTTGCAGGA
GCCTGTTCTTGGAAAACCAGAATTGTGGAGCCAGGATGAAATAGAGCAAGCTGGACGCCTAG
TATTCACTCTGTCTGCTGAGGCTATTTCCTCGATCCCCAGGGAGGCTTTGGGCCCAGAGACA
CTGGAGAGGCTTCTGGGAAAGCATCAAAGCTGGGAGCAGAGCAGAGTGGGCCATCTGTGTGG
GGAGTCACAGCTTGCCCACAAGAAAGCAGCTCTGGTAGCTGGGATTGTGCATCCAGCTGCTG
AGGGTCTCCAAGAGCCTGTACCAAACTGTGCAGACATACGGGGAACCTTCCCAGCGGCCTGG
TCTGCGACACAAATCTCAGAGATGGAACTCTCAGACTTTGAAGACTGCCTGTCACTATTTGC
TGGAGATCCAGGACTTGGTCCTGAGGAACTACGGGCAGCCATGGGCAAGGCCAAGCAGTTGT
GGGGTCCCCCTCGAGGATTCCGTCCTGAGCAGATCTTGCAGCTGGGCCGTCTCCTGATAGGT
CTAGGAGAACGGGAACTGCAGGAGCTTACCTTGGTGGACTGGGGTGTGCTGAGCAGCCTGGG
GCAAATAGATGGCTGGAGTTCCATGCAGCTCCGAGCCGTGGTCTCCAGTTTCCTAAGGCAGA
GTGGTCGGCATGTGAGCCACCTGGACTTCATTTATCTGACAGCACTGGGTTACACAGTCTGT
GGATTGCGACCAGAGGAGTTACAGCACATCAGCAGTTGGGAGTTTAGCCAAGCAGCTCTCTT
CCTGGGTAGCTTGCATCTCCCGTGCTCTGAGGAACAGCTGGAAGTTCTGGCCTATCTCCTTG
TGTTGCCTGGTGGCTTTGGCCCAGTCAGTAACTGGGGGCCTGAGATCTTCACTGAAATTGGC
ACAATAGCAGCTGGCATCCCAGACCTGGCTCTTTCAGCATTACTGCGGGGACAGATCCAAGG
CCTGACTCCTCTTGCCATTTCTGTCATTCCTGCTCCCAAGTTTGCAGTGGTCTTCAACCCCA
TCCAGTTATCTAGTCTCACCAGGGGTCAGGCCGTAGCTGTTACTCCTGAACAGCTGGCCTAT
CTGAGTCCTGAGCAGCGGCGAGCAGTTGCATGGGCCCAACACGAAGGGAAGGAGATCCCAGA
GCAGCTGGGTCGAAACTCAGCCTGGGGTCTCTACGACTGGTTCCAAGCCTCCTGGGCCCTGG
CATTGCCCGTCAGCATTTTTGGCCACCTATTA-3′

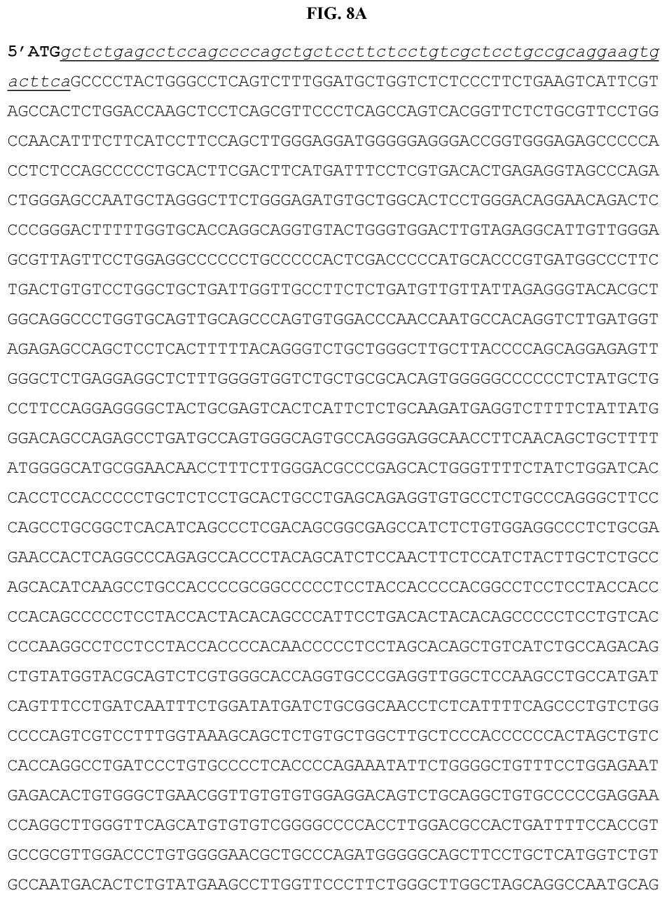

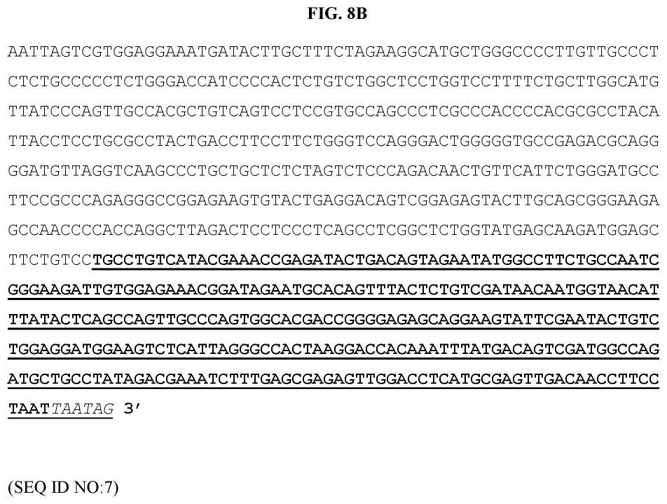

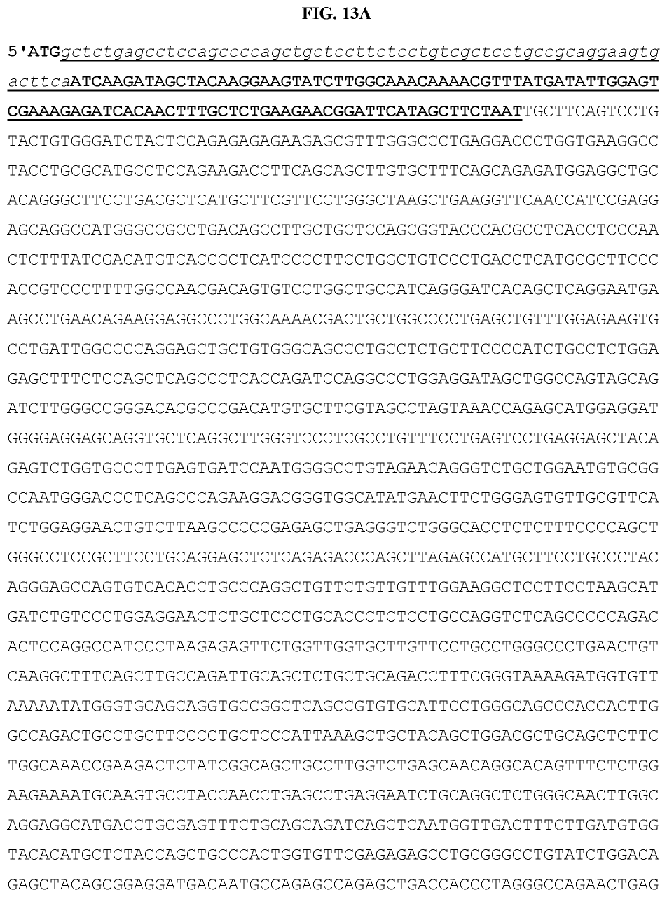

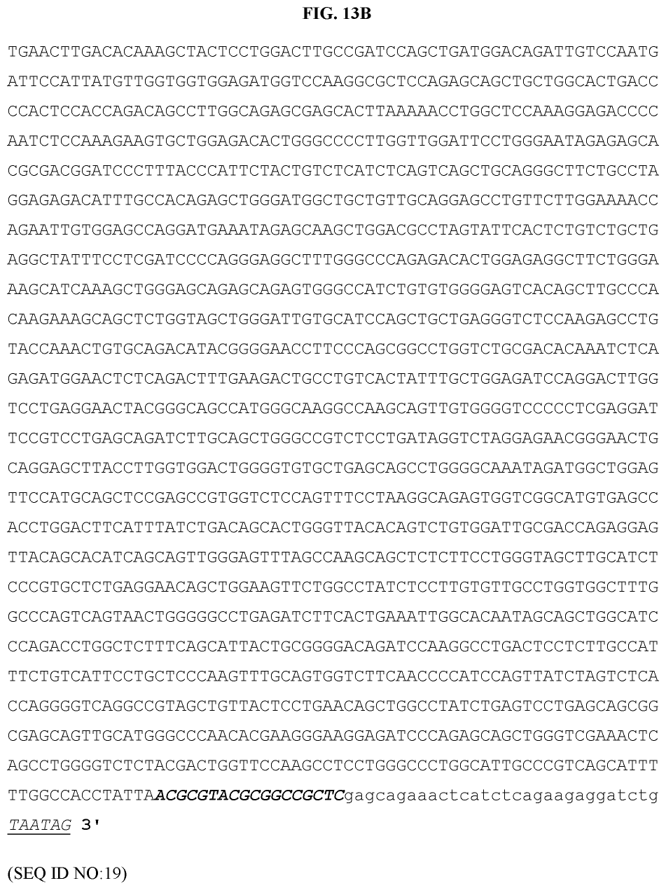

The murine STRC coding sequence may be found in the construct of FIGS. 4A-4D and portions of the murine STRC coding sequence may be found in the constructs of FIGS. 8A-8B and FIGS. 13A-13B.

The murine mRNA sequence (NCBI RefSeq: NM_080459; SEQ ID NO:32), encoding a murine STRC protein, is as follows:

5′-GCCCCGTCTTCACCTGGCTATCCCTTCATGGTGAGCATAGCCAGAACTCACCTCTAGGC
CCAGTGTGCACCTGGAAATATGGCTCTGAGCCTCCAGCCCCAGCTGCTCCTTCTCCTGTCGC
TCCTGCCGCAGGAAGTGACTTCAGCCCCTACTGGGCCTCAGTCTTTGGATGCTGGTCTCTCC
CTTCTGAAGTCATTCGTAGCCACTCTGGACCAAGCTCCTCAGCGTTCCCTCAGCCAGTCACG
GTTCTCTGCGTTCCTGGCCAACATTTCTTCATCCTTCCAGCTTGGGAGGATGGGGGAGGGAC
CGGTGGGAGAGCCCCCACCTCTCCAGCCCCCTGCACTTCGACTTCATGATTTCCTCGTGACA
CTGAGAGGTAGCCCAGACTGGGAGCCAATGCTAGGGCTTCTGGGAGATGTGCTGGCACTCCT
GGGACAGGAACAGACTCCCCGGGACTTTTTGGTGCACCAGGCAGGTGTACTGGGTGGACTTG
TAGAGGCATTGTTGGGAGCGTTAGTTCCTGGAGGCCCCCCTGCCCCCACTCGACCCCCATGC
ACCCGTGATGGCCCTTCTGACTGTGTCCTGGCTGCTGATTGGTTGCCTTCTCTGATGTTGTT
ATTAGAGGGTACACGCTGGCAGGCCCTGGTGCAGTTGCAGCCCAGTGTGGACCCAACCAATG
CCACAGGTCTTGATGGTAGAGAGCCAGCTCCTCACTTTTTACAGGGTCTGCTGGGCTTGCTT
ACCCCAGCAGGAGAGTTGGGCTCTGAGGAGGCTCTTTGGGGTGGTCTGCTGCGCACAGTGGG
GGCCCCCCTCTATGCTGCCTTCCAGGAGGGGCTACTGCGAGTCACTCATTCTCTGCAAGATG
AGGTCTTTTCTATTATGGGACAGCCAGAGCCTGATGCCAGTGGGCAGTGCCAGGGAGGCAAC
CTTCAACAGCTGCTTTTATGGGGCATGCGGAACAACCTTTCTTGGGACGCCCGAGCACTGGG
TTTTCTATCTGGATCACCACCTCCACCCCCTGCTCTCCTGCACTGCCTGAGCAGAGGTGTGC
CTCTGCCCAGGGCTTCCCAGCCTGCGGCTCACATCAGCCCTCGACAGCGGCGAGCCATCTCT
GTGGAGGCCCTCTGCGAGAACCACTCAGGCCCAGAGCCACCCTACAGCATCTCCAACTTCTC
CATCTACTTGCTCTGCCAGCACATCAAGCCTGCCACCCCGCGGCCCCCTCCTACCACCCCAC
GGCCTCCTCCTACCACCCCACAGCCCCCTCCTACCACTACACAGCCCATTCCTGACACTACA
CAGCCCCCTCCTGTCACCCCAAGGCCTCCTCCTACCACCCCACAACCCCCTCCTAGCACAGC
TGTCATCTGCCAGACAGCTGTATGGTACGCAGTCTCGTGGGCACCAGGTGCCCGAGGTTGGC
TCCAAGCCTGCCATGATCAGTTTCCTGATCAATTTCTGGATATGATCTGCGGCAACCTCTCA
TTTTCAGCCCTGTCTGGCCCCAGTCGTCCTTTGGTAAAGCAGCTCTGTGCTGGCTTGCTCCC
ACCCCCCACTAGCTGTCCACCAGGCCTGATCCCTGTGCCCCTCACCCCAGAAATATTCTGGG
GCTGTTTCCTGGAGAATGAGACACTGTGGGCTGAACGGTTGTGTGTGGAGGACAGTCTGCAG
GCTGTGCCCCCGAGGAACCAGGCTTGGGTTCAGCATGTGTGTCGGGGCCCCACCTTGGACGC
CACTGATTTTCCACCGTGCCGCGTTGGACCCTGTGGGGAACGCTGCCCAGATGGGGGCAGCT
TCCTGCTCATGGTCTGTGCCAATGACACTCTGTATGAAGCCTTGGTTCCCTTCTGGGCTTGG
CTAGCAGGCCAATGCAGAATTAGTCGTGGAGGAAATGATACTTGCTTTCTAGAAGGCATGCT
GGGCCCCTTGTTGCCCTCTCTGCCCCCTCTGGGACCATCCCCACTCTGTCTGGCTCCTGGTC
CTTTTCTGCTTGGCATGTTATCCCAGTTGCCACGCTGTCAGTCCTCCGTGCCAGCCCTCGCC
CACCCCACGCGCCTACATTACCTCCTGCGCCTACTGACCTTCCTTCTGGGTCCAGGGACTGG
GGGTGCCGAGACGCAGGGGATGTTAGGTCAAGCCCTGCTGCTCTCTAGTCTCCCAGACAACT
GTTCATTCTGGGATGCCTTCCGCCCAGAGGGCCGGAGAAGTGTACTGAGGACAGTCGGAGAG
TACTTGCAGCGGGAAGAGCCAACCCCACCAGGCTTAGACTCCTCCCTCAGCCTCGGCTCTGG
TATGAGCAAGATGGAGCTTCTGTCCTGCTTCAGTCCTGTACTGTGGGATCTACTCCAGAGAG
AGAAGAGCGTTTGGGCCCTGAGGACCCTGGTGAAGGCCTACCTGCGCATGCCTCCAGAAGAC
CTTCAGCAGCTTGTGCTTTCAGCAGAGATGGAGGCTGCACAGGGCTTCCTGACGCTCATGCT
TCGTTCCTGGGCTAAGCTGAAGGTTCAACCATCCGAGGAGCAGGCCATGGGCCGCCTGACAG
CCTTGCTGCTCCAGCGGTACCCACGCCTCACCTCCCAACTCTTTATCGACATGTCACCGCTC
ATCCCCTTCCTGGCTGTCCCTGACCTCATGCGCTTCCCACCGTCCCTTTTGGCCAACGACAG
TGTCCTGGCTGCCATCAGGGATCACAGCTCAGGAATGAAGCCTGAACAGAAGGAGGCCCTGG
CAAAACGACTGCTGGCCCCTGAGCTGTTTGGAGAAGTGCCTGATTGGCCCCAGGAGCTGCTG
TGGGCAGCCCTGCCTCTGCTTCCCCATCTGCCTCTGGAGAGCTTTCTCCAGCTCAGCCCTCA
CCAGATCCAGGCCCTGGAGGATAGCTGGCCAGTAGCAGATCTTGGGCCGGGACACGCCCGAC
ATGTGCTTCGTAGCCTAGTAAACCAGAGCATGGAGGATGGGGAGGAGCAGGTGCTCAGGCTT
GGGTCCCTCGCCTGTTTCCTGAGTCCTGAGGAGCTACAGAGTCTGGTGCCCTTGAGTGATCC
AATGGGGCCTGTAGAACAGGGTCTGCTGGAATGTGCGGCCAATGGGACCCTCAGCCCAGAAG
GACGGGTGGCATATGAACTTCTGGGAGTGTTGCGTTCATCTGGAGGAACTGTCTTAAGCCCC
CGAGAGCTGAGGGTCTGGGCACCTCTCTTTCCCCAGCTGGGCCTCCGCTTCCTGCAGGAGCT
CTCAGAGACCCAGCTTAGAGCCATGCTTCCTGCCCTACAGGGAGCCAGTGTCACACCTGCCC
AGGCTGTTCTGTTGTTTGGAAGGCTCCTTCCTAAGCATGATCTGTCCCTGGAGGAACTCTGC
TCCCTGCACCCTCTCCTGCCAGGTCTCAGCCCCCAGACACTCCAGGCCATCCCTAAGAGAGT
TCTGGTTGGTGCTTGTTCCTGCCTGGGCCCTGAACTGTCAAGGCTTTCAGCTTGCCAGATTG
CAGCTCTGCTGCAGACCTTTCGGGTAAAAGATGGTGTTAAAAATATGGGTGCAGCAGGTGCC
GGCTCAGCCGTGTGCATTCCTGGGCAGCCCACCACTTGGCCAGACTGCCTGCTTCCCCTGCT
CCCATTAAAGCTGCTACAGCTGGACGCTGCAGCTCTTCTGGCAAACCGAAGACTCTATCGGC
AGCTGCCTTGGTCTGAGCAACAGGCACAGTTTCTCTGGAAGAAAATGCAAGTGCCTACCAAC
CTGAGCCTGAGGAATCTGCAGGCTCTGGGCAACTTGGCAGGAGGCATGACCTGCGAGTTTCT
GCAGCAGATCAGCTCAATGGTTGACTTTCTTGATGTGGTACACATGCTCTACCAGCTGCCCA
CTGGTGTTCGAGAGAGCCTGCGGGCCTGTATCTGGACAGAGCTACAGCGGAGGATGACAATG
CCAGAGCCAGAGCTGACCACCCTAGGGCCAGAACTGAGTGAACTTGACACAAAGCTACTCCT
GGACTTGCCGATCCAGCTGATGGACAGATTGTCCAATGATTCCATTATGTTGGTGGTGGAGA
TGGTCCAAGGCGCTCCAGAGCAGCTGCTGGCACTGACCCCACTCCACCAGACAGCCTTGGCA
GAGCGAGCACTTAAAAACCTGGCTCCAAAGGAGACCCCAATCTCCAAAGAAGTGCTGGAGAC
ACTGGGCCCCTTGGTTGGATTCCTGGGAATAGAGAGCACGCGACGGATCCCTTTACCCATTC
TACTGTCTCATCTCAGTCAGCTGCAGGGCTTCTGCCTAGGAGAGACATTTGCCACAGAGCTG
GGATGGCTGCTGTTGCAGGAGCCTGTTCTTGGAAAACCAGAATTGTGGAGCCAGGATGAAAT
AGAGCAAGCTGGACGCCTAGTATTCACTCTGTCTGCTGAGGCTATTTCCTCGATCCCCAGGG
AGGCTTTGGGCCCAGAGACACTGGAGAGGCTTCTGGGAAAGCATCAAAGCTGGGAGCAGAGC
AGAGTGGGCCATCTGTGTGGGGAGTCACAGCTTGCCCACAAGAAAGCAGCTCTGGTAGCTGG
GATTGTGCATCCAGCTGCTGAGGGTCTCCAAGAGCCTGTACCAAACTGTGCAGACATACGGG
GAACCTTCCCAGCGGCCTGGTCTGCGACACAAATCTCAGAGATGGAACTCTCAGACTTTGAA
GACTGCCTGTCACTATTTGCTGGAGATCCAGGACTTGGTCCTGAGGAACTACGGGCAGCCAT
GGGCAAGGCCAAGCAGTTGTGGGGTCCCCCTCGAGGATTCCGTCCTGAGCAGATCTTGCAGC
TGGGCCGTCTCCTGATAGGTCTAGGAGAACGGGAACTGCAGGAGCTTACCTTGGTGGACTGG
GGTGTGCTGAGCAGCCTGGGGCAAATAGATGGCTGGAGTTCCATGCAGCTCCGAGCCGTGGT
CTCCAGTTTCCTAAGGCAGAGTGGTCGGCATGTGAGCCACCTGGACTTCATTTATCTGACAG
CACTGGGTTACACAGTCTGTGGATTGCGACCAGAGGAGTTACAGCACATCAGCAGTTGGGAG
TTTAGCCAAGCAGCTCTCTTCCTGGGTAGCTTGCATCTCCCGTGCTCTGAGGAACAGCTGGA
AGTTCTGGCCTATCTCCTTGTGTTGCCTGGTGGCTTTGGCCCAGTCAGTAACTGGGGGCCTG
AGATCTTCACTGAAATTGGCACAATAGCAGCTGGCATCCCAGACCTGGCTCTTTCAGCATTA
CTGCGGGGACAGATCCAAGGCCTGACTCCTCTTGCCATTTCTGTCATTCCTGCTCCCAAGTT
TGCAGTGGTCTTCAACCCCATCCAGTTATCTAGTCTCACCAGGGGTCAGGCCGTAGCTGTTA
CTCCTGAACAGCTGGCCTATCTGAGTCCTGAGCAGCGGCGAGCAGTTGCATGGGCCCAACAC
GAAGGGAAGGAGATCCCAGAGCAGCTGGGTCGAAACTCAGCCTGGGGTCTCTACGACTGGTT
CCAAGCCTCCTGGGCCCTGGCATTGCCCGTCAGCATTTTTGGCCACCTATTATGATAACTGT
TCCTTCAGTTGAGGGAGAAAATTTACATCATACTGAACAACTTGTAAATGGAAGTGCATACT
AATTATTCTCAGTAAGTGGATGAGGATTGTGGGTAAAATTCCAATGCATTCCACCCACCTGA
GAACTGTGCTCCTGGCATATACGCCTCTTGCCATCATGAATAACCTCACTGTTTCTTTTCAT
TTCCTACTTCCTCCTCACCACACCAATAGAAATAACACAGACAGCTGCAACATAAT-3′.

The murine STRC protein (1,809 amino acids; SEQ ID NO:26), comprising a signal peptide sequence (at amino acids 1-22; underlined) and no linker sequence or Myc tag sequence, where cleavage of the 22-amino acid signal peptide sequence leaves a protein having 1,787 amino acids with a predicted molecular mass of 194 kD, has the following sequence, where Ser746 and Cys747 and Ala969 and Cys970 splice sites are in bold, underlined text:

MALSLQPQLLLLLSLLPQEVTSAPTGPQSLDAGLSLLKSFVATLDQAPQRSLSQSRFSAFLA
NISSSFQLGRMGEGPVGEPPPLQPPALRLHDFLVTLRGSPDWEPMLGLLGDVLALLGQEQTP
RDFLVHQAGVLGGLVEALLGALVPGGPPAPTRPPCTRDGPSDCVLAADWLPSLMLLLEGTRW
QALVQLQPSVDPTNATGLDGREPAPHFLQGLLGLLTPAGELGSEEALWGGLLRTVGAPLYAA
FQEGLLRVTHSLQDEVFSIMGQPEPDASGQCQGGNLQQLLLWGMRNNLSWDARALGFLSGSP
PPPPALLHCLSRGVPLPRASQPAAHISPRQRRAISVEALCENHSGPEPPYSISNFSIYLLCQ
HIKPATPRPPPTTPRPPPTTPQPPPTTTQPIPDTTQPPPVTPRPPPTTPQPPPSTAVICQTA
VWYAVSWAPGARGWLQACHDQFPDQFLDMICGNLSFSALSGPSRPLVKQLCAGLLPPPTSCP
PGLIPVPLTPEIFWGCFLENETLWAERLCVEDSLQAVPPRNQAWVQHVCRGPTLDATDFPPC
RVGPCGERCPDGGSFLLMVCANDTLYEALVPFWAWLAGQCRISRGGNDTCFLEGMLGPLLPS
LPPLGPSPLCLAPGPFLLGMLSQLPRCQSSVPALAHPTRLHYLLRLLTFLLGPGTGGAETQG
MLGQALLLSSLPDNCSFWDAFRPEGRRSVLRTVGEYLQREEPTPPGLDSSLSLGSGMSKMEL

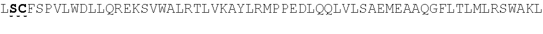

KVQPSEEQAMGRLTALLLQRYPRLTSQLFIDMSPLIPFLAVPDLMRFPPSLLANDSVLAAIR
DHSSGMKPEQKEALAKRLLAPELFGEVPDWPQELLWAALPLLPHLPLESFLQLSPHQIQALE

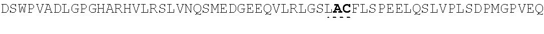

GLLECAANGTLSPEGRVAYELLGVLRSSGGTVLSPRELRVWAPLFPQLGLRFLQELSETQLR
AMLPALQGASVTPAQAVLLFGRLLPKHDLSLEELCSLHPLLPGLSPQTLQAIPKRVLVGACS
CLGPELSRLSACQIAALLQTFRVKDGVKNMGAAGAGSAVCIPGQPTTWPDCLLPLLPLKLLQ
LDAAALLANRRLYRQLPWSEQQAQFLWKKMQVPTNLSLRNLQALGNLAGGMTCEFLQQISSM
VDFLDVVHMLYQLPTGVRESLRACIWTELQRRMTMPEPELTTLGPELSELDTKLLLDLPIQL
MDRLSNDSIMLVVEMVQGAPEQLLALTPLHQTALAERALKNLAPKETPISKEVLETLGPLVG
FLGIESTRRIPLPILLSHLSQLQGFCLGETFATELGWLLLQEPVLGKPELWSQDEIEQAGRL
VFTLSAEAISSIPREALGPETLERLLGKHQSWEQSRVGHLCGESQLAHKKAALVAGIVHPAA
EGLQEPVPNCADIRGTFPAAWSATQISEMELSDFEDCLSLFAGDPGLGPEELRAAMGKAKQL
WGPPRGFRPEQILQLGRLLIGLGERELQELTLVDWGVLSSLGQIDGWSSMQLRAVVSSFLRQ
SGRHVSHLDFIYLTALGYTVCGLRPEELQHISSWEFSQAALFLGSLHLPCSEEQLEVLAYLL
VLPGGFGPVSNWGPEIFTEIGTIAAGIPDLALSALLRGQIQGLTPLAISVIPAPKFAVVFNP
IQLSSLTRGQAVAVTPEQLAYLSPEQRRAVAWAQHEGKEIPEQLGRNSAWGLYDWFQASWAL
ALPVSIFGHLL.

By “stereocilin (STRC) protein” is meant a polypeptide or fragment thereof having at least about 80% amino acid identity (e.g., 82%, 85%, 88%, 90%, 95%, 97%, 98%, 99%, 100%) to, for example, NCBI Accession No. NP_714544 or NP_536707; GenBank No. AAL35321.

“Detect” refers to identifying the presence, absence, or amount of an analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, e.g., green fluorescent protein, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include any pathology, such as a hearing disorder, including but not limited to hearing disorders associated with a recessive mutation, e.g., DFNB16.

By “effective amount” is meant the amount of an agent required to ameliorate the symptoms of a disease relative to an untreated patient. The effective amount of active compound(s) used to practice the therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid sequence or molecule. This portion may contain at least 10% or greater (e.g., 20%, 30%, 40%, 50%, 60%, 70%, 80%, 90%) of the entire length of a reference nucleic acid molecule or polypeptide. A fragment may contain 10 or more (e.g., 20, 30, 40, 50, 60, 70, 80, 90, or 100, 200, 300, 400, 500, 600, 700, 800, 900, 1000, 1100, 1200, 1300, 1400, 1500) nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “identity” is meant the amino acid or nucleic acid sequence identity between a sequence of interest and a reference sequence. By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence may have at least 60% or greater (e.g., 70%, 75%, 80%, 85%, 90%, 95%, 99%, 100%) identity at the amino acid level or nucleic acid level to the sequence or reference sequence used for comparison.

Sequence identity may be measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified (e.g., substantially free of cellular material, viral material, culture medium) when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) of the disclosure that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60% or greater (e.g., 75%, 80%, 90%, 95%, 99%), by weight, free from the proteins and naturally-occurring organic molecules with which it is naturally associated. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “mechanosensation” is meant a response to a mechanical stimulus. Touch, hearing, and balance of examples of the conversion of a mechanical stimulus into a neuronal signal. Mechanosensory input is converted into a response to a mechanical stimulus through a process termed “mechanotransduction.”

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “promoter” is meant a polynucleotide sufficient to direct transcription of a downstream polynucleotide. In some embodiments, polynucleotides described herein may comprise one or more regulatory elements. A person of ordinary skill in the art may select regulatory elements appropriate for use in cells, for example, mammalian or human host cells. Non-limiting examples of regulatory elements include promoters, transcription termination sequences, translation termination sequences, enhancers, and polyadenylation elements. A polynucleotide described herein may comprise a promoter sequence operably linked to a nucleotide sequence encoding a desired polypeptide, such as Stereocilin. Promoters contemplated for use in the subject invention include, but are not limited to, cytomegalovirus (CMV) promoter, SV40 promoter, Rous sarcoma virus (RSV) promoter, chimeric CMV/chicken β actin promoter (CBA), and the truncated form of CBA (smCBA) In some embodiments, the promoter is the CMV promoter.

The phrase “pharmaceutically-acceptable excipient” may include pharmaceutically-acceptable material, composition, or vehicle, such as a liquid or solid filler, diluent, carrier, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Additional suitable carriers and their formulations are described, for example, in the most recent edition of Remington's Pharmaceutical Sciences by E. W. Martin. The amount of the therapeutic agent to be administered varies depending upon the manner and mode of administration, the age and disease status (e.g., the extent of hearing loss present prior to treatment).

By “Stereocilin promoter” is meant a regulatory polynucleotide sequence derived from NCBI Reference Sequence: NG_011636.1 that is sufficient to direct expression of a downstream polynucleotide in an inner hair cell (IHC) or outer hair cell (OHC) in the mature cochlea, the horizontal top connectors joining the apical regions of adjacent stereocilia within a hair bundle, and the links that attach the tallest stereocilia to the overlying tectorial membrane (TM). Stereocilin may also be expressed around the kinocilium of vestibular hair cells and immature OHCs. One embodiment of the disclosure provides for the Stereocilin promoter comprising or consisting of at least 350 or more (e.g., 500, 1000, 2000, 3000, 4000, 5000) base pairs upstream of a Stereocilin coding sequence.

By “reduces” is meant a negative alteration of at least 5% or greater (e.g., 10%, 15%, 20%, 25%, 50%, 75%, 100%).

By “reference” is meant a standard or control condition.

A “reference sequence” is a sequence that is defined and may be used as a basis for sequence comparison. A reference sequence may be a portion of or the entirety of a particular sequence, for example, a fragment of a full-length cDNA or gene sequence, or the complete cDNA or gene sequence. The length of a reference polypeptide sequence may be at least 10 amino acids or greater (e.g., 15, 20, 25, 30, 35, 50, 100), or any integer thereabout or therebetween, for polypeptides. The length of a reference nucleic acid sequence may be at least 50 nucleotides or greater (e.g., 55, 60, 75, 90, 100, 200, 300), or any integer thereabout or therebetween, for nucleic acids.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment or portion thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

Hybridization may occur under, for example, stringent salt concentrations that may ordinarily be less than 750 mM NaCl (e.g., 500 mM; 250 mM) and less than 75 mM trisodium citrate (e.g., 50 mM; 25 mM). Low stringency hybridization may be obtained in the absence of organic solvent, e.g., formamide, while high stringency hybridization may be obtained in the presence of at least 35% formamide (e.g., 50% formamide). Stringent temperature conditions may ordinarily include temperatures of at least 30° C. (e.g., 370 C, 42° C.). Varying additional parameters, such as hybridization time, the concentration of detergent, e.g., sodium dodecyl sulfate (SDS), and the inclusion or exclusion of carrier DNA, are well known to those skilled in the art. Various levels of stringency may be accomplished by combining these various conditions as needed. In one embodiment, hybridization may occur at 30° C. in 750 mM NaCl, 75 mM trisodium citrate, and 1% SDS. In another embodiment, hybridization may occur at 37° C. in 500 mM NaCl, 50 mM trisodium citrate, 1% SDS, 35% formamide, and 100 μg/ml denatured salmon sperm DNA (ssDNA). In a further embodiment, hybridization may occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization may also vary in stringency. Wash stringency conditions may be defined by salt concentration and by temperature. As above, wash stringency may be increased by decreasing salt concentration or by increasing temperature. For example, stringent salt concentration for the wash steps may be less than 30 mM NaCl (e.g., 15 mM) and less than 3 mM trisodium citrate (e.g., 1.5 mM). Stringent temperature conditions for the wash steps may ordinarily include a temperature of at least 25° C. (e.g., 42° C., 68° C.). In yet another embodiment, wash steps may occur at 25° C. in 30 mM NaCl, 3 mM trisodium citrate, and 0.1% SDS. Another embodiment may provide wash steps that occur at 42° C. in 15 mM NaCl, 1.5 mM trisodium citrate, and 0.1% SDS. A further embodiment may provide wash steps that occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “subject” is meant a mammal, including, but not limited to, a human or non-human mammal, such as a bovine, equine, canine, ovine, feline, or murine.

By “STRC protein or polypeptide” is meant a polypeptide having at least about 85% or greater amino acid sequence identity to NCBI Accession No. NP_714544 or GenBank No. AAL35321, or a fragment thereof having sufficient activity to express STRC, which is essential for auditory function.

As used herein, the terms “treat,” “treating,” “treatment,” and the like refer to reducing or ameliorating some disorder and/or symptoms associated therewith. It will be appreciated that, although not precluded, treating a disorder or condition does not require that the disorder, condition, or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

In this disclosure, “comprises,” “comprising,” “containing” and “having” and the like may have the meaning ascribed to them in U.S. patent law and may mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. By “consist essentially” it is meant, for example, that the ingredients include only the listed components along with the normal impurities present in commercial materials and with any other additives present at levels which do not affect the operation of the embodiments disclosed herein, for instance at levels less than 5% by weight or less than 1% or even 0.5% by weight.

As used herein, all ranges of numeric values include the endpoints and all possible values disclosed between the disclosed values. The exact values of all half integral numeric values are also contemplated as specifically disclosed and as limits for all subsets of the disclosed range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Additionally, a range of from 0.1% to 3% specifically discloses a percentage of, for example, 0.1%, 1%, 1.5%, 2.0%, 2.5%, and 3%, or any other numeric value in between. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%. Ranges provided herein are understood to be shorthand for all of the values within the range.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The disclosure is directed to systems (e.g., vectors, recombinant viruses), nucleic acid sequences or constructs encoding split proteins of interest, such as STRC protein, and methods of delivering a protein of interest (e.g., STRC) into a host, host cell, or cell using the nucleic acid sequences or constructs described here. The split protein comprising an amino terminal (N-terminal) fragment of the protein and a carboxy terminal (C-terminal) fragment of the protein are each encoded on different and separate nucleic acid sequences or constructs for delivery into a cell, for example, using a vector (e.g., viral vector, such as adeno-associated virus (AAV) or lentivirus). The N-terminal and C-terminal polypeptide fragments of the protein of interest may be joined together to form a full-length protein of interest, for example, using intein-mediated protein splicing, where the N-terminal and -terminal polypeptide fragments of the protein of interest are joined by a peptide bond. The split site among different species would be located in similar or homologous regions.

### Vector System

Some embodiments may provide a vector (e.g., capsid, plasmid, transplicing plasmid, viral vector, Adenovirus, AAV, AAV genome, lentivirus) system for delivering a coding sequence of a desired full-length protein, comprising at least one vector containing a desired gene construct. FIG. 1 shows a schematic representation of such an exemplary AAV STRC construct. In other embodiments, the vector system may comprise an entire human STRC coding sequence (SEQ ID NO:1), where the nucleotide sequence (FIGS. 2A-2C; SEQ ID NO:33) at the 5′ end begins with an “ATG” start codon (bold) and ends with a stop codon (upper case, italicized, and underlined), and a signal peptide coding sequence (lower case, italicized, and underlined; SEQ ID NO:9) is located upstream of the STRC coding sequence. Optionally included may be a linker sequence (bold and italicized; SEQ ID NO:34) and sequence encoding a myc tag (lower case; SEQ ID NO:35) at the 3′ end, which may be used for subsequent studies, including but not limited to protein isolation (e.g., Western blotting, immunofluorescence, immunoprecipitation). FIG. 3 presents an amino acid sequence (SEQ ID NO:36) encoded by the human STRC nucleotide sequence of FIG. 2, the human STRC protein sequence comprises a methionine (M) encoded by the ATG codon (bold), a signal peptide sequence (lowercase, italicized, and underlined; SEQ ID NO:10), an optional linker sequence (bold and italicized) (N-term-TRTRPL-C-term; SEQ ID NO:37), and an optional Myc tag (lowercase; SEQ ID NO: 27).

In other embodiments, the vector system may comprise an entire murine STRC coding sequence (SEQ ID NO:3), where the nucleotide sequence (FIGS. 4A-4D; SEQ ID NO:38) at the 5′ end begins with an “ATG” start codon (bold) and ends with a stop codon (upper case, italicized, and underlined), and a signal peptide coding sequence (lower case, italicized, and underlined; SEQ ID NO: 11) is located upstream of the STRC coding sequence. Optionally included may be a linker sequence (bold and italicized; SEQ ID NO:34) and sequence encoding a myc tag (lower case; SEQ ID NO:35) at the 3′ end, which may be used for subsequent studies, including but not limited to protein isolation (e.g., Western blotting, immunofluorescence, immunoprecipitation). FIG. 5 presents an amino acid sequence encoded by the murine STRC nucleotide sequence of FIG. 4, the murine STRC protein sequence (SEQ ID NO:39) comprises a methionine (M) encoded by the ATG codon (bold), a signal peptide sequence (lowercase, italicized, and underlined), an optional linker sequence (bold and italicized) (SEQ ID NO:37), and an optional Myc tag (lowercase) (SEQ ID NO: 27).

It is understood that those having ordinary skill in the art would be sufficiently equipped to construct vectors (including, e.g., viruses (bacteriophage, animal viruses, and plant viruses, capsids), plasmids, cosmids, and artificial chromosomes (e.g., YACs)) through standard recombinant techniques, which are described in M R Green and J Sambrook, Molecular Cloning: A Laboratory Manual (2012), 4th Ed.; Ausubel et al., Current Protocols in Molecular Biology (2003), both of which are incorporated herein by reference. Additionally, methods of transferring or delivering DNA to cells are disclosed in, for example, Sung, Y., Kim, S. “Recent advances in the development of gene delivery systems.” Biomater Res 23, 8 (2019); Jin, Lian et al. “Current progress in gene delivery technology based on chemical methods and nano-carriers.” Theranostics 4(3):240-255, 2014; Nayerossadat N, Maedeh T, Ali P A. “Viral and nonviral delivery systems for gene delivery.” Adv Biomed Res. 1:27, 2012; Machida, Curtis A. Viral Vectors for Gene Therapy Methods and Protocols. Humana Press, 2003; Heiser, William C. Gene Delivery to Mammalian Cells. Humana Press, 2004, which are each hereby incorporated by reference.

### Intein-Mediated Protein Trans-Splicing

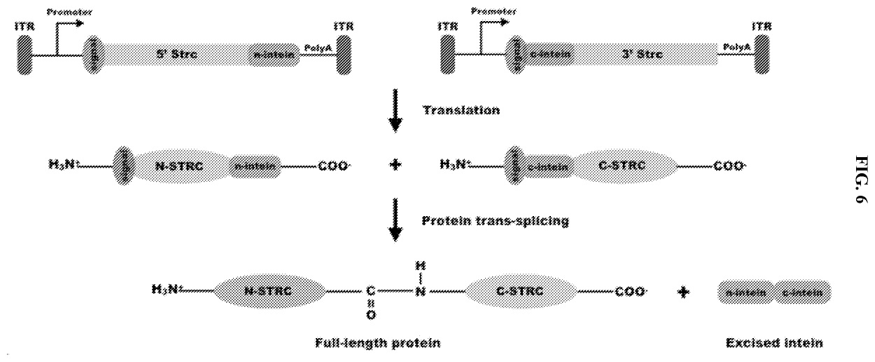

Other embodiments may provide a vector system, comprising a dual-vector system comprising two vectors for each delivering different portions of the same desired protein of interest using inteins. Inteins may be considered to be protein introns, where they are part of a protein that may excise themselves from an amino acid sequence and join the remaining flanking regions (exteins) with a peptide bond by a process known as protein splicing. See, e.g., Mills et al. “Protein Splicing: How Inteins Escape from Precursor Proteins” JBC. 289(21):14498-14505, 2014, incorporated by reference herein in its entirety for intein-mediated protein trans-splicing process. Intein-mediated protein splicing occurs after an mRNA containing an intein has been translated into a protein. See, FIG. 6.

Inteins are a class of enzymes that catalyze reactions of excising themselves out of a host protein-intein fusion, thereby resulting in a mature host protein (the extein) and a separated intein, where a peptide bond ligates the splice junctions between the donor and acceptor inteins. Any of the known inteins may be used in the embodiments of the disclosure including, but not limited to those identified by Perler, F. B. (InBase. The intein database. Nucleic Acids Res. 30:383-384, 2002), all of which may be incorporated by reference herein in their entirety (e.g., Npu-PCC73102 (DnaE-c Intein (Accession No. ZP_00108882); DnaE-n Intein (Accession No. ZP_00111398)) from Nostoc punctiforme PCC 73102 (ATCC® 29133™, all of which may be incorporated by reference herein in their entirety).

In some embodiments of the disclosure, a catalytic subunit of DNA polymerase III DnaE from the cyanobacteria Nostoc punctiforme (Npu) may be used in the split-intein-STRC dual-vector system described herein. For example, there is an alpha subunit of the DNA polymerase III dnaE from the cyanobacteria Nostoc punctiforme (Npu) that is naturally split and located in two genes, dnaE-n and dnaE-c. The N- and C-terminal portions of dnaE are encoded by two separate genes in the genome and on opposite DNA strands. The dnaE-n encoded protein contains an amino-terminal (N-terminal) dnaE fragment (e.g., N-extein) and the amino terminal intein (N-intein), while dnaE-c encodes a protein that contains a carboxy-terminal (C-terminal) dnaE fragment (e.g., C-extein) preceded by a carboxy-terminal intein (C-Intein) entity. N-intein and C-Intein recognize each other, splice themselves out of the amino acid sequence, and simultaneously ligate or fuse the flanking N- and C-terminal exteins of interest through a peptide bond, thereby resulting in the fusion to form the full-length protein of interest.

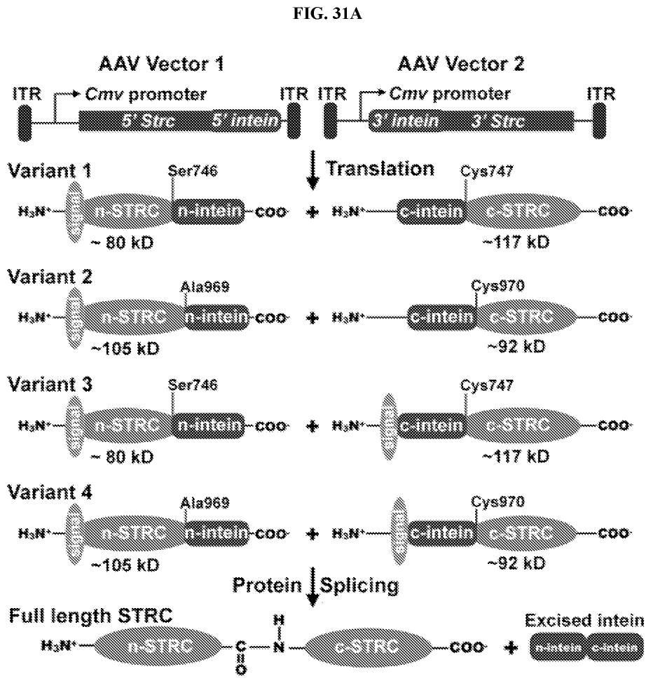

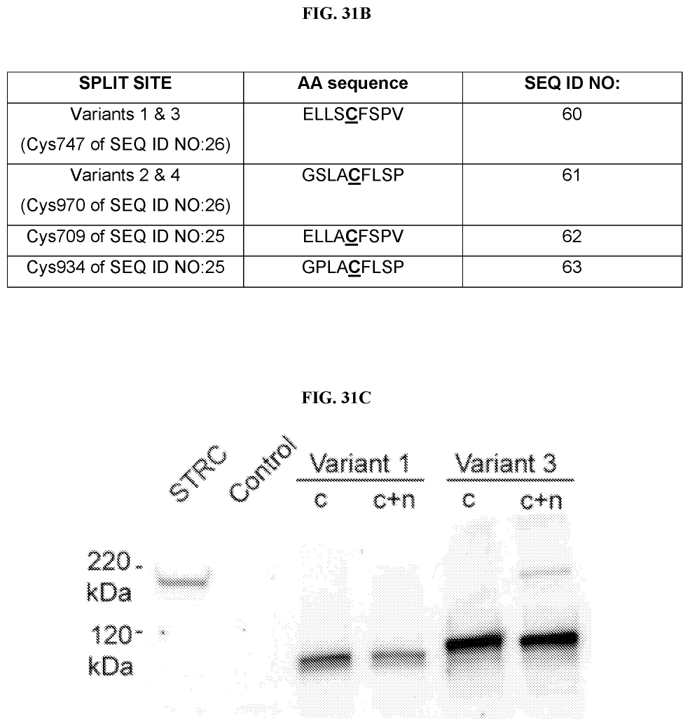

Intein activity may be context dependent, with certain peptide sequences surrounding their ligation or fusion junction (called N- and C-exteins) that may be required for efficient splicing to occur. For example, an amino acid containing a thiol or hydroxyl group (e.g., cysteine (Cys), serine (Ser), threonine (Thr)) as the first residue in the C-extein may be useful for efficient splicing. Native cysteines may be used as splice sites. For example, AAV vectors may take advantage of native cysteines at, for example, positions 747 (Cys747; variants 1 & 3) and 970 (Cys970; variants 2 &4) of SEQ ID NO:26, which may provide a splice site between Ser746 and Cys747 or Ala969 and Cys970 (FIGS. 31A-31B), where the split site may occur in amino acid sequence: ELLSCFSPV (SEQ ID NO:60) or GPLACFLSP (SEQ ID NO:61), or a portion thereof. Another example provides splice sites between Ala708 and Cys709 or Ala933 and Cys934 of SEQ ID NO:25, where the split site may occur in an amino acid sequence: ELLACFSPV (SEQ ID NO:62) or GPLACFLSP (SEQ ID NO:63), or a portion thereof.

One embodiment of the disclosure provides for extein regions with the respective halves of a protein of interest, e.g., stereocilin. For example, the extein regions may comprise an N-terminal stereocilin and a C-terminal stereocilin, which when fused through a peptide bond, make up a full-length stereocilin (e.g., NCBI: NM_153700; NP_714544; NM_080459; NP_536707; GenBank: BK000138; AF375594; DAA00085; AF375593; AAL35321). See, FIG. 6. Another embodiment provides for different split-sites for stereocilin, where the N-terminal portion and the C-terminal portion of the protein of interest (e.g., stereocilin) add up to 100%. In yet a further embodiment, the split site may occur within, after, before, or adjacent to a helix (e.g., alpha) of a protein secondary structure. One embodiment may provide for a split site that is not within a beta strand or bridge. Another embodiment provides for a split site within a helix (e.g., alpha) just upstream of a coil region. Yet another embodiment provides for a protein of interest (e.g., stereocilin) and fragments of the protein of interest outside of a transmembrane domain, i.e., not in the transmembrane portion.

For example, a split may occur such that the N-terminal portion of a protein of interest (e.g., stereocilin; human SEQ ID NO:15 or murine SEQ ID NO:16) comprises a length of at least 10% or greater (e.g., 15%, 20%, 25%, 30%, 40%, 45%, 50%, 55%, 60%, 65%, 70%, 75%, 80%, 85%, 90%, 95%, 100%) of the N-terminal end of the full-length protein of interest (e.g., full-length human SEQ ID NO:2 or SEQ ID NO:25 or murine SEQ ID NO:4 or SEQ ID NO:26). A further embodiment provides an N-terminal portion of a protein of interest (e.g., stereocilin; human SEQ ID NO:15 or murine SEQ ID NO:16) comprising a length of 100% or less (e.g., 99%, 97%, 95%, 90%, 85%, 80%, 75%, 70%, 65%, 60%, 55%, 50%, 45%, 40%, 35%, 30%, 25%, 20%, 15%, 10%) of the N-terminal end of the full-length protein of interest (e.g., full-length human SEQ ID NO:25 or murine SEQ ID NO:26). One other embodiment may provide an N-terminal portion of a protein of interest (e.g., stereocilin; human SEQ ID NO:15 or murine SEQ ID NO:16) comprising a length of 10%-100% (e.g., 15%-90%, 20%-80%, 30%-70%, 40%-60%, 50%) of the N-terminal end of the full-length protein of interest (e.g., full-length human SEQ ID NO:25 or murine SEQ ID NO:26). A further embodiment provides for an N-terminal portion of a protein of interest (e.g., stereocilin; human SEQ ID NO:15 or murine SEQ ID NO:16) comprising less than 54% (e.g., 53%, 52%, 51%, 50%, 45%, 43%, 41%, 40%) of and/or less than 54% identity to and/or less than 54% in length of the N-terminal end of the full-length protein of interest (e.g., full-length human SEQ ID NO:25 or murine SEQ ID NO:26). Yet a further embodiment may be directed to an N-terminal portion of a protein of interest (e.g., stereocilin; human SEQ ID NO:15 or murine SEQ ID NO:16) comprising a length that is less than 54% of the N-terminal end of the full-length protein of interest (e.g., full-length human SEQ ID NO:25 or murine SEQ ID NO:26). Another embodiment provides for an N-terminal portion of a protein of interest (e.g., stereocilin; human SEQ ID NO:15 or murine SEQ ID NO:16) comprising 40% or greater (e.g., 41%, 42%, 43%, 44%, 45%, 50%, 51%, 52%, 53%) of and/or 41% or greater identity to and/or 41% or greater in length of the N-terminal end of the full-length protein of interest (e.g., full-length human SEQ ID NO:25 or murine SEQ ID NO:26).

In yet another embodiment, the C-terminal portion of a protein of interest (e.g., stereocilin; human SEQ ID NO:23 or murine SEQ ID NO:24) comprises a length of at least 10% or greater (e.g., 15%, 20%, 25%, 30%, 40%, 50%, 60%, 70%, 80%, 90%; 100%)) of the C-terminal end of the full-length protein of interest (e.g., stereocilin; full-length human SEQ ID NO:25 or murine SEQ ID NO:26). A further embodiment provides a C-terminal portion of a protein of interest (e.g., stereocilin; human SEQ ID NO:23 or murine SEQ ID NO:24) comprising a length of 100% or less (e.g., 99%, 97%, 95%, 90%, 85%, 80%, 75%, 70%, 65%, 60%, 55%, 50%, 45%, 40%, 35%, 30%, 25%, 20%, 15%, 10%) of the C-terminal end of the full-length protein of interest (e.g., stereocilin; full-length human SEQ ID NO:25 or murine SEQ ID NO:26). One other embodiment may provide a C-terminal portion of a protein of interest (e.g., stereocilin; human SEQ ID NO:23 or murine SEQ ID NO:24) comprising a length of 10%-99% (e.g., 15%-90%, 20%-80%, 30%-70%, 40%-60%, 50%) of the C-terminal end of the full-length protein of interest (e.g., stereocilin; full-length human SEQ ID NO:25 or murine SEQ ID NO:26). A further embodiment provides for a C-terminal portion of a protein of interest (e.g., stereocilin; human SEQ ID NO:23 or murine SEQ ID NO:24) comprising 46% or greater of the C-terminal end of the full-length protein of interest (e.g., stereocilin; full-length human SEQ ID NO:25 or murine SEQ ID NO:26). Yet a further embodiment may be directed to a C-terminal portion of a protein of interest (e.g., stereocilin; human SEQ ID NO:23 or murine SEQ ID NO:24) comprising a length that is 46% or greater (e.g., 47%, 48%, 49%, 50%, 55%, 60%) of and/or 46% or greater identity to and/or 46% or greater in length of the C-terminal end of the full-length protein of interest (e.g., stereocilin; full-length human SEQ ID NO:25 or murine SEQ ID NO:26). Yet a further embodiment may be directed to a C-terminal portion of a protein of interest (e.g., stereocilin; human SEQ ID NO:23 or murine SEQ ID NO:24) comprising a length that is 46% or greater (e.g., 47%, 48%, 49%, 50%, 55%, 60%) of the N-terminal end of the full-length protein of interest (e.g., stereocilin; full-length human SEQ ID NO:25 or murine SEQ ID NO:26). Another embodiment provides for a C-terminal portion of a protein of interest (e.g., stereocilin; human SEQ ID NO:23 or murine SEQ ID NO:24) comprising 60% or less (e.g., 59%, 58%, 57%, 56%, 55%, 50%, 45%) of and/or 60% or less identity to and/or 60% or less in length of the C-terminal end of the full-length protein of interest (e.g., stereocilin; full-length human SEQ ID NO:25 or murine SEQ ID NO:26).

A further embodiment may provide for a split of a full-length wild-type stereocilin protein between, for example, Ala708 and Cys709 or Ala933 and Cys934 of human SEQ ID NO:25 or a split between Ser746 and Cys747 or Ala969 and Cys970 or murine SEQ ID NO:26 to form N-terminal and C-terminal fragments of stereocilin.

### Dual-Vector System

One embodiment of the disclosure provides a dual-vector system for expressing a protein of interest in a cell, where the dual-vector system comprises:
a) a first vector (e.g., capsid, plasmid, transplicing plasmid, viral vector, Adenovirus, AAV, AAV genome, lentivirus) comprising a first nucleotide sequence comprising, in a 5′ to 3′ direction:
   a signal sequence at the 5′-end of a partial coding sequence (e.g., SEQ ID NO:11; encoding SEQ ID NO:12);
   the partial coding sequence encoding an amino terminal (N-terminal) portion of the protein of interest (e.g., STRC; SEQ ID NO:15; SEQ ID NO:16);
   a sequence encoding a splice donor sequence (e.g., an amino terminal fragment of intein (N-intein); SEQ ID NO:13; encoding SEQ ID NO:14); and
b) a second vector (e.g., capsid, plasmid, transplicing plasmid, viral vector, Adenovirus, AAV, AAV genome, lentivirus) comprising a second nucleotide sequence comprising, in a 5′ to 3′ direction:
   a signal sequence that may be upstream of a splice acceptor sequence (e.g., SEQ ID NO:11; encoding SEQ ID NO:12);
   a sequence encoding the splice acceptor sequence (e.g., carboxy terminal fragment of intein (C-intein); SEQ ID NO:21; encoding SEQ ID NO:22);
   a partial coding sequence encoding a carboxy terminal (C-terminal) portion of the protein of interest (e.g., STRC; SEQ ID NO:23; SEQ ID NO:24).

A further embodiment provides a dual-vector system for expressing a protein of interest in a cell, where the dual-vector system comprises (see, e.g., FIG. 6):
a) a first vector comprising a first nucleotide sequence comprising, in a 5′ to 3′ direction:
   a 5′-inverted terminal repeat (5′-ITR) sequence;
   a promoter sequence;
   a signal sequence (e.g., SEQ ID NO:11; encoding SEQ ID NO:12), wherein the signal sequence is operably linked to and under control of the promoter;
   a partial coding sequence encoding an amino terminal (N-terminal) portion of the protein of interest (e.g., STRC; SEQ ID NO:15; SEQ ID NO:16), wherein the partial coding sequence is operably linked to and under control of the promoter;
   a sequence encoding a splice donor sequence (e.g., an amino terminal fragment of intein (N-intein); SEQ ID NO:13; encoding SEQ ID NO:14), wherein the splice donor sequence (e.g., encoding N-intein) is operably linked to and under control of the promoter;
   a poly-adenylation (polyA) signal sequence;
   a 3′-inverted terminal repeat (3′-ITR) sequence; and
b) a second vector comprising a second nucleotide sequence comprising, in a 5′ to 3′ direction:
   a 5′-inverted terminal repeat (5′-ITR) sequence;
   a promoter sequence;
   a signal sequence (e.g., SEQ ID NO:11; encoding SEQ ID NO:12), wherein the signal sequence is operably linked to and under control of the promoter;
   a sequence encoding the splice acceptor sequence (e.g., carboxy terminal fragment of intein (C-intein); SEQ ID NO:21; encoding SEQ ID NO:22), wherein the splice acceptor sequence (e.g., encoding C-intein) is operably linked to and under control of the promoter;
   a partial coding sequence encoding a carboxy terminal (C-terminal) portion of the protein of interest (e.g., STRC; SEQ ID NO:23; SEQ ID NO:24), wherein the partial coding sequence is operably linked to and under control of the promoter;
   a poly-adenylation (polyA) signal sequence;
   a 3′-inverted terminal repeat (3′-ITR) sequence.

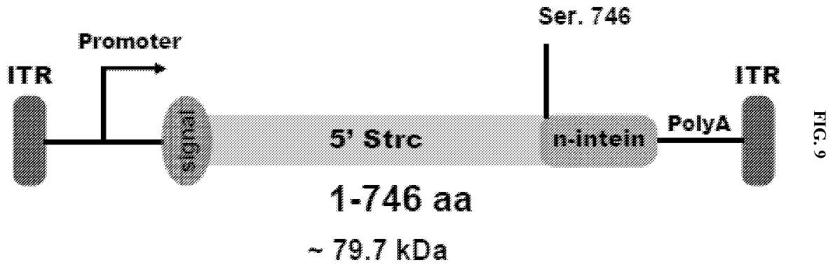

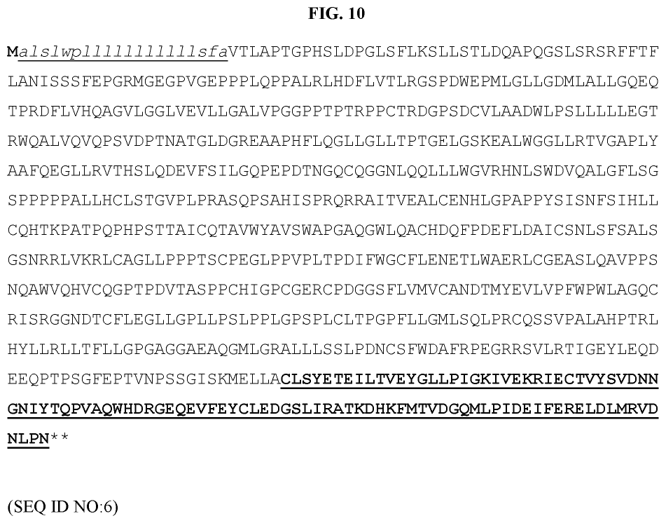

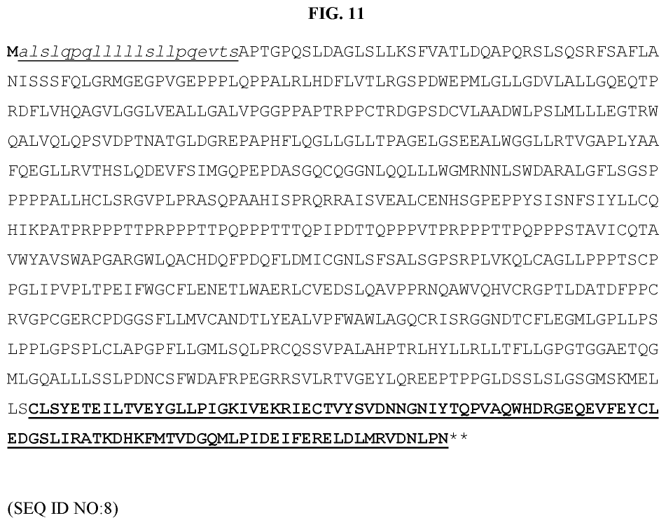

Another embodiment may be directed to a dual-vector system, where a first vector may comprise a first nucleotide sequence comprising, in a 5′ to 3′ direction, a sequence of SEQ ID NO:5 or 7. See, e.g., FIG. 7A, FIG. 7B, FIG. 8A, FIG. 8B, and FIG. 9. FIGS. 7A-7B (SEQ ID NO:5) and FIGS. 8A-8B (SEQ ID NO:7) depict a nucleotide sequence comprising, in a 5′ to 3′ direction, a start codon (ATG, bold), a signal sequence (italicized, underlined) (Signal sequence: 5′-GCTCTCAGCCTCTGGCCCCTGCTGCTGCTGCTGCTGCTGCTGC TGCTGCTGTCCTTTGCA-3′; SEQ ID NO:40; 5′-GCTCTGAGCCTCCAGCCCCAGCTG CTCCTTCTCCTGTCGCTCCTGCCGCAGGAAGTGACTTCA-3′; SEQ ID NO:41), a coding sequence of an N-terminal portion of the STRC gene (black), and N-intein (underlined) (N-intein sequence: 5′-TGCCTGTCATACGAAACCGAGATACTGACAGTAGAATATGG CCTTCTGCCAATCGGGAAGATTGTGGAGAAACGGATAGAATGCACAGTTTACTCT GTCGATAACAATGGTAACATTTATACTCAGCCAGTTGCCCAGTGGCACGACCGG GGAGAGCAGGAAGTATTCGAATACTGTCTGGAGGATGGAAGTCTCATTAGGGCC ACTAAGGACCACAAATTTATGACAGTCGATGGCCAGATGCTGCCTATAGACGAA ATCTTTGAGCGAGAGTTGGACCTCATGCGAGTTGACAACCTTCCTAATTAATAG-3′; SEQ ID NO:42), where the coding sequence encodes the stereocilin (STRC) protein. FIG. 9 shows additional elements, including the ITRs, promoter, and polyA tail, where the murine 5′-STRC encodes an N-terminal portion (1-746 (Ser) amino acids; 79.7 kDa) of a full-length stereocilin (STRC) protein. FIGS. 10 and 11 show the amino acid sequence encoded by the first nucleotide sequence, where the amino acid sequence containing the N-terminal portion of STRC protein (SEQ ID NO:6; SEQ ID NO:8, respectively) comprises a methionine (M) encoded by the ATG codon (bold), a signal peptide sequence (lower case, italicized, underlined) (Signal peptide sequence: N-ALSLWPLLLLLLLLLLLSFA-C; SEQ ID NO:43; N-ALSLQPQLLLLLSLLPQEVTS-C; SEQ ID NO:44), a N-terminal portion of stereocilin protein (black), and an N-intein (bold, underlined) (N-CLSYETEILTVEYGLLPIGKIVEKRI ECTVYSVDNNGNIYTQPVAQWHDRGEQEVFEYCLEDGSLIRATKDHKFMTVDGQM LPIDEIFERELDLMRVDNLPN-C; SEQ ID NO:45).

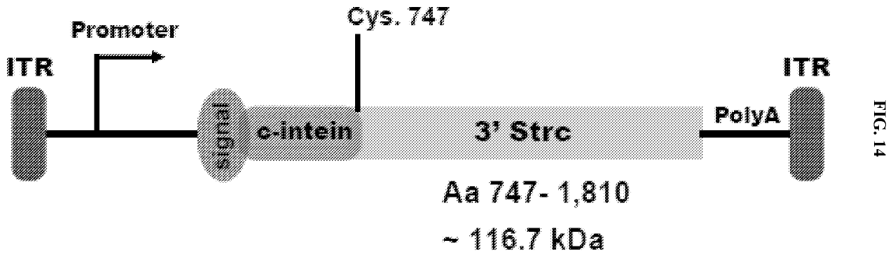

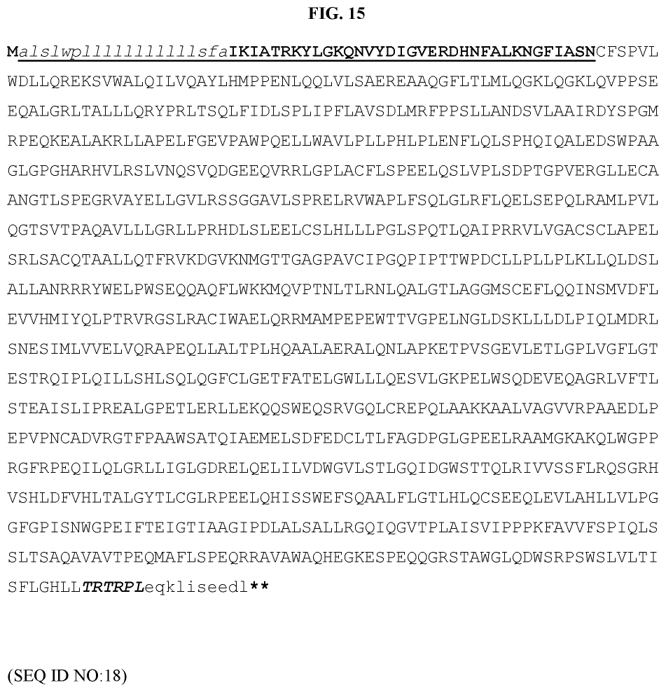

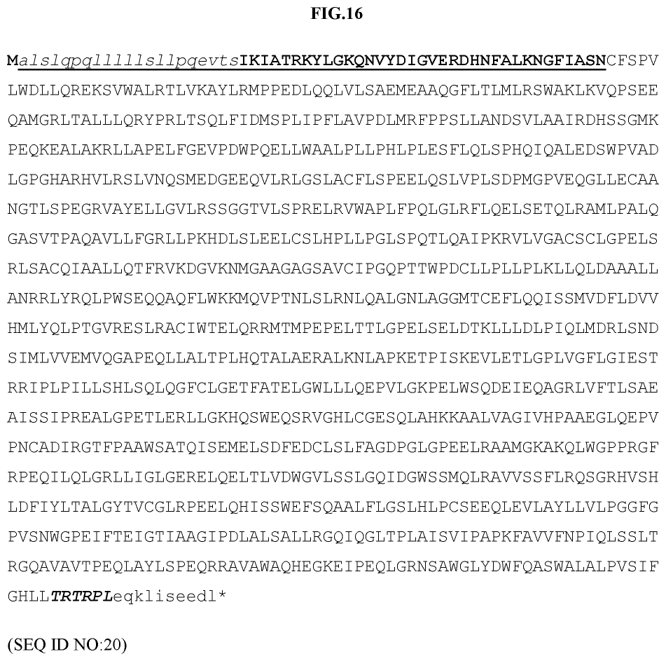

A further embodiment may provide a dual-vector system, where a second vector may comprise a second nucleotide sequence comprising, in a 5′ to 3′ direction, a sequence of SEQ ID NO: 17 or 19. See, e.g., FIG. 12A, FIG. 12B, FIG. 13A, FIG. 13B, and FIG. 14. FIGS. 12A-12B (SEQ ID NO:17) and FIGS. 13A-13B (SEQ ID NO: 19) depict a nucleotide sequence comprising, in a 5′ to 3′ direction, a start codon (bold ATG), a signal sequence (lower case, italicized, and underlined; SEQ ID NO:9; SEQ ID NO:11, respectively), a C-intein sequence (bold and underlined) (5′-ATCAAGATAGCTACAAGGAAGTATCTTGGCAAACAAAA CGTTTATGATATTGGAGTCGAAAGAGATCACAACTTTGCTCTGAAGAACGGATTC ATAGCTTCTAAT-3′; SEQ ID NO:46), a coding sequence of a C-terminal portion of the STRC gene (black), where the coding sequence encodes the stereocilin (STRC) protein, a linker sequence (bold, and italicized) (5′-ACGCGTACGCGGCCGCTC-3′; SEQ ID NO:47), and a Myc tag sequence (lowercase) (5′-GAGCAGAAACTCATCTCAGAAGAGGATCTGT AATAG-3′; SEQ ID NO:48), and a stop codon (italicized and underlined). FIG. 14 shows additional elements, including the ITRs, promoter, and polyA tail, where the murine 3′-STRC encodes a C-terminal portion (747 (Cys)-1,810 amino acids; 116.7 kDa) of a full-length stereocilin (STRC) protein. FIGS. 15 and 16 show the amino acid sequence encoded by the second nucleotide sequence, where the amino acid sequence of the C-terminal portion (SEQ ID NO: 18; SEQ ID NO:20) comprises a methionine (M) encoded by the ATG codon (bold), a signal peptide sequence (lowercase, italicized, and underlined) (SEQ ID NO:10; SEQ ID NO:44), a C-intein (bold and underlined) (N-IKIATRKYLGKQNVYDIGVERDHNFALKN GFIASN-C; SEQ ID NO:49), a C-terminal portion of stereocilin protein (black), a linker sequence (bold and italicized) (N-TRTRPL-C; SEQ ID NO:50), and a Myc tag (lowercase) (SEQ ID NO: 27). One embodiment may provide a full-length STRC protein, where the C-terminal portion of stereocilin protein begins with cysteine (C; Cys), phenylalanine (F; Phe), and serine (S; Ser) (FIG. 17).

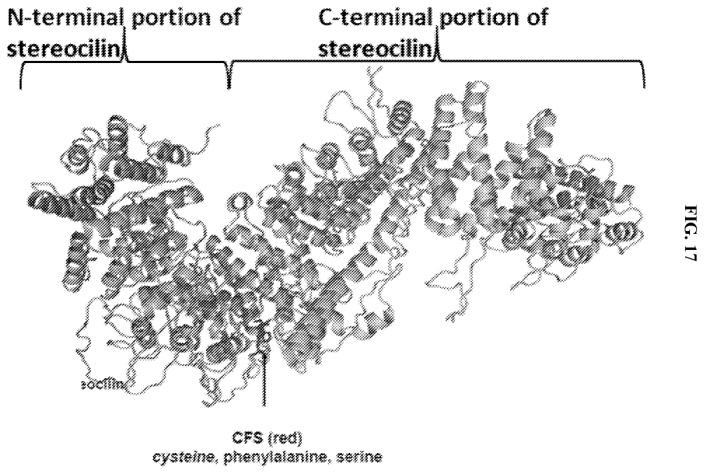

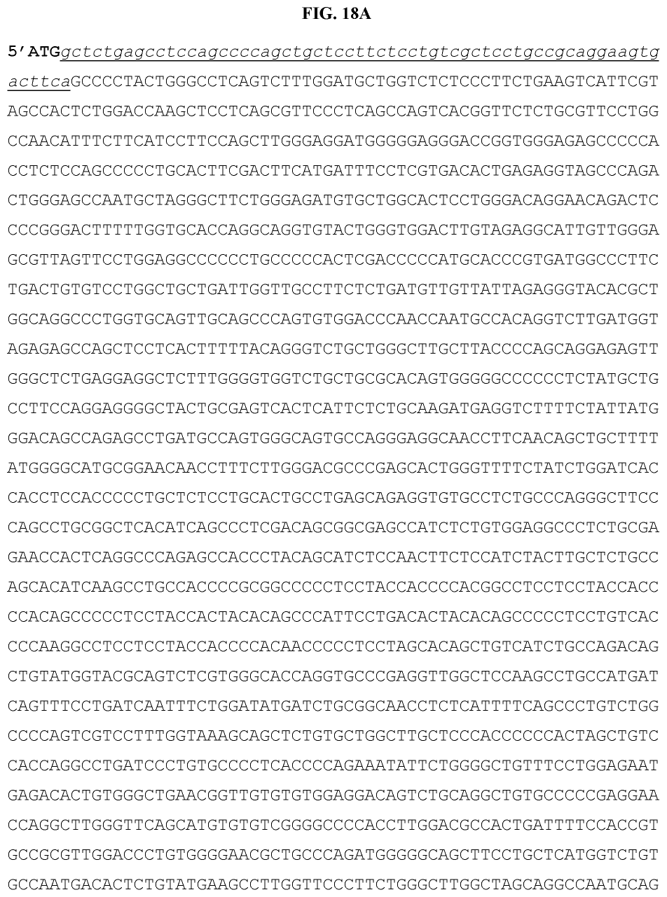

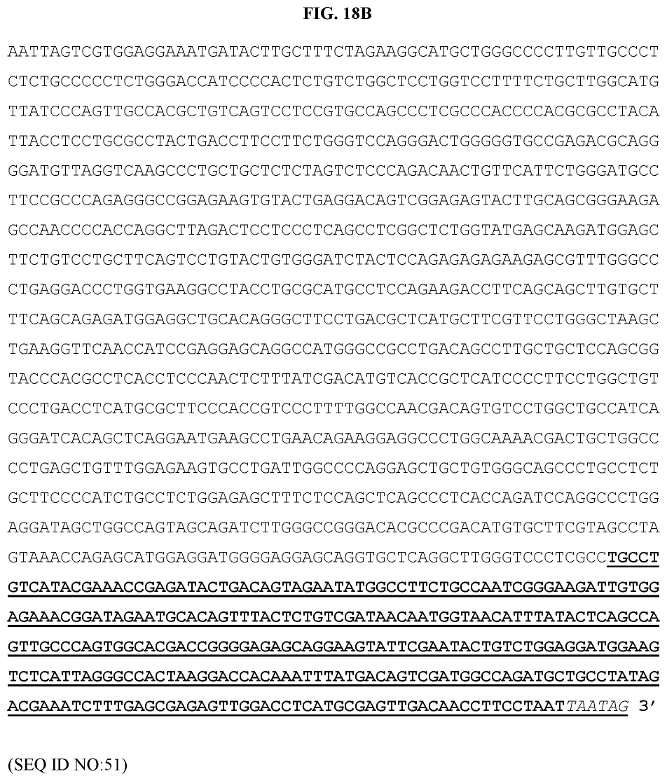

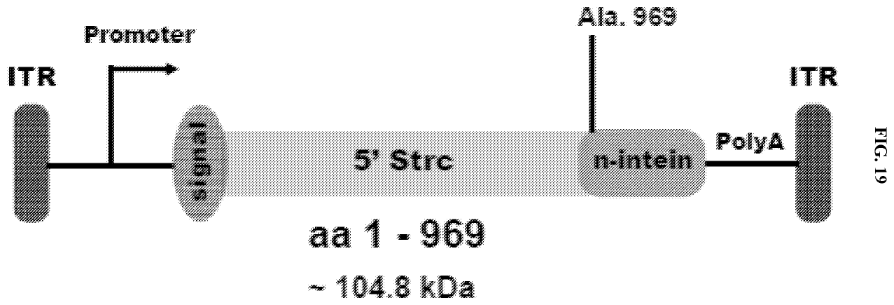

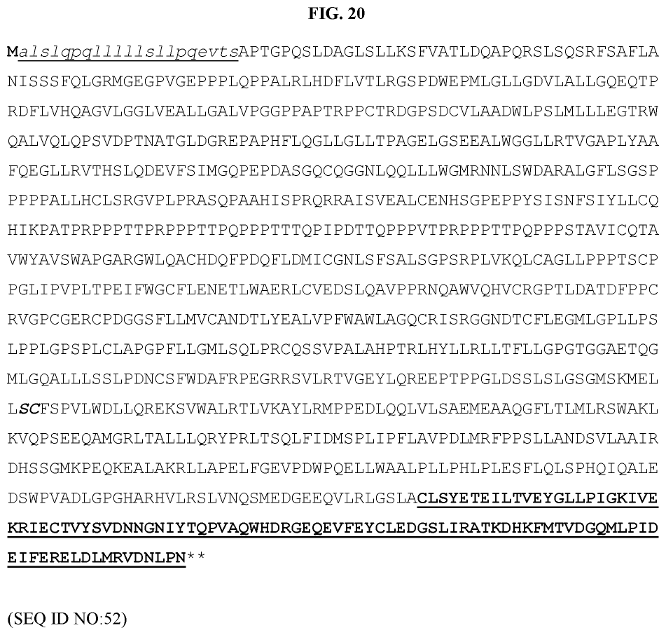

In yet a further embodiment, a dual-vector system may not provide a first vector comprising a first nucleotide sequence comprising, in a 5′ to 3′ direction, a sequence of SEQ ID NO: 51. See, e.g., FIG. 18A, FIG. 18B, and FIG. 19. FIGS. 18A-18B (SEQ ID NO:51) depict a nucleotide sequence comprising, in a 5′ to 3′ direction, a start codon (ATG, bold), a signal sequence (lowercase, italicized, and underlined) (SEQ ID NO:11), a coding sequence of an N-terminal portion of the STRC gene (black), and N-intein (bold and underlined) (SEQ ID NO:42), where the coding sequence encodes the stereocilin (STRC) protein, and a Stop codon (italicized and underlined). FIG. 19 shows additional elements, including the ITRs, promoter, and polyA tail, where the 5′-STRC encodes an N-terminal portion (1-969 (Ala) amino acids; 104.8 kDa) of a full-length stereocilin (STRC) protein. FIG. 20 shows the amino acid sequence encoded by the first nucleotide sequence, where the amino acid sequence of the N-terminal portion (SEQ ID NO:52) comprises a methionine (M) encoded by the ATG codon (bold), a signal peptide sequence (lowercase, italicized, and underlined) (SEQ ID NO:44), an N-terminal portion of stereocilin protein (black), and an N-intein (bold and underlined) (SEQ ID NO: 45).

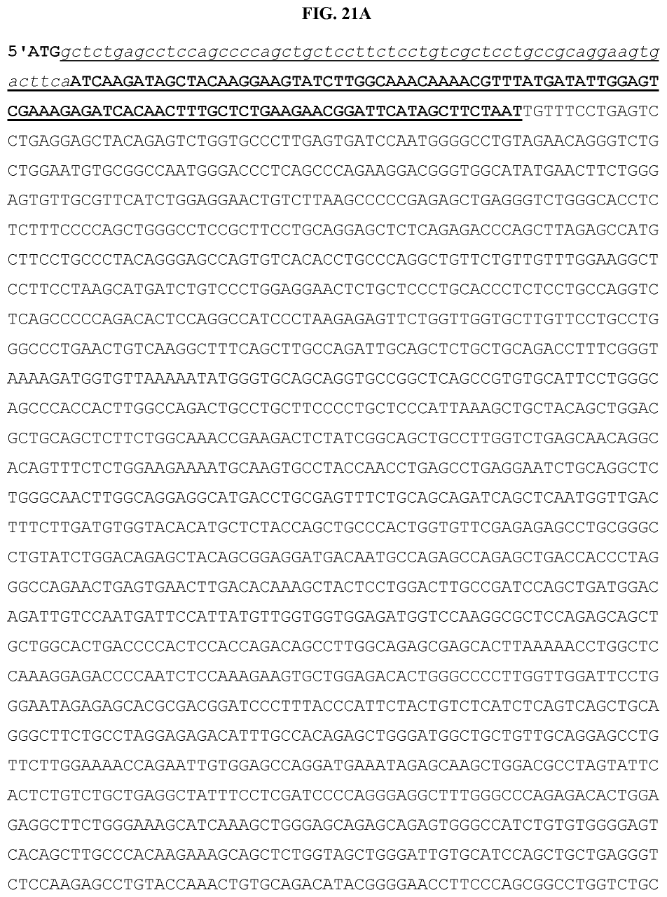

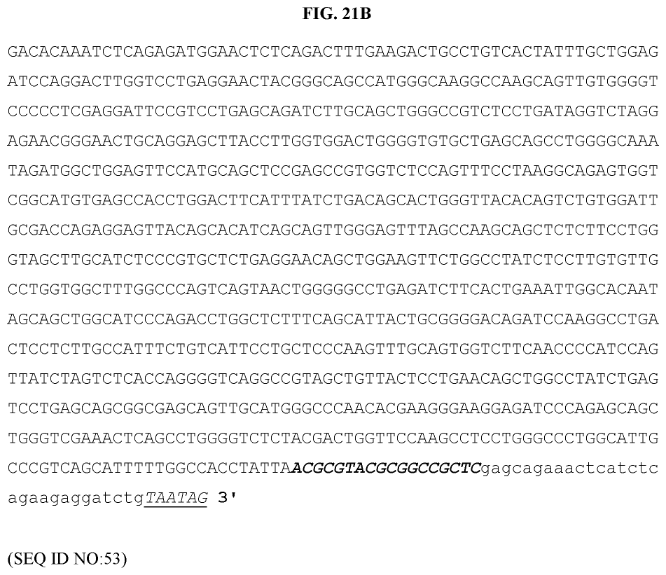

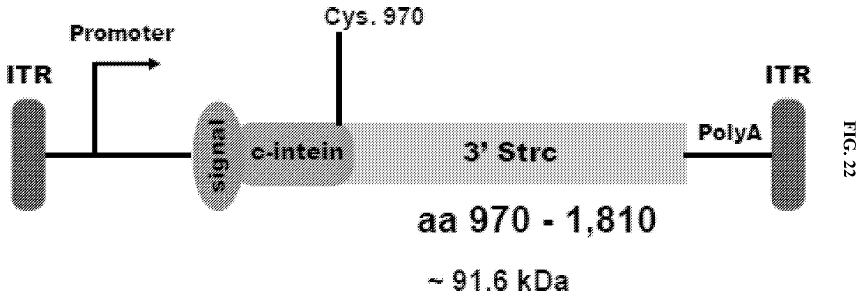

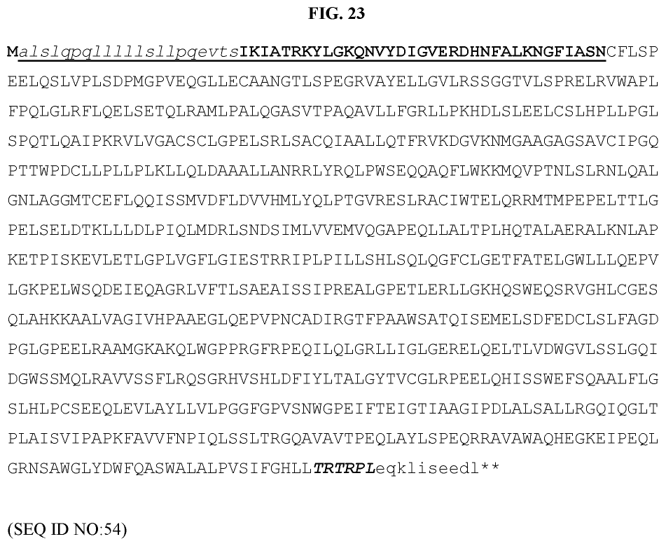

A further embodiment may not provide a dual-vector system having a second vector comprising a second nucleotide sequence comprising, in a 5′ to 3′ direction, a sequence of SEQ ID NO:53. See, e.g., FIG. 21A, FIG. 21B, and FIG. 22. FIGS. 21A-21B (SEQ ID NO:53) depict a nucleotide sequence comprising, in a 5′ to 3′ direction, a start codon (bold ATG), a signal sequence (lowercase, italicized, and underlined) (SEQ ID NO:11), a C-intein sequence (bold and underlined) (SEQ ID NO:21), a coding sequence of a C-terminal portion of the STRC gene (black), where the coding sequence encodes the stereocilin (STRC) protein, a linker sequence (bold and italicized) (SEQ ID NO:47), a Myc tag sequence (lowercase) (SEQ ID NO:48), and a Stop codon (italicized and underlined). FIG. 22 shows additional elements, including the ITRs, promoter, and polyA tail, where the 3′-STRC encodes a C-terminal portion (970 (Cys)-1,810 amino acids; 91.6 kDa) of a full-length stereocilin (STRC) protein. FIG. 23 shows the amino acid sequence encoded by the second nucleotide sequence, where the amino acid sequence of the C-terminal portion (SEQ ID NO:54) comprises a methionine (M) encoded by the ATG codon (bold), a signal peptide sequence (lowercase, italicized, and underlined) (SEQ ID NO:44), a C-intein (bold and underlined) (SEQ ID NO:49), a C-terminal portion of stereocilin protein (black), a linker sequence (bold and italicized) (SEQ ID NO:50), and a Myc tag (lowercase) (SEQ ID NO:27).

One embodiment of the dual-vector system provides for a vector for each divided portion of a protein of interest (e.g., stereocilin), i.e., an N-terminal portion and a C-terminal portion. The divided portions of a protein of interest (e.g., stereocilin) and any additional regions necessary for regulating, producing, or expressing the protein of interest are such that each portion and associated regions do not exceed the cargo capacity of their respective vectors (e.g., virus (e.g., viral vectors, bacteriophage, phage, retrovirus), plasmid, cosmid, bacterial artificial chromosome, yeast artificial chromosome, human artificial chromosome). The divided portions of the same protein of interest and additional regions should not be of a size that exceeds the cargo capacity of the selected vector.

One embodiment of the disclosure provides a vector system (e.g., dual-vector system) for delivering genes with large coding sequences, including those 4 kB or greater (e.g., 4.5 kB, 5 kB, 5.5 kB, 5.8 kB, 6 kB, 6.5 kB, 7 kB, 7.5 kB, 8 kB, 8.5 kB, 9 kB, 9.5 kB, 10 kB, 11 kB, 12 kB). The vector (e.g., first vector and second vector) of the disclosure may each be a viral vector (e.g., adenovirus, adeno-associated virus (AAV), lentivirus, herpes simplex virus I, vaccinia virus), where in some embodiments, the viral vector may be an AAV vector or recombinant AAV vector. Another embodiment may provide for viral vectors of the same or different serotypes (e.g., AAV1, AAV2, AAV3, AAV4, AAV5, AAV6, AAV7, AAV8, AAV9, AAV10, AAV11, synthetic serotype). AAV serotypes useful in the disclosure described here may include, but are not limited to, AAV1, AAV2, AAV5, AAV6, AAV8, and AAV9.

In one embodiment, the first and second vectors are viral vectors, e.g., adeno-associated virus (AAV) or recombinant AAV (rAAV), used interchangeably herein, which lack viral DNA. A first vector of the dual-vector system comprises a nucleotide sequence containing in a 5′ to 3′ direction: a 5′-inverted terminal repeat (5′-ITR) sequence; a promoter sequence that may drive transcription of a downstream polynucleotide of interest (e.g., STRC); a signal sequence that is operably linked to and under control of the promoter; a partial coding sequence encoding an amino terminal (N-terminal) portion of a protein of interest (e.g., STRC), where the partial coding sequence is operably linked to and under control of the promoter; a sequence encoding an amino terminal fragment of intein (N-intein), wherein the sequence encoding N-intein is operably linked to and under control of the promoter; a poly-adenylation (polyA) signal sequence; and a 3′-ITR sequence.

In another embodiment, a second vector comprises, in a 5′ to 3′ direction, a 5′-inverted terminal repeat (5′-ITR) sequence; a promoter sequence that may drive transcription of a downstream polynucleotide of interest (e.g., STRC); a signal sequence that is operably linked to and under control of the promoter; a sequence encoding a carboxy terminal fragment of intein (C-intein), wherein the sequence encoding C-intein is operably linked to and under control of the promoter; a partial coding sequence encoding a carboxy terminal (C-terminal) portion of a protein of interest (e.g, STRC), where the partial coding sequence is operably linked to and under control of the promoter; a poly-adenylation (polyA) signal sequence; and a 3′-ITR sequence.

Yet in a further embodiment, when the first vector and the second vector of the disclosure are inserted into a cell(s) (e.g., host cell, mammalian cell, human cell, bacterial cell) by any means, including but not limited to, viral transduction, bacterial transformation using calcium chloride, bacterial transformation or transduction by bacterial mating or conjugation, transfection (e.g., electroporation, calcium phosphate, liposome-based transfection), gene gun, and the like, the vectors express, respectively, a first protein sequence comprising, in an N-terminal to C-terminal direction, a signal peptide sequence linked to an N-terminal portion of the protein of interest sequence (e.g., STRC) fused at its C-terminal end to an N-intein protein sequence; and a second protein sequence comprising, in an N-terminal to C-terminal direction, a signal peptide sequence linked to a C-intein protein sequence fused to the N-terminal end of a C-terminal portion of the protein of interest sequence (e.g., STRC). Intein-mediated protein splicing of an N-terminal portion of a protein of interest (e.g., STRC) and a C-terminal portion of the same protein of interest (e.g., STRC) results in the expression of a full-length protein of interest (e.g., STRC).

Another embodiment provides a signal peptide sequence of the dual-vector system, where the signal peptide sequence may be located at the N-terminal ends of each protein sequence encoded by the dual-vector system. A first protein sequence comprising the N-terminal portion of a protein of interest (e.g., STRC), the signal peptide sequence may be upstream of the coding region of the N-terminal portion of the protein of interest as well as an N-intein. A second protein sequence comprising the C-terminal portion of a protein of interest (e.g., STRC), the signal peptide sequence may be upstream of the C-intein as well as the coding region of the C-terminal portion of the protein of interest (e.g., STRC).

Another embodiment of the disclosure provides a dual-vector system, where a first vector and a second vector in a cell express a first protein sequence and the second protein sequence, respectively, each containing the same signal peptide sequence. Accordingly, the same signal peptide sequence allows the first protein sequence and the second protein sequence to be transported to the same cellular compartment. In a different embodiment, the signal peptide sequences of the first and second protein sequences may be different, yet these signal peptide sequences direct each respective protein sequence to the same cellular compartment. The signal peptide sequences of the first and second protein sequences may be configured to transport the first protein sequence and the second protein sequence to the same cellular compartment. In doing so, each of the protein sequences may be in sufficient proximity for the intein-mediated protein fusing to occur, thereby forming a full-length protein of interest (e.g., STRC). The signal peptide sequence may be associated with the protein of interest (e.g., STRC). A further embodiment may provide for a signal sequence encoding a signal peptide sequence that is associated with a protein other than the protein of interest, where the signal sequence of a first nucleotide sequence and the signal sequence of a second nucleotide sequence are different, and the signal sequences encode signal peptide sequences that are different as well, yet the signal sequences are configured to transport the first protein sequence and the second protein sequence to the same cellular compartment. Another embodiment of the disclosure provides for a signal sequence or signal sequences such that the signal sequence directs the two fragments to the same cellular or intracellular compartment without disrupting intein-mediated trans-splicing. The signal sequence may be particularly useful to ensure that the first protein sequence and the second protein sequence are in sufficient proximity to each other to allow for the N-terminal portion of the protein of interest (e.g., STRC) and the C-terminal portion of the protein of interest (e.g., STRC) to form the full-length protein of interest (e.g., STRC) through a peptide bond.

In an embodiment of the disclosure, the signal sequence may comprise a nucleic acid sequence of at least 80% (e.g., 85%, 90%, 95%, 97%, 99%, 100%) identity to the signal sequence that encodes a signal peptide sequence of a protein of interest (e.g., STRC). For example, the signal sequence may comprise a nucleic acid sequence of at least 80% (e.g., 85%, 90%, 95%, 97%, 99%, 100%) identity to SEQ ID NO:11 of a gene sequence encoding the STRC protein signal peptide, or the signal sequence may comprise a nucleic acid sequence consisting of, for example, any signal sequence that directs the protein of interest, and each of the portions of the protein of interest, to the same cellular compartment (e.g., SEQ ID NO:9 or SEQ ID NO:11. Another embodiment may provide a signal sequence that encodes a signal peptide sequence having an amino acid sequence of at least 80% (e.g., 85%, 90%, 95%, 97%, 99%, 100%) identity to a signal peptide sequence of a protein of interest (e.g., STRC; SEQ ID NO:10; SEQ ID NO:12). For example, the signal peptide sequence may comprise an amino acid sequence of at least 80% (e.g., 85%, 90%, 95%, 97%, 99%, 100%) identity to SEQ ID NO:10 or SEQ ID NO: 12 of the STRC protein signal peptide, or the signal peptide sequence may comprise an amino acid sequence consisting of SEQ ID NO: 10 or SEQ ID NO:12.

Yet a further embodiment may provide a partial coding sequence encoding an N-terminal portion of a protein of interest (e.g., STRC) where the partial coding sequence comprises a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the coding sequence encoding the N-terminal portion of the protein of interest (e.g., STRC; SEQ ID NO:6, 8, 15, 16, 25, or 26). For example, the partial coding sequence encoding an N-terminal portion of STRC protein (comprising a signal sequence, which may be exchangeable with a different signal sequence) may comprise a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the following sequences.

The partial coding sequence encoding a human N-terminal portion of STRC protein (and including a start codon, ATG (bold), and signal sequence (lowercase, italicized, and underlined)) may be as follows:

(SEQ ID NO: 55))
5′ ATGgctctcagcctctggcccctgctgctgctgctgctgctgctgctgctgctgtccttt
gcaGTGACTCTGGCCCCTACTGGGCCTCATTCCCTGGACCCTGGTCTCTCCTTCCTGAAGTC
ATTGCTCTCCACTCTGGACCAGGCTCCCCAGGGCTCCCTGAGCCGCTCACGGTTCTTTACAT
TCCTGGCCAACATTTCTTCTTCCTTTGAGCCTGGGAGAATGGGGGAAGGACCAGTAGGAGAG
CCCCCACCTCTCCAGCCGCCTGCTCTGCGGCTCCATGATTTTCTAGTGACACTGAGAGGTAG
CCCCGACTGGGAGCCAATGCTAGGGCTGCTAGGGGATATGCTGGCACTGCTGGGACAGGAGC
AGACTCCCCGAGATTTCCTGGTGCACCAGGCAGGGGTGCTGGGTGGACTTGTGGAGGTGCTG
CTGGGAGCCTTAGTTCCTGGGGGCCCCCCTACCCCAACTCGGCCCCCATGCACCCGTGATGG
GCCGTCTGACTGTGTCCTGGCTGCTGACTGGTTGCCTTCTCTGCTGCTGTTGTTAGAGGGCA
CACGCTGGCAAGCTCTGGTGCAGGTGCAGCCCAGTGTGGACCCCACCAATGCCACAGGCCTC
GATGGGAGGGAGGCAGCTCCTCACTTTTTGCAGGGTCTGTTGGGTTTGCTTACCCCAACAGG
GGAGCTAGGCTCCAAGGAGGCTCTTTGGGGCGGTCTGCTACGCACAGTGGGGGCCCCCCTCT
ATGCTGCCTTTCAGGAGGGGCTGCTCCGTGTCACTCACTCCCTGCAGGATGAGGTCTTCTCC
ATTTTGGGGCAGCCAGAGCCTGATACCAATGGGCAGTGCCAGGGAGGTAACCTTCAACAGCT
GCTCTTATGGGGCGTCCGGCACAACCTTTCCTGGGATGTCCAGGCGCTGGGCTTTCTGTCTG
GATCACCACCCCCACCCCCTGCCCTCCTTCACTGCCTGAGCACGGGCGTGCCTCTGCCCAGA
GCTTCTCAGCCGTCAGCCCACATCAGCCCACGCCAACGGCGAGCCATCACTGTGGAGGCCCT
CTGTGAGAACCACTTAGGCCCAGCACCACCCTACAGCATTTCCAACTTCTCCATCCACTTGC
TCTGCCAGCACACCAAGCCTGCCACTCCACAGCCCCATCCCAGCACCACTGCCATCTGCCAG
ACAGCTGTGTGGTATGCAGTGTCCTGGGCACCAGGTGCCCAAGGCTGGCTACAGGCCTGCCA
CGACCAGTTTCCTGATGAGTTTTTGGATGCGATCTGCAGTAACCTCTCCTTTTCAGCCCTGT
CTGGCTCCAACCGCCGCCTGGTGAAGCGGCTCTGTGCTGGCCTGCTCCCACCCCCTACCAGC
TGCCCTGAAGGCCTGCCCCCTGTTCCCCTCACCCCAGACATCTTTTGGGGCTGCTTCTTGGA
GAATGAGACTCTGTGGGCTGAGCGACTGTGTGGGGAGGCAAGTCTACAGGCTGTGCCCCCCA
GCAACCAGGCTTGGGTCCAGCATGTGTGCCAGGGCCCCACCCCAGATGTCACTGCCTCCCCA
CCATGCCACATTGGACCCTGTGGGGAACGCTGCCCGGATGGGGGCAGCTTCCTGGTGATGGT
CTGTGCCAATGACACCATGTATGAGGTCCTGGTGCCCTTCTGGCCTTGGCTAGCAGGCCAAT
GCAGGATAAGTCGTGGGGGCAATGACACTTGCTTCCTAGAAGGGCTGCTGGGCCCCCTTCTG
CCCTCTCTGCCACCACTGGGACCATCCCCACTCTGTCTGACCCCTGGCCCCTTCCTCCTTGG
CATGCTATCCCAGTTGCCACGCTGTCAGTCCTCTGTCCCAGCTCTTGCTCACCCCACACGCC
TACACTATCTCCTCCGCCTGCTGACCTTCCTCTTGGGTCCAGGGGCTGGGGGCGCTGAGGCC
CAGGGGATGCTGGGTCGGGCCCTACTGCTCTCCAGTCTCCCAGACAACTGCTCCTTCTGGGA
TGCCTTTCGCCCAGAGGGCCGGCGCAGTGTGCTACGGACGATTGGGGAATACCTGGAACAAG
ATGAGGAGCAGCCAACCCCATCAGGCTTTGAACCCACTGTCAACCCCAGCTCTGGTATAAGC
AAGATGGAGCTGCTGGCC.

The partial coding sequence encoding a murine N-terminal portion of STRC protein (and including a start codon, ATG (bold), and signal sequence (lowercase, italicized, and underlined) may be as follows:

(SEQ ID NO: 56)
5′-ATGgctctgagcctccagccccagctgctccttctcctgtcgctcctgccgcaggaagt
gacttcaGCCCCTACTGGGCCTCAGTCTTTGGATGCTGGTCTCTCCCTTCTGAAGTCATTCG
TAGCCACTCTGGACCAAGCTCCTCAGCGTTCCCTCAGCCAGTCACGGTTCTCTGCGTTCCTG
GCCAACATTTCTTCATCCTTCCAGCTTGGGAGGATGGGGGAGGGACCGGTGGGAGAGCCCCC
ACCTCTCCAGCCCCCTGCACTTCGACTTCATGATTTCCTCGTGACACTGAGAGGTAGCCCAG
ACTGGGAGCCAATGCTAGGGCTTCTGGGAGATGTGCTGGCACTCCTGGGACAGGAACAGACT
CCCCGGGACTTTTTGGTGCACCAGGCAGGTGTACTGGGTGGACTTGTAGAGGCATTGTTGGG
AGCGTTAGTTCCTGGAGGCCCCCCTGCCCCCACTCGACCCCCATGCACCCGTGATGGCCCTT
CTGACTGTGTCCTGGCTGCTGATTGGTTGCCTTCTCTGATGTTGTTATTAGAGGGTACACGC
TGGCAGGCCCTGGTGCAGTTGCAGCCCAGTGTGGACCCAACCAATGCCACAGGTCTTGATGG
TAGAGAGCCAGCTCCTCACTTTTTACAGGGTCTGCTGGGCTTGCTTACCCCAGCAGGAGAGT
TGGGCTCTGAGGAGGCTCTTTGGGGTGGTCTGCTGCGCACAGTGGGGGCCCCCCTCTATGCT
GCCTTCCAGGAGGGGCTACTGCGAGTCACTCATTCTCTGCAAGATGAGGTCTTTTCTATTAT
GGGACAGCCAGAGCCTGATGCCAGTGGGCAGTGCCAGGGAGGCAACCTTCAACAGCTGCTTT
TATGGGGCATGCGGAACAACCTTTCTTGGGACGCCCGAGCACTGGGTTTTCTATCTGGATCA
CCACCTCCACCCCCTGCTCTCCTGCACTGCCTGAGCAGAGGTGTGCCTCTGCCCAGGGCTTC
CCAGCCTGCGGCTCACATCAGCCCTCGACAGCGGCGAGCCATCTCTGTGGAGGCCCTCTGCG
AGAACCACTCAGGCCCAGAGCCACCCTACAGCATCTCCAACTTCTCCATCTACTTGCTCTGC
CAGCACATCAAGCCTGCCACCCCGCGGCCCCCTCCTACCACCCCACGGCCTCCTCCTACCAC
CCCACAGCCCCCTCCTACCACTACACAGCCCATTCCTGACACTACACAGCCCCCTCCTGTCA
CCCCAAGGCCTCCTCCTACCACCCCACAACCCCCTCCTAGCACAGCTGTCATCTGCCAGACA
GCTGTATGGTACGCAGTCTCGTGGGCACCAGGTGCCCGAGGTTGGCTCCAAGCCTGCCATGA
TCAGTTTCCTGATCAATTTCTGGATATGATCTGCGGCAACCTCTCATTTTCAGCCCTGTCTG
GCCCCAGTCGTCCTTTGGTAAAGCAGCTCTGTGCTGGCTTGCTCCCACCCCCCACTAGCTGT
CCACCAGGCCTGATCCCTGTGCCCCTCACCCCAGAAATATTCTGGGGCTGTTTCCTGGAGAA
TGAGACACTGTGGGCTGAACGGTTGTGTGTGGAGGACAGTCTGCAGGCTGTGCCCCCGAGGA
ACCAGGCTTGGGTTCAGCATGTGTGTCGGGGCCCCACCTTGGACGCCACTGATTTTCCACCG
TGCCGCGTTGGACCCTGTGGGGAACGCTGCCCAGATGGGGGCAGCTTCCTGCTCATGGTCTG
TGCCAATGACACTCTGTATGAAGCCTTGGTTCCCTTCTGGGCTTGGCTAGCAGGCCAATGCA
GAATTAGTCGTGGAGGAAATGATACTTGCTTTCTAGAAGGCATGCTGGGCCCCTTGTTGCCC
TCTCTGCCCCCTCTGGGACCATCCCCACTCTGTCTGGCTCCTGGTCCTTTTCTGCTTGGCAT
GTTATCCCAGTTGCCACGCTGTCAGTCCTCCGTGCCAGCCCTCGCCCACCCCACGCGCCTAC
ATTACCTCCTGCGCCTACTGACCTTCCTTCTGGGTCCAGGGACTGGGGGTGCCGAGACGCAG
GGGATGTTAGGTCAAGCCCTGCTGCTCTCTAGTCTCCCAGACAACTGTTCATTCTGGGATGC
CTTCCGCCCAGAGGGCCGGAGAAGTGTACTGAGGACAGTCGGAGAGTACTTGCAGCGGGAAG
AGCCAACCCCACCAGGCTTAGACTCCTCCCTCAGCCTCGGCTCTGGTATGAGCAAGATGGAG
CTTCTGTCC-3′,

or a partial coding sequence encoding an N-terminal portion of STRC protein comprising a nucleic acid sequence consisting of SEQ ID NO:55 or 56. Another embodiment may provide an N-terminal portion of a protein of interest (e.g., STRC) (including methionine corresponding to a start codon, ATG, and a signal peptide sequence) having an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the protein of interest (e.g., STRC; SEQ ID NO:25 or 26). For example, the N-terminal portion of the STRC protein (including methionine corresponding to a start codon, ATG, and a STRC signal peptide sequence, which may be exchangeable) may comprise an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the sequence of SEQ ID NO:15 or 16, or an N-terminal portion of the STRC protein comprising an amino acid sequence consisting of SEQ ID NO: 15 or 16. A first nucleotide sequence of a first vector may comprise a partial coding sequence encoding an N-terminal portion of a protein of interest (e.g., STRC; SEQ ID NO: 15 or 16) comprising its own signal sequence (e.g., SEQ ID NO:10 or 12) or alternatively, a different signal sequence, and a splice donor sequence (e.g., an N-terminal intein (N-intein); SEQ ID NO:14).

In one embodiment, a partial coding sequence encoding a C-terminal portion of a protein of interest (e.g., STRC) (including methionine corresponding to a start codon, ATG, and a signal sequence, which may be exchangeable; and optionally including a linker sequence and a Myc tag sequence) where the partial coding sequence comprises a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the protein of interest (e.g., STRC; SEQ ID NO:18, 20, 23, 24, 25, or 26). For example, the partial coding sequence encoding a C-terminal portion of STRC protein may comprise a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the following sequences.

The partial coding sequence encoding a human C-terminal portion of STRC protein may be as follows (i.e., without an ATG start codon, signal sequence, or splice acceptor sequence).

(SEQ ID NO: 57)
5′-TGCTTTAGTCCTGTGCTGTGGGATCTGCTCCAGAGGGAAAAGAGTGTTTGGGCCCTGCA
GATTCTAGTGCAGGCGTACCTGCATATGCCCCCAGAAAACCTCCAGCAGCTGGTGCTTTCAG
CAGAGAGGGAGGCTGCACAGGGCTTCCTGACACTCATGCTGCAGGGGAAGCTGCAGGGGAAG
CTGCAGGTACCACCATCCGAGGAGCAGGCCCTGGGTCGCCTGACAGCCCTGCTGCTCCAGCG
GTACCCACGCCTCACCTCCCAGCTCTTCATTGACCTGTCACCACTCATCCCTTTCTTGGCTG
TCTCTGACCTGATGCGCTTCCCACCATCCCTGTTAGCCAACGACAGTGTCCTGGCTGCCATC
CGGGATTACAGCCCAGGAATGAGGCCTGAACAGAAGGAGGCTCTGGCAAAGCGACTGCTGGC
CCCTGAACTGTTTGGGGAAGTGCCTGCCTGGCCCCAGGAGCTGCTGTGGGCAGTGCTGCCCC
TGCTCCCCCACCTCCCTCTGGAGAACTTTTTGCAGCTCAGCCCTCACCAGATCCAGGCCCTG
GAGGATAGCTGGCCAGCAGCAGGTCTGGGGCCAGGGCATGCCCGCCATGTGCTGCGCAGCCT
GGTAAACCAGAGTGTCCAGGATGGTGAGGAGCAGGTACGCAGGCTTGGGCCCCTCGCCTGTT
TCCTGAGCCCTGAGGAGCTGCAGAGCCTAGTGCCCCTGAGTGATCCAACGGGGCCAGTAGAA
CGGGGGCTGCTGGAATGTGCAGCCAATGGGACCCTCAGCCCAGAAGGACGGGTGGCATATGA
ACTTCTGGGTGTGTTGCGCTCATCTGGAGGAGCGGTGCTGAGCCCCCGGGAGCTGCGGGTCT
GGGCCCCTCTCTTCTCTCAGCTGGGCCTCCGCTTCCTTCAGGAGCTGTCAGAGCCCCAGCTT
AGAGCCATGCTTCCTGTCCTGCAGGGAACTAGTGTTACACCTGCTCAGGCTGTCCTGCTGCT
TGGACGGCTCCTTCCTAGGCACGATCTATCCCTGGAGGAACTCTGCTCCTTGCACCTTCTGC
TACCAGGCCTCAGCCCCCAGACACTCCAGGCCATCCCTAGGCGAGTCCTGGTCGGGGCTTGT
TCCTGCCTGGCCCCTGAACTGTCACGCCTCTCAGCCTGCCAGACCGCAGCACTGCTGCAGAC
CTTTCGGGTTAAAGATGGTGTTAAAAATATGGGTACAACAGGTGCTGGTCCAGCTGTGTGTA
TCCCTGGTCAGCCTATTCCCACCACCTGGCCAGACTGCCTGCTTCCCCTGCTCCCATTAAAG
CTGCTACAACTGGATTCCTTGGCTCTTCTGGCAAATCGAAGACGCTACTGGGAGCTGCCCTG
GTCTGAGCAGCAGGCACAGTTTCTCTGGAAGAAGATGCAAGTACCCACCAACCTTACCCTCA
GGAATCTGCAGGCTCTGGGCACCCTGGCAGGAGGCATGTCCTGTGAGTTTCTGCAGCAGATC
AACTCCATGGTAGACTTCCTTGAAGTGGTGCACATGATCTATCAGCTGCCCACTAGAGTTCG
AGGGAGCCTGAGGGCCTGTATCTGGGCAGAGCTACAGCGGAGGATGGCAATGCCAGAACCAG
AATGGACAACTGTAGGGCCAGAACTGAACGGGCTGGATAGCAAGCTACTCCTGGACTTACCG
ATCCAGTTGATGGACAGACTATCCAATGAATCCATTATGTTGGTGGTGGAGCTGGTGCAAAG
AGCTCCAGAGCAGCTGCTGGCACTGACCCCCCTCCACCAGGCAGCCCTGGCAGAGAGGGCAC
TACAAAACCTGGCTCCAAAGGAGACTCCAGTCTCAGGGGAAGTGCTGGAGACCTTAGGCCCT
TTGGTTGGATTCCTGGGGACAGAGAGCACACGACAGATCCCCCTACAGATCCTGCTGTCCCA
TCTCAGTCAGCTGCAAGGCTTCTGCCTAGGAGAGACATTTGCCACAGAGCTGGGATGGCTGC
TATTGCAGGAGTCTGTTCTTGGGAAACCAGAGTTGTGGAGCCAGGATGAAGTAGAGCAAGCT
GGACGCCTAGTATTCACTCTGTCTACTGAGGCAATTTCCTTGATCCCCAGGGAGGCCTTGGG
TCCAGAGACCCTGGAGCGGCTTCTAGAAAAGCAGCAGAGCTGGGAGCAGAGCAGAGTTGGAC
AGCTGTGTAGGGAGCCACAGCTTGCTGCCAAGAAAGCAGCCCTGGTAGCAGGGGTGGTGCGA
CCAGCTGCTGAGGATCTTCCAGAACCTGTGCCAAATTGTGCAGATGTACGAGGGACATTCCC
AGCAGCCTGGTCTGCAACCCAGATTGCAGAGATGGAGCTCTCAGACTTTGAGGACTGCCTGA
CATTATTTGCAGGAGACCCAGGACTTGGGCCTGAGGAACTGCGGGCAGCCATGGGCAAAGCA
AAACAGTTGTGGGGTCCCCCCCGGGGATTTCGTCCTGAGCAGATCCTGCAGCTTGGTAGGCT
CTTAATAGGTCTAGGAGATCGGGAACTACAGGAGCTGATCCTAGTGGACTGGGGAGTGCTGA
GCACCCTGGGGCAGATAGATGGCTGGAGCACCACTCAGCTCCGCATTGTGGTCTCCAGTTTC
CTACGGCAGAGTGGTCGGCATGTGAGCCACCTGGACTTCGTTCATCTGACAGCGCTGGGTTA
TACTCTCTGTGGACTGCGGCCAGAGGAGCTCCAGCACATCAGCAGTTGGGAGTTCAGCCAAG
CAGCTCTCTTCCTCGGCACCCTGCATCTCCAGTGCTCTGAGGAACAACTGGAGGTTCTGGCC
CACCTACTTGTACTGCCTGGTGGGTTTGGCCCAATCAGTAACTGGGGGCCTGAGATCTTCAC
TGAAATTGGCACCATAGCAGCTGGGATCCCAGACCTGGCTCTTTCAGCACTGCTGCGGGGAC
AGATCCAGGGCGTTACTCCTCTTGCCATTTCTGTCATCCCTCCTCCTAAATTTGCTGTGGTG
TTTAGTCCCATCCAACTATCTAGTCTCACCAGTGCTCAGGCTGTGGCTGTCACTCCTGAGCA
AATGGCCTTTCTGAGTCCTGAGCAGCGACGAGCAGTTGCATGGGCCCAACATGAGGGAAAGG
AGAGCCCAGAACAGCAAGGTCGAAGTACAGCCTGGGGCCTCCAGGACTGGTCACGACCTTCC
TGGTCCCTGGTATTGACTATCAGCTTCCTTGGCCACCTGCTA-3′.

The partial coding sequence encoding a murine C-terminal portion of STRC protein may be as follows:

(SEQ ID NO: 58)
5′-TGCTTCAGTCCTGTACTGTGGGATCTACTCCAGAGAGAGAAGAGCGTTTGGGCCCTGAG
GACCCTGGTGAAGGCCTACCTGCGCATGCCTCCAGAAGACCTTCAGCAGCTTGTGCTTTCAG
CAGAGATGGAGGCTGCACAGGGCTTCCTGACGCTCATGCTTCGTTCCTGGGCTAAGCTGAAG
GTTCAACCATCCGAGGAGCAGGCCATGGGCCGCCTGACAGCCTTGCTGCTCCAGCGGTACCC
ACGCCTCACCTCCCAACTCTTTATCGACATGTCACCGCTCATCCCCTTCCTGGCTGTCCCTG
ACCTCATGCGCTTCCCACCGTCCCTTTTGGCCAACGACAGTGTCCTGGCTGCCATCAGGGAT
CACAGCTCAGGAATGAAGCCTGAACAGAAGGAGGCCCTGGCAAAACGACTGCTGGCCCCTGA
GCTGTTTGGAGAAGTGCCTGATTGGCCCCAGGAGCTGCTGTGGGCAGCCCTGCCTCTGCTTC
CCCATCTGCCTCTGGAGAGCTTTCTCCAGCTCAGCCCTCACCAGATCCAGGCCCTGGAGGAT
AGCTGGCCAGTAGCAGATCTTGGGCCGGGACACGCCCGACATGTGCTTCGTAGCCTAGTAAA
CCAGAGCATGGAGGATGGGGAGGAGCAGGTGCTCAGGCTTGGGTCCCTCGCCTGTTTCCTGA
GTCCTGAGGAGCTACAGAGTCTGGTGCCCTTGAGTGATCCAATGGGGCCTGTAGAACAGGGT
CTGCTGGAATGTGCGGCCAATGGGACCCTCAGCCCAGAAGGACGGGTGGCATATGAACTTCT
GGGAGTGTTGCGTTCATCTGGAGGAACTGTCTTAAGCCCCCGAGAGCTGAGGGTCTGGGCAC
CTCTCTTTCCCCAGCTGGGCCTCCGCTTCCTGCAGGAGCTCTCAGAGACCCAGCTTAGAGCC
ATGCTTCCTGCCCTACAGGGAGCCAGTGTCACACCTGCCCAGGCTGTTCTGTTGTTTGGAAG
GCTCCTTCCTAAGCATGATCTGTCCCTGGAGGAACTCTGCTCCCTGCACCCTCTCCTGCCAG
GTCTCAGCCCCCAGACACTCCAGGCCATCCCTAAGAGAGTTCTGGTTGGTGCTTGTTCCTGC
CTGGGCCCTGAACTGTCAAGGCTTTCAGCTTGCCAGATTGCAGCTCTGCTGCAGACCTTTCG
GGTAAAAGATGGTGTTAAAAATATGGGTGCAGCAGGTGCCGGCTCAGCCGTGTGCATTCCTG
GGCAGCCCACCACTTGGCCAGACTGCCTGCTTCCCCTGCTCCCATTAAAGCTGCTACAGCTG
GACGCTGCAGCTCTTCTGGCAAACCGAAGACTCTATCGGCAGCTGCCTTGGTCTGAGCAACA
GGCACAGTTTCTCTGGAAGAAAATGCAAGTGCCTACCAACCTGAGCCTGAGGAATCTGCAGG
CTCTGGGCAACTTGGCAGGAGGCATGACCTGCGAGTTTCTGCAGCAGATCAGCTCAATGGTT
GACTTTCTTGATGTGGTACACATGCTCTACCAGCTGCCCACTGGTGTTCGAGAGAGCCTGCG
GGCCTGTATCTGGACAGAGCTACAGCGGAGGATGACAATGCCAGAGCCAGAGCTGACCACCC
TAGGGCCAGAACTGAGTGAACTTGACACAAAGCTACTCCTGGACTTGCCGATCCAGCTGATG
GACAGATTGTCCAATGATTCCATTATGTTGGTGGTGGAGATGGTCCAAGGCGCTCCAGAGCA
GCTGCTGGCACTGACCCCACTCCACCAGACAGCCTTGGCAGAGCGAGCACTTAAAAACCTGG
CTCCAAAGGAGACCCCAATCTCCAAAGAAGTGCTGGAGACACTGGGCCCCTTGGTTGGATTC
CTGGGAATAGAGAGCACGCGACGGATCCCTTTACCCATTCTACTGTCTCATCTCAGTCAGCT
GCAGGGCTTCTGCCTAGGAGAGACATTTGCCACAGAGCTGGGATGGCTGCTGTTGCAGGAGC
CTGTTCTTGGAAAACCAGAATTGTGGAGCCAGGATGAAATAGAGCAAGCTGGACGCCTAGTA
TTCACTCTGTCTGCTGAGGCTATTTCCTCGATCCCCAGGGAGGCTTTGGGCCCAGAGACACT
GGAGAGGCTTCTGGGAAAGCATCAAAGCTGGGAGCAGAGCAGAGTGGGCCATCTGTGTGGGG
AGTCACAGCTTGCCCACAAGAAAGCAGCTCTGGTAGCTGGGATTGTGCATCCAGCTGCTGAG
GGTCTCCAAGAGCCTGTACCAAACTGTGCAGACATACGGGGAACCTTCCCAGCGGCCTGGTC
TGGGACACAAATCTCAGAGATGGAACTCTCAGACTTTGAAGACTGCCTGTCACTATTTGCTG
GAGATCCAGGACTTGGTCCTGAGGAACTACGGGCAGCCATGGGCAAGGCCAAGCAGTTGTGG
GGTCCCCCTCGAGGATTCCGTCCTGAGCAGATCTTGCAGCTGGGCCGTCTCCTGATAGGTCT
AGGAGAACGGGAACTGCAGGAGCTTACCTTGGTGGACTGGGGTGTGCTGAGCAGCCTGGGGC
AAATAGATGGCTGGAGTTCCATGCAGCTCCGAGCCGTGGTCTCCAGTTTCCTAAGGCAGAGT
GGTCGGCATGTGAGCCACCTGGACTTCATTTATCTGACAGCACTGGGTTACACAGTCTGTGG
ATTGCGACCAGAGGAGTTACAGCACATCAGCAGTTGGGAGTTTAGCCAAGCAGCTCTCTTCC
TGGGTAGCTTGCATCTCCCGTGCTCTGAGGAACAGCTGGAAGTTCTGGCCTATCTCCTTGTG
TTGCCTGGTGGCTTTGGCCCAGTCAGTAACTGGGGGCCTGAGATCTTCACTGAAATTGGCAC
AATAGCAGCTGGCATCCCAGACCTGGCTCTTTCAGCATTACTGCGGGGACAGATCCAAGGCC
TGACTCCTCTTGCCATTTCTGTCATTCCTGCTCCCAAGTTTGCAGTGGTCTTCAACCCCATC
CAGTTATCTAGTCTCACCAGGGGTCAGGCCGTAGCTGTTACTCCTGAACAGCTGGCCTATCT
GAGTCCTGAGCAGCGGCGAGCAGTTGCATGGGCCCAACACGAAGGGAAGGAGATCCCAGAGC
AGCTGGGTCGAAACTCAGCCTGGGGTCTCTACGACTGGTTCCAAGCCTCCTGGGCCCTGGCA
TTGCCCGTCAGCATTTTTGGCCACCTATTA-3′,

or a partial coding sequence encoding a C-terminal portion of STRC protein comprising a nucleic acid sequence consisting of SEQ ID NO:57 or 58. Another embodiment may provide a C-terminal portion of a protein of interest (e.g., STRC) (including methionine corresponding to a start codon, ATG, a signal peptide sequence; optionally a linker sequence, and a Myc tag sequence) having an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the protein of interest (e.g., STRC; SEQ ID NO:25 or 26). For example, the C-terminal portion of the STRC protein (including methionine corresponding to a start codon, ATG, STRC signal peptide sequence; optionally, a linker sequence and Myc tag sequence) may comprise an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to the sequence of SEQ ID NO:23 or 24, or a C-terminal portion of the STRC protein comprising an amino acid sequence consisting of SEQ ID NO: 23 or 24. A second nucleotide sequence of a second vector may comprise a partial coding sequence encoding a C-terminal portion of a protein of interest (e.g., STRC; SEQ ID NO:23 or 24) comprising its own signal sequence (e.g., SEQ ID NO:10 or 12) or alternatively, a different signal sequence, and a splice acceptor sequence (e.g., a C-terminal intein (C-intein); SEQ ID NO:22).

One embodiment may provide an N-intein sequence comprising a nucleic acid sequence of at least 80% (e.g., 85%, 90%, 95%, 97%, 99%, 100%) identity to SEQ ID NO:42, or an N-intein sequence encoding an amino acid sequence of at least 80% (e.g., 85%, 90%, 95%, 97%, 99%, 100%) identity to SEQ ID NO: 45. Other embodiments may be directed to an N-intein sequence consisting of a nucleic acid sequence of SEQ ID NO:42, or an N-intein sequence encoding an amino acid sequence consisting of SEQ ID NO: 45.

Another embodiment provides a C-intein sequence comprising a nucleic acid sequence of at least 80% (e.g., 85%, 90%, 95%, 97%, 99%, 100%) identity to SEQ ID NO:46, or a C-intein sequence encoding an amino acid sequence of at least 80% (e.g., 85%, 90%, 95%, 97%, 99%, 100%) identity to SEQ ID NO:49. Other embodiments may be directed to a C-intein sequence consisting of a nucleic acid sequence of SEQ ID NO:46, or a C-intein sequence encoding an amino acid sequence consisting of SEQ ID NO:49.

In a further embodiment, the dual-vector system of the disclosure provides a first nucleotide sequence encoding an N-terminal portion of a protein of interest (e.g., STRC; SEQ ID NO:15 or 16), wherein the first nucleotide sequence includes, but is not limited to, a signal sequence of the protein of interest, which may form a part of or be separate from, a partial coding sequence (5′) of the N-terminal portion of the protein of interest (e.g., STRC), and splice donor sequence (e.g., an N-intein sequence), which may form a part of or be separate from the partial coding sequence of the N-terminal portion of the protein of interest (e.g., STRC), where the first nucleotide sequence may comprise a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to a nucleotide sequence encoding an N-terminal portion of the protein of interest (e.g., STRC) and including, but not limited to, an endogenous signal sequence of the protein of interest or exogenous signal sequence, a partial coding sequence (5′) of the N-terminal portion of the protein of interest, and a splice donor sequence (e.g., an N-intein sequence) (SEQ ID NO:5 or 7). One embodiment may provide a first nucleotide sequence comprising a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to, for example, SEQ ID NO:5 or 7, or the first nucleotide sequence may comprise a nucleic acid sequence consisting of SEQ ID NO: 5 or 7.

Another embodiment provides a first nucleotide sequence encoding an amino acid sequence of at least 80% (e.g., 85%, 90%, 95%, 97%, 99%, 100%) identity to a sequence comprising an N-terminal portion of the protein of interest (e.g., STRC), wherein the first nucleotide sequence includes but is not limited to, an endogenous signal sequence of the protein of interest or exogenous signal sequence, which may form a part of or be separate from, a partial coding sequence (5′) of the N-terminal portion of the protein of interest (e.g., STRC), and splice donor sequence (e.g., an N-intein sequence), which may form a part of or be separate from the partial coding sequence of the N-terminal portion of the protein of interest (e.g., STRC). A further embodiment may provide a first nucleotide sequence encoding an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to, for example, SEQ ID NO:6 or 8, or the first nucleotide sequence may encode an amino acid sequence consisting of SEQ ID NO: 6 or 8.

Yet a further embodiment of a dual-vector system of the disclosure provides a second nucleotide sequence encoding a C-terminal portion of a protein of interest (e.g., STRC; SEQ ID NO:23 or 24), wherein the second nucleotide sequence includes, but is not limited to, an endogenous signal sequence of the protein of interest or exogenous signal sequence, which may form a part of or be separate from, a partial coding sequence (3′) of the C-terminal portion of the protein of interest (e.g., STRC), and a splice acceptor sequence (e.g., a C-intein sequence), which may form a part of or be separate from the partial coding sequence of the C-terminal portion of the protein of interest (e.g., STRC), (where the second nucleotide sequence may optionally include a linker sequence and a Myc-tag sequence in some embodiments), where the second nucleotide sequence may comprise a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to a nucleotide sequence encoding a C-terminal portion of the protein of interest (e.g., STRC) and including, but not limited to, an endogenous signal sequence of the protein of interest or exogenous signal sequence, a partial coding sequence (3′) of the C-terminal portion of the protein of interest, and a splice acceptor sequence (e.g., C-intein sequence (and optionally including a linker sequence and a Myc-tag sequence) (SEQ ID NO:17 or 19). One embodiment may provide a second nucleotide sequence comprising a nucleic acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to, for example, SEQ ID NO:17 or 19, or the second nucleotide sequence may comprise a nucleic acid sequence consisting of SEQ ID NO: 17 or 19.

Another embodiment provides a second nucleotide sequence encoding an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to a sequence comprising a C-terminal portion of the protein of interest (e.g., STRC), including but not limited to, a signal sequence of the protein of interest, a C-intein sequence, and a partial coding sequence (3′) of the C-terminal portion of the protein of interest (and optionally including a linker sequence and Myc-tag sequence). A further embodiment may provide a second nucleotide sequence encoding an amino acid sequence of at least 5% (e.g., 6%, 7%, 8%, 9%, 10%, 20%, 30%, 40%, 50%, 60%, 70%, 80%, 85%, 90%, 95%, 97%, 99%, 100%) identity to SEQ ID NO:18 or 20, or the second nucleotide sequence may encode an amino acid sequence consisting of SEQ ID NO:18 or 20.

In a further embodiment, a dual-vector system of the disclosure provides in a 5′ to 3′ direction, a first nucleotide sequence (e.g., AAV Vector 1) having an ITR, a promoter (e.g., CMV promoter), a partial coding sequence of interest (e.g., 5′ Strc), a splice donor sequence (e.g., 5′ intein), and an ITR; and a second nucleotide sequence (e.g., AAV Vector 2) having an ITR, a promoter (e.g., CMV promoter), a splice acceptor sequence (e.g., 3′ intein), a partial coding sequence of interest (e.g., 3′ Strc), and an ITR (FIG. 31A). Upon undergoing translation, the first and second nucleotide sequences may generate multiple variants, which when spliced utilizing natural cysteines at positions 747 and 970, i.e., Cys747 and Cys970 of SEQ ID NO:26 (or at positions 709 and 934, i.e., Cys709 and Cys934 of SEQ ID NO:25), form a full-length STRC protein and an excised intein comprising n-intein and c-intein. FIG. 31A illustrates eight AAV2 plasmids that include four different dual vector variants.

For example, Variant 1 comprises a first protein sequence in an N-terminal to C-terminal direction, a signal peptide sequence, an N-terminal portion of a protein of interest (e.g., n-STRC of ˜80 kD), a splice site (e.g., Ser746), and a splice donor sequence (n-intein); and a second protein sequence in an N-terminal to C-terminal direction, a splice acceptor sequence (c-intein), a splice site (e.g., Cys747), and a C-terminal portion of a protein of interest (e.g., c-STRC of ˜117 kD). Variant 2, for example, comprises a first protein sequence in an N-terminal to C-terminal direction, a signal peptide sequence, an N-terminal portion of a protein of interest (e.g., n-STRC of ˜105 kD), a splice site (e.g., Ala969), and a splice donor sequence (n-intein); and a second protein sequence in an N-terminal to C-terminal direction, a splice acceptor sequence (c-intein), a splice site (e.g., Cys970), and a C-terminal portion of a protein of interest (e.g., c-STRC of ˜92 kD). For example, Variant 3 comprises a first protein sequence in an N-terminal to C-terminal direction, a signal peptide sequence, an N-terminal portion of a protein of interest (e.g., n-STRC of ˜80 kD), a splice site (e.g., Ser746), and a splice donor sequence (n-intein); and a second protein sequence in an N-terminal to C-terminal direction, a signal peptide sequence, a splice acceptor sequence (c-intein), a splice site (e.g., Cys747), and a C-terminal portion of a protein of interest (e.g., c-STRC of ˜117 kD). Variant 4, for example, comprises a first protein sequence in an N-terminal to C-terminal direction, a signal peptide sequence, an N-terminal portion of a protein of interest (e.g., n-STRC of ˜105 kD), a splice site (e.g., Ala969), and a splice donor sequence (n-intein); and a second protein sequence in an N-terminal to C-terminal direction, a signal peptide sequence, a splice acceptor sequence (c-intein), a splice site (e.g., Cys970), and a C-terminal portion of a protein of interest (e.g., c-STRC of ˜92 kD).

Upon protein splicing of the translated variant sequences, a full-length protein of interest (e.g., STRC) forms. For example, in an N-terminal to C-terminal direction, an N-terminal portion of a protein of interest (n-STRC) may be linked to a C-terminal portion of a protein of interest (e.g., c-STRC). Splicing the N-terminal and C-terminal ends results in the excision of the splice donor sequence and the splice acceptor sequence. For example, the excised splice sequences may form a full-length splice sequence (e.g., excised intein of n-intein and c-intein) (FIG. 31A).

FIG. 31C demonstrates that HEK cells transfected with both the N-terminal portion and the C-terminal portion of variant 3 (c+n) resulted in the expression of full length STRC, as opposed to variant 3 with only the C-terminal portion (c) or either the C-terminal portion alone (c) or together with the N-terminal portion (c+n) of variant 1. The only difference between variant 1 and variant 3 is the presence of a signal sequence in C-terminal portion. Accordingly, FIG. 31C demonstrates that a signal sequence in both the N-terminal portion and the C-terminal portion directs each portion to the same cellular compartment of the cell, thereby enabling protein splicing to form the full length STRC.

A further embodiment provides a cell (e.g., host cell, mammalian cell, human cell, bacterial cell) containing the dual-vector system described herein, comprising a first vector and a second vector. In one embodiment, the cell may be an inner ear cell, an inner hair cell, or an outer hair cell. Some embodiments may be directed to a cell, where the cell is a mammalian cell (e.g., human, canine, feline, equine, murine). Other embodiments may provide an ear cell (e.g., inner ear cell, outer ear cell, inner hair cell, outer hair cell). The cell of the disclosure may be in vivo or in vitro. In some embodiments, the cell may be transfected or transformed with the first vector and the second vector of the dual-vector system of the disclosure using any of a number of known transfection and transformation techniques generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197, all of which are incorporated herein by reference in their entireties. The first vector and the second vector of the dual-vector system of the disclosure may be inserted into the cell(s) described here by any means, but not limited to, viral transduction, bacterial transformation using calcium chloride, bacterial transformation or transduction by bacterial mating or conjugation, transfection (e.g., electroporation, calcium phosphate, liposome-based transfection), gene gun, and the like.

Yet another embodiment may be directed to a composition or pharmaceutical composition comprising the dual-vector system described herein, and a pharmaceutically- or physiologically-acceptable vehicle (e.g., diluent, carrier, excipient). Compositions contemplated herein for the treatment of diseases or conditions associated with a mutation may comprise the dual-vector system described herein. For therapeutic purposes, compositions comprising a polynucleotide of interest (e.g., STRC) or fragments thereof, which when properly processed in accordance with the methods disclosed herein, result in the expression of a full-length protein of interest (e.g., STRC protein) in a genome that comprises a mutation that causes or contributes to a disease or condition (e.g., autosomal recessive DFNB16 hearing loss) as described herein may be administered directly to a region of the body (e.g., cochlea, inner ear) that is affected by the disease or condition. In some embodiments, the compositions are formulated in a pharmaceutically-acceptable buffer such as physiological saline. Non-limiting methods of administration include injecting into the ear, inner ear, cochlear duct, or the perilymph-filled spaces surrounding the cochlear duct (e.g., scala tympani and scala vestibuli). Injecting into the cochlear duct, which is filled with high potassium endolymph fluid, could provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed from the middle ear, either through the oval or round window membrane. The round window membrane, which is the only non-bony opening into the inner ear, is relatively easily accessible in many animal models and administration of viral vector using this route is well tolerated. In humans, cochlear implant placement routinely relies on surgical electrode insertion through the round window membrane.

### Methods of Use

One embodiment may provide methods of using the vector system (e.g., dual-vector system; capsid, plasmid, transplicing plasmid, viral vector, Adenovirus, AAV, AAV genome, lentivirus) described herein, where the method may treat and/or reduce and/or prevent a disease, condition, or symptom thereof resulting from a defective or mutated gene, comprising administering to a subject in need thereof, an effective amount of the dual-vector system described herein.

Another embodiment may be directed to a method, comprising: contacting a cell (e.g., of a subject) with a composition comprising the vector system (e.g., dual-vector system) described herein, and a pharmaceutically- or physiologically-acceptable vehicle (e.g., carrier, diluent, excipient). The contacting step with a cell (e.g., of a subject) may result in the delivery of a nucleotide sequence of a vector (e.g., plasmid, transplicing plasmid, viral vector, Adenovirus, AAV, AAV genome) comprising, for example, SEQ ID NO:33 (FIGS. 2A-2C) or SEQ ID NO:38 (FIGS. 4A-4D), where the cell may express a full-length protein of interest (e.g., STRC; human SEQ ID NO:2 or 25; murine SEQ ID NO:4 or 26). The contacting step with a cell of a subject may result in the delivery of a first nucleotide sequence and the second nucleotide sequence (of a first vector and a second vector, respectively), where the cell may express an N-terminal portion of a protein of interest (e.g., STRC) and a C-terminal portion of the protein of interest, and the N-terminal portion of a protein of interest (e.g., STRC) and a C-terminal portion of the protein of interest are joined by a peptide bond to form a full-length protein of interest (e.g., STRC; human SEQ ID NO:2 or 25; murine SEQ ID NO:4 or 26).

One embodiment may provide a method for treating autosomal recessive hearing loss in a subject, comprising administering to the subject in need thereof, an effective amount of the vector system (e.g., dual-vector system) described herein or composition described herein or cell containing the vector system (e.g., dual-vector system) of the disclosure or composition of the disclosure or a vector or composition comprising at least one nucleotide sequence (e.g., STRC; SEQ ID NO:5; SEQ ID NO:7; SEQ ID NO:17; SEQ ID NO:19; SEQ ID NO:30; SEQ ID NO:32) encoding a protein of interest (e.g., STRC; SEQ ID NO:25; SEQ ID NO:26) or the protein of interest (e.g., STRC; SEQ ID NO:25; SEQ ID NO:26) itself or portions of the protein of interest (e.g., SEQ ID NO:6-SEQ ID NO:16; SEQ ID NO:18-SEQ ID NO:24), where administration may result in the reduction or restoration of the autosomal recessive hearing loss or symptoms thereof. In some embodiments, the subject in need thereof will have been successfully treated if the subject after treatment has a hearing level of 69 dB or less (e.g., 60 dB, 55 dB, 50 dB, 45 dB, 40 dB, 35 dB, 30 dB, 26 dB, 25 dB, 20 dB, 15 dB, 10 dB, 5 dB, 0 dB). Generally, those subjects with profound hearing loss cannot hear sounds lower than 95 dB; severe hearing loss, subjects cannot hear sounds lower than 70 dB to 94 dB; moderate hearing loss subjects cannot hear sounds lower than 40 dB to 69 dB; and mild hearing loss, subjects cannot hear sounds lower than 26 dB to 40 dB. However, those subjects suffering from hearing loss, including for example, autosomal recessive hearing loss, may be treated by any of the methods described herein, thus resulting in the reduction of hearing loss or symptoms thereof and/or the restoration of or improved hearing (or auditory function in the subject) and/or the maintenance of hearing. A subject having normal hearing may be characterized as hearing sounds of 25 dB or less (e.g., 20 dB, 15 dB, 10 dB, 5 dB, 0 dB). In some embodiments, the autosomal recessive hearing loss is DFNB16.

A further embodiment may provide a method for treating and/or preventing a pathology or disease characterized by a hearing loss comprising administering to a subject in need thereof an effective amount of the dual-vector system described herein, the cell according to the disclosure, or the composition or pharmaceutical composition described herein. The cell of the disclosure may be an inner ear cell, an inner hair cell, an outer hair cell, in vivo, in vitro, or the like, or combinations of any of the foregoing.

### Polynucleotide Delivery

Therapeutic success in these approaches relies significantly on the safe and efficient delivery of exogenous gene constructs to the relevant therapeutic cell targets in the organ of Corti in the cochlea. The organ of Corti includes two classes of sensory hair cells: inner hair cells, which convert mechanical information carried by sound into electrical signals transmitted to neuronal structures and outer hair cells which serve to amplify and tune the cochlear response, a process required for complex hearing function.

Methods of delivering nucleic acids to cells generally are known in the art, and methods of delivering viruses (which also can be referred to as viral particles) containing a transgene to inner ear cells in vivo are described herein. As described herein, about 10⁸ to about 10¹² viral particles can be administered to a subject, and the virus can be suspended within a suitable volume (e.g., 10 μL, 50 μL, 100 μL, 500 μL, or 1000 μL) of, for example, artificial perilymph solution.

Viruses containing inverted terminal repeats (ITRs), a promoter (e.g., an Espin promoter, a PCDH15 promoter, a PTPRQ promoter, a Myo6 promoter, a KCNQ4 promoter, a Myo7a promoter, a synapsin promoter, a GFAP promoter, a CMV promoter, a CAG promoter, a CBH promoter, a CBA promoter, a U6 promoter, and a TMHS (LHFPL5) promoter), a signal sequence, a polynucleotide encoding a protein of interest (e.g., STRC protein), and a poly adenylation (polyA) sequence, and in some embodiments, a linker sequence for linking a c-myc tag, as described herein can be delivered to inner ear cells (e.g., cells in the cochlea) using any number of means. For example, a therapeutically effective amount of a composition including virus particles containing the dual-vector intein-mediated protein trans-splicing system as described herein can be injected through the round window or the oval window, or the utricle, typically in a relatively simple (e.g., outpatient) procedure. In some embodiments, a composition comprising a therapeutically effective number of virus particles containing dual-vector intein-mediated protein trans-splicing system (e.g., a dual-AAV intein-mediated STRC protein system), or containing one or more sets of different virus particles, as described herein may be delivered to the appropriate position within the ear during surgery (e.g., a cochleostomy or a canalostomy).

In addition, delivery vehicles (e.g., polymers) are available that facilitate the transfer of agents across the tympanic membrane and/or through the round window or utricle, and any such delivery vehicles can be used to deliver the viruses described herein. See, for example, Arnold et al., 2005, Audiol. Neurootol., 10:53-63, incorporated herein by reference in its entirety for delivery vehicles.

The compositions and methods described herein enable the highly efficient delivery of nucleic acids to inner ear cells, e.g., cochlear cells. For example, a polynucleotide encoding a protein of interest (e.g., STRC protein), or a fragment thereof, may be cloned into a viral vector and expression may be driven from its endogenous promoter, from the viral inverted terminal repeat, or from a promoter specific for a target cell type of interest. Other viral vectors that can be used include, for example, a vaccinia virus, a bovine papilloma virus, or a herpes virus, such as Epstein-Barr Virus. Viral vectors have been used in clinical settings. In some embodiments, a viral vector (e.g., rAAV) may be used to administer a large (e.g., Strc gene) polynucleotide in fragments. In some embodiments, a viral vector may be used to administer the Strc polynucleotide fragments to a particular region of the body.

For example, the compositions and methods described herein enable the delivery to, and expression of, a polynucleotide of interest (e.g., Strc) in at least 65% or greater (e.g., 70%, 75%, 80%, 85%, 90%, 91%, 92%, 93%, 94%, 95%, 96%, 97%, 98%, 99%) of inner and/or outer hair cells or delivery to, and expression in, at least 65% or greater (e.g., 70%, 75%, 80%, 85%, 90%, 91%, 92%, 93%, 94%, 95%, 96%, 97%, 98%, 99%) of outer hair cells.

Expression of a STRC polynucleotide delivered using the dual-vector intein-mediated system described herein may result in improved structure and function of inner and outer hair cells, such that hearing is restored for an extended period of time (e.g., days, weeks, months, years, decades, a life time). In one embodiment, hearing loss may be recovered in those subjects suffering from an autosomal recessive type of non-syndromic deafness, DFNB16, caused by mutations in the STRC gene. Normal expression of STRC and stereocilin (STRC) protein are essential for auditory function.

As described herein, an adeno-associated virus (AAV) are particularly efficient at delivering nucleic acids (e.g., polynucleotides encoding a STRC polypeptide) to inner ear cells. The Anc80 vector is an example of an Inner Ear Hair Cell Targeting AAV that advantageously transduced 60% or greater (e.g., 70%, 80%, 90%, 95%, 100%) of inner or outer hair cells. One embodiment may utilize an ancestral capsid protein that falls within the class of Anc80 ancestral capsid protein, e.g., Anc80-0065, described in International Publication No. WO 2018/145111 (PCT/US2018/017104), which is incorporated herein by reference in its entirety regarding Anc80. WO 2015/054653, which is also incorporated herein by reference in its entirety, describes a number of additional ancestral capsid proteins that fall within the class of Anc80 ancestral capsid proteins.

In particular embodiments, the adeno-associated virus (AAV) contains an ancestral AAV capsid protein that has a natural or engineered tropism for hair cells. In some embodiments, the virus is an Inner Ear Hair Cell Targeting AAV, which delivers a polynucleotide of interest encoding a polypeptide of interest (e.g., STRC protein) to the inner ear in a subject (e.g., subject suffering from DFNB16 and/or mutations in the STRC gene). In some embodiments, the virus is an AAV that comprises purified capsid polypeptides. In some embodiments, the virus is artificial. In some embodiments, the virus is an AAV that has lower seroprevalence than AAV2. In some embodiments, the virus is an exome-associated AAV. In some embodiments, the virus is an exome-associated AAV1. In some embodiments, the virus comprises a capsid protein with at least 95% amino acid sequence identity or homology to Anc80 capsid proteins.

Expression of a polynucleotide of interest (e.g., STRC) may be directed by a heterologous promoter (e.g., CMV promoter, Espin promoter, a PCDH15 promoter, a PTPRQ promoter, a TMHS (LHFPL5) promoter). As used herein, a “heterologous promoter” refers to a promoter that does not naturally direct expression of that sequence (i.e., is not found with that sequence in nature).

Methods for packaging a transgene into a virus that contains, for example, an Anc80 capsid protein are known in the art, and utilize conventional molecular biology and recombinant nucleic acid techniques. In one embodiment, a construct that includes a nucleic acid sequence encoding an Anc80 capsid protein and constructs carrying fragments of the polynucleotide encoding N-terminal and C-terminal portions of a STRC protein flanked by suitable Inverted Terminal Repeats (ITRs) are provided, which allows for packaging within the Anc80 capsid protein.

The polynucleotide of interest (e.g., STRC) may be packaged into AAV containing an Anc80 capsid protein using, for example, a packaging host cell. The components of a virus particle (e.g., rep sequences, cap sequences, inverted terminal repeat (ITR) sequences) may be introduced, transiently or stably, into a packaging host cell using one or more constructs as described herein. The polynucleotide of interest may generally be a large gene (e.g., 4 kB or greater) which may require being split and packaged into more than one AAV.

In some embodiments, AAVs containing a AAV9-php.b vector may be used to efficiently target inner ear cells. AAV9-php.b is described in International Publication No. WO 2019/173367 (PCT/US2019/020794), the contents of which are incorporated herein by reference in their entirety. AAV-PHP.B encodes the 7-mer sequence TLAVPFK (SEQ ID NO:59) and efficiently delivers transgenes to the cochlea, where it showed remarkably specific and robust expression in the inner and outer hair cells. An AAV-PHP.B vector may comprise, but is not limited to, any of the promoters described herein.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types may be used to direct the expression of a nucleic acid. The enhancers used may include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

### Therapeutic Methods

Another therapeutic approach included in the disclosure may involve administration of a recombinant therapeutic (e.g., recombinant STRC protein, variant, or fragment thereof), either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

Some embodiments of the disclosure may provide methods of treating or preventing or reducing a disease and/or disorder or symptoms thereof in a subject in need thereof, which comprise administering a therapeutically effective amount of a pharmaceutical composition comprising the vector system (e.g., dual-vector intein-mediated system) containing nucleotide sequence encoding a full-length protein of interest (e.g., STRC) in a cell (e.g., of a subject), where the genome of the cell may comprise a mutation. In embodiments using a dual-vector intein-mediated system of the disclosure, methods of treating or preventing or reducing a disease and/or disorder or symptoms thereof in a subject in need thereof, may comprise administering a therapeutically effective amount of a pharmaceutical composition comprising a dual-vector intein-medicated system containing a first nucleotide sequence encoding a portion of a protein of interest (e.g., N-STRC) and a second nucleotide sequence encoding a remaining portion of a protein of interest (e.g., C-STRC) in the genome comprising a mutation in the subject in need thereof, where the subject (e.g., mammalian, such as a human). Thus, one embodiment is a method of treating a subject suffering from or susceptible to a disease or disorder or symptom thereof associated with a mutation. The method includes administering to the subject a therapeutic amount of a composition herein sufficient to treat or prevent, or reduce the disease or disorder or symptom. In some embodiments, the mutation is a recessive mutation.

The therapeutic methods of the invention (which include prophylactic treatment), in general, comprise administration of a therapeutically effective amount of the compounds or compositions herein, such as a compound of the formulae herein to a subject (e.g., animal, human) in need thereof, including a mammal, e.g., a human. Such treatment will be suitably administered to subjects, particularly humans, suffering from, having, susceptible to, or at risk for a disease, disorder, or symptom thereof. Determination of those subjects “at risk” may be made by any objective or subjective determination by a diagnostic test or opinion of a subject or health care provider (e.g., genetic test, enzyme or protein marker, Marker (as defined herein), family history, and the like).

Treatment of human patients or non-human animals may be carried out using a therapeutically effective amount of a combination therapeutic in a physiologically-acceptable carrier. The phrase “pharmaceutically acceptable” refers to those compounds of the disclosure, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compositions may conveniently be presented in unit dosage form and may be prepared by any methods well known in the art. The amount of composition (e.g., vectors containing sequences encoding N-terminal or C-terminal portions of the protein of interest (e.g., STRC)) which can be combined with a carrier material to produce a single dosage form will vary depending upon the host being treated, the particular mode of administration. The amount of composition which can be combined with a carrier material to produce a single dosage form will generally be that amount of the composition which produces a therapeutic effect. Generally, out of one hundred percent, this amount will range from 1% to 99% (e.g., 5%-70%, 10%-30%) of composition.

Compositions may be administered at a dosage that controls the clinical or physiological symptoms of the disease or condition, as may in some cases be determined by a diagnostic method known to one skilled in the art.

Therapeutic compositions and therapeutic combinations are administered in an effective amount. For example, about 10⁸ to about 10¹² viral particles may be administered to a subject, and the virus may be suspended within a suitable volume (e.g., 10 μL, 50 μL, 100 μL, 500 μL, or 1000 μL) of, for example, artificial perilymph solution.

### Methods of Treating Autosomal Recessive Hearing Loss

Compositions and methods for treating autosomal recessive hearing loss (e.g., Deafness, Autosomal Recessive 16 of non-syndromic deafness (DFNB16)) are provided.

Briefly, DFNB16 is associated with mutations in the STRC gene of affected individuals. Normal expression of STRC, encoding stereocilin (STRC) extracellular structural protein, in the inner ear is essential for auditory function. In order to induce recovery of hearing loss, the wild-type STRC gene is administered to a subject using the vector system (e.g., dual-AAV intein-mediated STRC protein trans-splicing system) as described herein, namely by packaging the wild-type STRC gene sequence or fragments thereof, in order for the full-length mRNA and full-length STRC protein to be expressed.

In some embodiments, a vector system encoding a STRC protein, including, for example, dual-AAV intein-mediated STRC protein trans-splicing vectors, may be administered to a subject having DFNB16 hearing loss by directly injecting the at least one vector (e.g., 5′ STRC and 3′ STRC vectors) encoding the stereocilin (STRC) protein into the cochlea of a subject. In some embodiments, one vector only encodes the N-STRC protein and one vector only encodes the C-STRC protein. For therapeutic purposes, compositions comprising a STRC polypeptide, or a STRC polynucleotide encoding a STRC polypeptide may be administered directly to a region of the body (e.g., cochlea) that is affected by the disease or condition, where the subject's genome comprises a STRC mutation that causes or contributes to hearing loss (e.g., DFNB16) as described herein.

One embodiment may provide a method for treating autosomal recessive hearing loss in a subject, comprising administering to a subject in need thereof, an effective amount of: a vector system (e.g., dual-vector system) described herein; a cell containing the vector system (e.g., dual-vector system) described herein; or a pharmaceutical composition comprising the vector system (e.g., dual-vector system) described herein and a pharmaceutically-acceptable vehicle. Some embodiments may be directed to methods of treating an autosomal recessive hearing loss that is DFNB16.

Another embodiment of the disclosure provides a method comprising, contacting a cell of a subject with a composition comprising the vector system (e.g., dual-vector system) described herein and a pharmaceutically-acceptable vehicle, wherein the contacting results in the delivery of the first nucleotide sequence which expresses an N-terminal portion of a protein and the second nucleotide sequence which expresses a C-terminal portion of the protein into the cell, wherein the cell expresses the N-terminal portion of the protein and the C-terminal portion of the protein joined by a peptide bond to form a full-length protein.

A further embodiment of the disclosure provides a method for treating and/or preventing a pathology or disease characterized by a hearing loss comprising administering to a subject in need thereof an effective amount of the vector system (e.g., dual-vector system) described herein; a cell containing the vector system (e.g., dual-vector system) described herein; or a pharmaceutical composition comprising the vector system (e.g., dual-vector system) described herein and a pharmaceutically-acceptable vehicle, wherein the administering step occurs in at least one cell of the subject (e.g., an inner ear cell, inner hair cell, outer hair cell). In one embodiment, the method of contacting a cell or administering to a cell an effective amount of the vector system (e.g., dual-vector system) described herein; a cell containing the vector system (e.g., dual-vector system) described herein; or a pharmaceutical composition comprising the vector system (e.g., dual-vector system) described herein and a pharmaceutically-acceptable vehicle occurs in vivo, ex vivo, and/or in vitro. Another embodiment provides for any of the methods described herein, where the method improves or restores auditory function in a subject.

Non-limiting methods of administration may include injecting into the cochlear duct or the perilymph-filled spaces surrounding the cochlear duct (e.g., scala tympani and scala vestibuli). Injecting into the cochlear duct, which is filled with high potassium endolymph fluid, could provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed from the middle ear, either through the oval or round window membrane. The round window membrane, which is the only non-bony opening into the inner ear, is relatively easily accessible in many animal models and administration of viral vector using this route is well tolerated. In humans, cochlear implant placement routinely relies on surgical electrode insertion through the round window membrane.

In some embodiments, expressing the protein of interest (e.g., wild-type STRC) may restore auditory function in a subject. In some embodiments, the auditory function restored to a subject may be 10% or greater (e.g., 20%, 30%, 40%, 50%, 60%, 70%, 80%, 90%, 100%); 100% or less (e.g., 95%, 85%, 75%, 65%, 55%, 45%, 35%, 25%, 15%).

### EXAMPLES

The following examples illustrate specific aspects of the instant description. The examples should not be construed as limiting, as the example merely provides specific understanding and practice of the embodiments and its various aspects.

### Example 1: Dual-Vector System Transduces Large Genes In Vitro

### AAV Vector Production

The dual-vector or dual-trans-splicing system for delivery of a protein of interest disclosed herein was demonstrated using, for example, a STRC coding sequence in two independent adeno-associated virus, serotype 2 (AAV2)/adeno-associated virus, serotype 9 (AAV9)-Php.B vectors into the inner ear. A first AAV genome with a splice donor sequence (e.g., N-Intein) located immediately after the first 2,247 base pairs of the STRC coding sequence (i.e., N-terminal portion of STRC) and followed by the 3′-ITR was produced (e.g., AAV2/AAV9-Php.B-STRC-trans/donor). A second AAV genome was produced with a corresponding splice acceptor sequence located after the 5′-ITR and immediately prior to the remaining STRC coding sequence (i.e., C-terminal portion of STRC) with a C-terminal myc tag (e.g., AAV2/AAV9-Php.B-STRC-trans/acceptor). Since AAV2 genomes are known to form concatemers with inverted terminal repeats (ITRs) at each end of the viral genome, the STRC coding sequence was divided into two fragments and packaged into separate AAV capsids (e.g., synthetic AAV: Anc80 which was shown to transduce inner and outer hair cells with efficiency).

To confirm intein-mediated trans-splicing and processing, HEK293 cells infected with AAV2/AAV9-Php.B vectors encoding for portions of full-length STRC (5 days post-infection) were analyzed by Western blot (FIG. 24). Specifically, FIG. 24 shows the following: Lane 1: Control or untransfected HEK293T cells; Lane 2: Full-length Stereocilin (STRC) (196.4 kDa); Lane 3: pCFS-#2, C portion (vector with construct #2 having only C-portion (116.7 kDa)); Lane 4: pCFS-#2, N+C portions (vectors with construct #2 having N- and C-portions); Lane 5: pCFS-#1, C portion (vector with construct #1 having only C-portion (91.6 kDa)); Lane 6: pCFS-#1, N+C portions (vectors with construct #1 having N- and C-portions). The arrow on the right side of the Western blot points to a full-length STRC protein in Lane 4 demonstrating that both the N-terminal portion and the C-terminal portion of STRC was formed when the two AAV2/AAV9-Php.B vectors respectively containing a sequence encoding the N-terminal portion and the C-terminal portion of STRC were transfected into HEK293 cells.

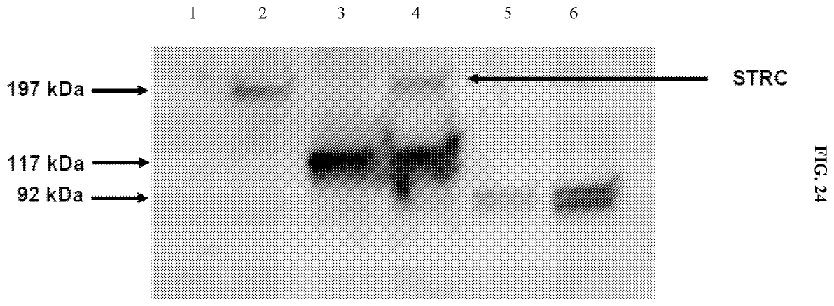

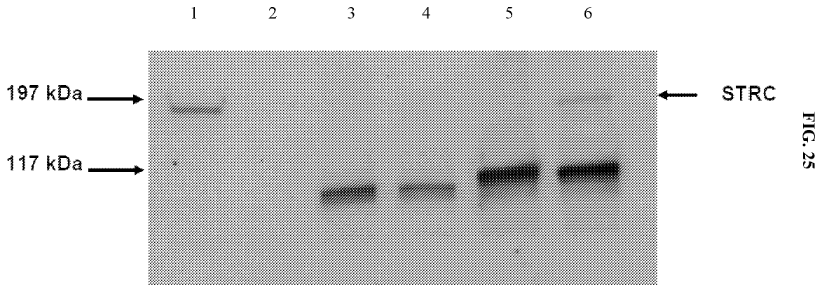

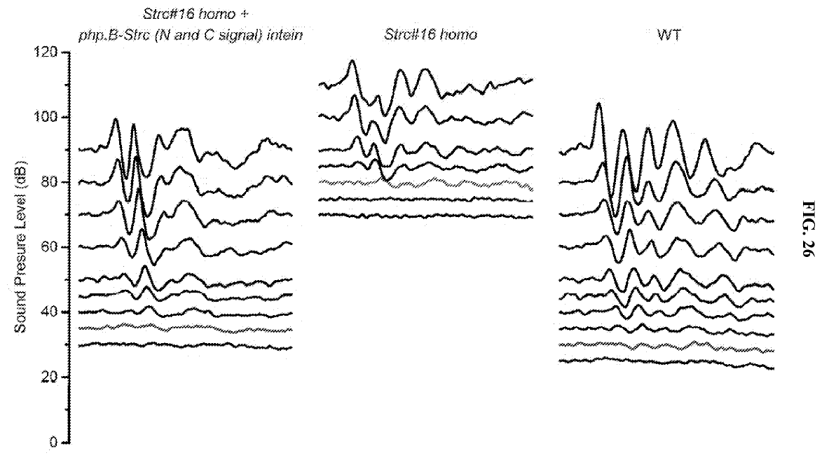

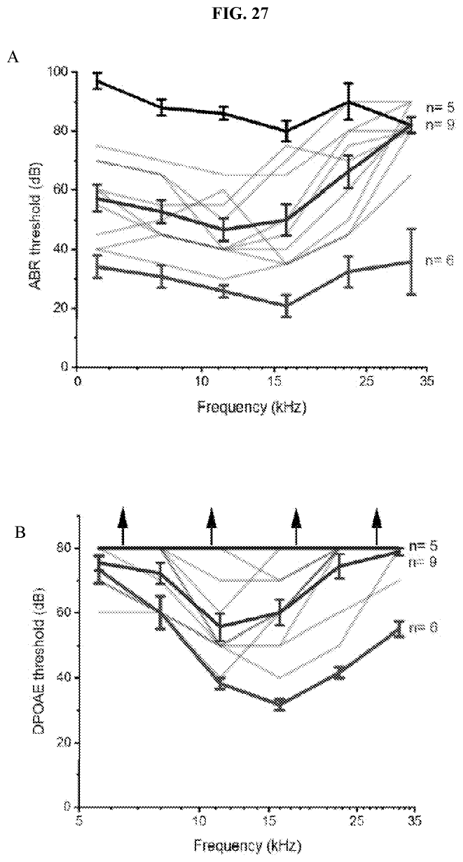

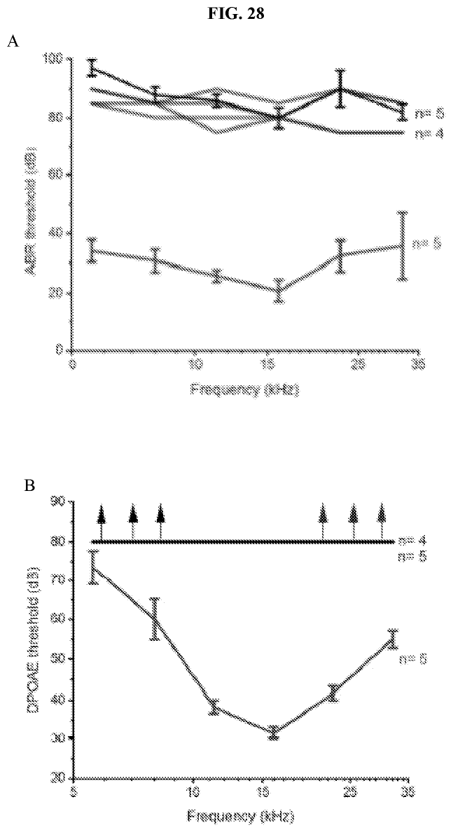

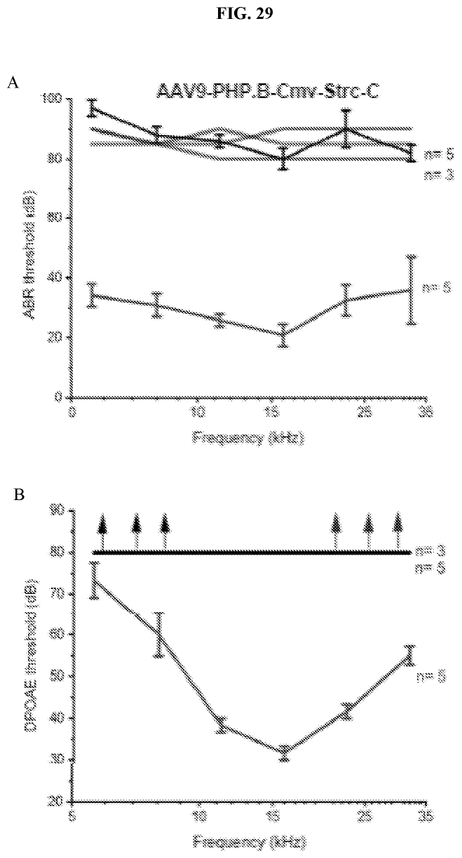

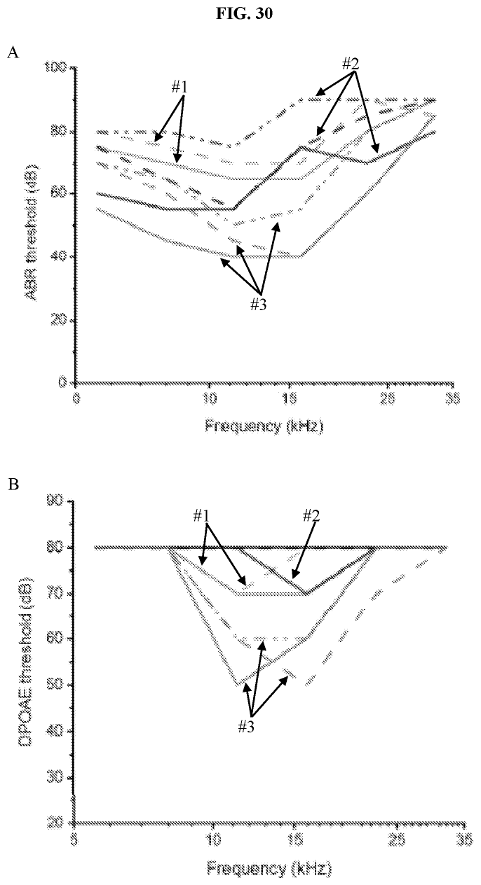

To confirm the usefulness of the signal sequence, a further Western blot was performed. FIG. 25 shows the following: Lane 1: Full-length Stereocilin (STRC) (196.4 kDa); Lane 2: Control or untransfected HEK293T cells; Lane 3: pCFS-#2, C portion, (−) signal (vector with construct #2 having only C-portion (116.7 kDa) without signal sequence); Lane 4: pCFS-#2, N+C portions, (−) signal (vectors with construct #2 having N- and C-portions without signal sequences); Lane 5: pCFS-#2, C portion, (+) signal (vector with construct #2 having only C-portion (91.6 kDa) with signal sequence); Lane 6: pCFS-#2, N+C portions, (+) signal (vectors with construct #2 having N- and C-portions with signal sequences). The arrow on the right side of the Western blot points to a full-length STRC protein in Lane 6 demonstrating that the signal sequence was necessary in order to result in the formation of the full-length STRC protein.

AAV vectors were produced by the Boston Children's Hospital Viral Core (Boston, Mass., USA). Plasmid containing STRC and intein sequenced before packaging (MGH DNA Core, complete plasmid sequencing) into AAV9-php.b-cmv. Vector titer was 4.8×10¹⁴ gc/ml as determined by qPCR specific for the inverted terminal repeat (AAV2) of the virus.

### Example 2: Analysis of Dual-Vector System in Strc Knockout Mice In Vivo

### Animals

All animals were bred and housed in facilities. All studies involving animals were approved by the HMS Standing Committee on Animals (Protocol No. 03524) and the Boston Children's Hospital Institutional Animal Care and Use Committee (Protocol Nos. 2878 and 3396). All experiments were conducted in accordance with the animal protocols.

Null allele (“knockout”) mice that were stereocilin deficient (STRC^{-/-}; Strc^{Δ/Δ}) were generated and served as a mouse model for human hearing loss DFNB16 phenotype caused by STRC mutations, which lead to absent or non-functional stereocilin protein. Strc homozygous mutant mice (STRC #16 homo) exhibited severe hearing loss by 4 weeks of age as determined by auditory brainstem responses (ABRs), and by 6 weeks of age, the mutant mice were completely deaf. Strc homozygous mutant mice also lacked detectable distortion product otoacoustic emissions (DPOAEs) up to 80 dB sound pressure level which reflects the absence of normal outer hair cells (OHCs) function.

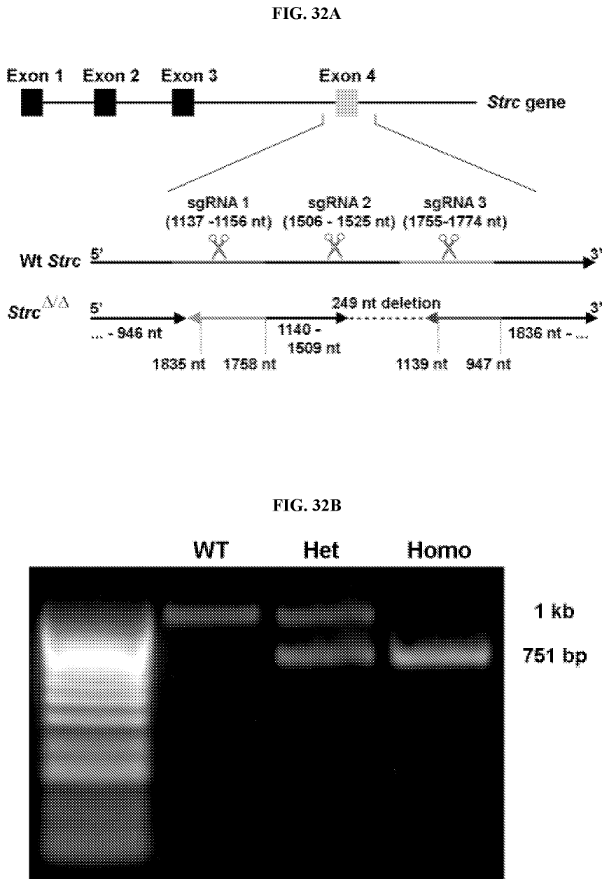

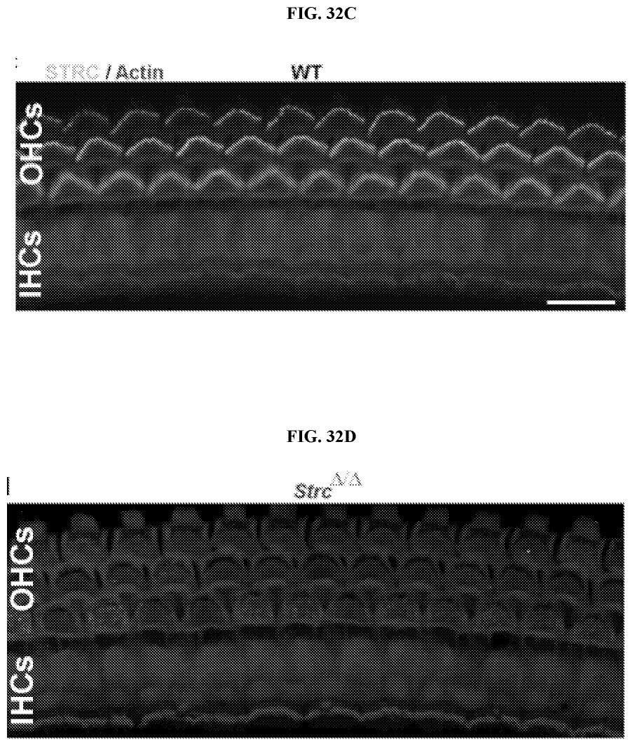

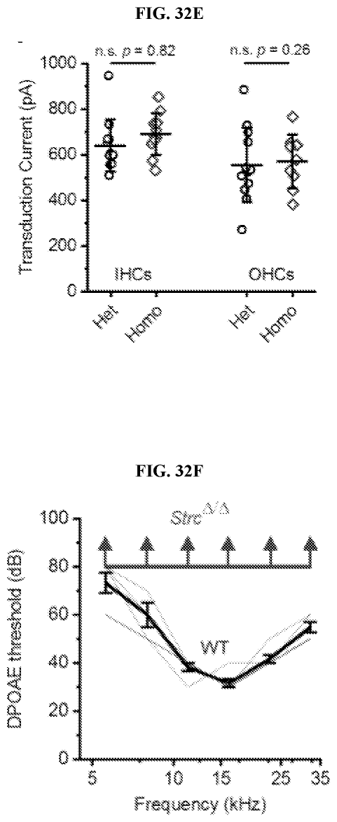

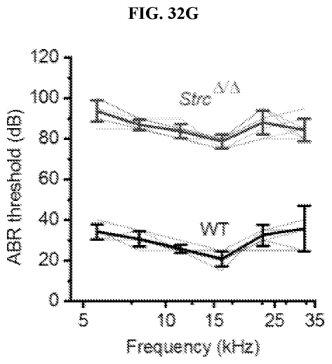

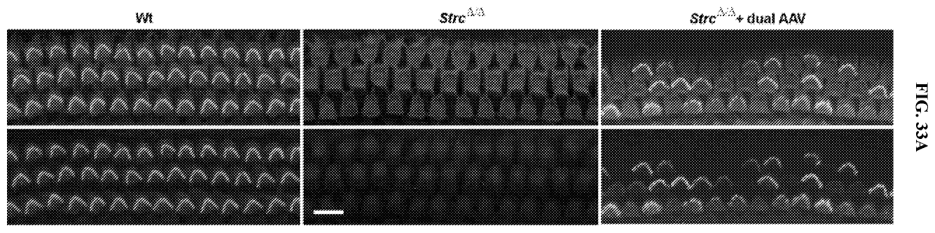

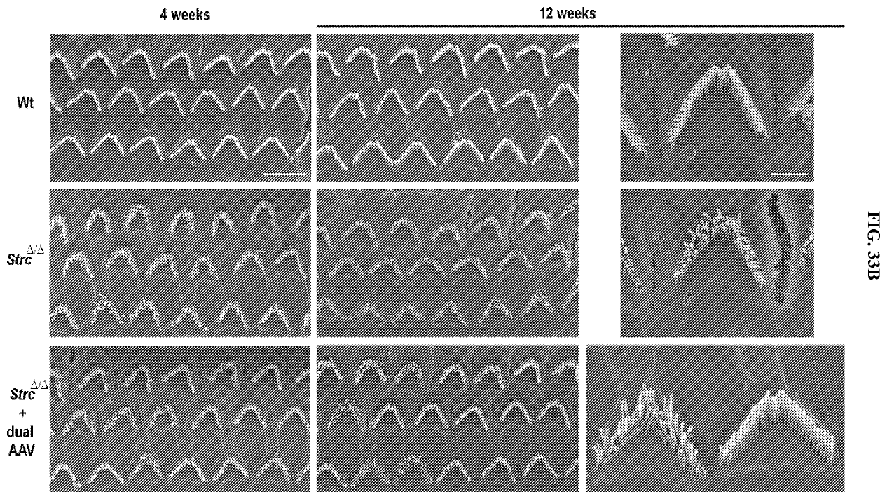

Strc^{Δ/Δ} mice were generated and characterized in FIGS. 32A-32G. A wild-type (WT) protein of interest, for example, Strc was disrupted using the CRISPR/Cas9 strategy by designing three guide RNAs (sgRNA) to target exon 4 of the Strc gene. The disruption resulted in a 249 nucleotide deletion (positions 1509-1758) and two transpositions and inversions (positions 947-1139 closer to the 3′ end; positions 1758-1835 closer to the 5′ end).

### Inner Ear Injections

Inner ears of Strc^{-/-} or Strc^{WT/WT} mouse pups were injected at postnatal day 1 (P1) with 1 μl of AAV9-php.b-cmv-STRC intein virus at a rate of 60 nl/min. Pups were anesthetized using hypothermia exposure in ice water for 2-3 minutes. Upon anesthesia, a post-auricular incision was made to expose the otic bulla and visualize the cochlea. Injections were made manually with a glass micropipette. After injection, a suture was used to close the skin cut. Then, the injected mice were placed on a 42° C. heating pad for recovery. Pups were returned to the mother after they recovered fully within ˜10 minutes. Standard post-operative care was applied after surgery. Sample sizes for in vivo studies were determined on a continuing basis to optimize the sample size and decrease the variance. At P5 to P7, organs of Corti were excised from injected ears. Organ of Corti tissues were incubated at 37° C., 5% CO₂ for 8-10 days, and the tectorial membrane was removed immediately before electrophysiology recording.

### Hearing Tests

To determine whether the dual-vector system using AAV vectors comprising sequences encoding the N-STRC with N-Intein and the C-STRC with C-Intein, respectively were capable of and the extent of recovering hearing loss, Auditory Brainstem Responses (ABRs) and Distortion Product Otoacoustic Emissions (DPOAEs) were measured in the stereocilin knockout (STRC^{-/-}) mice. ABR and DPOAE measurements were recorded using the EPL Acoustic system (Massachusetts Eye and Ear, Boston). Acoustic stimuli were generated with 24-bit digital Input/Output cards (National Instruments PXI-4461) in a PXI-1042Q chassis, amplified by a SA-1 speaker driver (Tucker-Davis Technologies, Inc.), and delivered from two electrostatic drivers (CUI CDMG15008-03A) in a custom acoustic system. An electret microphone (Knowles FG-23329-P07) at the end of a small probe tube was used to monitor ear-canal sound pressure. ABRs and DPOAEs were recorded from mice during the same session. ABR signals were collected using subcutaneous needle electrodes inserted at the pinna (active electrode), vertex (reference electrode), and rump (ground electrode). ABR potentials were amplified (10,000×), pass-filtered (0.3-10 kHz), and digitized using custom data acquisition software (LabVIEW) from the Eaton-Peabody Laboratories Cochlear Function Test Suite. Sound stimuli and electrode voltage were sampled at 40-μs intervals using a digital I-O board (National Instruments) and stored for offline analysis. Threshold was defined visually as the lowest decibel level at which peak 1 could be detected and reproduced with increasing sound intensities. ABR thresholds were averaged within each experimental group and used for statistical analysis. ABR and DPOAE measurements were performed by investigators blinded to the genotype.

Mice were anesthetized with intraperitoneal (i.p.) injection of xylazine (5-10 mg/kg) and ketamine (60-100 mg/kg), and the base of the pinna was trimmed away to expose the ear canal. Three subcutaneous needle electrodes were inserted into the skin, including a) dorsally between the two ears (reference electrode); b) behind the left pinna (recording electrode); and c) dorsally at the rump of the animal (ground electrode). Additional aliquots of ketamine (60-100 mg/kg i.p.) were given throughout the session to maintain anesthesia if needed. Prior to ABR testing, the sound pressure at the entrance of the ear canal was calibrated for each individual test subject at all stimulus frequencies. ABR and DPOAE data were collected under the same conditions and during the same recording sessions.

DPOAEs were recorded first. Primary tones were produced at a frequency ratio of 1.2 (the frequency ratio of f1 and f2 primary tones (f2/f1=1.2)) for generating DPOAEs at 2f1-f2, where the f2 level was 10 dB sound pressure level below f1 level for each f2/f1 pair. The tones were presented with f2 varied between 5.6 and 32.0 kHz in half-octave steps and L1−L2=10 decibel sound pressure level (dB SPL). At each f2, L2 was varied between 10 and 80 dB in 10 dB increments. DPOAE threshold was defined from the average spectra as the L2-level eliciting a DPOAE of magnitude 5 dB above the noise floor. The mean noise floor level was under 0 dB across all frequencies. At each level, waveform and spectral averaging were used in order to increase the signal-to-noise (s/n) ratio of the recorded ear-canal sound pressure. DPOAE at 2f1-f2 had an amplitude that was extracted from the averaged spectra, as well as the noise floor at neighboring points in the spectrum. Interpolation from plots of DPOAE amplitude versus sound level resulted in iso-response curves. Threshold was defined as the f2 level required to produce DPOAEs above 0 dB.

ABR experiments were then performed at 32° C. in a sound-proof chamber. To test hearing function, mice were presented with stimuli of broadband “click” tones as well as the pure tones between 5.6 and 32.0 kHz in half-octave steps, all presented as 5-ms tone pips. The responses were amplified (10,000 times), filtered (0.1-3 kHz), and averaged with an analog-to-digital board in a PC-based data-acquisition system (EPL, Cochlear function test suite, MEE, Boston). Across various trials, the sound level was raised in 5 to 10 dB steps from 0 to 110 dB SPL. At each level, 512 responses were collected and averaged for each sound pressure level (with stimulus polarity alternated) after “artifact rejection.” Threshold was determined by visual inspection of the appearance of Peak 1 relative to background noise. Data were analyzed and plotted using Origin-2015 (OriginLab Corporation, MA). Thresholds averages±standard deviations are presented unless otherwise stated. The majority of these experiments were not performed under blind conditions.

The knockout mouse lacking STRC was generated by disrupting the Strc gene coding sequence with NHEJ-mediated Cas9-generated breaks. A ˜200 base pair deletion was generated within exon 4 of the STRC gene, which disrupted the synthesis of the functional protein. This mouse model was found to accurately recapitulate human hearing loss of the DFNB16 phenotype caused by STRC mutations that result in the absence or non-functional stereocilin protein. Week four aged STRC homozygous mutant mice exhibited severe hearing loss based on auditory brainstem responses (ABR), and by week 6, these mice were completely deaf. An ABR threshold may be the lowest level at which a clear response (CR) is present. Distortion product otoacoustic emissions (DPOAEs) reflect outer hair cell integrity and cochlear function. These STRC homozygous mutant mice also had detectable DPOAE of up to 80 decibels (dB) sound pressure level, which reflects the absence of normal outer hair cells (OHCs) function.

FIG. 3 shows the sound pressure levels from ABR waveform results. The center and left-hand waveforms show the results of STRC KO mice, STRC^{-/-} mice, (Strc #16 homo) alone (n=5) and injected with the dual-vector system described here comprising AAV vectors where each AAV vector contains sequences encoding the signal sequence, N-Intein or C-Intein, and N-terminal or C-terminal portions of STRC protein (e.g., AAV2/AAV9-Php.B-Cmv-Strc-N; AAV2/AAV9-Php.B-Cmv-Strc-C) (n=9), respectively, and the right-hand waveforms represents wild-type (WT) mice (STRC^{WT/WT}) having an intact STRC gene (n=6). The WT mice demonstrate a sound pressure level ranging from 30 dB to 100 dB. The STRC KO mice (Strc #16 homo) in the center showed a limited sound pressure level ranging from 70 dB to 120 dB, demonstrating hearing loss below 70 dB. However, the left-hand waveforms demonstrate that the STRC KO mice injected with AAV2/AAV9-Php.B-Cmv-Strc-N; AAV2/AAV9-Php.B-Cmv-Strc-C were able to recover hearing loss to levels that correspond to those of the WT STRC mice.

The mean hearing thresholds or ABRs were tested across all frequencies in 4-week-old mice. The ABR results in FIG. 4A, showed that STRC knockout (KO) mice (Strc^{-/-}) injected with the dual-vector system described here comprising AAV vectors where each AAV vector contains sequences encoding the signal sequence, N-Intein or C-Intein, and N-terminal or C-terminal portions of STRC protein (e.g., AAV2/AAV9-Php.B-Cmv-Strc-N; AAV2/AAV9-Php.B-Cmv-Strc-C) (n=9; middle lines), respectively, demonstrated a recovery of hearing loss where the lowest ABR thresholds went as low as 30 dB at some frequencies. The ABR responses from wild-type mice (n=6; lowest line) had the lowest ABR thresholds going as low as 20 dB at some frequencies. However, the STRC KO mice (n=5; upper line) had severe hearing loss with thresholds of greater than 80 dB.

The ABR results in FIG. 5A and FIG. 6A show STRC KO mice (n=5; upper line with error bars) that had severe hearing loss with thresholds of greater than 80 dB. STRC KO mice injected with either of the intein AAV encoding portions of the wild-type STRC, encoding N-STRC (n=4; AAV2/AAV9-Php.B-Cmv-Strc-N) or C-STRC (n=3; AAV2/AAV9-Php.B-Cmv-Strc-C), showed hearing thresholds similar to the STRC KO mice results, i.e., greater than 80 dB. However, the ABR responses from wild-type mice (n=5; lowest line) had the lowest ABR thresholds going as low as 20 dB at some frequencies.

The DPOAE results in FIG. 4B (using the same STRC KO mice used in FIG. 45A) demonstrated that the STRC KO mice injected dual-vector system described here comprising AAV vectors where each AAV vector contains sequences encoding the signal sequence, N-Intein or C-Intein, and N-terminal or C-terminal portions of STRC protein (e.g., AAV2/AAV9-Php.B-Cmv-Strc-N; AAV2/AAV9-Php.B-Cmv-Strc-C, respectively; n=9; middle lines), showed a recovery of hearing loss as demonstrated by the DPOAE responses going as low as 30 dB at some frequencies. Similarly, the wild-type mice (n=6; lowest line) showed DPOAE response with the lowest DPOAE thresholds going as low as 30 dB at some frequencies. However, the STRC KO mice (n=5; upper line) showed no DPOAE responses under the tested conditions (up to 80 dB).

The DPOAE results in FIG. 5B and FIG. 6B (using the same STRC KO mice used in FIGS. 5A and 6A) demonstrated that the STRC KO mice (n=5; upper line) showed no DPOAE responses under the tested conditions (up to 80 dB). Similarly, the STRC KO mice injected with either of the intein AAV encoding portions of the wild-type STRC, encoding N-STRC (n=4; AAV2/AAV9-Php.B-Cmv-Strc-N) or C-STRC (n=3; AAV2/AAV9-Php.B-Cmv-Strc-C), showed no DPOAE responses. However, the wild-type mice (n=5; lowest line) showed DPOAE response with the lowest DPOAE thresholds going as low as 30 dB at some frequencies.

FIGS. 7A and 7B demonstrate the results of monitoring over time, three STRC KO mice injected dual-vector system described here comprising AAV vectors where each AAV vector contains sequences encoding the signal sequence, N-Intein or C-Intein, and N-terminal or C-terminal portions of STRC protein (e.g., AAV2/AAV9-Php.B-Cmv-Strc-N; AAV2/AAV9-Php.B-Cmv-Strc-C, respectively). ABR responses for each of the mice generally showed the lowest thresholds at some frequencies for mice at 4 weeks (solid lines). Generally, for each of the mice over time, the thresholds increased at 6 weeks (dashed lines) and 8 weeks (dashed/dotted lines) as compared to those achieved at 4 weeks. For example, FIG. 7A show that at 4 weeks (solid), mouse #3 had ABR thresholds ranging from 40 dB to 55 dB at frequencies of less than 10 kHz, and at 6 weeks (dash), mouse #3 had ABR thresholds ranging from 50 dB to 70 dB at frequencies of less than 10 kHz, which were at decibels greater than those observed at 4 weeks. For DPOAE responses, there was an observed shift from lower frequencies to higher frequencies over time. In FIG. 7B, the lowest DPOAE threshold response (50 dB) occurred at a frequency of 11 kHz at 4 weeks and at 16 kHz at 6 weeks for mouse #3.

### Example 3: Restoration of Morphology Using the Dual-Vector System

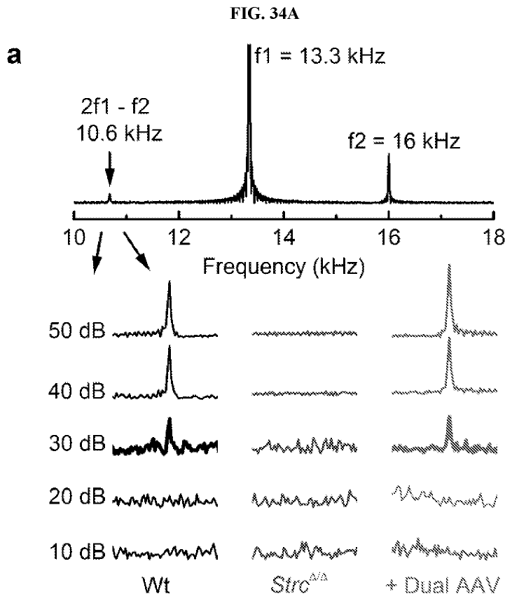

The dual AAV delivery system restored STRC expression and hair bundle morphology as demonstrated by the visual observation of cochleas stained with an anti-STRC antibody with Alex488 conjugated secondary antibody (green) and Alexa546-phalloidin (red). For example, FIG. 33A presents confocal images of cochleas injected with wild-type (WT) Strc or Strc^{Δ/Δ}, and dual AAV vector injected Strc^{Δ/Δ} cochleas. STRC and Actin were stained and both were observed in the WT (upper left) and partially observed in the Strc^{Δ/Δ}+ dual AAV vector sample (upper right), while disrupted actin outer hair cell (OHC) bundles were observed in Strc^{Δ/Δ} (upper middle). STRC localization was shown by the green stain and formation of inverted V hair bundles in WT (lower left) and partial presentation or recovery in the Strc^{Δ/Δ}+ dual AAV vector sample (lower right) and Strc^{Δ/Δ} failed to demonstrate any green stained hair bundles (lower center).

The dual AAV vector delivery system of the disclosure was observed in scanning electron microscopy images to restore hair bundle morphology in FIG. 33B (bottom panels) to almost WT levels (top panels). Strc^{Δ/Δ} injected outer hair cell bundles results in OHC bundles in disarray or disorganized (middle panels) as opposed to the wild-type organized OHC bundles.

### Example 4: Restoration of Auditory Function Using the Dual-Vector System

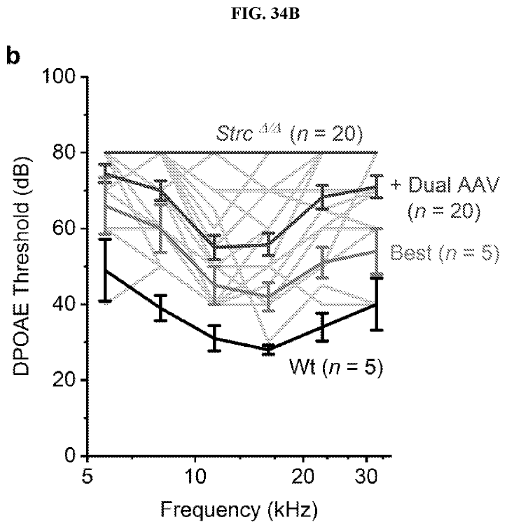

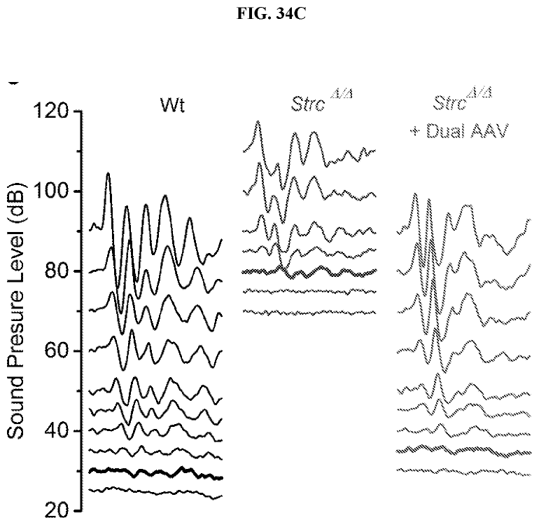

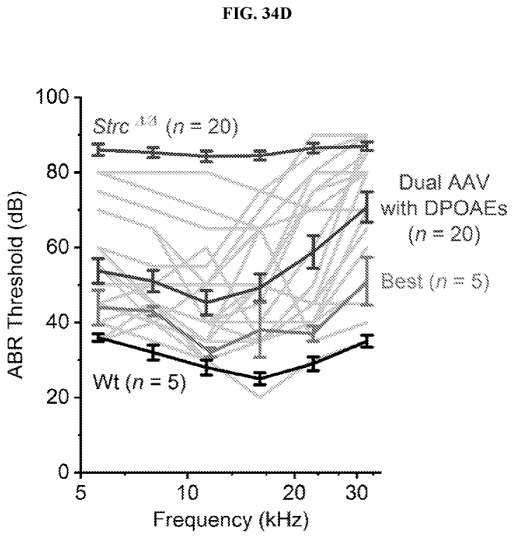

The dual AAV vector system also restored DPOAE and ABR thresholds as demonstrated by Fourier analysis of DPOAE waveforms ranging from sound pressure levels from 10 dB to 50 dB, where the Strc^{Δ/Δ}+ dual AAV vector sample (FIG. 34A, right) was observed to have similar auditory function patterns to those of the wild-type (FIG. 34A, left) and DPOAE thresholds show that the dual AAV vector injected Strc^{Δ/Δ} mice restored auditory function (FIG. 34B). ABR traces recorded for wild-type (FIG. 34C, left) and Strc^{Δ/Δ}+ dual AAV vector (FIG. 34C, right) injected cochleas resulted in similar sound pressure levels ranging from 25 dB to 110 dB; whereas Strc^{Δ/Δ} injected cochleas and sound pressure levels from 70 dB to 120 dB (FIG. 34C, middle). ABR threshold was shown to demonstrate recovery when mice were injected with the Strc^{Δ/Δ}+ dual AAV vector as compared to Strc^{Δ/Δ} alone at a frequency ranging from 5 kHz to 30 kHz (FIG. 34D).

### Specific Embodiments

Non-limiting specific embodiments are described below each of which is considered to be within the present disclosure.

Specific embodiment 1. A dual-vector system for expressing a protein of interest in a cell, the dual-vector system comprising:
a) a first vector comprising a first nucleotide sequence comprising, in a 5′ to 3′ direction:
   a signal sequence at the 5′-end of a partial coding sequence encoding an amino terminal (N-terminal) portion of the protein of interest;
   the partial coding sequence encoding the N-terminal portion of the protein of interest;
   a sequence encoding a splice donor sequence adjacent to and downstream of the partial coding sequence; and
b) a second vector comprising a second nucleotide sequence comprising, in a 5′ to 3′ direction:
   a signal sequence at the 5′-end of a partial coding sequence encoding a carboxy terminal (C-terminal) portion of the protein of interest;
   a sequence encoding splice acceptor sequence, wherein the splice acceptor sequence is flanked by the signal sequence and the partial coding sequence encoding the C-terminal portion of the protein of interest;
   the partial coding sequence encoding the C-terminal portion of the protein of interest.

Specific embodiment 2. The dual-vector system of specific embodiment 1, the dual-vector system comprising:
a) a first vector comprising a first nucleotide sequence comprising, in a 5′ to 3′ direction:
   a 5′-inverted terminal repeat (5′-ITR) sequence;
   a promoter sequence;
   a signal sequence, wherein the signal sequence is operably linked to and under control of the promoter;
   a partial coding sequence encoding an amino terminal (N-terminal) portion of the protein of interest, wherein the partial coding sequence is operably linked to and under control of the promoter;
   a sequence encoding an amino terminal fragment of intein (N-intein), wherein the sequence encoding N-intein is operably linked to and under control of the promoter;
   a poly-adenylation (polyA) signal sequence;
   a 3′-inverted terminal repeat (3′-ITR) sequence; and
b) a second vector comprising a second nucleotide sequence comprising, in a 5′ to 3′ direction:
   a 5′-inverted terminal repeat (5′-ITR) sequence;
   a promoter sequence;
   a signal sequence, wherein the signal sequence is operably linked to and under control of the promoter;
   a sequence encoding a carboxy terminal fragment of intein (C-intein), wherein the sequence encoding C-intein is operably linked to and under control of the promoter;
   a partial coding sequence encoding a carboxy terminal (C-terminal) portion of the protein of interest, wherein the partial coding sequence is operably linked to and under control of the promoter;
   a poly-adenylation (polyA) signal sequence;
   a 3′-inverted terminal repeat (3′-ITR) sequence.

Specific embodiment 3. The dual-vector system of specific embodiment 1 or 2, wherein the first vector and the second vector in the cell, express respectively:
a) a first protein sequence comprising in an N-terminal to C-terminal direction:
   a signal peptide sequence linked to an N-terminal portion of the protein of interest sequence fused at its C-terminal end to an N-intein protein sequence; and
b) a second protein sequence comprising in an N-terminal to C-terminal direction:
   a signal peptide sequence linked to a C-intein protein sequence fused to the N-terminal end of a C-terminal portion of the protein of interest sequence.

Specific embodiment 4. The dual-vector system of any one of specific embodiments 1-3, wherein the N-terminal portion of the protein of interest and the C-terminal portion of the protein of interest are configured to form a full-length protein of interest.

Specific embodiment 5. The dual-vector system of any one of specific embodiments 1-4, wherein the signal peptide sequence of the first protein sequence and the signal peptide sequence of the second protein sequence are the same.

Specific embodiment 6. The dual-vector system of any one of specific embodiments 1-4, wherein the signal peptide sequence of the first protein sequence and the signal peptide sequence of the second protein sequence are configured to transport the first protein sequence and the second protein sequence to the same cellular compartment.

Specific embodiment 7. The dual-vector system of any one of specific embodiments 1-4, wherein the signal peptide sequence of the first protein sequence and the signal peptide sequence of the second protein sequence are different, and each signal peptide sequence directs each respective protein sequence to the same cellular compartment.

Specific embodiment 8. The dual-vector system of specific embodiment 1-7, wherein the first vector and the second vector are each a viral vector.

Specific embodiment 9. The dual-vector system of specific embodiment 8, wherein the viral vector is an adeno-associated virus (AAV) vector.

Specific embodiment 10. The dual-vector system of specific embodiment 8 or specific embodiment 9, wherein the viral vectors are the same or different serotypes.

Specific embodiment 11. The dual-vector system of any one of specific embodiments 1-10, wherein the N-terminal portion and the C-terminal portion are configured to form the full-length protein of interest through a peptide bond.

Specific embodiment 12. The dual-vector system of any one of specific embodiments 1-11, wherein the protein of interest is an STRC protein.

Specific embodiment 13. The dual-vector system of any one of specific embodiments 12, wherein the STRC protein is encoded by the STRC gene.

Specific embodiment 14. The dual-vector system of any one of specific embodiments 1-13, wherein the signal sequence comprises a nucleic acid sequence at least 80% identity (e.g., 85%, 90%, 95%, 96%, 97%, 98%, 99%) to SEQ ID NO:9 or SEQ ID NO:11.

Specific embodiment 15. The dual-vector system of any one of specific embodiments 1-14, wherein the signal sequence encodes a signal peptide sequence having an amino acid sequence of at least 80% identity (e.g., 85%, 90%, 95%, 96%, 97%, 98%, 99%) to SEQ ID NO:10 or SEQ ID NO:12.

Specific embodiment 16. The dual-vector system of any one of specific embodiments 1-15, wherein the N-terminal portion of the protein of interest comprises a nucleic acid sequence of at least 70% identity (e.g., 75%, 80%, 85%, 90%, 95%, 96%, 97%, 98%, 99%) to SEQ ID NO:5 or SEQ ID NO:7 or to a nucleic acid sequence encoding SEQ ID NO:15 or SEQ ID NO: 16.

Specific embodiment 17. The dual-vector system of any one of specific embodiments 1-16, wherein the N-terminal portion of the protein of interest encodes an amino acid sequence of at least 70% identity (e.g., 75%, 80%, 85%, 90%, 95%, 96%, 97%, 98%, 99%) to SEQ ID NO: 15 or SEQ ID NO: 16.

Specific embodiment 18. The dual-vector system of any one of specific embodiments 1-17, wherein the N-intein sequence comprises a nucleic acid sequence of at least 80% identity (e.g., 85%, 90%, 95%, 96%, 97%, 98%, 99%) to SEQ ID NO:13.

Specific embodiment 19. The dual-vector system of any one of specific embodiments 1-18, wherein the N-intein sequence encodes an amino acid sequence of at least 80% identity (e.g., 85%, 90%, 95%, 96%, 97%, 98%, 99%) to SEQ ID NO:14.

Specific embodiment 20. The dual-vector system of any one of specific embodiments 1-19, wherein the C-terminal portion of the protein of interest comprises a nucleic acid sequence of at least 70% identity (e.g., 75%, 80%, 85%, 90%, 95%, 96%, 97%, 98%, 99%) to a nucleic acid sequence encoding SEQ ID NO:23 or SEQ ID NO:24.

Specific embodiment 21. The dual-vector system of any one of specific embodiments 1-20, wherein the C-terminal portion of the protein of interest encodes an amino acid sequence of at least 70% identity (e.g., 75%, 80%, 85%, 90%, 95%, 96%, 97%, 98%, 99%) to SEQ ID NO:23 or SEQ ID NO:24.

Specific embodiment 22. The dual-vector system of any one of specific embodiments 1-21, wherein the C-intein sequence comprises a nucleic acid sequence of at least 80% identity (e.g., 85%, 90%, 95%, 96%, 97%, 98%, 99%) to SEQ ID NO:21 or SEQ ID NO:46.

Specific embodiment 23. The dual-vector system of any one of specific embodiments 1-22, wherein the C-intein sequence encodes an amino acid sequence of at least 80% identity (e.g., 85%, 90%, 95%, 96%, 97%, 98%, 99%) to SEQ ID NO:22 or SEQ ID NO:49.

Specific embodiment 24. The dual-vector system of any one of specific embodiments 1-23, wherein the first nucleotide sequence comprises a nucleic acid sequence of at least 70% identity (e.g., 75%, 80%, 85%, 90%, 95%, 96%, 97%, 98%, 99%) to SEQ ID NO:5 or SEQ ID NO:7.

Specific embodiment 25. The dual-vector system of any one of specific embodiments 1-24, wherein the first nucleotide sequence encodes an amino acid sequence of at least 70% identity (e.g., 75%, 80%, 85%, 90%, 95%, 96%, 97%, 98%, 99%) to SEQ ID NO:6 or SEQ ID NO: 8.

Specific embodiment 26. The dual-vector system of any one of specific embodiments 1-25, wherein the second nucleotide sequence comprises a nucleic acid sequence of at least 70% identity (e.g., 75%, 80%, 85%, 90%, 95%, 96%, 97%, 98%, 99%) to SEQ ID NO:17 or SEQ ID NO:19.

Specific embodiment 27. The dual-vector system of any one of specific embodiments 1-26, wherein the second nucleotide sequence encodes an amino acid sequence of at least 70% identity (e.g., 75%, 80%, 85%, 90%, 95%, 96%, 97%, 98%, 99%) to SEQ ID NO:18 or SEQ ID NO:20.

Specific embodiment 28. A vector system for expressing a coding sequence of a STRC gene in a host cell, wherein the coding sequence comprises at least one vector comprising the STRC gene of SEQ ID NO:1, SEQ ID NO:3, SEQ ID NO:5, SEQ ID NO:7, SEQ ID NO:17, SEQ ID NO:19, SEQ ID NO:29, SEQ ID NO:31, SEQ ID NO:33 or SEQ ID NO:38, mRNA sequence of SEQ ID NO:30 or SEQ ID NO:32, or fragments thereof

Specific embodiment 29. The vector system of specific embodiment 28, wherein the STRC gene encodes the STRC protein of SEQ ID NO:2, SEQ ID NO:4, SEQ ID NO:6, SEQ ID NO:8, SEQ ID NO:15, SEQ ID NO:16, SEQ ID NO:18, SEQ ID NO:20, SEQ ID NO:23, SEQ ID NO:24, SEQ ID NO:25, SEQ ID NO:26, SEQ ID NO:36, or SEQ ID NO:39, or combinations thereof.

Specific embodiment 30. The vector system of specific embodiment 28, comprising a dual-vector system for expressing a coding sequence of the STRC gene in a host cell, wherein the coding sequence comprises a 5′ end fragment and a 3′ end fragment, the dual-vector system comprising:
a) a first vector comprising a first nucleotide sequence comprising, in a 5′ to 3′ direction:
   a 5′-inverted terminal repeat (5′-ITR) sequence;
   a promoter sequence;
   a signal sequence, wherein the signal sequence is operably linked to and under control of the promoter;
   the 5′ end fragment of the STRC gene coding sequence, wherein the 5′ end fragment is operably linked to and under control of the promoter;
   a sequence encoding an amino terminal fragment of intein (N-intein), wherein the sequence coding N-intein is operably linked to and under control of the promoter;
   a poly-adenylation (polyA) signal sequence; and
   a 3′-inverted terminal repeat (3′-ITR) sequence; and
b) a second vector comprising a second nucleotide sequence comprising, in a 5′ to 3′ direction:
   a 5′-inverted terminal repeat (5′-ITR) sequence;
   a promoter sequence;
   a signal sequence, wherein the signal sequence is operably linked to and under control of the promoter;
   a sequence encoding a carboxy terminal fragment of intein (C-intein), wherein the sequence coding C-intein is operably linked to and under control of the promoter;
   the 3′ end fragment of the STRC gene coding sequence, wherein the 3′ end fragment is operably linked to and under control of the promoter;
   a poly-adenylation (polyA) signal sequence; and
   a 3′-inverted terminal repeat (3′-ITR) sequence.

Specific embodiment 31. The dual-vector system of specific embodiment 30, wherein the first vector and the second vector in the cell, express respectively:
a) a first protein sequence comprising in an N-terminal to C-terminal direction:
   a signal peptide sequence linked to an N-terminal portion of the STRC protein sequence fused at its C-terminal end to an N-intein protein sequence; and
b) a second protein sequence comprising in an N-terminal to C-terminal direction:
   a signal peptide sequence linked to a C-intein protein sequence fused to the N-terminal end of a C-terminal portion of the STRC protein sequence.

Specific embodiment 32. The dual-vector system of any one of specific embodiments 30-31, wherein the N-terminal portion of the STRC protein and the C-terminal portion of the STRC protein form a full-length STRC protein.

Specific embodiment 33. The dual-vector system of any one of specific embodiments 30-32, wherein the signal peptide sequence of the first protein sequence and the signal peptide sequence of the second protein sequence are the same.

Specific embodiment 34. The dual-vector system of any one of specific embodiments 30-33, wherein the signal peptide sequence of the first protein sequence and the signal peptide sequence of the second protein sequence are configured to transport the first protein sequence and second protein sequence to the same cellular compartment.

Specific embodiment 35. The dual-vector system of any one of specific embodiments 30-34, wherein the signal peptide sequence of the first protein sequence and the signal peptide sequence of the second protein sequence are different, and each signal peptide sequence directs each respective protein sequence to the same cellular compartment.

Specific embodiment 36. The dual-vector system of any one of specific embodiments 30-35, wherein the first vector and the second vector are each a viral vector.

Specific embodiment 37. The dual-vector system of specific embodiment 36, wherein the viral vector is an adeno-associated virus (AAV) vector.

Specific embodiment 38. The dual-vector system of specific embodiment 36 or specific embodiment 37, wherein the viral vectors have the same serotype.

Specific embodiment 39. The dual-vector system of specific embodiment 36 or specific embodiment 37, wherein the viral vectors have different serotypes.

Specific embodiment 40. The dual-vector system of any one of specific embodiments 30-39, wherein the N-terminal portion and the C-terminal portion form the full-length STRC protein through a peptide bond.

Specific embodiment 41. The dual-vector system of any one of specific embodiments 30-40, wherein the STRC protein is encoded by the STRC gene.

Specific embodiment 42. The dual-vector system of any one of specific embodiments 30-41, wherein the signal sequence comprises a nucleic acid sequence of at least 80% identity (e.g., 85%, 90%, 95%, 96%, 97%, 98%, 99%) to SEQ ID NO: 9 or SEQ ID NO:11.

Specific embodiment 43. The dual-vector system of any one of specific embodiments 30-42, wherein the signal sequence encodes an amino acid sequence of at least 80% identity (e.g., 85%, 90%, 95%, 96%, 97%, 98%, 99%) to SEQ ID NO:10 or SEQ ID NO:12.

Specific embodiment 44. The dual-vector system of any one of specific embodiments 30-43, wherein the N-terminal portion of the STRC protein comprises a nucleic acid sequence of at least 70% identity (e.g., 75%, 80%, 85%, 90%, 95%, 96%, 97%, 98%, 99%) to SEQ ID NO:5 or SEQ ID NO:7 or to a nucleic acid sequence encoding SEQ ID NO:15 or SEQ ID NO: 16.

Specific embodiment 45. The dual-vector system of any one of specific embodiments 30-44, wherein the N-terminal portion of the STRC protein encodes an amino acid sequence of at least 70% identity (e.g., 75%, 80%, 85%, 90%, 95%, 96%, 97%, 98%, 99%) to SEQ ID NO:6, SEQ ID NO:8, SEQ ID NO:15 or SEQ ID NO:16.

Specific embodiment 46. The dual-vector system of any one of specific embodiments 30-45, wherein the N-terminal portion of the STRC protein comprises less than 54% (e.g., 53.8%, 53.6%, 53.4%, 53.2%, 53%, 52%, 50%, 45%) of the N-terminal end portion of the full-length STRC protein.

Specific embodiment 47. The dual-vector system of any one of specific embodiments 30-46, wherein the N-intein sequence comprises a nucleic acid sequence of at least 80% identity (e.g., 85%, 90%, 95%, 96%, 97%, 98%, 99%) to SEQ ID NO:13.

Specific embodiment 48. The dual-vector system of any one of specific embodiments 30-47, wherein the N-intein sequence encodes an amino acid sequence of at least 80% identity (e.g., 85%, 90%, 95%, 96%, 97%, 98%, 99%) to SEQ ID NO:14.

Specific embodiment 49. The dual-vector system of any one of specific embodiments 30-48, wherein the C-terminal portion of the STRC protein comprises a nucleic acid sequence of at least 70% (e.g., 75%, 80%, 85%, 90%, 95%, 96%, 97%, 98%, 99%) identity to SEQ ID NO:17, SEQ ID NO:19 or to a nucleic acid sequence encoding SEQ ID NO:18, SEQ ID NO:20, SEQ ID NO:23 or SEQ ID NO:24.

Specific embodiment 50. The dual-vector system of any one of specific embodiments 30-49, wherein the C-terminal portion of the STRC protein encodes an amino acid sequence of at least 70% identity (e.g., 75%, 80%, 85%, 90%, 95%, 96%, 97%, 98%, 99%) to SEQ ID NO:18, SEQ ID NO:20, SEQ ID NO:23 or SEQ ID NO:24.

Specific embodiment 51. The dual-vector system of any one of specific embodiments 30-50, wherein the C-terminal portion of the STRC protein comprises 46% or greater (e.g., 46.2%, 46.4%, 46.6%, 46.8%, 47%, 48%, 50%, 55%) of the C-terminal end portion of the full-length STRC protein.

Specific embodiment 52. The dual-vector system of any one of specific embodiments 30-51, wherein the C-intein sequence comprises a nucleic acid sequence at least 80% identical (e.g., 85%, 90%, 95%, 96%, 97%, 98%, 99%) to SEQ ID NO:21.

Specific embodiment 53. The dual-vector system of any one of specific embodiments 30-52, wherein the C-intein sequence encodes an amino acid sequence at least 80% identical (e.g., 85%, 90%, 95%, 96%, 97%, 98%, 99%) to SEQ ID NO:22.

Specific embodiment 54. The dual-vector system of any one of specific embodiments 30-53, wherein the first nucleotide sequence comprises a nucleic acid sequence of at least 70% identity (e.g., 75%, 80%, 85%, 90%, 95%, 96%, 97%, 98%, 99%) to SEQ ID NO:5 or SEQ ID NO:7.

Specific embodiment 55. The dual-vector system of any one of specific embodiments 30-54, wherein the first nucleotide sequence encodes an amino acid sequence of at least 70% identity (e.g., 75%, 80%, 85%, 90%, 95%, 96%, 97%, 98%, 99%) to SEQ ID NO:6, SEQ ID NO:8, SEQ ID NO:15, or SEQ ID NO:16.

Specific embodiment 56. The dual-vector system of any one of specific embodiments 30-55, wherein the second nucleotide sequence comprises a nucleic acid sequence of at least 70% identity (e.g., 75%, 80%, 85%, 90%, 95%, 96%, 97%, 98%, 99%) to SEQ ID NO:17 or SEQ ID NO:19.

Specific embodiment 57. The dual-vector system of any one of specific embodiments 30-56, wherein the second nucleotide sequence encodes an amino acid sequence of at least 70% identity (e.g., 75%, 80%, 85%, 90%, 95%, 96%, 97%, 98%, 99%) to SEQ ID NO:18, SEQ ID NO:20, SEQ ID NO:23, or SEQ ID NO:24.

Specific embodiment 58. At least one cell (e.g., 10, 20, 50, 100, 200, 500, 1000, or any number of cells sufficient to successfully express a large protein that biological activity) containing the vector system of any one of specific embodiments 1-57, where the at least one cell may be for treating, inhibiting, or reducing hearing loss in a subject, where the hearing loss may be autosomal recessive hearing loss.

Specific embodiment 59. A pharmaceutical composition comprising the vector system of any one of specific embodiments 1-57, and a pharmaceutically acceptable vehicle, for treating, inhibiting, or reducing hearing loss in a subject, where the hearing loss may be autosomal recessive hearing loss.

Specific embodiment 60. A method for treating, inhibiting, or reducing autosomal recessive hearing loss in a subject, comprising administering to a subject in need thereof, an effective amount of the dual-vector system of any one of specific embodiments 1-57.

Specific embodiment 61. A method for treating, inhibiting, or reducing autosomal recessive hearing loss in a subject, comprising administering to a subject in need thereof, an effective amount of the at least one cell of specific embodiment 58.

Specific embodiment 62. A method for treating, inhibiting, or reducing autosomal recessive hearing loss in a subject, comprising administering to a subject in need thereof, an effective amount of the pharmaceutical composition of specific embodiment 59.

Specific embodiment 63. The method of any one of specific embodiments 60-62, wherein the autosomal recessive hearing loss is DFNB16.

Specific embodiment 64. A method, comprising:

contacting at least one cell of a subject with the pharmaceutical composition of specific embodiment 59, wherein the contacting delivers the vector system comprising the first nucleotide sequence and the second nucleotide sequence into the at least one cell of the subject, wherein the contacted at least one cell expresses an N-terminal portion of the protein and a C-terminal portion of the protein joined by a peptide bond to form a full-length protein.

Specific embodiment 65. A method for treating and/or preventing a pathology or disease characterized by a hearing loss comprising administering to a subject in need thereof an effective amount of the vector system according to any one of specific embodiments 1-57, at least one cell according to specific embodiment 58, or the pharmaceutical composition according to specific embodiment 59.

Specific embodiment 66. The method of any one of specific embodiments 64-65, wherein the at least one cell is an inner ear cell.

Specific embodiment 67. The method of any one of specific embodiments 64-66, wherein the at least one cell is an inner hair cell or an outer hair cell.

Specific embodiment 68. The method of any one of specific embodiments 60-67, wherein the at least one cell is in vivo or in vitro.

Specific embodiment 69. The method of any one of specific embodiments 60-68, wherein the method improves or restores auditory function in the subject.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference in their entireties to the same extent as if each independent patent and publication were specifically and individually indicated to be incorporated by reference.

1. A dual-vector system for expressing a protein of interest in a cell, the dual-vector system comprising:
a) a first vector comprising a first nucleotide sequence comprising, in a 5′ to 3′ direction:
a signal sequence at the 5′-end of a partial coding sequence encoding an amino terminal (N-terminal) portion of the protein of interest;
the partial coding sequence encoding the N-terminal portion of the protein of interest;
a sequence encoding an amino carboxy terminal fragment of intein (N-intein) adjacent to and downstream of the partial coding sequence; and
b) a second vector comprising a second nucleotide sequence comprising, in a 5′ to 3′ direction:
a signal sequence at the 5′-end of a partial coding sequence encoding a carboxy terminal (C-terminal) portion of the protein of interest;
a sequence encoding a carboxy terminal fragment of intein (C-intein), wherein the C-intein is flanked by the signal sequence and the partial coding sequence encoding the C-terminal portion of the protein of interest;
the partial coding sequence encoding the C-terminal portion of the protein of interest.

2. A dual-vector system, comprising:
a) a first vector comprising a first nucleotide sequence comprising, in a 5′ to 3′ direction:
a 5′-inverted terminal repeat (5′-ITR) sequence;
a promoter sequence;
a signal sequence, wherein the signal sequence is operably linked to and under control of the promoter;
a partial coding sequence encoding an amino terminal (N-terminal) portion of the protein of interest, wherein the partial coding sequence is operably linked to and under control of the promoter;
a sequence encoding a split intein-N, wherein the sequence encoding the split intein-N is operably linked to and under control of the promoter;
a poly-adenylation (polyA) signal sequence;
a 3′-inverted terminal repeat (3′-ITR) sequence; and
b) a second vector comprising a second nucleotide sequence comprising, in a 5′ to 3′ direction:
a 5′-inverted terminal repeat (5′-ITR) sequence;
a promoter sequence;
a signal sequence, wherein the signal sequence is operably linked to and under control of the promoter;
a sequence encoding a split intein-C, wherein the sequence encoding the split intein-C is operably linked to and under control of the promoter;
a partial coding sequence encoding a carboxy terminal (C-terminal) portion of the protein of interest, wherein the partial coding sequence is operably linked to and under control of the promoter;
a poly-adenylation (polyA) signal sequence;
a 3′-inverted terminal repeat (3′-ITR) sequence.

3. The dual-vector system of claim 1, wherein the first vector and the second vector in the cell, express respectively:
a) a first protein sequence comprising in an N-terminal to C-terminal direction:
a signal peptide sequence linked to an N-terminal portion of the protein of interest sequence fused at its C-terminal end to an N-intein protein sequence; and
b) a second protein sequence comprising in an N-terminal to C-terminal direction:
a signal peptide sequence linked to a C-intein protein sequence fused to the N-terminal end of a C-terminal portion of the protein of interest sequence.

4. The dual-vector system of claim 1, wherein the N-terminal portion of the protein of interest and the C-terminal portion of the protein of interest are configured to form a full-length protein of interest.

5. The dual-vector system of claim 3, wherein the signal peptide sequence of the first protein sequence and the signal peptide sequence of the second protein sequence are the same.

6. The dual-vector system of claim 3, wherein the signal peptide sequence of the first protein sequence and the signal peptide sequence of the second protein sequence are configured to transport the first protein sequence and the second protein sequence to the same cellular compartment of the cell.

7. (canceled)

8. The dual-vector system of claim 1, wherein the first vector and the second vector are each a viral vector.

9. The dual-vector system of claim 8, wherein the viral vector is an adeno-associated virus (AAV) vector or lentivirus.

10. The dual-vector system of claim 1, wherein the protein of interest is an STRC protein.

11-17. (canceled)

18. A vector system for expressing a coding sequence of a STRC gene in a host cell, wherein the coding sequence comprises at least one vector comprising the STRC gene of SEQ ID NO:1, SEQ ID NO:3, SEQ ID NO:5, SEQ ID NO:7, SEQ ID NO:17, SEQ ID NO:19, SEQ ID NO:29, SEQ ID NO:31, SEQ ID NO:33, or SEQ ID NO:38, mRNA sequence of SEQ ID NO:30 or SEQ ID NO:32, or fragments thereof.

19. (canceled)

20. The vector system of claim 18, comprising a dual-vector system for expressing a coding sequence of the STRC gene in a host cell, wherein the coding sequence comprises a 5′ end fragment and a 3′ end fragment, the dual-vector system comprising:
a) a first vector comprising a first nucleotide sequence comprising, in a 5′ to 3′ direction:
a 5′-inverted terminal repeat (5′-ITR) sequence;
a promoter sequence;
a signal sequence, wherein the signal sequence is operably linked to and under control of the promoter;
the 5′ end fragment of the STRC gene coding sequence, wherein the 5′ end fragment is operably linked to and under control of the promoter;
a sequence encoding an amino terminal fragment of intein (N-intein), wherein the sequence coding N-intein is operably linked to and under control of the promoter;
a poly-adenylation (polyA) signal sequence; and
a 3′-inverted terminal repeat (3′-ITR) sequence; and
b) a second vector comprising a second nucleotide sequence comprising, in a 5′ to 3′ direction:
a 5′-inverted terminal repeat (5′-ITR) sequence;
a promoter sequence;
a signal sequence, wherein the signal sequence is operably linked to and under control of the promoter;
a sequence encoding a carboxy terminal fragment of intein (C-intein), wherein the sequence coding C-intein is operably linked to and under control of the promoter;
the 3′ end fragment of the STRC gene coding sequence, wherein the 3′ end fragment is operably linked to and under control of the promoter;
a poly-adenylation (polyA) signal sequence; and
a 3′-inverted terminal repeat (3′-ITR) sequence.

21. The dual-vector system of claim 20, wherein the first vector and the second vector in the cell, express respectively:
a) a first protein sequence comprising in an N-terminal to C-terminal direction:
a signal peptide sequence linked to an N-terminal portion of the STRC protein sequence fused at its C-terminal end to an N-intein protein sequence; and
b) a second protein sequence comprising in an N-terminal to C-terminal direction:
a signal peptide sequence linked to a C-intein protein sequence fused to the N-terminal end of a C-terminal portion of the STRC protein sequence.

22. The dual-vector system of claim 20, wherein the N-terminal portion of the STRC protein and the C-terminal portion of the STRC protein are configured to form a full-length STRC protein.

23-35. (canceled)

36. A cell containing the vector system of claim 1.

37. A pharmaceutical composition comprising the vector system of claim 1.

38. A method for treating autosomal recessive hearing loss in a subject, comprising administering to a subject in need thereof, an effective amount of the dual-vector system of claim 1.

39. A method for treating autosomal recessive hearing loss in a subject, comprising administering to a subject in need thereof, the cell of claim 36.

40. A method for treating autosomal recessive hearing loss in a subject, comprising administering to a subject in need thereof, the pharmaceutical composition of claim 37.

41. The method of claim 38, wherein the autosomal recessive hearing loss is DFNB16.

42. A method, comprising:
contacting at least one cell of a subject with the pharmaceutical composition of claim 37, wherein the contacting delivers the vector system comprising the first nucleotide sequence and the second nucleotide sequence into the at least one cell of the subject, wherein the contacted at least one cell expresses an N-terminal portion of the protein and a C-terminal portion of the protein joined by a peptide bond to form a full-length protein.

43. A method for treating and/or preventing a pathology or disease characterized by a hearing loss comprising administering to a subject in need thereof an effective amount of the vector system according to claim 1.

44. The method of claim 42, wherein the at least one cell is an inner ear cell.

45. The method of claim 42, wherein the at least one cell is an inner hair cell or an outer hair cell.

46-47. (canceled)


SEQUENCE LISTING (63 sequences)

SEQ ID NO: 1 - DNA, 5262 bp, Homo sapiens
gtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgcta

SEQ ID NO: 2 - protein, 1754 aa, Homo sapiens
VTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 3 - DNA, 5361 bp, Mus musculus
gcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctatta

SEQ ID NO: 4 - protein, 1787 aa, Mus musculus
APTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLL

SEQ ID NO: 5 - DNA, 2436 bp, Artificial Sequence
  Description of Artificial Sequence Synthetic polynucleotide
atggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaattaatag

SEQ ID NO: 6 - protein, 810 aa, Artificial Sequence
  Description of Artificial Sequence Synthetic polypeptide
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 7 - DNA, 2550 bp, Artificial Sequence
  Description of Artificial Sequence Synthetic polynucleotide
atggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaattaatag

SEQ ID NO: 8 - protein, 848 aa, Artificial Sequence
  Description of Artificial Sequence Synthetic polypeptide
MALSLQPQLLLLLSLLPQEVTSAPTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 9 - DNA, 60 bp, Artificial Sequence
  Description of Artificial Sequence Synthetic oligonucleotide
gctctcagcctctggcccctgctgctgctgctgctgctgctgctgctgctgtcctttgca

SEQ ID NO: 10 - protein, 20 aa, Artificial Sequence
  Description of Artificial Sequence Synthetic peptide
ALSLWPLLLLLLLLLLLSFA

SEQ ID NO: 11 - DNA, 63 bp, Artificial Sequence
  Description of Artificial Sequence Synthetic oligonucleotide
gctctgagcctccagccccagctgctccttctcctgtcgctcctgccgcaggaagtgacttca

SEQ ID NO: 12 - protein, 21 aa, Artificial Sequence
  Description of Artificial Sequence Synthetic peptide
ALSLQPQLLLLLSLLPQEVTS

SEQ ID NO: 13 - DNA, 306 bp, Artificial Sequence
  Description of Artificial Sequence Synthetic polynucleotide
tgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaat

SEQ ID NO: 14 - protein, 102 aa, Artificial Sequence
  Description of Artificial Sequence Synthetic polypeptide
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 15 - protein, 687 aa, Homo sapiens
VTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLA

SEQ ID NO: 16 - protein, 724 aa, Mus musculus
APTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLS

SEQ ID NO: 17 - DNA, 3423 bp, Artificial Sequence
  Description of Artificial Sequence Synthetic polynucleotide
atggctctcagcctctggcccctgctgctgctgctgctgctgctgctgctgctgtcctttgcaatcaagatagctacaaggaagtatcttggcaaacaaaacgtttatgatattggagtcgaaagagatcacaactttgctctgaagaacggattcatagcttctaattgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctaacgcgtacgcggccgctcgagcagaaactcatctcagaagaggatctgtaatag

SEQ ID NO: 18 - protein, 1139 aa, Artificial Sequence
  Description of Artificial Sequence Synthetic polypeptide
MALSLWPLLLLLLLLLLLSFAIKIATRKYLGKQNVYDIGVERDHNFALKNGFIASNCFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLLTRTRPLEQKLISEEDL

SEQ ID NO: 19 - DNA, 3414 bp, Artificial Sequence
  Description of Artificial Sequence Synthetic polynucleotide
atggctctgagcctccagccccagctgctccttctcctgtcgctcctgccgcaggaagtgacttcaatcaagatagctacaaggaagtatcttggcaaacaaaacgtttatgatattggagtcgaaagagatcacaactttgctctgaagaacggattcatagcttctaattgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattaacgcgtacgcggccgctcgagcagaaactcatctcagaagaggatctgtaatag

SEQ ID NO: 20 - protein, 1136 aa, Artificial Sequence
  Description of Artificial Sequence Synthetic polypeptide
MALSLQPQLLLLLSLLPQEVTSIKIATRKYLGKQNVYDIGVERDHNFALKNGFIASNCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLLTRTRPLEQKLISEEDL

SEQ ID NO: 21 - DNA, 105 bp, Artificial Sequence
  Description of Artificial Sequence Synthetic polynucleotide
atcaagatagctacaaggaagtatcttggcaaacaaaacgtttatgatattggagtcgaaagagatcacaactttgctctgaagaacggattcatagcttctaat

SEQ ID NO: 22 - protein, 35 aa, Artificial Sequence
  Description of Artificial Sequence Synthetic polypeptide
IKIATRKYLGKQNVYDIGVERDHNFALKNGFIASN

SEQ ID NO: 23 - protein, 1067 aa, Homo sapiens
CFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 24 - protein, 1063 aa, Mus musculus
CFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLL

SEQ ID NO: 25 - protein, 1775 aa, Homo sapiens
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLL

SEQ ID NO: 26 - protein, 1809 aa, Mus musculus
MALSLQPQLLLLLSLLPQEVTSAPTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLL

SEQ ID NO: 27 - protein, 10 aa, Artificial Sequence
  Description of Artificial Sequence Synthetic peptide
EQKLISEEDL

SEQ ID NO: 28 - DNA, 8543 bp, Artificial Sequence
  Description of Artificial Sequence Synthetic polynucleotide
ccaatgatacgcgtcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacatcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgcccaaactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatcacatgtcctaggaactatcgatccatcacactggcggccgctcgactagagcggccgccaccgcggtggagctccagcttttgcggaccgaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 29 - DNA, 5262 bp, Homo sapiens; identical to SEQ ID NO: 1

SEQ ID NO: 30 - DNA, 5515 bp, Homo sapiens
gccctgccctcacctggctatcccacacaggtgagaataaccagaactcacctccggtaccagtgttcacttggaaacatggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctatgagcctgtctctacagtagaaggagattgtggggagagaaatcttaagtcataatgaataaagtgcaaacagaagtgcatcctgattattttcagaagctgatgaggaata

SEQ ID NO: 31 - DNA, 5361 bp, Mus musculus; identical to SEQ ID NO: 3

SEQ ID NO: 32 - DNA, 5757 bp, Mus musculus
gccccgtcttcacctggctatcccttcatggtgagcatagccagaactcacctctaggcccagtgtgcacctggaaatatggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattatgataactgttccttcagttgagggagaaaatttacatcatactgaacaacttgtaaatggaagtgcatactaattattctcagtaagtggatgaggattgtgggtaaaattccaatgcattccacccacctgagaactgtgctcctggcatatacgcctcttgccatcatgaataacctcactgtttcttttcatttcctacttcctcctcaccacaccaatagaaataacacagacagctgcaacataat

SEQ ID NO: 33 - DNA, 5379 bp, Artificial Sequence
  Description of Artificial Sequence Synthetic polynucleotide
atggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcctgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgctaacgcgtacgcggccgctcgagcagaaactcatctcagaagaggatctgtaatag

SEQ ID NO: 34 - DNA, 18 bp, Artificial Sequence
  Description of Artificial Sequence Synthetic oligonucleotide
acgcgtacgcggccgctc

SEQ ID NO: 35 - DNA, 30 bp, Artificial Sequence
  Description of Artificial Sequence Synthetic oligonucleotide
gagcagaaactcatctcagaagaggatctg

SEQ ID NO: 36 - protein, 1791 aa, Artificial Sequence
  Description of Artificial Sequence Synthetic polypeptide
MALSLWPLLLLLLLLLLLSFAVTLAPTGPHSLDPGLSFLKSLLSTLDQAPQGSLSRSRFFTFLANISSSFEPGRMGEGPVGEPPPLQPPALRLHDFLVTLRGSPDWEPMLGLLGDMLALLGQEQTPRDFLVHQAGVLGGLVEVLLGALVPGGPPTPTRPPCTRDGPSDCVLAADWLPSLLLLLEGTRWQALVQVQPSVDPTNATGLDGREAAPHFLQGLLGLLTPTGELGSKEALWGGLLRTVGAPLYAAFQEGLLRVTHSLQDEVFSILGQPEPDTNGQCQGGNLQQLLLWGVRHNLSWDVQALGFLSGSPPPPPALLHCLSTGVPLPRASQPSAHISPRQRRAITVEALCENHLGPAPPYSISNFSIHLLCQHTKPATPQPHPSTTAICQTAVWYAVSWAPGAQGWLQACHDQFPDEFLDAICSNLSFSALSGSNRRLVKRLCAGLLPPPTSCPEGLPPVPLTPDIFWGCFLENETLWAERLCGEASLQAVPPSNQAWVQHVCQGPTPDVTASPPCHIGPCGERCPDGGSFLVMVCANDTMYEVLVPFWPWLAGQCRISRGGNDTCFLEGLLGPLLPSLPPLGPSPLCLTPGPFLLGMLSQLPRCQSSVPALAHPTRLHYLLRLLTFLLGPGAGGAEAQGMLGRALLLSSLPDNCSFWDAFRPEGRRSVLRTIGEYLEQDEEQPTPSGFEPTVNPSSGISKMELLACFSPVLWDLLQREKSVWALQILVQAYLHMPPENLQQLVLSAEREAAQGFLTLMLQGKLQGKLQVPPSEEQALGRLTALLLQRYPRLTSQLFIDLSPLIPFLAVSDLMRFPPSLLANDSVLAAIRDYSPGMRPEQKEALAKRLLAPELFGEVPAWPQELLWAVLPLLPHLPLENFLQLSPHQIQALEDSWPAAGLGPGHARHVLRSLVNQSVQDGEEQVRRLGPLACFLSPEELQSLVPLSDPTGPVERGLLECAANGTLSPEGRVAYELLGVLRSSGGAVLSPRELRVWAPLFSQLGLRFLQELSEPQLRAMLPVLQGTSVTPAQAVLLLGRLLPRHDLSLEELCSLHLLLPGLSPQTLQAIPRRVLVGACSCLAPELSRLSACQTAALLQTFRVKDGVKNMGTTGAGPAVCIPGQPIPTTWPDCLLPLLPLKLLQLDSLALLANRRRYWELPWSEQQAQFLWKKMQVPTNLTLRNLQALGTLAGGMSCEFLQQINSMVDFLEVVHMIYQLPTRVRGSLRACIWAELQRRMAMPEPEWTTVGPELNGLDSKLLLDLPIQLMDRLSNESIMLVVELVQRAPEQLLALTPLHQAALAERALQNLAPKETPVSGEVLETLGPLVGFLGTESTRQIPLQILLSHLSQLQGFCLGETFATELGWLLLQESVLGKPELWSQDEVEQAGRLVFTLSTEAISLIPREALGPETLERLLEKQQSWEQSRVGQLCREPQLAAKKAALVAGVVRPAAEDLPEPVPNCADVRGTFPAAWSATQIAEMELSDFEDCLTLFAGDPGLGPEELRAAMGKAKQLWGPPRGFRPEQILQLGRLLIGLGDRELQELILVDWGVLSTLGQIDGWSTTQLRIVVSSFLRQSGRHVSHLDFVHLTALGYTLCGLRPEELQHISSWEFSQAALFLGTLHLQCSEEQLEVLAHLLVLPGGFGPISNWGPEIFTEIGTIAAGIPDLALSALLRGQIQGVTPLAISVIPPPKFAVVFSPIQLSSLTSAQAVAVTPEQMAFLSPEQRRAVAWAQHEGKESPEQQGRSTAWGLQDWSRPSWSLVLTISFLGHLLTRTRPLEQKLISEEDL

SEQ ID NO: 37 - protein, 6 aa, Artificial Sequence
  Description of Artificial Sequence Synthetic peptide
TRTRPL

SEQ ID NO: 38 - DNA, 5481 bp, Artificial Sequence
  Description of Artificial Sequence Synthetic polynucleotide
atggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattaacgcgtacgcggccgctcgagcagaaactcatctcagaagaggatctgtaatag

SEQ ID NO: 39 - protein, 1825 aa, Artificial Sequence
  Description of Artificial Sequence Synthetic polypeptide
MALSLQPQLLLLLSLLPQEVTSAPTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLLTRTRPLEQKLISEEDL

SEQ ID NO: 40 - DNA, 60 bp, Artificial Sequence; identical to SEQ ID NO: 9
  Description of Artificial Sequence Synthetic oligonucleotide

SEQ ID NO: 41 - DNA, 63 bp, Artificial Sequence; identical to SEQ ID NO: 11
  Description of Artificial Sequence Synthetic oligonucleotide

SEQ ID NO: 42 - DNA, 312 bp, Artificial Sequence
  Description of Artificial Sequence Synthetic polynucleotide
tgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaattaatag

SEQ ID NO: 43 - protein, 20 aa, Artificial Sequence
  Description of Artificial Sequence Synthetic peptide
ALSLWPLLLLLLLLLLLSFA

SEQ ID NO: 44 - protein, 21 aa, Artificial Sequence
  Description of Artificial Sequence Synthetic peptide
ALSLQPQLLLLLSLLPQEVTS

SEQ ID NO: 45 - protein, 102 aa, Artificial Sequence; identical to SEQ ID NO: 14
  Description of Artificial Sequence Synthetic polypeptide

SEQ ID NO: 46 - DNA, 105 bp, Artificial Sequence; identical to SEQ ID NO: 21
  Description of Artificial Sequence Synthetic polynucleotide

SEQ ID NO: 47 - DNA, 18 bp, Artificial Sequence
  Description of Artificial Sequence Synthetic oligonucleotide
acgcgtacgcggccgctc

SEQ ID NO: 48 - DNA, 36 bp, Artificial Sequence
  Description of Artificial Sequence Synthetic oligonucleotide
gagcagaaactcatctcagaagaggatctgtaatag

SEQ ID NO: 49 - protein, 35 aa, Artificial Sequence; identical to SEQ ID NO: 22
  Description of Artificial Sequence Synthetic polypeptide

SEQ ID NO: 50 - protein, 6 aa, Artificial Sequence
  Description of Artificial Sequence Synthetic peptide
TRTRPL

SEQ ID NO: 51 - DNA, 3219 bp, Artificial Sequence
  Description of Artificial Sequence Synthetic polynucleotide
atggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcctgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgcctgtcatacgaaaccgagatactgacagtagaatatggccttctgccaatcgggaagattgtggagaaacggatagaatgcacagtttactctgtcgataacaatggtaacatttatactcagccagttgcccagtggcacgaccggggagagcaggaagtattcgaatactgtctggaggatggaagtctcattagggccactaaggaccacaaatttatgacagtcgatggccagatgctgcctatagacgaaatctttgagcgagagttggacctcatgcgagttgacaaccttcctaattaatag

SEQ ID NO: 52 - protein, 1071 aa, Artificial Sequence
  Description of Artificial Sequence Synthetic polypeptide
MALSLQPQLLLLLSLLPQEVTSAPTGPQSLDAGLSLLKSFVATLDQAPQRSLSQSRFSAFLANISSSFQLGRMGEGPVGEPPPLQPPALRLHDFLVTLRGSPDWEPMLGLLGDVLALLGQEQTPRDFLVHQAGVLGGLVEALLGALVPGGPPAPTRPPCTRDGPSDCVLAADWLPSLMLLLEGTRWQALVQLQPSVDPTNATGLDGREPAPHFLQGLLGLLTPAGELGSEEALWGGLLRTVGAPLYAAFQEGLLRVTHSLQDEVFSIMGQPEPDASGQCQGGNLQQLLLWGMRNNLSWDARALGFLSGSPPPPPALLHCLSRGVPLPRASQPAAHISPRQRRAISVEALCENHSGPEPPYSISNFSIYLLCQHIKPATPRPPPTTPRPPPTTPQPPPTTTQPIPDTTQPPPVTPRPPPTTPQPPPSTAVICQTAVWYAVSWAPGARGWLQACHDQFPDQFLDMICGNLSFSALSGPSRPLVKQLCAGLLPPPTSCPPGLIPVPLTPEIFWGCFLENETLWAERLCVEDSLQAVPPRNQAWVQHVCRGPTLDATDFPPCRVGPCGERCPDGGSFLLMVCANDTLYEALVPFWAWLAGQCRISRGGNDTCFLEGMLGPLLPSLPPLGPSPLCLAPGPFLLGMLSQLPRCQSSVPALAHPTRLHYLLRLLTFLLGPGTGGAETQGMLGQALLLSSLPDNCSFWDAFRPEGRRSVLRTVGEYLQREEPTPPGLDSSLSLGSGMSKMELLSCFSPVLWDLLQREKSVWALRTLVKAYLRMPPEDLQQLVLSAEMEAAQGFLTLMLRSWAKLKVQPSEEQAMGRLTALLLQRYPRLTSQLFIDMSPLIPFLAVPDLMRFPPSLLANDSVLAAIRDHSSGMKPEQKEALAKRLLAPELFGEVPDWPQELLWAALPLLPHLPLESFLQLSPHQIQALEDSWPVADLGPGHARHVLRSLVNQSMEDGEEQVLRLGSLACLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 53 - DNA, 2745 bp, Artificial Sequence
  Description of Artificial Sequence Synthetic polynucleotide
atggctctgagcctccagccccagctgctccttctcctgtcgctcctgccgcaggaagtgacttcaatcaagatagctacaaggaagtatcttggcaaacaaaacgtttatgatattggagtcgaaagagatcacaactttgctctgaagaacggattcatagcttctaattgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctattaacgcgtacgcggccgctcgagcagaaactcatctcagaagaggatctgtaatag

SEQ ID NO: 54 - protein, 913 aa, Artificial Sequence
  Description of Artificial Sequence Synthetic polypeptide
MALSLQPQLLLLLSLLPQEVTSIKIATRKYLGKQNVYDIGVERDHNFALKNGFIASNCFLSPEELQSLVPLSDPMGPVEQGLLECAANGTLSPEGRVAYELLGVLRSSGGTVLSPRELRVWAPLFPQLGLRFLQELSETQLRAMLPALQGASVTPAQAVLLFGRLLPKHDLSLEELCSLHPLLPGLSPQTLQAIPKRVLVGACSCLGPELSRLSACQIAALLQTFRVKDGVKNMGAAGAGSAVCIPGQPTTWPDCLLPLLPLKLLQLDAAALLANRRLYRQLPWSEQQAQFLWKKMQVPTNLSLRNLQALGNLAGGMTCEFLQQISSMVDFLDVVHMLYQLPTGVRESLRACIWTELQRRMTMPEPELTTLGPELSELDTKLLLDLPIQLMDRLSNDSIMLVVEMVQGAPEQLLALTPLHQTALAERALKNLAPKETPISKEVLETLGPLVGFLGIESTRRIPLPILLSHLSQLQGFCLGETFATELGWLLLQEPVLGKPELWSQDEIEQAGRLVFTLSAEAISSIPREALGPETLERLLGKHQSWEQSRVGHLCGESQLAHKKAALVAGIVHPAAEGLQEPVPNCADIRGTFPAAWSATQISEMELSDFEDCLSLFAGDPGLGPEELRAAMGKAKQLWGPPRGFRPEQILQLGRLLIGLGERELQELTLVDWGVLSSLGQIDGWSSMQLRAVVSSFLRQSGRHVSHLDFIYLTALGYTVCGLRPEELQHISSWEFSQAALFLGSLHLPCSEEQLEVLAYLLVLPGGFGPVSNWGPEIFTEIGTIAAGIPDLALSALLRGQIQGLTPLAISVIPAPKFAVVFNPIQLSSLTRGQAVAVTPEQLAYLSPEQRRAVAWAQHEGKEIPEQLGRNSAWGLYDWFQASWALALPVSIFGHLLTRTRPLEQKLISEEDL

SEQ ID NO: 55 - DNA, 2124 bp, Artificial Sequence
  Description of Artificial Sequence Synthetic polynucleotide
atggctctcagcctctggcccctgctgctgctgctgctgctgctgctgctgctgtcctttgcagtgactctggcccctactgggcctcattccctggaccctggtctctccttcctgaagtcattgctctccactctggaccaggctccccagggctccctgagccgctcacggttctttacattcctggccaacatttcttcttcctttgagcctgggagaatgggggaaggaccagtaggagagcccccacctctccagccgcctgctctgcggctccatgattttctagtgacactgagaggtagccccgactgggagccaatgctagggctgctaggggatatgctggcactgctgggacaggagcagactccccgagatttcctggtgcaccaggcaggggtgctgggtggacttgtggaggtgctgctgggagccttagttcctgggggcccccctaccccaactcggcccccatgcacccgtgatgggccgtctgactgtgtcctggctgctgactggttgccttctctgctgctgttgttagagggcacacgctggcaagctctggtgcaggtgcagcccagtgtggaccccaccaatgccacaggcctcgatgggagggaggcagctcctcactttttgcagggtctgttgggtttgcttaccccaacaggggagctaggctccaaggaggctctttggggcggtctgctacgcacagtgggggcccccctctatgctgcctttcaggaggggctgctccgtgtcactcactccctgcaggatgaggtcttctccattttggggcagccagagcctgataccaatgggcagtgccagggaggtaaccttcaacagctgctcttatggggcgtccggcacaacctttcctgggatgtccaggcgctgggctttctgtctggatcaccacccccaccccctgccctccttcactgcctgagcacgggcgtgcctctgcccagagcttctcagccgtcagcccacatcagcccacgccaacggcgagccatcactgtggaggccctctgtgagaaccacttaggcccagcaccaccctacagcatttccaacttctccatccacttgctctgccagcacaccaagcctgccactccacagccccatcccagcaccactgccatctgccagacagctgtgtggtatgcagtgtcctgggcaccaggtgcccaaggctggctacaggcctgccacgaccagtttcctgatgagtttttggatgcgatctgcagtaacctctccttttcagccctgtctggctccaaccgccgcctggtgaagcggctctgtgctggcctgctcccaccccctaccagctgccctgaaggcctgccccctgttcccctcaccccagacatcttttggggctgcttcttggagaatgagactctgtgggctgagcgactgtgtggggaggcaagtctacaggctgtgccccccagcaaccaggcttgggtccagcatgtgtgccagggccccaccccagatgtcactgcctccccaccatgccacattggaccctgtggggaacgctgcccggatgggggcagcttcctggtgatggtctgtgccaatgacaccatgtatgaggtcctggtgcccttctggccttggctagcaggccaatgcaggataagtcgtgggggcaatgacacttgcttcctagaagggctgctgggcccccttctgccctctctgccaccactgggaccatccccactctgtctgacccctggccccttcctccttggcatgctatcccagttgccacgctgtcagtcctctgtcccagctcttgctcaccccacacgcctacactatctcctccgcctgctgaccttcctcttgggtccaggggctgggggcgctgaggcccaggggatgctgggtcgggccctactgctctccagtctcccagacaactgctccttctgggatgcctttcgcccagagggccggcgcagtgtgctacggacgattggggaatacctggaacaagatgaggagcagccaaccccatcaggctttgaacccactgtcaaccccagctctggtataagcaagatggagctgctggcc

SEQ ID NO: 56 - DNA, 2238 bp, Artificial Sequence
  Description of Artificial Sequence Synthetic polynucleotide
atggctctgagcctccagccccagctgctccttctcctgtcgctcctgccgcaggaagtgacttcagcccctactgggcctcagtctttggatgctggtctctcccttctgaagtcattcgtagccactctggaccaagctcctcagcgttccctcagccagtcacggttctctgcgttcctggccaacatttcttcatccttccagcttgggaggatgggggagggaccggtgggagagcccccacctctccagccccctgcacttcgacttcatgatttcctcgtgacactgagaggtagcccagactgggagccaatgctagggcttctgggagatgtgctggcactcctgggacaggaacagactccccgggactttttggtgcaccaggcaggtgtactgggtggacttgtagaggcattgttgggagcgttagttcctggaggcccccctgcccccactcgacccccatgcacccgtgatggcccttctgactgtgtcctggctgctgattggttgccttctctgatgttgttattagagggtacacgctggcaggccctggtgcagttgcagcccagtgtggacccaaccaatgccacaggtcttgatggtagagagccagctcctcactttttacagggtctgctgggcttgcttaccccagcaggagagttgggctctgaggaggctctttggggtggtctgctgcgcacagtgggggcccccctctatgctgccttccaggaggggctactgcgagtcactcattctctgcaagatgaggtcttttctattatgggacagccagagcctgatgccagtgggcagtgccagggaggcaaccttcaacagctgcttttatggggcatgcggaacaacctttcttgggacgcccgagcactgggttttctatctggatcaccacctccaccccctgctctcctgcactgcctgagcagaggtgtgcctctgcccagggcttcccagcctgcggctcacatcagccctcgacagcggcgagccatctctgtggaggccctctgcgagaaccactcaggcccagagccaccctacagcatctccaacttctccatctacttgctctgccagcacatcaagcctgccaccccgcggccccctcctaccaccccacggcctcctcctaccaccccacagccccctcctaccactacacagcccattcctgacactacacagccccctcctgtcaccccaaggcctcctcctaccaccccacaaccccctcctagcacagctgtcatctgccagacagctgtatggtacgcagtctcgtgggcaccaggtgcccgaggttggctccaagcctgccatgatcagtttcctgatcaatttctggatatgatctgcggcaacctctcattttcagccctgtctggccccagtcgtcctttggtaaagcagctctgtgctggcttgctcccaccccccactagctgtccaccaggcctgatccctgtgcccctcaccccagaaatattctggggctgtttcctggagaatgagacactgtgggctgaacggttgtgtgtggaggacagtctgcaggctgtgcccccgaggaaccaggcttgggttcagcatgtgtgtcggggccccaccttggacgccactgattttccaccgtgccgcgttggaccctgtggggaacgctgcccagatgggggcagcttcctgctcatggtctgtgccaatgacactctgtatgaagccttggttcccttctgggcttggctagcaggccaatgcagaattagtcgtggaggaaatgatacttgctttctagaaggcatgctgggccccttgttgccctctctgccccctctgggaccatccccactctgtctggctcctggtccttttctgcttggcatgttatcccagttgccacgctgtcagtcctccgtgccagccctcgcccaccccacgcgcctacattacctcctgcgcctactgaccttccttctgggtccagggactgggggtgccgagacgcaggggatgttaggtcaagccctgctgctctctagtctcccagacaactgttcattctgggatgccttccgcccagagggccggagaagtgtactgaggacagtcggagagtacttgcagcgggaagagccaaccccaccaggcttagactcctccctcagcctcggctctggtatgagcaagatggagcttctgtcc

SEQ ID NO: 57 - DNA, 3201 bp, Homo sapiens
tgctttagtcctgtgctgtgggatctgctccagagggaaaagagtgtttgggccctgcagattctagtgcaggcgtacctgcatatgcccccagaaaacctccagcagctggtgctttcagcagagagggaggctgcacagggcttcctgacactcatgctgcaggggaagctgcaggggaagctgcaggtaccaccatccgaggagcaggccctgggtcgcctgacagccctgctgctccagcggtacccacgcctcacctcccagctcttcattgacctgtcaccactcatccctttcttggctgtctctgacctgatgcgcttcccaccatccctgttagccaacgacagtgtcctggctgccatccgggattacagcccaggaatgaggcctgaacagaaggaggctctggcaaagcgactgctggcccctgaactgtttggggaagtgcctgcctggccccaggagctgctgtgggcagtgctgcccctgctcccccacctccctctggagaactttttgcagctcagccctcaccagatccaggccctggaggatagctggccagcagcaggtctggggccagggcatgcccgccatgtgctgcgcagcctggtaaaccagagtgtccaggatggtgaggagcaggtacgcaggcttgggcccctcgcctgtttcctgagccctgaggagctgcagagcctagtgcccctgagtgatccaacggggccagtagaacgggggctgctggaatgtgcagccaatgggaccctcagcccagaaggacgggtggcatatgaacttctgggtgtgttgcgctcatctggaggagcggtgctgagcccccgggagctgcgggtctgggcccctctcttctctcagctgggcctccgcttccttcaggagctgtcagagccccagcttagagccatgcttcctgtcctgcagggaactagtgttacacctgctcaggctgtcctgctgcttggacggctccttcctaggcacgatctatccctggaggaactctgctccttgcaccttctgctaccaggcctcagcccccagacactccaggccatccctaggcgagtcctggtcggggcttgttcctgcctggcccctgaactgtcacgcctctcagcctgccagaccgcagcactgctgcagacctttcgggttaaagatggtgttaaaaatatgggtacaacaggtgctggtccagctgtgtgtatccctggtcagcctattcccaccacctggccagactgcctgcttcccctgctcccattaaagctgctacaactggattccttggctcttctggcaaatcgaagacgctactgggagctgccctggtctgagcagcaggcacagtttctctggaagaagatgcaagtacccaccaaccttaccctcaggaatctgcaggctctgggcaccctggcaggaggcatgtcctgtgagtttctgcagcagatcaactccatggtagacttccttgaagtggtgcacatgatctatcagctgcccactagagttcgagggagcctgagggcctgtatctgggcagagctacagcggaggatggcaatgccagaaccagaatggacaactgtagggccagaactgaacgggctggatagcaagctactcctggacttaccgatccagttgatggacagactatccaatgaatccattatgttggtggtggagctggtgcaaagagctccagagcagctgctggcactgacccccctccaccaggcagccctggcagagagggcactacaaaacctggctccaaaggagactccagtctcaggggaagtgctggagaccttaggccctttggttggattcctggggacagagagcacacgacagatccccctacagatcctgctgtcccatctcagtcagctgcaaggcttctgcctaggagagacatttgccacagagctgggatggctgctattgcaggagtctgttcttgggaaaccagagttgtggagccaggatgaagtagagcaagctggacgcctagtattcactctgtctactgaggcaatttccttgatccccagggaggccttgggtccagagaccctggagcggcttctagaaaagcagcagagctgggagcagagcagagttggacagctgtgtagggagccacagcttgctgccaagaaagcagccctggtagcaggggtggtgcgaccagctgctgaggatcttccagaacctgtgccaaattgtgcagatgtacgagggacattcccagcagcctggtctgcaacccagattgcagagatggagctctcagactttgaggactgcctgacattatttgcaggagacccaggacttgggcctgaggaactgcgggcagccatgggcaaagcaaaacagttgtggggtcccccccggggatttcgtcctgagcagatcctgcagcttggtaggctcttaataggtctaggagatcgggaactacaggagctgatcctagtggactggggagtgctgagcaccctggggcagatagatggctggagcaccactcagctccgcattgtggtctccagtttcctacggcagagtggtcggcatgtgagccacctggacttcgttcatctgacagcgctgggttatactctctgtggactgcggccagaggagctccagcacatcagcagttgggagttcagccaagcagctctcttcctcggcaccctgcatctccagtgctctgaggaacaactggaggttctggcccacctacttgtactgcctggtgggtttggcccaatcagtaactgggggcctgagatcttcactgaaattggcaccatagcagctgggatcccagacctggctctttcagcactgctgcggggacagatccagggcgttactcctcttgccatttctgtcatccctcctcctaaatttgctgtggtgtttagtcccatccaactatctagtctcaccagtgctcaggctgtggctgtcactcctgagcaaatggcctttctgagtcctgagcagcgacgagcagttgcatgggcccaacatgagggaaaggagagcccagaacagcaaggtcgaagtacagcctggggcctccaggactggtcacgaccttcctggtccctggtattgactatcagcttccttggccacctgcta

SEQ ID NO: 58 - DNA, 3189 bp, Mus musculus
tgcttcagtcctgtactgtgggatctactccagagagagaagagcgtttgggccctgaggaccctggtgaaggcctacctgcgcatgcctccagaagaccttcagcagcttgtgctttcagcagagatggaggctgcacagggcttcctgacgctcatgcttcgttcctgggctaagctgaaggttcaaccatccgaggagcaggccatgggccgcctgacagccttgctgctccagcggtacccacgcctcacctcccaactctttatcgacatgtcaccgctcatccccttcctggctgtccctgacctcatgcgcttcccaccgtcccttttggccaacgacagtgtcctggctgccatcagggatcacagctcaggaatgaagcctgaacagaaggaggccctggcaaaacgactgctggcccctgagctgtttggagaagtgcctgattggccccaggagctgctgtgggcagccctgcctctgcttccccatctgcctctggagagctttctccagctcagccctcaccagatccaggccctggaggatagctggccagtagcagatcttgggccgggacacgcccgacatgtgcttcgtagcctagtaaaccagagcatggaggatggggaggagcaggtgctcaggcttgggtccctcgcctgtttcctgagtcctgaggagctacagagtctggtgcccttgagtgatccaatggggcctgtagaacagggtctgctggaatgtgcggccaatgggaccctcagcccagaaggacgggtggcatatgaacttctgggagtgttgcgttcatctggaggaactgtcttaagcccccgagagctgagggtctgggcacctctctttccccagctgggcctccgcttcctgcaggagctctcagagacccagcttagagccatgcttcctgccctacagggagccagtgtcacacctgcccaggctgttctgttgtttggaaggctccttcctaagcatgatctgtccctggaggaactctgctccctgcaccctctcctgccaggtctcagcccccagacactccaggccatccctaagagagttctggttggtgcttgttcctgcctgggccctgaactgtcaaggctttcagcttgccagattgcagctctgctgcagacctttcgggtaaaagatggtgttaaaaatatgggtgcagcaggtgccggctcagccgtgtgcattcctgggcagcccaccacttggccagactgcctgcttcccctgctcccattaaagctgctacagctggacgctgcagctcttctggcaaaccgaagactctatcggcagctgccttggtctgagcaacaggcacagtttctctggaagaaaatgcaagtgcctaccaacctgagcctgaggaatctgcaggctctgggcaacttggcaggaggcatgacctgcgagtttctgcagcagatcagctcaatggttgactttcttgatgtggtacacatgctctaccagctgcccactggtgttcgagagagcctgcgggcctgtatctggacagagctacagcggaggatgacaatgccagagccagagctgaccaccctagggccagaactgagtgaacttgacacaaagctactcctggacttgccgatccagctgatggacagattgtccaatgattccattatgttggtggtggagatggtccaaggcgctccagagcagctgctggcactgaccccactccaccagacagccttggcagagcgagcacttaaaaacctggctccaaaggagaccccaatctccaaagaagtgctggagacactgggccccttggttggattcctgggaatagagagcacgcgacggatccctttacccattctactgtctcatctcagtcagctgcagggcttctgcctaggagagacatttgccacagagctgggatggctgctgttgcaggagcctgttcttggaaaaccagaattgtggagccaggatgaaatagagcaagctggacgcctagtattcactctgtctgctgaggctatttcctcgatccccagggaggctttgggcccagagacactggagaggcttctgggaaagcatcaaagctgggagcagagcagagtgggccatctgtgtggggagtcacagcttgcccacaagaaagcagctctggtagctgggattgtgcatccagctgctgagggtctccaagagcctgtaccaaactgtgcagacatacggggaaccttcccagcggcctggtctgcgacacaaatctcagagatggaactctcagactttgaagactgcctgtcactatttgctggagatccaggacttggtcctgaggaactacgggcagccatgggcaaggccaagcagttgtggggtccccctcgaggattccgtcctgagcagatcttgcagctgggccgtctcctgataggtctaggagaacgggaactgcaggagcttaccttggtggactggggtgtgctgagcagcctggggcaaatagatggctggagttccatgcagctccgagccgtggtctccagtttcctaaggcagagtggtcggcatgtgagccacctggacttcatttatctgacagcactgggttacacagtctgtggattgcgaccagaggagttacagcacatcagcagttgggagtttagccaagcagctctcttcctgggtagcttgcatctcccgtgctctgaggaacagctggaagttctggcctatctccttgtgttgcctggtggctttggcccagtcagtaactgggggcctgagatcttcactgaaattggcacaatagcagctggcatcccagacctggctctttcagcattactgcggggacagatccaaggcctgactcctcttgccatttctgtcattcctgctcccaagtttgcagtggtcttcaaccccatccagttatctagtctcaccaggggtcaggccgtagctgttactcctgaacagctggcctatctgagtcctgagcagcggcgagcagttgcatgggcccaacacgaagggaaggagatcccagagcagctgggtcgaaactcagcctggggtctctacgactggttccaagcctcctgggccctggcattgcccgtcagcatttttggccacctatta

SEQ ID NO: 59 - protein, 7 aa, Artificial Sequence
  Description of Artificial Sequence Synthetic peptide
TLAVPFK

SEQ ID NO: 60 - protein, 9 aa, Artificial Sequence
  Description of Artificial Sequence Synthetic peptide
ELLSCFSPV

SEQ ID NO: 61 - protein, 9 aa, Artificial Sequence
  Description of Artificial Sequence Synthetic peptide
GSLACFLSP

SEQ ID NO: 62 - protein, 9 aa, Artificial Sequence
  Description of Artificial Sequence Synthetic peptide
ELLACFSPV

SEQ ID NO: 63 - protein, 9 aa, Artificial Sequence
  Description of Artificial Sequence Synthetic peptide
GPLACFLSP